(19) United States
(12) Patent Application Publication
(10) Pub. No.: US 2025/0031566 A1
(43) Pub. Date: Jan. 23, 2025

(54) ORGANIC OPTOELECTRONIC DEVICE AND DISPLAY DEVICE

(71) Applicant: SAMSUNG SDI CO., LTD., Yongin-si (KR)

(72) Inventors: Sunwoong SHIN, Suwon-si (KR); Youngmook Lim, Suwon-si (KR); Jongwoo Won, Suwon-si (KR); Junghoon Kim, Suwon-si (KR); Gibeom Park, Suwon-si (KR); Kyoungrok Kim, Suwon-si (KR); Ho Kuk Jung, Suwon-si (KR); Youngkyoung Jo, Suwon-si (KR); Jihun Shin, Suwon-si (KR); Dalho Huh, Suwon-si (KR); Hyung Sun Kim, Suwon-si (KR)

(21) Appl. No.: 18/743,291

(22) Filed: Jun. 14, 2024

(30) Foreign Application Priority Data
Jun. 15, 2023 (KR) 10-2023-0076850
Jun. 12, 2024 (KR) 10-2024-0076527

(51) Int. Cl. H10K 85/60; C07C 211/61; C07D 307/91; C07D 333/76; C07F 7/08; C09K 11/06; H10K 50/15; H10K 85/40

(52) U.S. Cl. CPC H10K 85/633; C07C 211/61; C07D 307/91; C07D 333/76; C07F 7/081; C09K 11/06; H10K 85/40; H10K 85/636; C07C 2603/18; C07C 2603/42; C09K 2211/1003; C09K 2211/1018; H10K 50/156; H10K 85/622; H10K 85/626; H10K 85/6574; H10K 85/6576

(57) ABSTRACT

An organic optoelectronic device, including an anode and a cathode facing each other; a light emitting layer between the anode and the cathode; a hole transport layer between the anode and the light emitting layer; and a hole transport auxiliary layer between the hole transport layer and the light emitting layer, wherein the hole transport auxiliary layer includes a first hole transport auxiliary layer adjacent to the hole transport layer and a second hole transport auxiliary layer adjacent to the light emitting layer, the first hole transport auxiliary layer includes a first compound represented by Chemical Formula 1, and the second hole transport auxiliary layer includes a second compound represented by Chemical Formula 2:

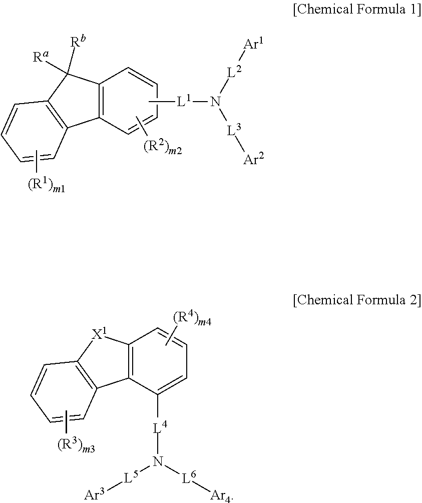

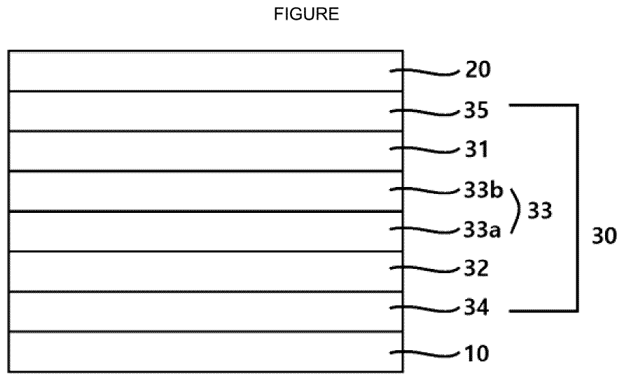

### CROSS-REFERENCE TO RELATED APPLICATION

This application claims priority to and the benefit of Korean Patent Applications No. 10-2023-0076850 filed in the Korean Intellectual Property Office on Jun. 15, 2023, and No. 10-2024-0076527 filed in the Korean Intellectual Property Office on Jun. 12, 2024, the entire contents of which are incorporated herein by reference.

### BACKGROUND

### 1. Field

Embodiments relate to an organic optoelectronic device and a display device.

### 2. Description of the Related Art

An organic optoelectronic device (e.g., organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric device, an organic light emitting diode, an organic solar cell, and an organic photo conductor drum.

Among them, the organic light emitting diode (OLED) has recently drawn attention due to an increase in demand for flat panel displays. The organic light emitting diode converts electrical energy into light, and the performance of organic light emitting diode is greatly influenced by the organic materials disposed between electrodes.

### SUMMARY

Embodiments are directed to an organic optoelectronic device, including an anode and a cathode facing each other; a light emitting layer between the anode and the cathode; a hole transport layer between the anode and the light emitting layer; and a hole transport auxiliary layer between the hole transport layer and the light emitting layer, wherein the hole transport auxiliary layer includes a first hole transport auxiliary layer adjacent to the hole transport layer and a second hole transport auxiliary layer adjacent to the light emitting layer, the first hole transport auxiliary layer includes a first compound represented by Chemical Formula 1, and the second hole transport auxiliary layer includes a second compound represented by Chemical Formula 2,

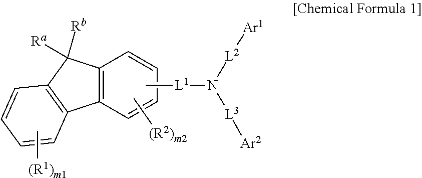

in Chemical Formula 1, R^{a} and R^{b} are each independently a substituted or unsubstituted C1 to C30 alkyl group or a substituted or unsubstituted C6 to C30 aryl group, L¹ to L³ are each independently a single bond, a substituted or unsubstituted C6 to C20 arylene group, or a substituted or unsubstituted C2 to C30 heterocyclic group, R¹ and R² are each independently hydrogen, deuterium, a cyano group, a substituted or unsubstituted C1 to C30 alkyl group, a substituted or unsubstituted C6 to C30 aryl group, or a substituted or unsubstituted C2 to C30 heterocyclic group, Ar¹ and Ar² are each independently a substituted or unsubstituted C3 to C40 cycloalkyl group or a substituted or unsubstituted C6 to C20 aryl group, m1 is an integer of 1 to 4, and m2 is an integer of 1 to 3;

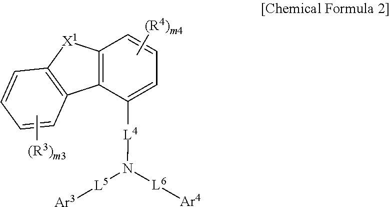

in Chemical Formula 2, X¹ is O or S, L⁴ to L⁶ are each independently a single bond, a substituted or unsubstituted C6 to C20 arylene group, or a substituted or unsubstituted C2 to C30 heterocyclic group, R³ and R⁴ are each independently hydrogen, deuterium, a cyano group, a substituted or unsubstituted C1 to C30 alkyl group, a substituted or unsubstituted C6 to C30 aryl group, or a substituted or unsubstituted C2 to C30 heterocyclic group, Ar³ and Ar⁴ are each independently a substituted or unsubstituted C6 to C20 aryl group or a substituted or unsubstituted C2 to C30 heterocyclic group, at least one of Ar³ and Ar⁴ is a substituted or unsubstituted 9-fluorenyl group, m3 is an integer of 1 to 4, and m4 is an integer of 1 to 3.

The embodiments may be realized by providing a display device comprising the organic optoelectronic device according to an embodiment.

### BRIEF DESCRIPTION OF THE DRAWING

Features will become apparent to those of skill in the art by describing in detail exemplary embodiments with reference to the attached drawing in which:

the FIGURE is a cross-sectional view illustrating an organic light emitting diode according to some embodiments.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. Like reference numerals refer to like elements throughout. As used herein, the term “or” is not necessarily an exclusive term, e.g., “A or B” would include A, B, or A and B.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C40 cycloalkyl group, a C3 to C30 hetero cycloalkyl group, a C6 to C30 aryl group, a C2 to C30 heteroaryl group, a C1 to C20 alkoxy group, a C1 to C10 trifluoroalkyl group, a cyano group, or a combination thereof.

In an implementation, “substituted” may refer to replacement of at least one hydrogen of a substituent or a compound by deuterium, a C1 to C30 alkyl group, a C1 to C10 alkylsilyl group, a C6 to C30 arylsilyl group, a C3 to C40 cycloalkyl group, a C3 to C30 hetero cycloalkyl group, a C6 to C30 aryl group, a C2 to C30 heteroaryl group, or a cyano group. In addition, in an implementation, “substituted” may refer to replacement of at least one hydrogen of a substituent or a compound by deuterium, a C1 to C20 alkyl group, a C3 to C30 cycloalkyl group, a C6 to C30 aryl group, or a cyano group. In addition, in an implementation, “substituted” may refer to replacement of at least one hydrogen of a substituent or a compound by deuterium, a C1 to C5 alkyl group, a C3 to C20 cycloalkyl group, a C6 to C18 aryl group, or a cyano group. In addition, in an implementation, “substituted” may refer to replacement of at least one hydrogen of a substituent or a compound by deuterium, a cyano group, a methyl group, an ethyl group, a propyl group, an iso-propyl group, a butyl group, a tert-butyl group, a cyclohexyl group, bicycloheptyl group, adamantly group, a phenyl group, a biphenyl group, a terphenyl group, or a naphthyl group.

As used herein, “unsubstituted” refers to non-replacement of a hydrogen atom by another substituent and remaining of the hydrogen atom.

As used herein, “hydrogen (—H)” may include “deuterium substitution (-D)” or “tritium substitution (-T). For example, any hydrogen in any compound described herein may be protium, deuterium, or tritium (e.g., based on natural or artificial substitution).

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quarterphenyl group, and the like, and a group in which two or more hydrocarbon aromatic moieties are fused directly or indirectly to provide a non-aromatic fused ring, for example a fluorenyl group, and the like.

The aryl group may include a monocyclic, polycyclic or fused ring polycyclic (i.e., rings sharing adjacent pairs of carbon atoms) functional group.

As used herein, “heterocyclic group” has a generic concept of a heteroaryl group, and may include at least one heteroatom, e.g., N, O, S, P, and Si instead of carbon (C) in a cyclic compound such as an aryl group, a cycloalkyl group, a fused ring thereof, or a combination thereof. In an implementation, the heterocyclic group may be a fused ring and the entire ring or each ring of the heterocyclic group may include one or more heteroatoms.

For example, “heteroaryl group” refers to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

For example, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof.

For example, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzothiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof.

As used herein, hole characteristics refers to an ability to donate an electron to form a hole when an electric field is applied and that a hole formed in the anode may be easily injected into the light emitting layer and transported in the light emitting layer due to conductive characteristics according to the highest occupied molecular orbital (HOMO) level.

In addition, electron characteristics refers to an ability to accept an electron when an electric field is applied and that electron formed in the cathode may be easily injected into the light emitting layer and transported in the light emitting layer due to conductive characteristics according to the lowest unoccupied molecular orbital (LUMO) level.

The organic optoelectronic device may be any device to convert electrical energy into photoenergy and vice versa, and may be, e.g., an organic photoelectric device, an organic light emitting diode, an organic solar cell, and an organic photo conductor drum, and the like.

Herein, an organic light emitting diode, which is an example of an organic optoelectronic device, is exemplarily described.

The FIGURE is a cross-sectional view illustrating an organic light emitting diode according to some embodiments. Hereinafter, an organic light emitting diode according to some embodiments will be described with reference to the FIGURE.

Referring to the FIGURE, an organic light emitting diode according to some embodiments may include, e.g., an anode 10 and a cathode 20 facing each other, and an organic layer 30 between the anode 10 and the cathode 20.

The anode 10 may be made of a conductor having a large work function to help hole injection, and may be, e.g., a metal, a metal oxide or a conductive polymer. The anode 10 may be, e.g., a metal, e.g., nickel, platinum, vanadium, chromium, copper, zinc, gold, or the like or an alloy thereof; a metal oxide, e.g., zinc oxide, indium oxide, indium tin oxide (ITO), indium zinc oxide (IZO), or the like; a combination of a metal and an oxide such as ZnO and Al or SnO₂ and Sb; a conductive polymer, e.g., poly(3-methylthiophene), poly(3,4-(ethylene-1,2-dioxy)thiophene) (PEDOT), polypyrrole, or polyaniline.

The cathode 20 may be made of a conductor having a small work function to help electron injection, and may be, e.g., a metal, a metal oxide or a conductive polymer. The cathode 20 may be, e.g., a metal such as magnesium, calcium, sodium, potassium, titanium, indium, yttrium, lithium, gadolinium, aluminum silver, tin, lead, cesium, barium, or the like or an alloy thereof; a multi-layer structured material, e.g., LiF/Al, LiO₂/Al, LiF/Ca, or BaF₂/Ca.

The organic layer 30 may include a light emitting layer 31, a hole transport layer 32, and a hole transport auxiliary layer 33 between the hole transport layer 32 and the light emitting layer 31. The light emitting layer 31 may include at least two types of host and dopant, and the host may be, e.g., a phosphorescent host. The dopant may be, e.g., a phosphorescent dopant, e.g., a red, green or blue phosphorescent dopant, e.g., a red or green phosphorescent dopant.

The dopant may be a material mixed with the host in a small amount to cause light emission and generally a material such as a metal complex that emits light by multiple excitation into a triplet or more. The dopant may be, e.g., an inorganic, organic, or organic/inorganic compound, and one or more types thereof may be used.

In an implementation, the dopant may be a phosphorescent dopant and examples of the phosphorescent dopant may be an organometal compound including Ir, Pt, Os, Ti, Zr, Hf, Eu, Tb, Tm, Fe, Co, Ni, Ru, Rh, Pd, or a combination thereof. The phosphorescent dopant may be or include, e.g., a compound represented by Chemical Formula Z.

L⁷MX³ [Chemical Formula Z]

In Chemical Formula Z, M may be a metal, and L⁷ and X³ may each independently be a ligand forming a complex compound with M.

M may be, e.g., Ir, Pt, Os, Ti, Zr, Hf, Eu, Tb, Tm, Fe, Co, Ni, Ru, Rh, Pd or a combination thereof and L⁷ and X³ may be, e.g., a bidentate ligand.

In an implementation, the ligand represented by L⁷ and X³ may be a ligand of Group A.

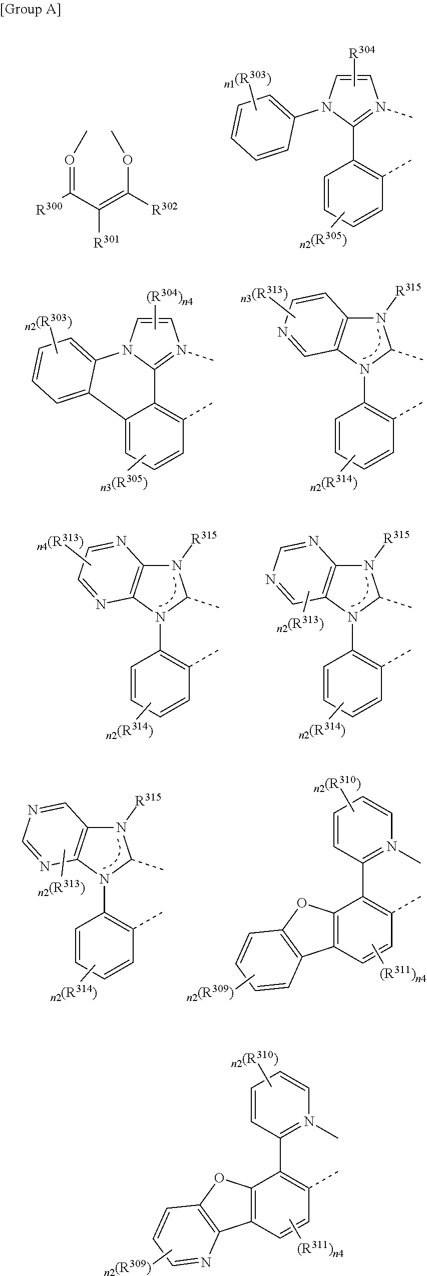

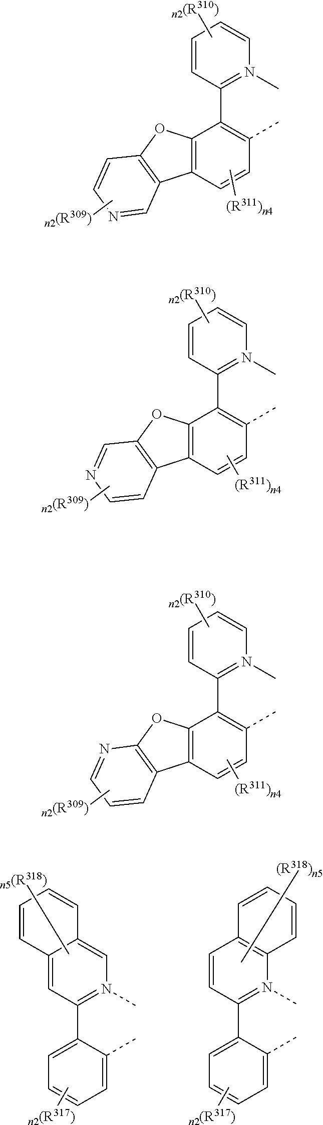

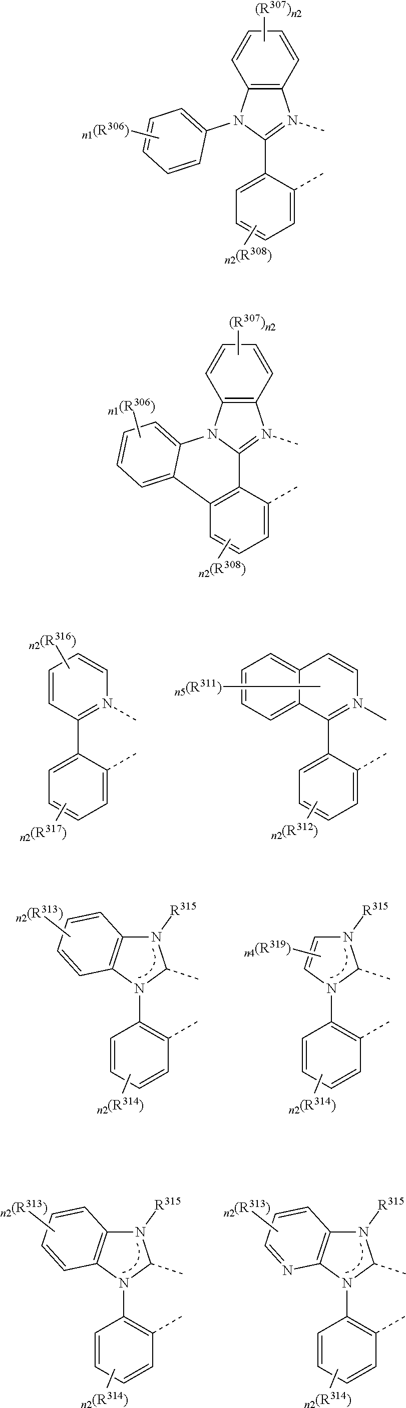

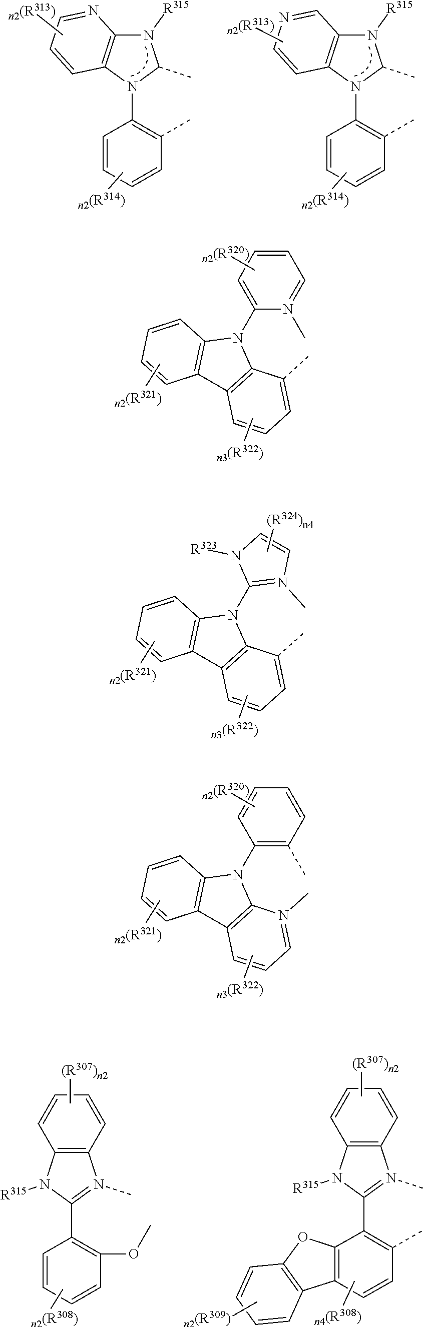

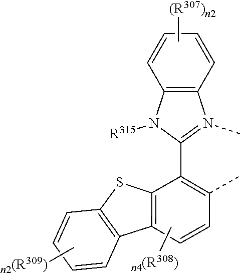

In Group A, R³⁰⁰ to R³⁰² may each independently be, e.g., hydrogen, deuterium, a C1 to C30 alkyl group that is substituted or unsubstituted with a halogen, a C6 to C30 aryl group that is substituted or unsubstituted with a C1 to C30 alkyl, or a halogen.

R³⁰³ to R³²⁴ may each independently be, e.g., hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and C6 to C30 aryl group, or a triarylsilyl group having a substituted or unsubstituted C6 to C30 aryl group.

n1 may be, e.g., an integer of 1 to 5.

n2 may be, e.g., an integer of 1 to 4.

n3 may be, e.g., an integer of 1 to 3.

n4 may be, e.g., an integer of 1 or 2.

n5 may be, e.g., an integer of 1 to 6.

In an implementation, n1 may be 2, 3, 4, or 5 and each substituent may be the same or different from each other.

In an implementation, n2 may be 2, 3, or 4 and each substituent may be the same or different from each other.

In an implementation, n3 may be 2 or 3 and each substituent may be the same or different from each other.

In an implementation, n4 may be 2 and each substituent may be the same or different from each other.

In an implementation, n5 may be 2, 3, 4, 5, or 6 and each substituent may be the same or different from each other.

In an implementation, the dopant may be represented by Chemical Formula V.

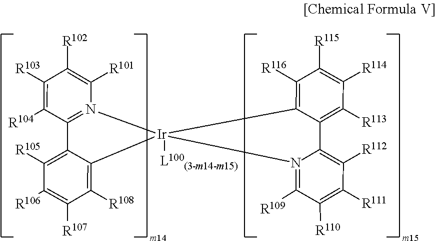

In Chemical Formula V, R¹⁰¹ to R¹¹⁶ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a substituted or unsubstituted C1 to C6 alkyl group.

At least one of R¹⁰¹ to R¹¹⁶ may be a functional group represented by Chemical Formula V-1.

L¹⁰⁰ may be a bidentate ligand of a monovalent anion, and may be a ligand that coordinates to iridium through a lone pair of carbons or heteroatoms.

m14 and m15 may each independently be, e.g., an integer of 0 to 3 and m14+m15 may be, e.g., an integer of 1 to 3.

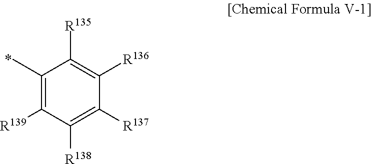

In Chemical Formula V-1, R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

* indicates a portion linked to a carbon atom.

As an example, a dopant represented by Chemical Formula Z-1 may be included.

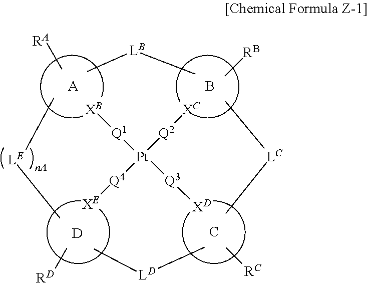

In Chemical Formula Z-1, rings A, B, C, and D may each independently be, e.g., a 5-membered or 6-membered carbocyclic or heterocyclic ring.

R^{A}, R^{B}, R^{C}, and R^{D} may each independently be mono-, di-, tri-, or tetra-substitution, or unsubstitution.

L^{B}, L^{C}, and L^{D} may each independently be, e.g., a direct bond, BR, NR, PR, O, S, Se, C═O, S═O, SO₂, CRR′, SiRR′, GeRR′, or a combination thereof;

In an implementation, nA may be 1, L^{E} may be, e.g., a direct bond, BR, NR, PR, O, S, Se, C═O, S═O, SO₂, CRR′, SiRR′, GeRR′, or a combination thereof. In an implementation, nA may be 0 and L^{E} does not exist.

R^{A}, R^{B}, R^{C}, R^{D}, R, and R′ may each independently be, e.g., hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof. In an implementation, any adjacent ones of R^{A}, R^{B}, R^{C}, R^{D}, R, and R′ may be separate or may be linked to each other to provide a ring. X^{B}, X^{C}, X^{D}, and X^{E} may each independently be, e.g., carbon and nitrogen; and Q¹, Q², Q³, and Q⁴ may each independently be, e.g., oxygen or a direct bond.

The dopant according to some embodiments may be a platinum complex, and may be, e.g., represented by Chemical Formula VI.

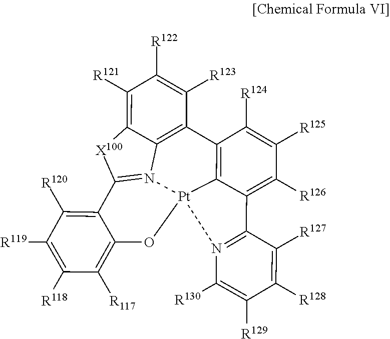

In Chemical Formula VI, X¹⁰⁰ may be, e.g., O, S, or NR¹³¹.

R¹¹⁷ to R¹³¹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a substituted or unsubstituted C1 to C6 alkyl group.

In an implementation, at least one of R¹¹⁷ to R¹³¹ may be —SiR¹³²R¹³³R¹³⁴ or a tert-butyl group.

R¹³² to R¹³⁴ may each independently be, e.g., a substituted or unsubstituted C1 to C6 alkyl group.

The composition further including a dopant may be, e.g., a green light emitting composition.

The hole transport layer 32 may be a layer to facilitate hole transfer from the anode 10 to the light emitting layer 31 and to block electrons, and may be, e.g., an amine compound.

The amine compound may include, e.g., at least one aryl group or heteroaryl group. The amine compound may be represented, e.g., by Chemical Formula a or Chemical Formula b.

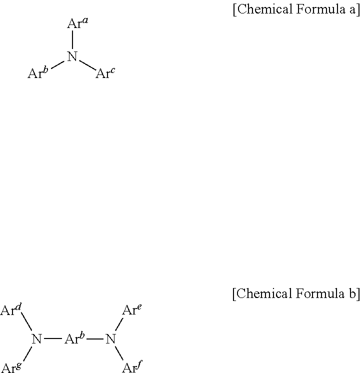

In Chemical Formulas a or b, Ar^{a} to Ar^{g} may each independently be, e.g., hydrogen, deuterium, a substituted or unsubstituted C1 to C20 alkyl group, a substituted or unsubstituted C6 to C30 aryl group, a substituted or unsubstituted C2 to C30 heteroaryl group, or a combination thereof.

At least one of Ar^{a} to Ar^{c} and at least one of Ar^{d} to Ar^{g} may be, e.g., a substituted or unsubstituted C6 to C30 aryl group, a substituted or unsubstituted C2 to C30 heteroaryl group, or a combination thereof.

Ar^{h} may be, e.g., a single bond, a substituted or unsubstituted C1 to C20 alkylene group, a substituted or unsubstituted C6 to C30 arylene group, a substituted or unsubstituted C2 to C30 heteroarylene group or a combination thereof.

The organic light emitting diode may further include a hole injection layer 34 in addition to the light emitting layer.

The hole injection layer 34 may be a layer for facilitating hole injection from the anode 10 to the light emitting layer 31 and blocking electrons, and may be between the anode 10 and the hole transport layer 32.

In an implementation, at least one of the hole transport layer 32 and the hole injection layer 34 may include a compound listed in Group B, which may be different from the materials of the first hole transport auxiliary layer and the second hole transport auxiliary layer described later.

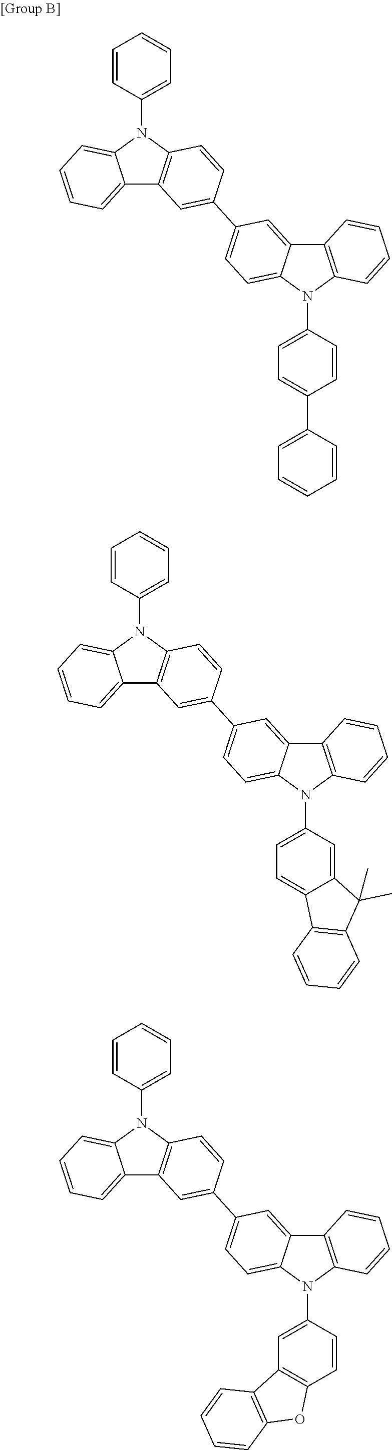 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

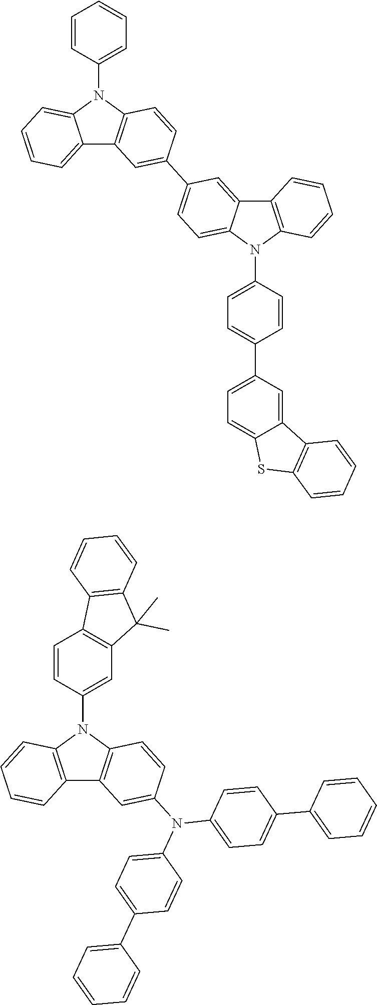 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; CC1(C)c2ccccc2-c2ccc(-n3c4ccccc4c4cc(N(c5ccc(-c6ccccc6)cc5)c5ccc(-c6ccccc6)cc5)ccc43)cc21]

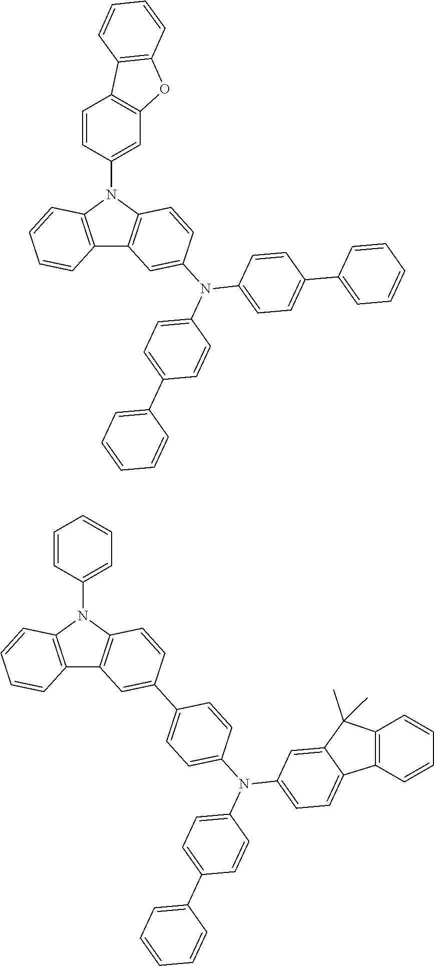 [SMILES: c1ccc(-c2ccc(N(c3ccc(-c4ccccc4)cc3)c3ccc4c(c3)c3ccccc3n4-c3ccc4c(c3)oc3ccccc34)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21]

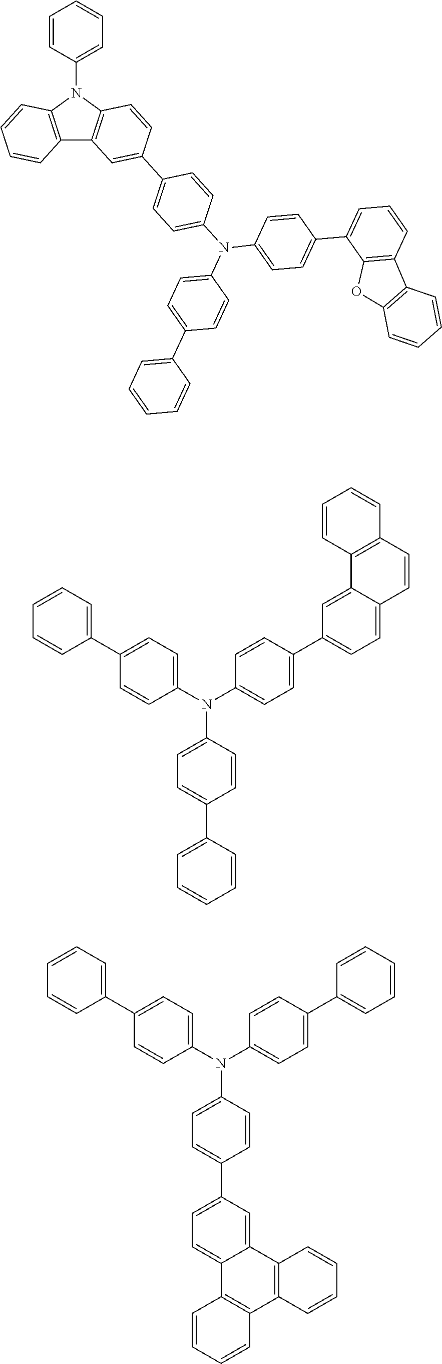 [SMILES: c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1 ; c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1]

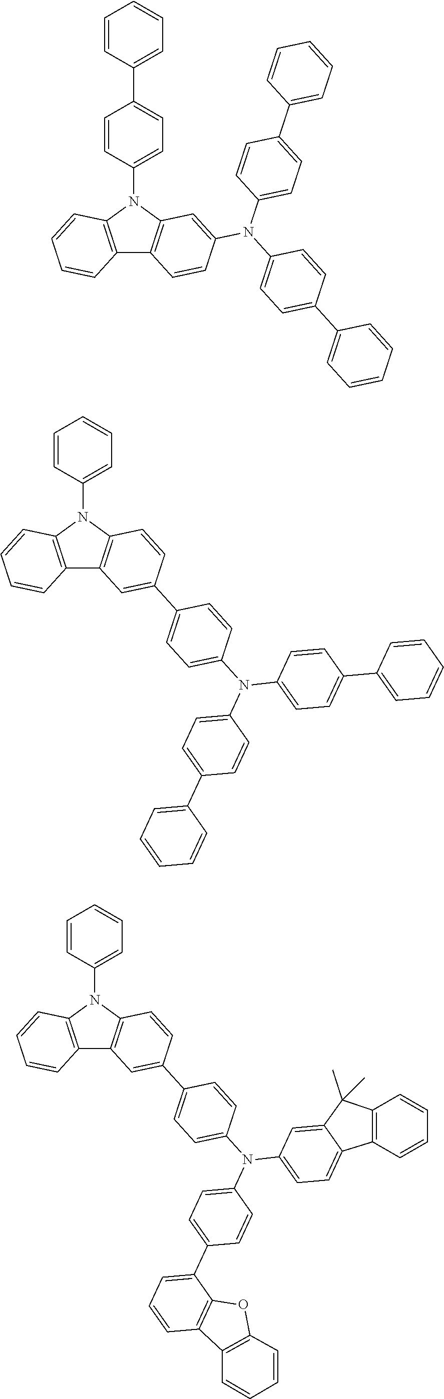 [SMILES: c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21]

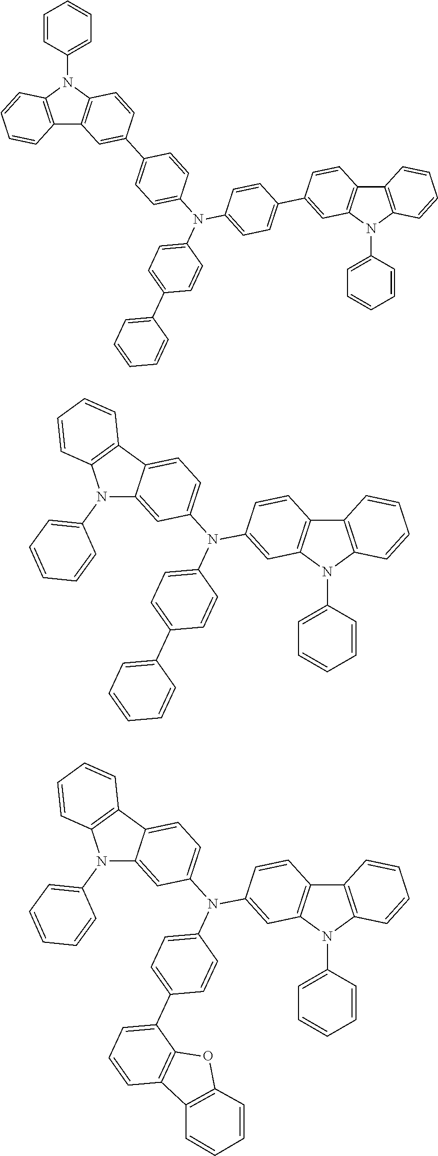 [SMILES: c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1 ; c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1]

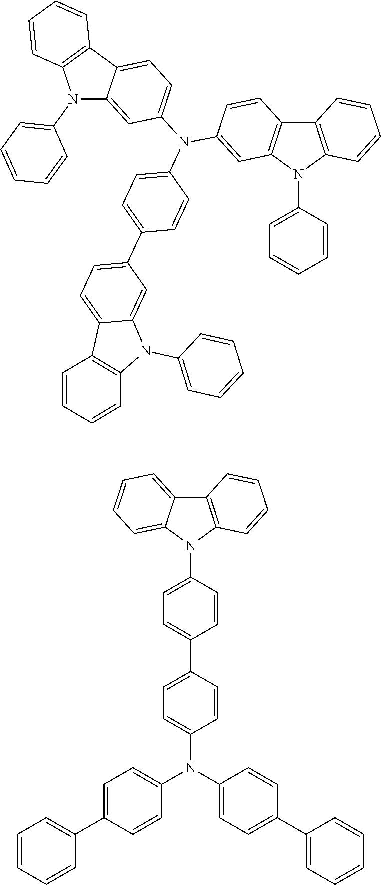 [SMILES: c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1 ; c1ccc(-c2ccc(N(c3ccc(-c4ccccc4)cc3)c3ccc(-c4ccc(-n5c6ccccc6c6ccccc65)cc4)cc3)cc2)cc1]

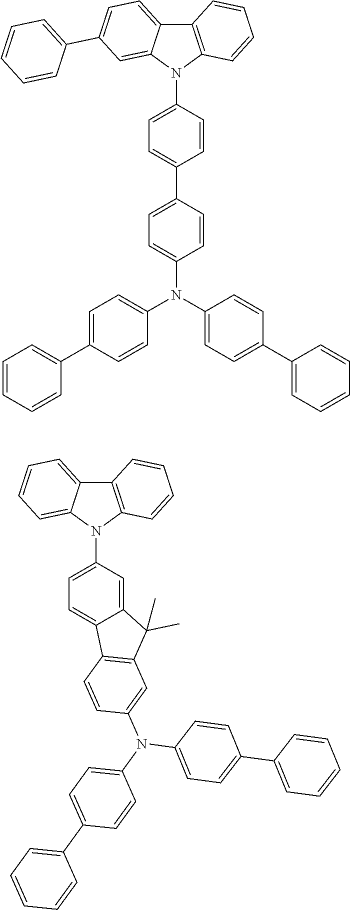 [SMILES: c1ccc(-c2ccc(N(c3ccc(-c4ccccc4)cc3)c3ccc(-c4ccc(-n5c6ccccc6c6ccc(-c7ccccc7)cc65)cc4)cc3)cc2)cc1 ; CC1(C)c2cc(N(c3ccc(-c4ccccc4)cc3)c3ccc(-c4ccccc4)cc3)ccc2-c2ccc(-n3c4ccccc4c4ccccc43)cc21]

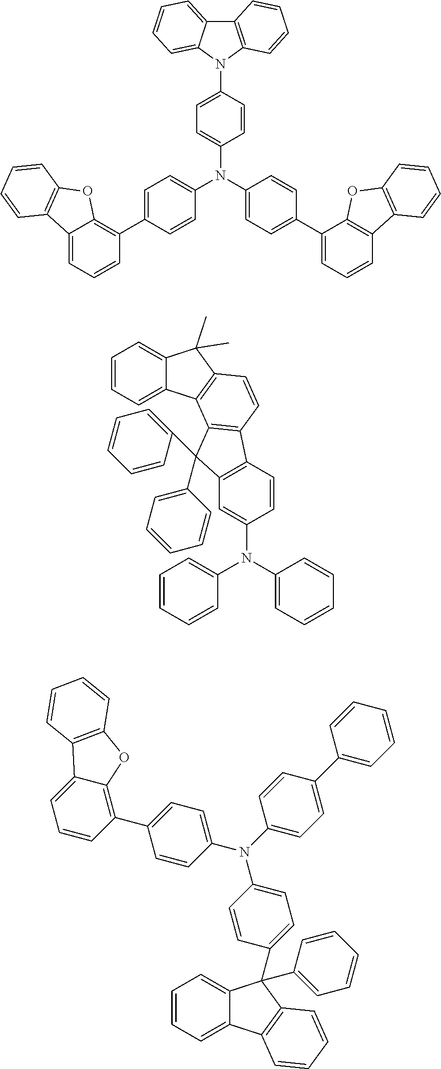 [SMILES: c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; CC1(C)c2ccccc2-c2c1ccc1c2C(c2ccccc2)(c2ccccc2)c2cc(N(c3ccccc3)c3ccccc3)ccc2-1 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1]

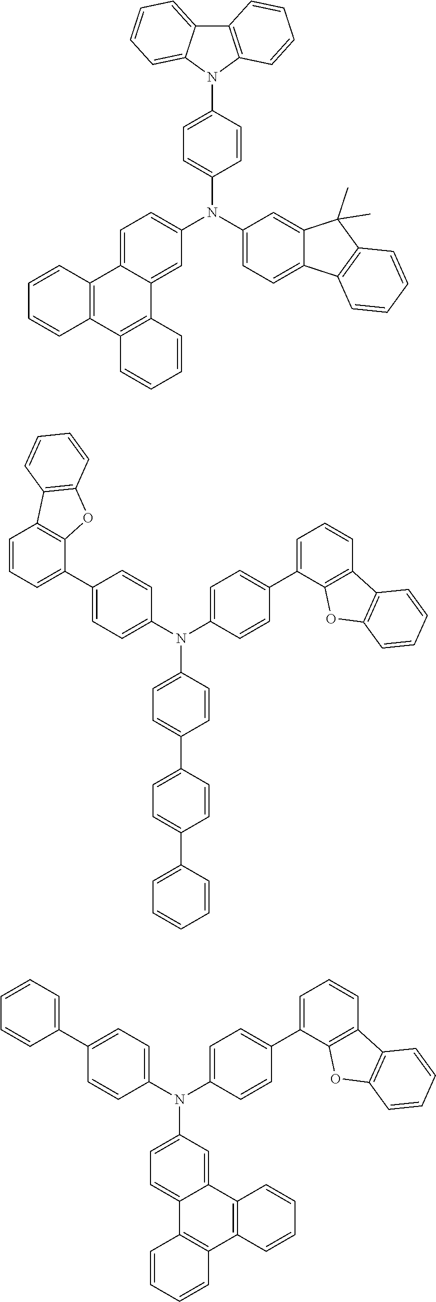 [SMILES: CC1(C)c2ccccc2-c2ccc(N(c3ccc(-n4c5ccccc5c5ccccc54)cc3)c3ccc4c5ccccc5c5ccccc5c4c3)cc21 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1]

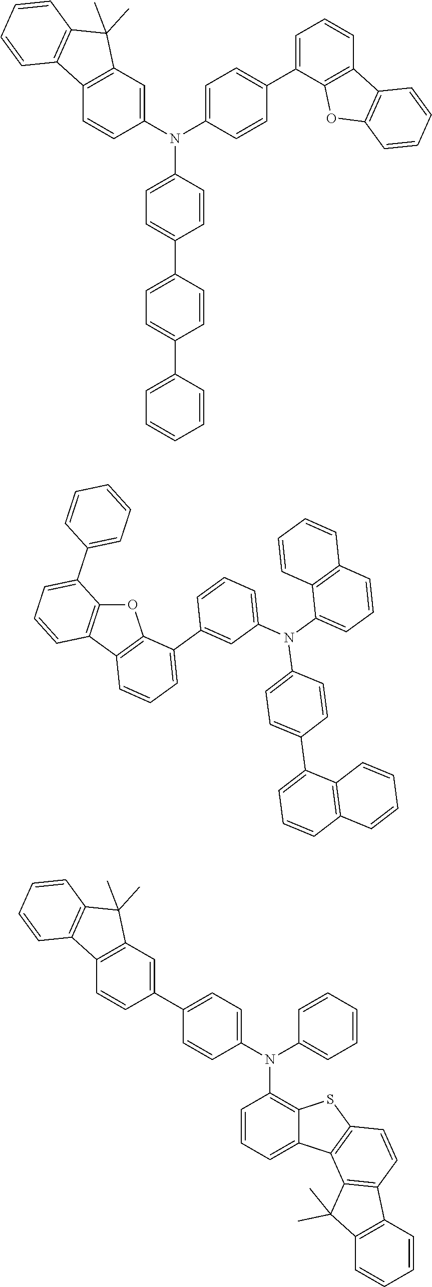 [SMILES: CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21]

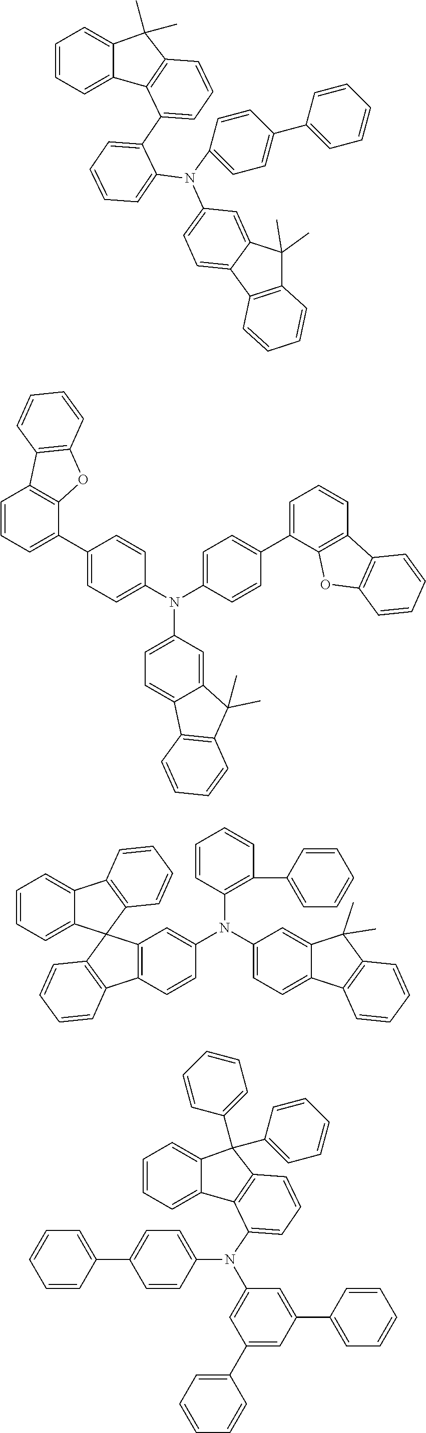

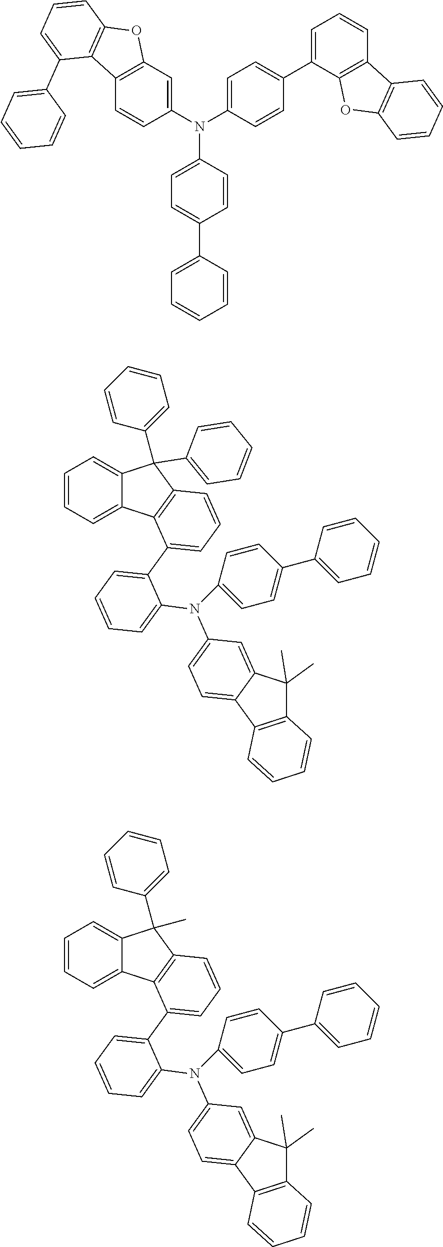 [SMILES: c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21]

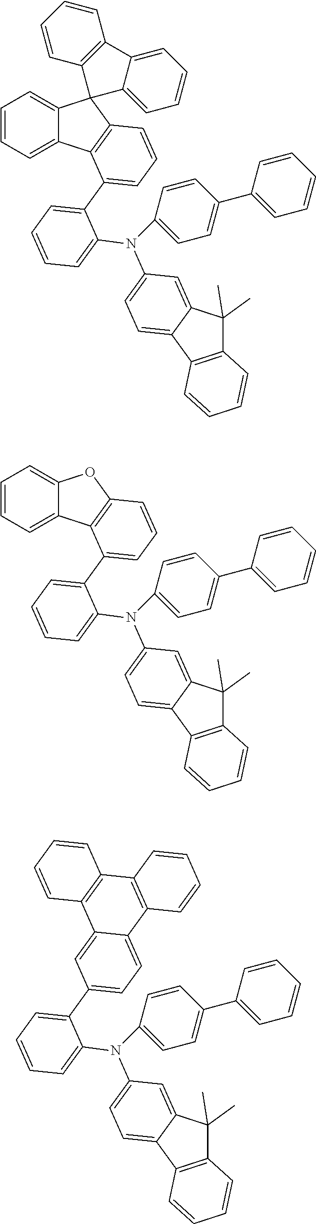 [SMILES: CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21 ; CC1(C)c2ccccc2-c2ccc(N(c3ccc(-c4ccccc4)cc3)c3ccccc3-c3ccc4c5ccccc5c5ccccc5c4c3)cc21]

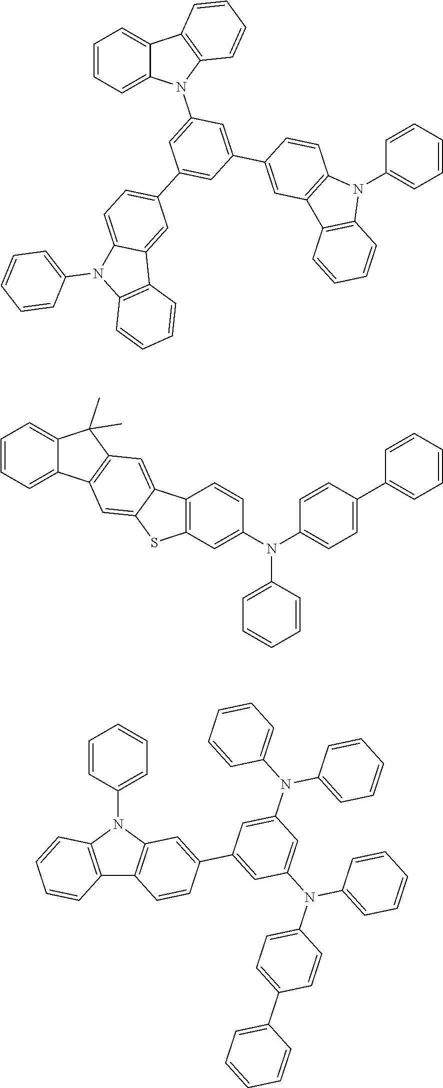 [SMILES: c1ccc(-n2c3ccccc3c3cc(-c4cc(-c5ccc6c(c5)c5ccccc5n6-c5ccccc5)cc(-n5c6ccccc6c6ccccc65)c4)ccc32)cc1 ; CC1(C)c2ccccc2-c2cc3sc4cc(N(c5ccccc5)c5ccc(-c6ccccc6)cc5)ccc4c3cc21 ; c1ccc(-c2ccc(N(c3ccccc3)c3cc(-c4ccc5c6ccccc6n(-c6ccccc6)c5c4)cc(N(c4ccccc4)c4ccccc4)c3)cc2)cc1]

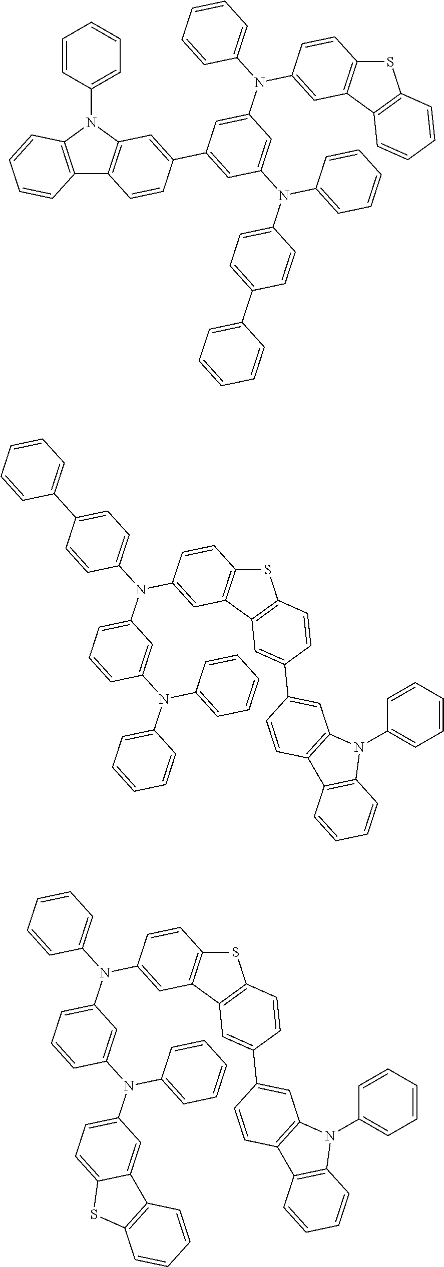 [SMILES: c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1 ; c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1]

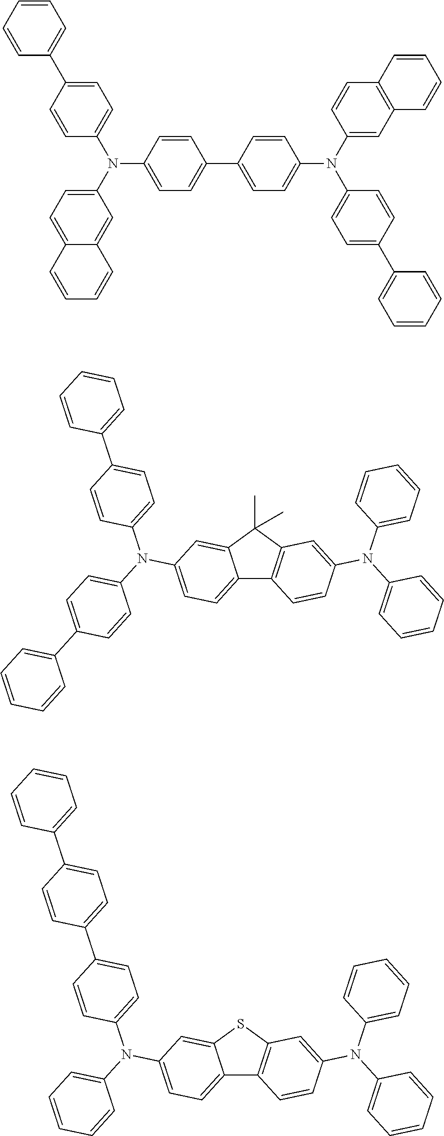 [SMILES: c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1 ; CC1(C)c2cc(N(c3ccccc3)c3ccccc3)ccc2-c2ccc(N(c3ccc(-c4ccccc4)cc3)c3ccc(-c4ccccc4)cc3)cc21 ; c1ccc(-c2ccc(-c3ccc(N(c4ccccc4)c4ccc5c(c4)sc4cc(N(c6ccccc6)c6ccccc6)ccc45)cc3)cc2)cc1]

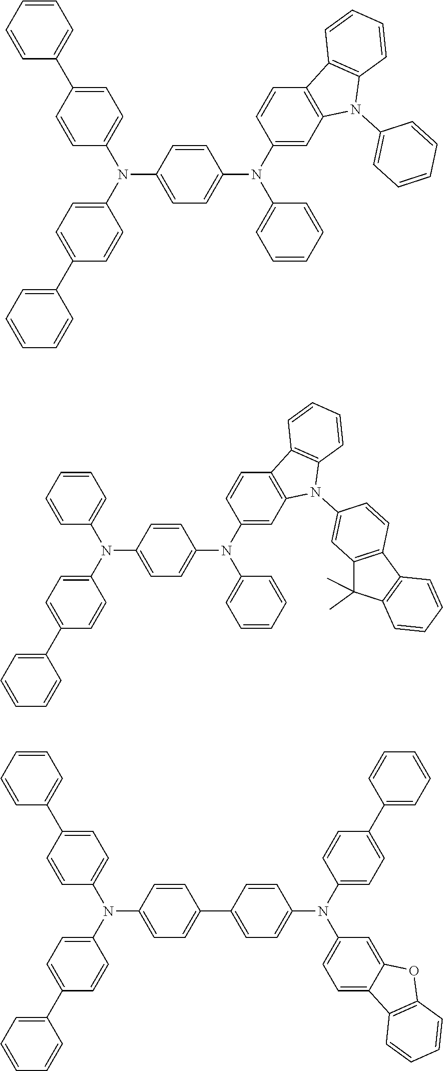 [SMILES: c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1 ; CC1(C)c2ccccc2-c2ccc(-n3c4ccccc4c4ccc(N(c5ccccc5)c5ccc(N(c6ccccc6)c6ccc(-c7ccccc7)cc6)cc5)cc43)cc21 ; c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1]

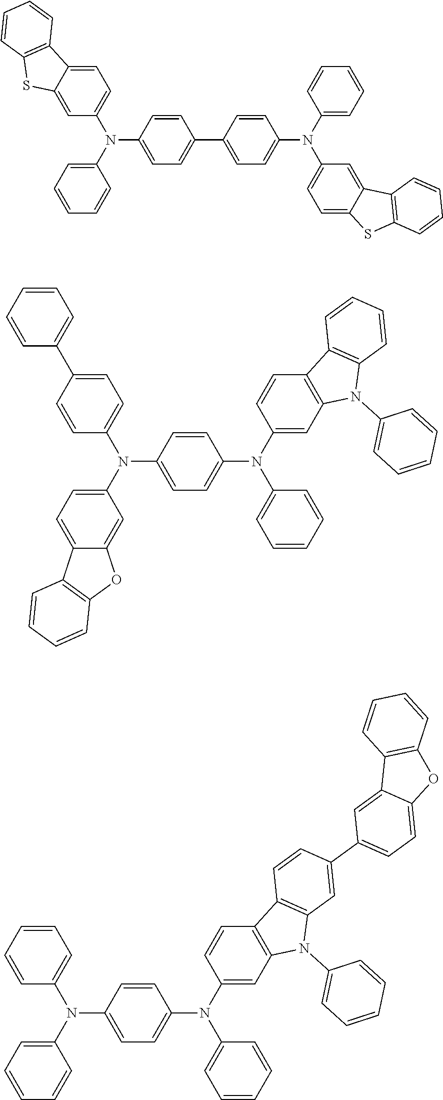 [SMILES: c1ccc(N(c2ccc(-c3ccc(N(c4ccccc4)c4ccc5sc6ccccc6c5c4)cc3)cc2)c2ccc3c(c2)sc2ccccc23)cc1 ; c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1 ; c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1]

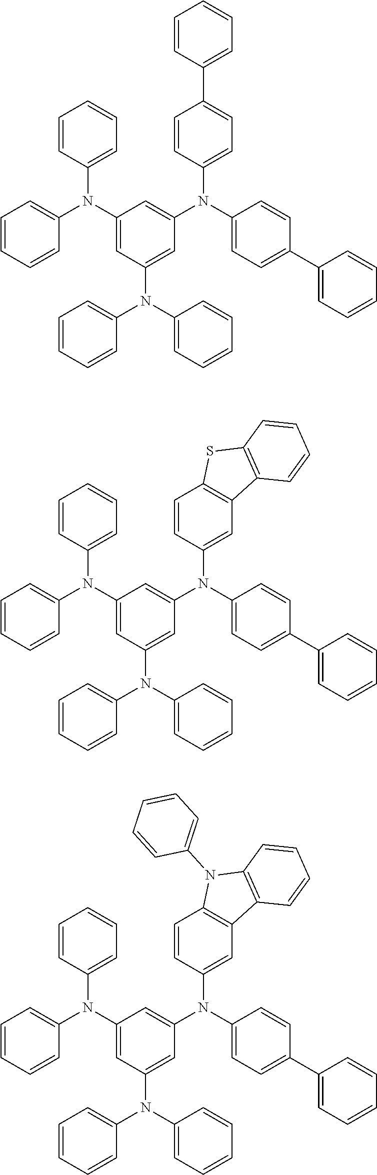 [SMILES: c1ccc(-c2ccc(N(c3ccc(-c4ccccc4)cc3)c3cc(N(c4ccccc4)c4ccccc4)cc(N(c4ccccc4)c4ccccc4)c3)cc2)cc1 ; c1ccc(-c2ccc(N(c3cc(N(c4ccccc4)c4ccccc4)cc(N(c4ccccc4)c4ccccc4)c3)c3ccc4sc5ccccc5c4c3)cc2)cc1 ; c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1]

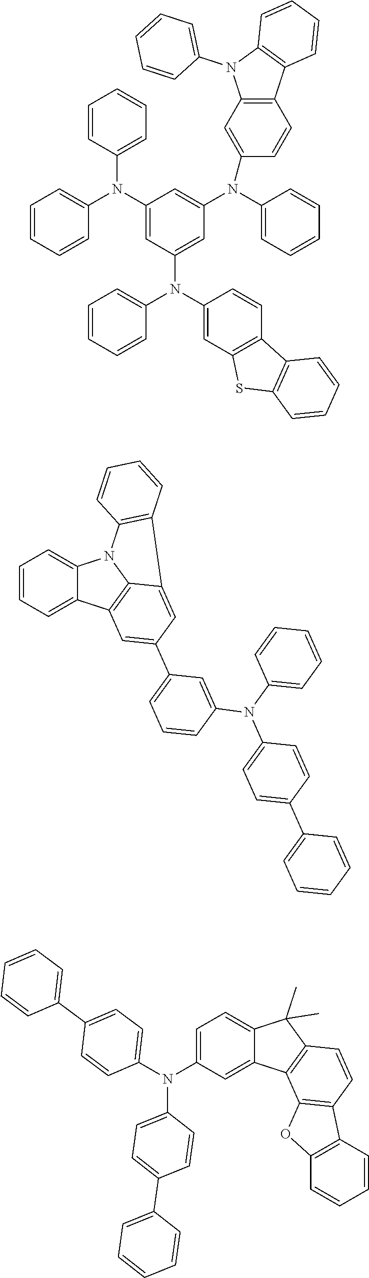 [SMILES: c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1 ; CC1(C)c2ccc(N(c3ccc(-c4ccccc4)cc3)c3ccc(-c4ccccc4)cc3)cc2-c2c1ccc1c2oc2ccccc21]

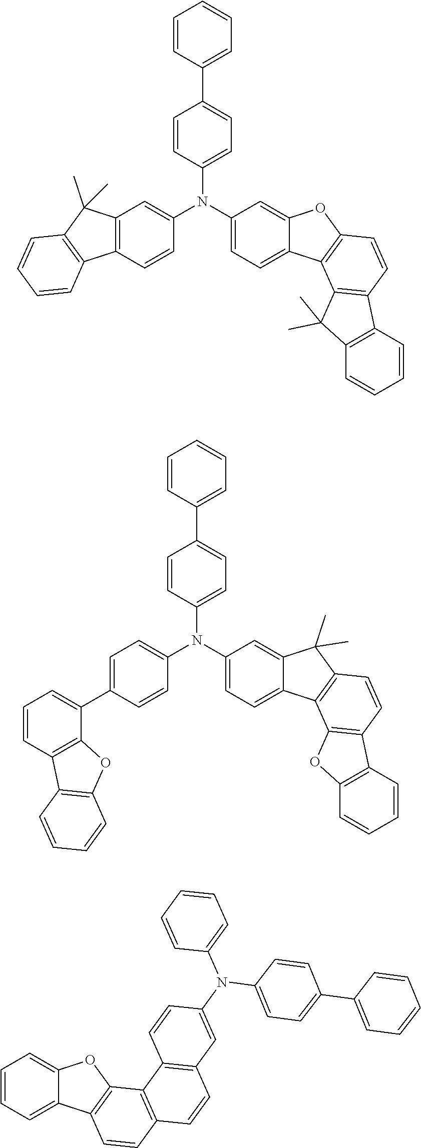 [SMILES: CC1(C)c2ccccc2-c2ccc(N(c3ccc(-c4ccccc4)cc3)c3ccc4c(c3)oc3ccc5c(c34)C(C)(C)c3ccccc3-5)cc21 ; CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1]

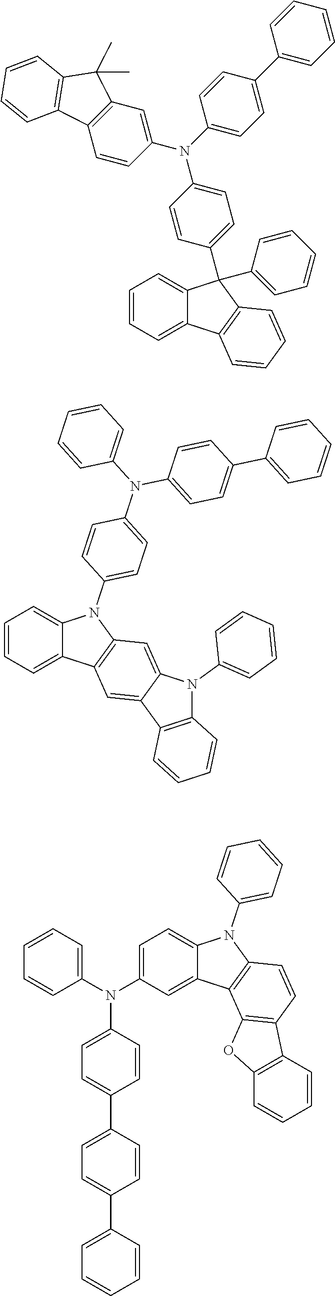 [SMILES: CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1 ; c1ccc(-c2ccc(-c3ccc(N(c4ccccc4)c4ccc5c(c4)c4c6oc7ccccc7c6ccc4n5-c4ccccc4)cc3)cc2)cc1]

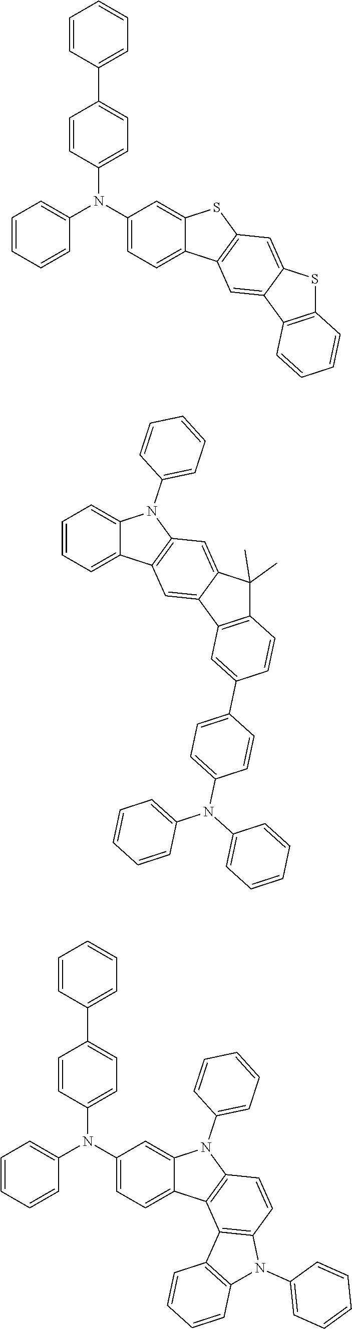 [SMILES: c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1]

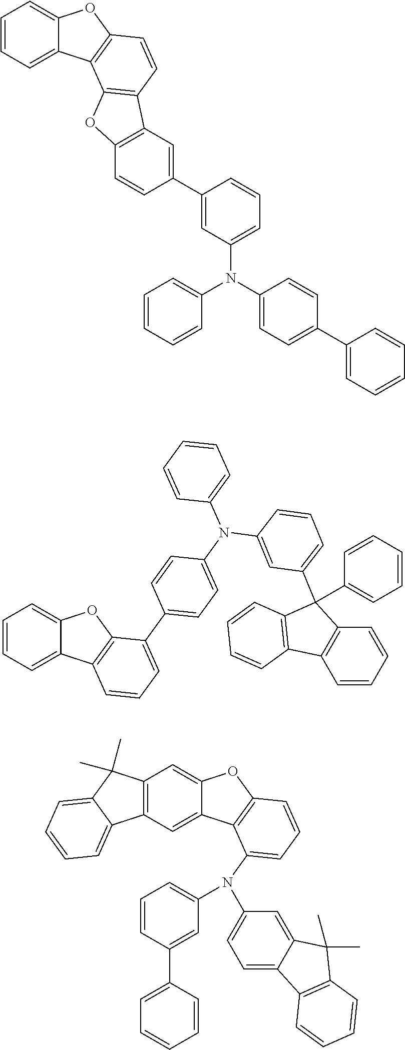

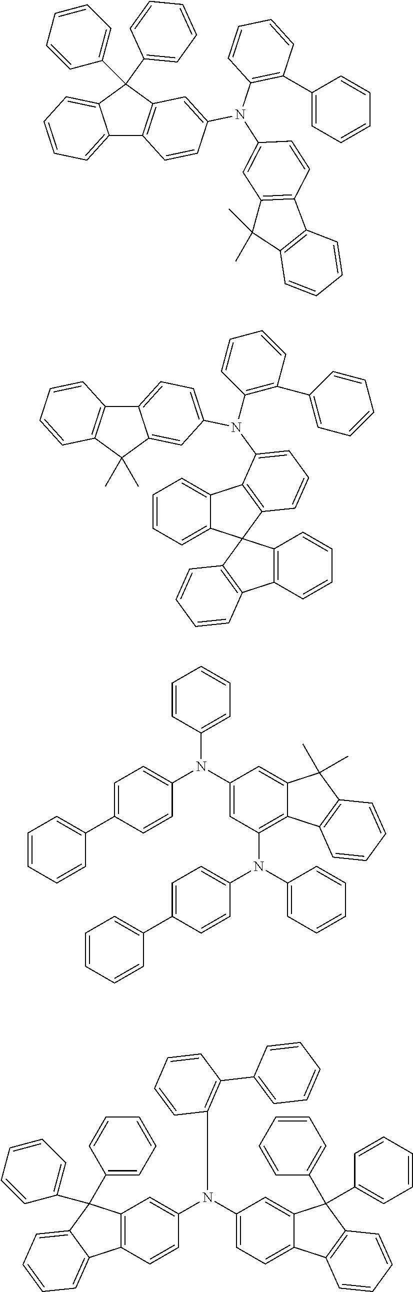

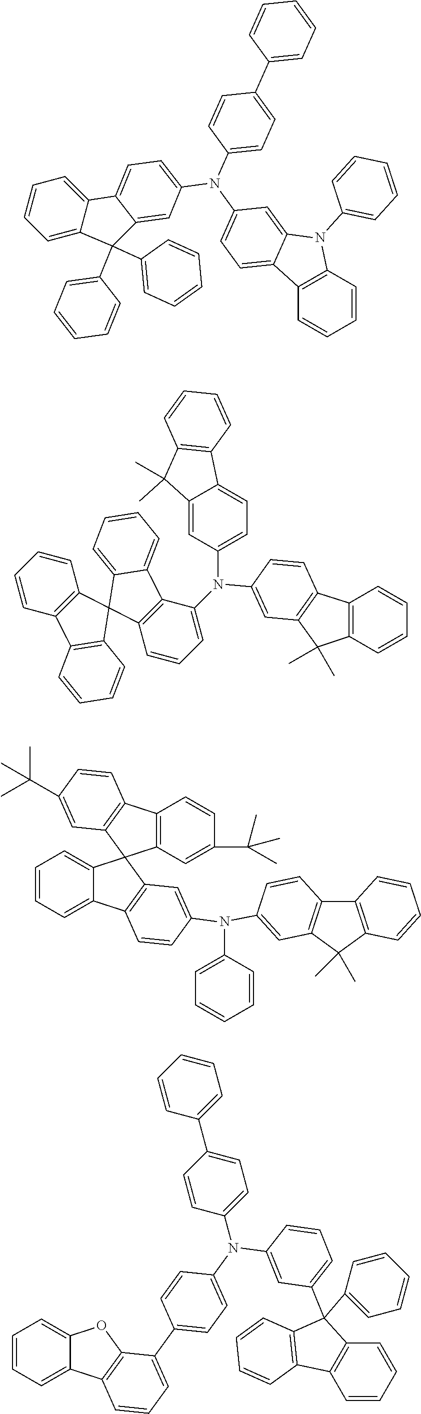

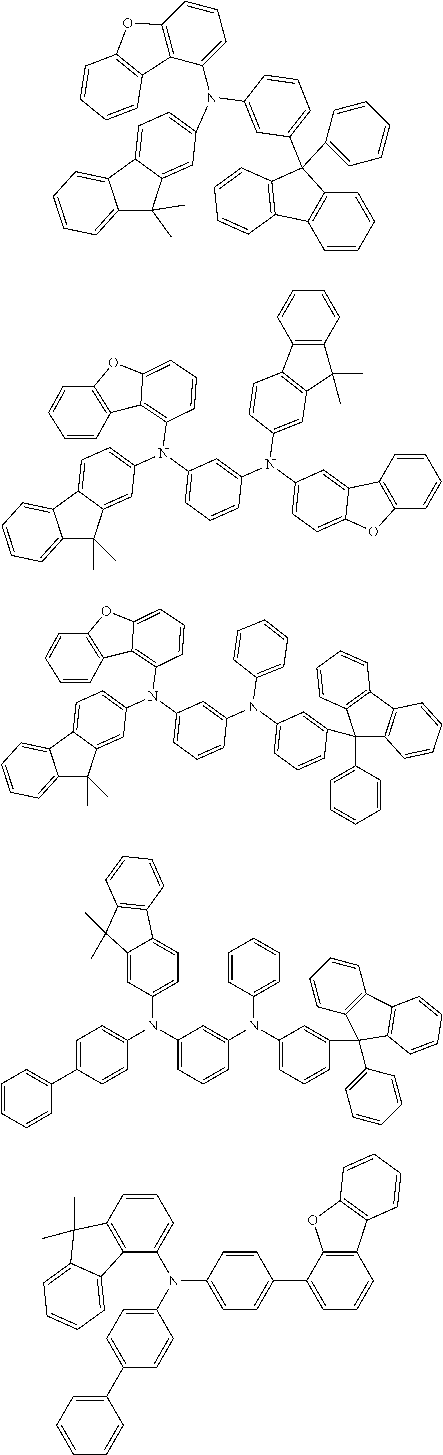

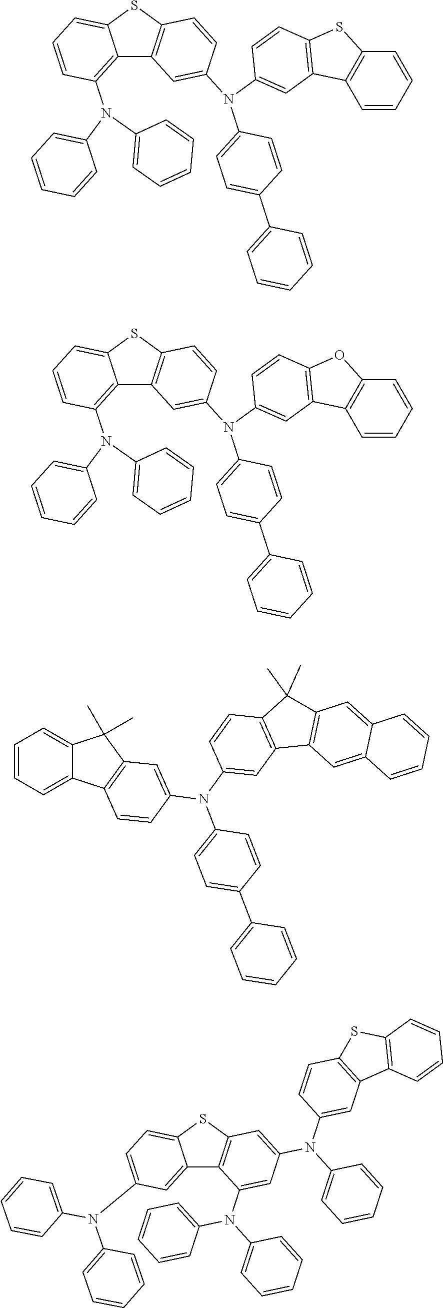 [SMILES: c1ccc(-c2ccc(N(c3ccc4sc5ccccc5c4c3)c3ccc4sc5cccc(N(c6ccccc6)c6ccccc6)c5c4c3)cc2)cc1 ; c1ccc(-c2ccc(N(c3ccc4oc5ccccc5c4c3)c3ccc4sc5cccc(N(c6ccccc6)c6ccccc6)c5c4c3)cc2)cc1 ; CC1(C)c2ccccc2-c2ccc(N(c3ccc(-c4ccccc4)cc3)c3ccc4c(c3)-c3cc5ccccc5cc3C4(C)C)cc21 ; c1ccc(N(c2cc(N(c3ccccc3)c3ccccc3)c3c(c2)sc2ccc(N(c4ccccc4)c4ccccc4)cc23)c2ccc3sc4ccccc4c3c2)cc1]

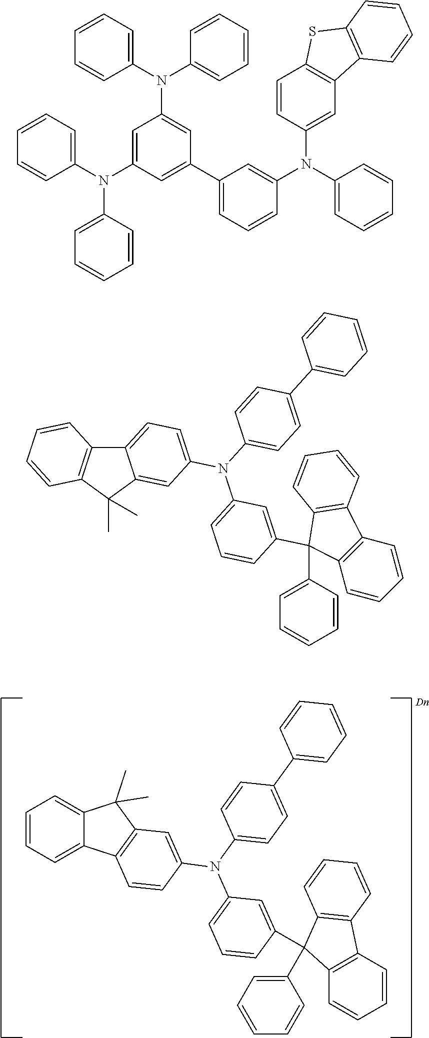

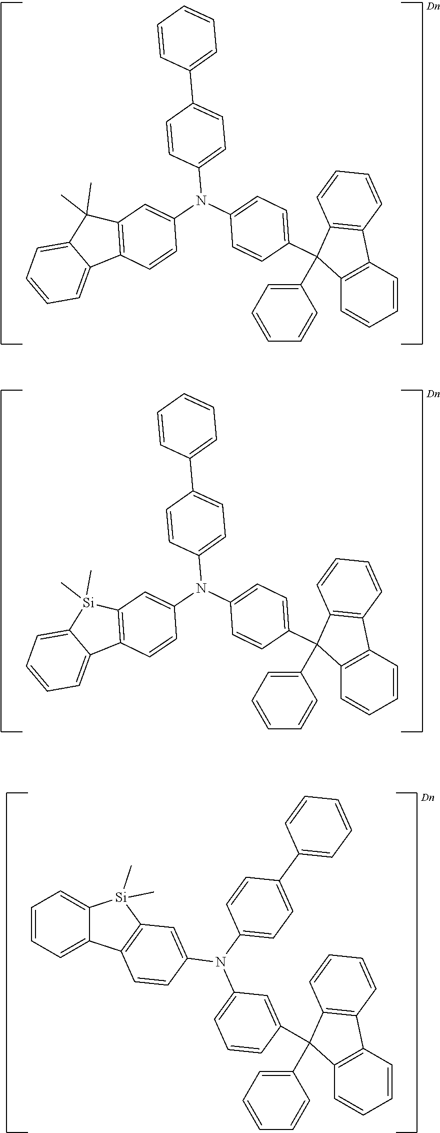

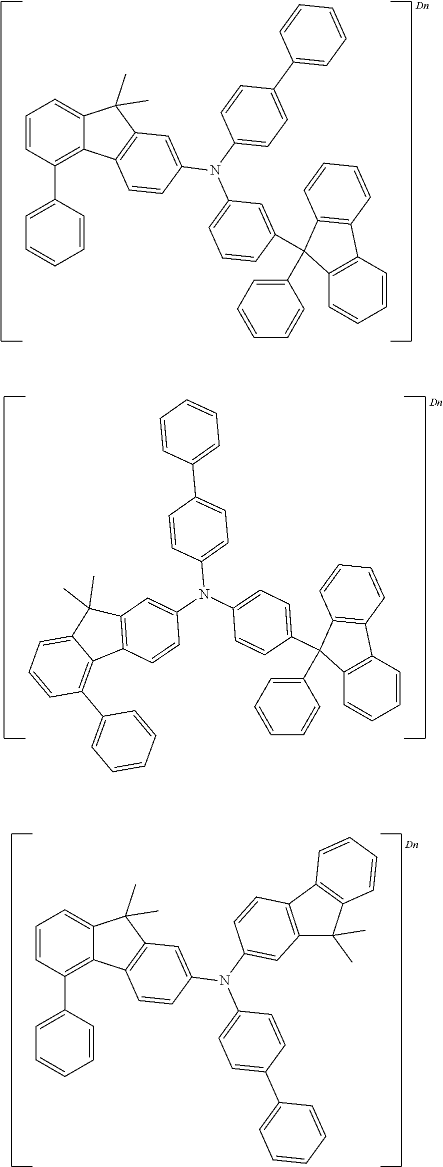

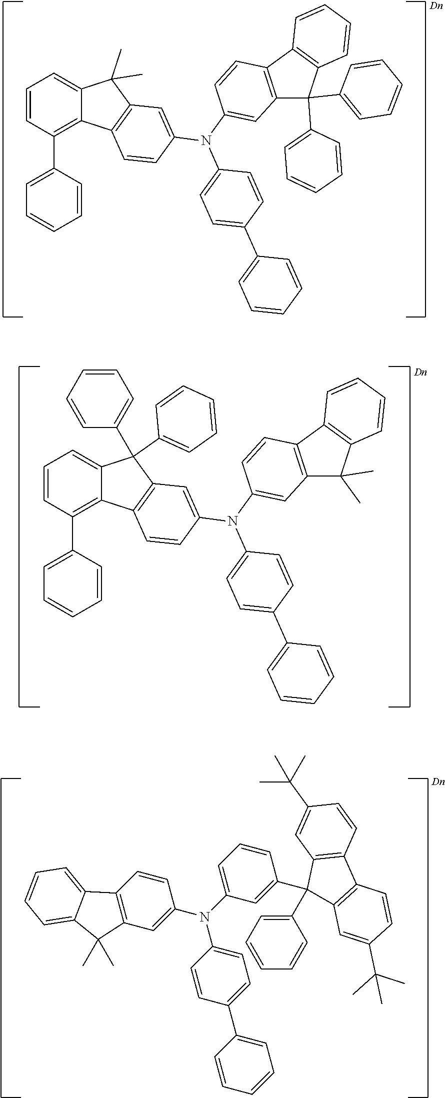

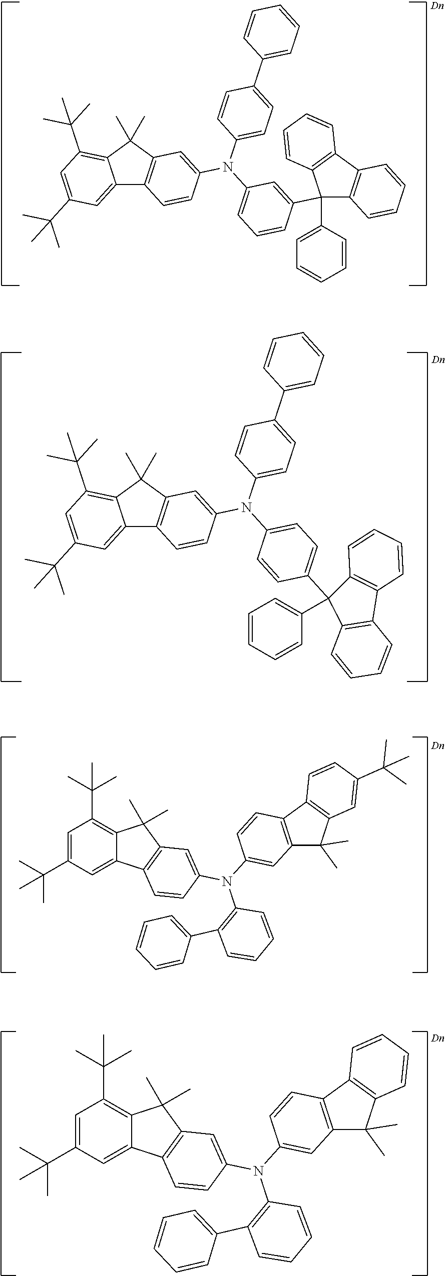

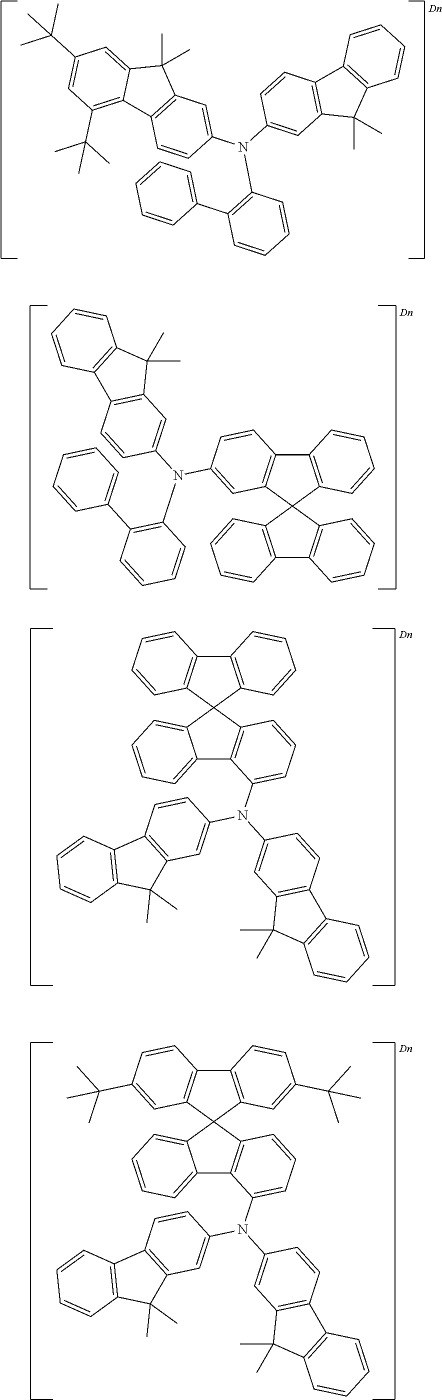

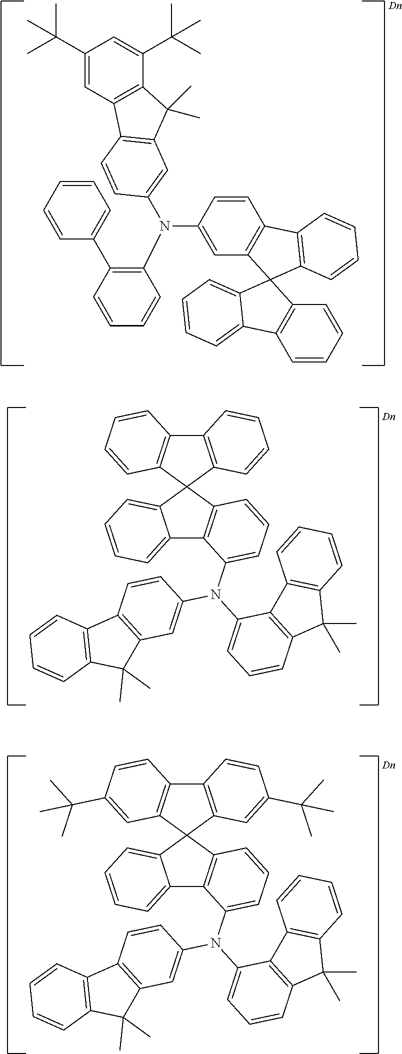

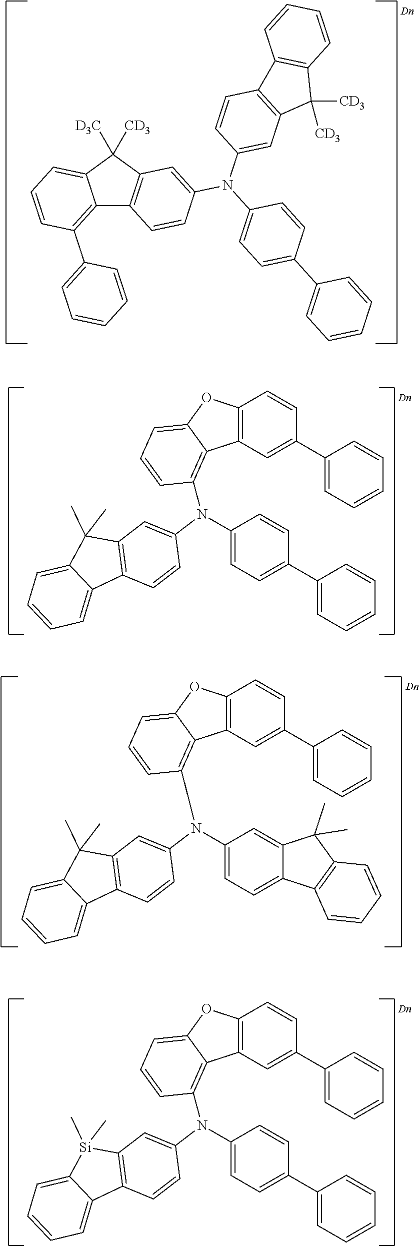

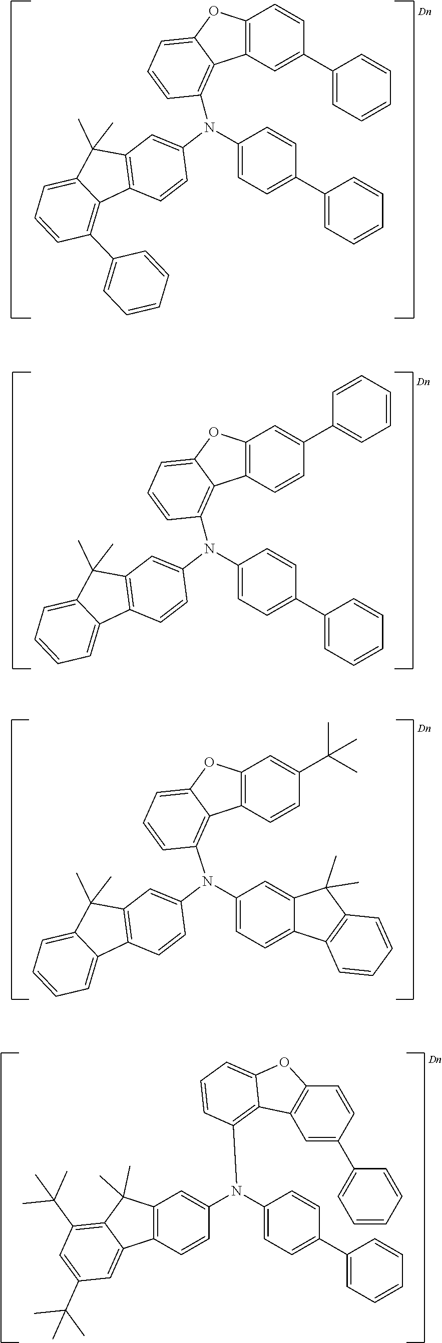

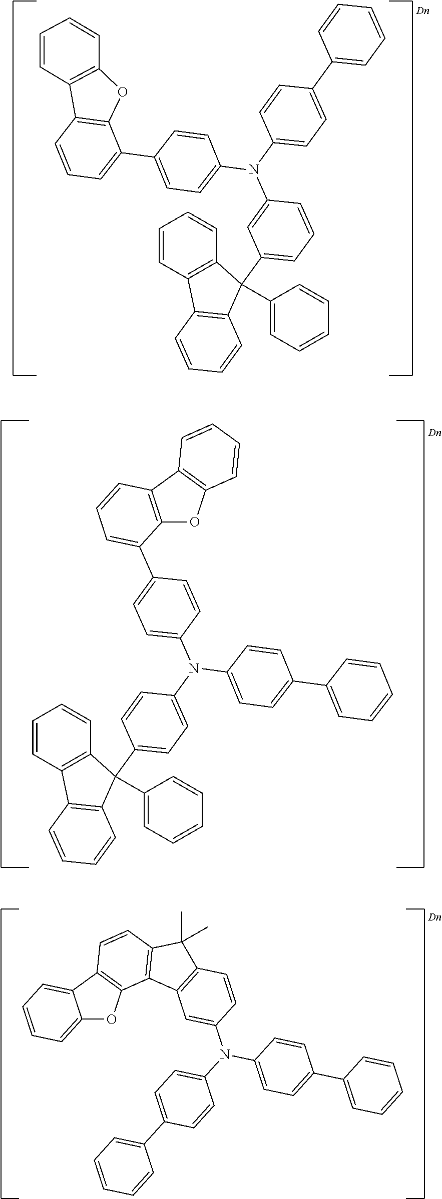

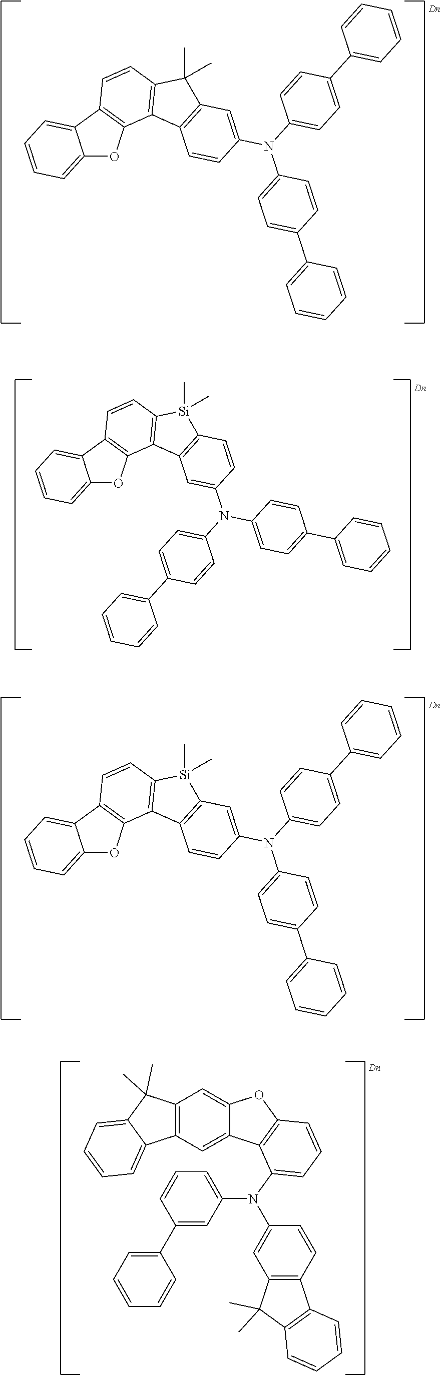

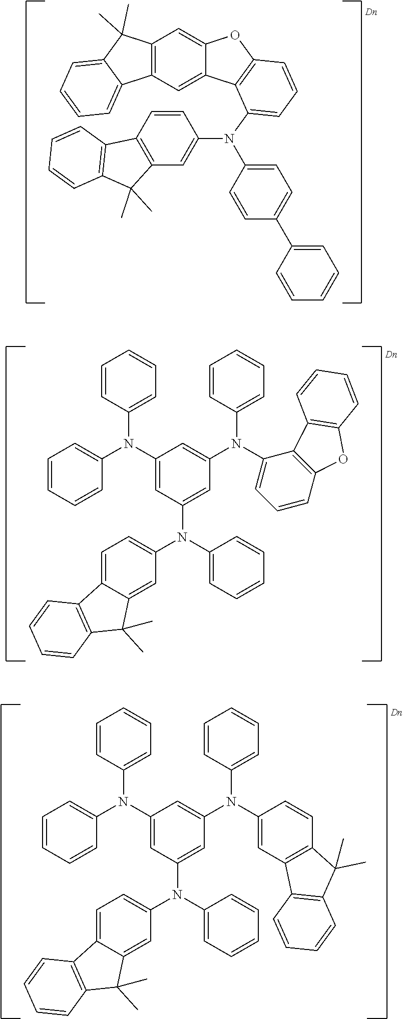

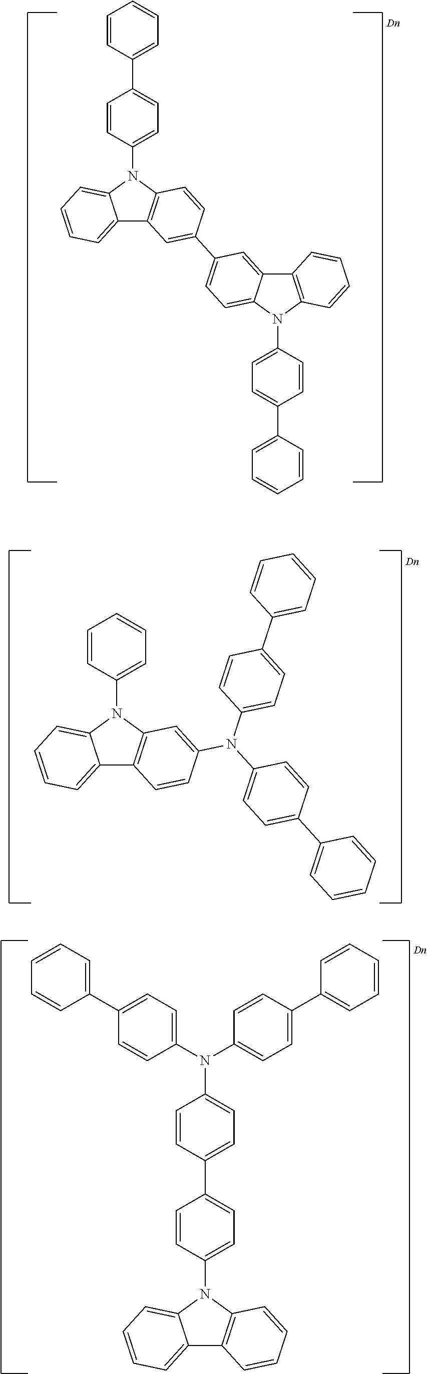

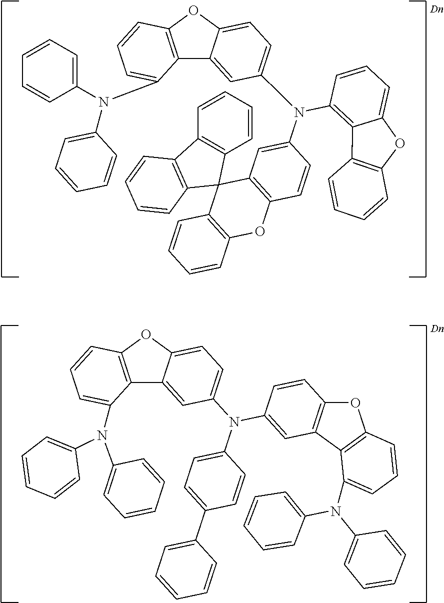

(Dn refers to the number of hydrogens replaced by deuterium and indicates a structure with one or more deuterium substitutions)

In addition to the aforementioned compounds, other suitable compounds may also be used in the hole transport layer 32 and hole injection layer 34.

The hole transport auxiliary layer 33 may include a first hole transport auxiliary layer 33a adjacent to the hole transport layer 32 and a second hole transport auxiliary layer 33b adjacent to the light emitting layer 31.

The first hole transport auxiliary layer 33a may include a first compound represented by Chemical Formula 1, and the second hole transport auxiliary layer 33b may include a second compound represented by Chemical Formula 2.

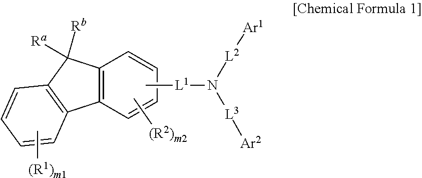

In Chemical Formula 1, R^{a} and R^{b} may each independently be, e.g., a substituted or unsubstituted C1 to C30 alkyl group, or a substituted or unsubstituted C6 to C30 aryl group.

L¹ to L³ may each independently be, e.g., a single bond, a substituted or unsubstituted C6 to C20 arylene group, or a substituted or unsubstituted C2 to C30 heterocyclic group.

R¹ and R² may each independently be, e.g., hydrogen, deuterium, a cyano group, a substituted or unsubstituted C1 to C30 alkyl group, a substituted or unsubstituted C6 to C30 aryl group, or a substituted or unsubstituted C2 to C30 heterocyclic group.

Ar¹ and Ar² may each independently be, e.g., a substituted or unsubstituted C3 to C40 cycloalkyl group or a substituted or unsubstituted C6 to C20 aryl group.

m1 may be, e.g., an integer of 1 to 4.

m2 may be, e.g., an integer of 1 to 3.

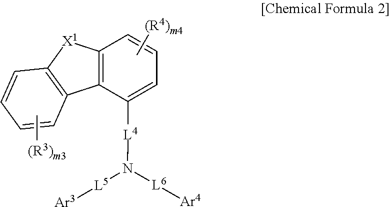

In Chemical Formula 2, X¹ may be, e.g., O or S.

L⁴ to L⁶ may each independently be, e.g., a single bond, a substituted or unsubstituted C6 to C20 arylene group, or a substituted or unsubstituted C2 to C30 heterocyclic group.

R³ and R⁴ may each independently be, e.g., hydrogen, deuterium, a cyano group, a substituted or unsubstituted C1 to C30 alkyl group, a substituted or unsubstituted C6 to C30 aryl group, or a substituted or unsubstituted C2 to C30 heterocyclic group.

Ar³ and Ar⁴ may each independently be, e.g., a substituted or unsubstituted C6 to C20 aryl group or a substituted or unsubstituted C2 to C30 heterocyclic group.

At least one of Ar³ and Art may be, e.g., a substituted or unsubstituted 9-fluorenyl group.

m3 may be, e.g., an integer of 1 to 4.

m4 may be, e.g., an integer of 1 to 3.

The organic light emitting diode according to some embodiments may apply a combination of two hole transport auxiliary layers at the interface between the hole transport layer and the light emitting layer, thereby adjusting the hole injection ability in the first hole transport auxiliary layer to balance the charge in the light emitting layer, and electrons in the second hole transport auxiliary layer may be blocked and the hole and electron densities may be balanced, thereby realizing a low driving voltage, high efficiency, and improved life-span characteristics.

In an implementation, m1 may be, e.g., 2, 3, or 4 and each R¹ may be the same or different from each other.

In an implementation, m2 may be, e.g., 2 or 3 and each R² may be the same or different from each other.

In an implementation, Chemical Formula 1 may be represented, e.g., by one of Chemical Formula 1-1 to Chemical Formula 1-4.

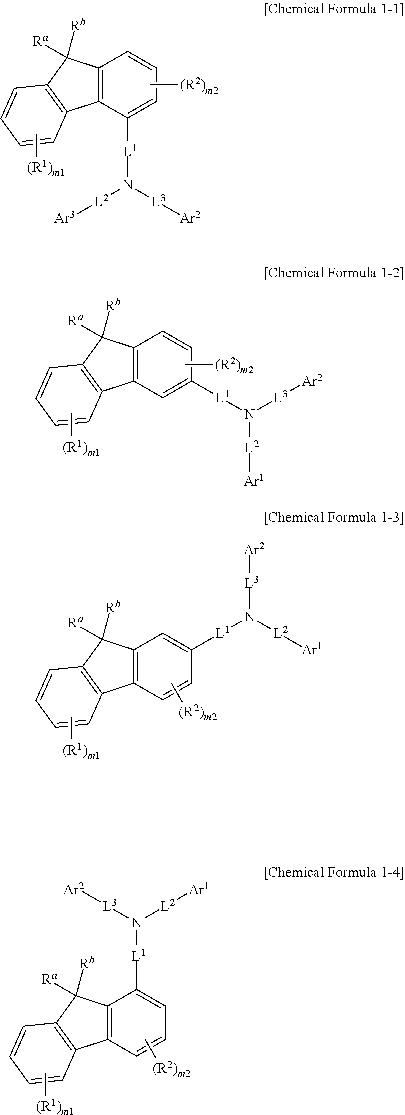

In Chemical Formula 1-1 to Chemical Formula 1-4, the definitions of L¹ to L³, R^{a}, R^{b}, R¹, R², Ar¹, Ar², m1, and m2 may be defined the same as those in Chemical Formula 1.

In an implementation, Ar¹ and Ar² in Chemical Formula 1 may each independently be, e.g., a substituted or unsubstituted cyclopentyl group, a substituted or unsubstituted cyclohexyl group, a substituted or unsubstituted bicyclo[2,2,1]heptyl group, a substituted or unsubstituted adamantyl group, a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, or a substituted or unsubstituted phenanthrenyl group, a substituted or unsubstituted triphenylene group, a substituted or unsubstituted fluorenyl group, or a substituted or unsubstituted dibenzosilolyl group.

In an implementation, Ar¹ and Ar² in Chemical Formula 1 may each independently be, e.g., a substituted or unsubstituted cyclopentyl group, a substituted or unsubstituted cyclohexyl group, a substituted or unsubstituted bicyclo[2,2,1]heptyl group, a substituted or unsubstituted adamantyl group, a substituted or unsubstituted phenyl group, a substituted or unsubstituted biphenyl group, a substituted or unsubstituted triphenylene group, a substituted or unsubstituted fluorenyl group, or a substituted or unsubstituted dibenzosilolyl group.

In an implementation, R¹ and R² in Chemical Formula 1 may each independently be, e.g., hydrogen, deuterium, a substituted or unsubstituted C1 to C10 alkyl group, or a substituted or unsubstituted C6 to C12 aryl group.

In an implementation, R¹ and R² in Chemical Formula 1 may each independently be, e.g., hydrogen, deuterium, a substituted or unsubstituted tert-butyl group, or a substituted or unsubstituted phenyl group.

In an implementation, L¹ to L³ in Chemical Formula 1 may each independently be, e.g., a single bond or a substituted or unsubstituted phenylene group.

In an implementation, moieties L²-Ar¹, and L³-Ar² may each be independently a moiety of Group I.

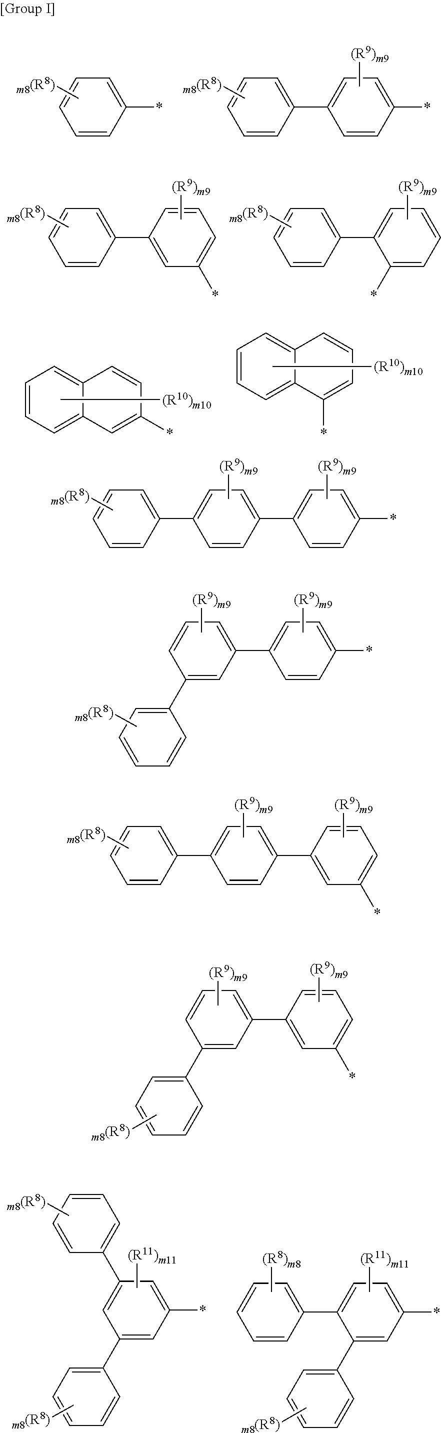

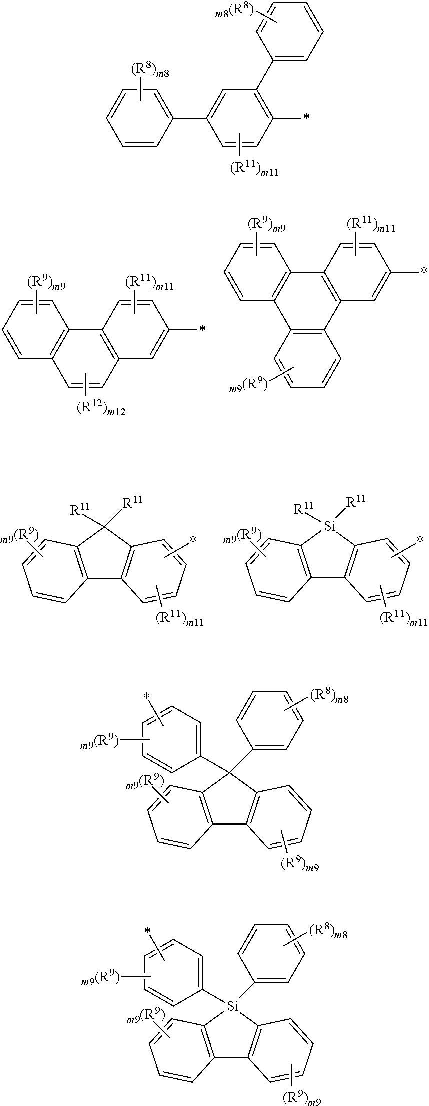

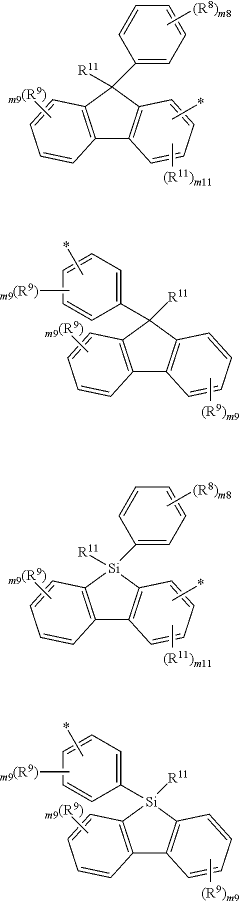

In Group I, R⁸ to R¹² may each independently be, e.g., hydrogen, deuterium, a cyano group, a substituted or unsubstituted C1 to C10 alkyl group, or a substituted or unsubstituted C6 to C12 aryl group.

m8 may be, e.g., an integer of 1 to 5.

m9 may be, e.g., an integer of 1 to 4.

m10 may be, e.g., an integer of 1 to 7.

m11 may be, e.g., an integer of 1 to 3.

m12 may be, e.g., an integer of 1 or 2.

* is a linking point.

In an implementation, m8 may be, e.g., 2, 3, 4, or 5 and each R⁸ may be the same or different from each other.

In an implementation, m9 may be, e.g., 2, 3, or 4 and each R⁹ may be the same or different from each other.

In an implementation, m10 may be, e.g., 2, 3, 4, 5, 6, or 7 and each R¹⁰ may be the same or different from each other.

In an implementation, m11 may be, e.g., 2 or 3 and each R¹¹ may be the same or different from each other.

In an implementation, m12 may be, e.g., 2 and each R¹² may be the same or different from each other.

In an implementation, the first compound may be, e.g., a compound of Group 1.

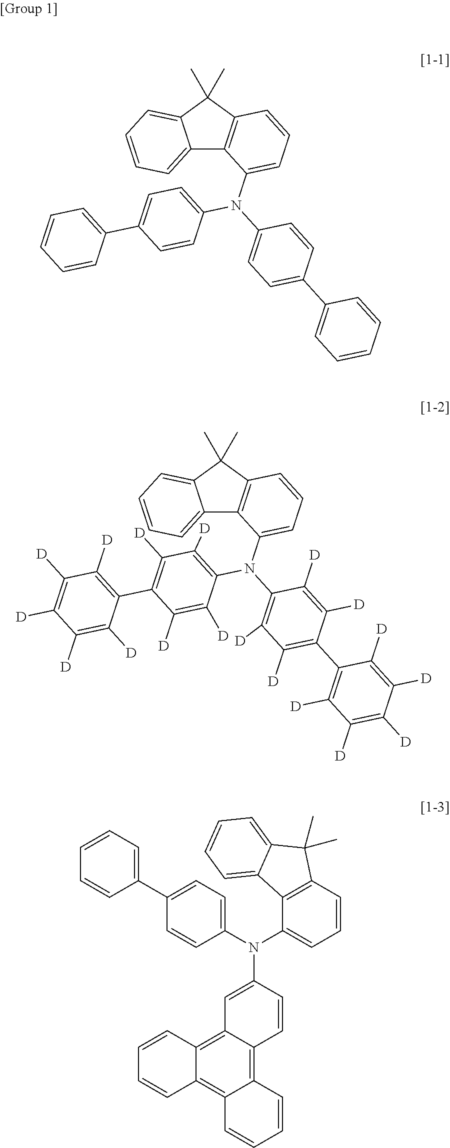 [SMILES: CC1(C)c2ccccc2-c2c(N(c3ccc(-c4ccccc4)cc3)c3ccc(-c4ccccc4)cc3)cccc21 ; [2H]c1c([2H])c([2H])c(-c2c([2H])c([2H])c(N(c3cccc4c3-c3ccccc3C4(C)C)c3c([2H])c([2H])c(-c4c([2H])c([2H])c([2H])c([2H])c4[2H])c([2H])c3[2H])c([2H])c2[2H])c([2H])c1[2H] ; CC1(C)c2ccccc2-c2c(N(c3ccc(-c4ccccc4)cc3)c3ccc4c5ccccc5c5ccccc5c4c3)cccc21]

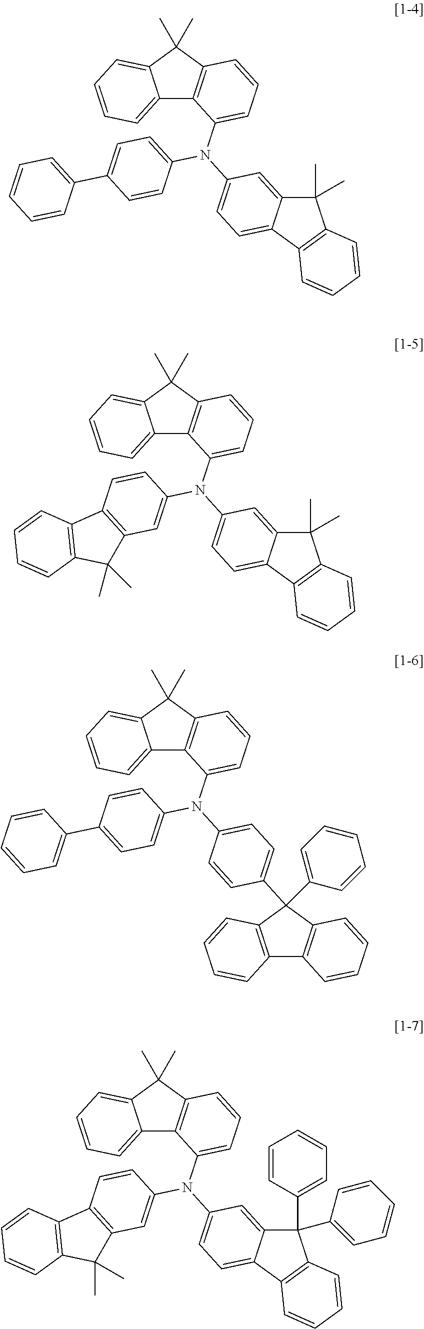 [SMILES: CC1(C)c2ccccc2-c2ccc(N(c3ccc(-c4ccccc4)cc3)c3cccc4c3-c3ccccc3C4(C)C)cc21 ; CC1(C)c2ccccc2-c2ccc(N(c3ccc4c(c3)C(C)(C)c3ccccc3-4)c3cccc4c3-c3ccccc3C4(C)C)cc21 ; CC1(C)c2ccccc2-c2c(N(c3ccc(-c4ccccc4)cc3)c3ccc(C4(c5ccccc5)c5ccccc5-c5ccccc54)cc3)cccc21 ; CC1(C)c2ccccc2-c2ccc(N(c3ccc4c(c3)C(c3ccccc3)(c3ccccc3)c3ccccc3-4)c3cccc4c3-c3ccccc3C4(C)C)cc21]

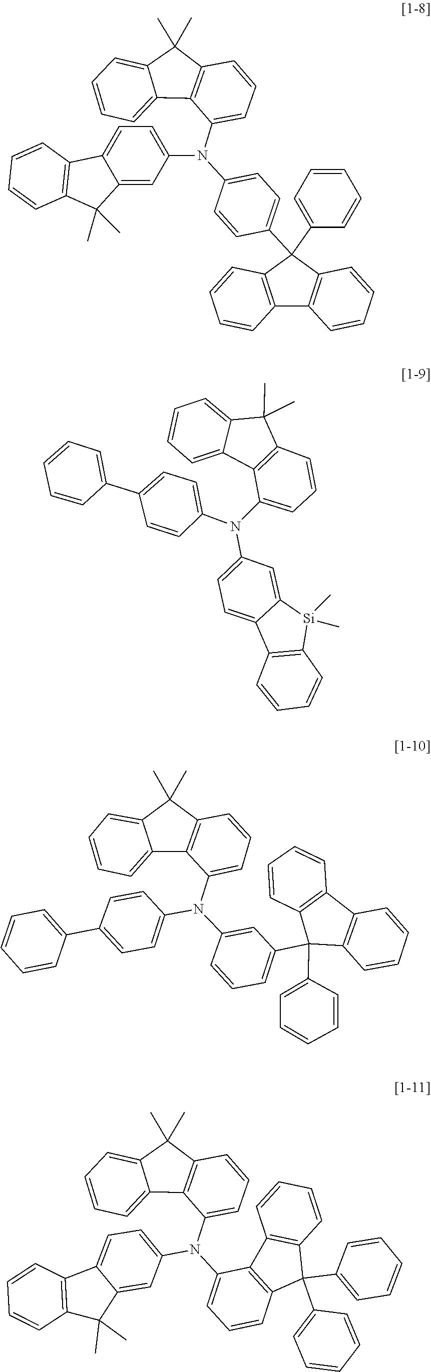 [SMILES: CC1(C)c2ccccc2-c2ccc(N(c3ccc(C4(c5ccccc5)c5ccccc5-c5ccccc54)cc3)c3cccc4c3-c3ccccc3C4(C)C)cc21 ; CC1(C)c2ccccc2-c2c(N(c3ccc(-c4ccccc4)cc3)c3ccc4c(c3)[Si](C)(C)c3ccccc3-4)cccc21 ; CC1(C)c2ccccc2-c2c(N(c3ccc(-c4ccccc4)cc3)c3cccc(C4(c5ccccc5)c5ccccc5-c5ccccc54)c3)cccc21 ; CC1(C)c2ccccc2-c2ccc(N(c3cccc4c3-c3ccccc3C4(C)C)c3cccc4c3-c3ccccc3C4(c3ccccc3)c3ccccc3)cc21]

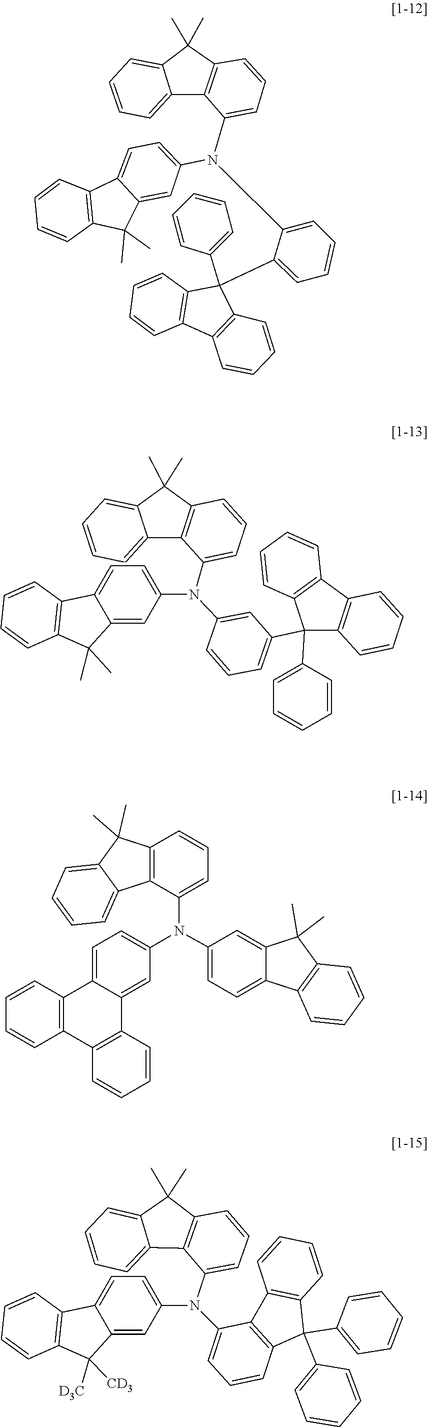 [SMILES: CC1(C)c2ccccc2-c2ccc(N(c3ccccc3C3(c4ccccc4)c4ccccc4-c4ccccc43)c3cccc4c3-c3ccccc3C4(C)C)cc21 ; CC1(C)c2ccccc2-c2ccc(N(c3cccc(C4(c5ccccc5)c5ccccc5-c5ccccc54)c3)c3cccc4c3-c3ccccc3C4(C)C)cc21 ; CC1(C)c2ccccc2-c2ccc(N(c3ccc4c5ccccc5c5ccccc5c4c3)c3cccc4c3-c3ccccc3C4(C)C)cc21 ; [2H]C([2H])([2H])C1(C([2H])([2H])[2H])c2ccccc2-c2ccc(N(c3cccc4c3-c3ccccc3C4(C)C)c3cccc4c3-c3ccccc3C4(c3ccccc3)c3ccccc3)cc21]

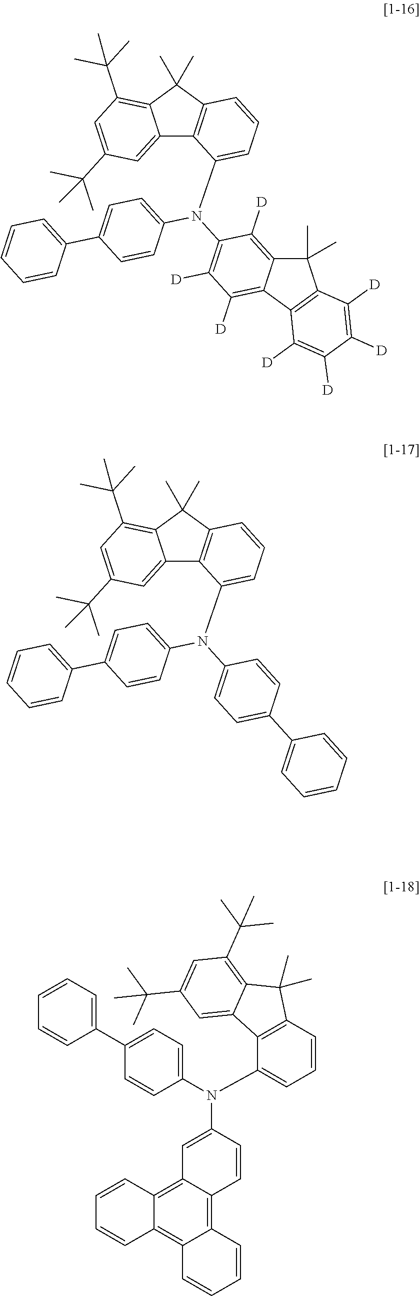 [SMILES: [2H]c1c([2H])c([2H])c2c(c1[2H])-c1c([2H])c([2H])c(N(c3ccc(-c4ccccc4)cc3)c3cccc4c3-c3cc(C(C)(C)C)cc(C(C)(C)C)c3C4(C)C)c([2H])c1C2(C)C ; CC(C)(C)c1cc2c(c(C(C)(C)C)c1)C(C)(C)c1cccc(N(c3ccc(-c4ccccc4)cc3)c3ccc(-c4ccccc4)cc3)c1-2 ; CC(C)(C)c1cc2c(c(C(C)(C)C)c1)C(C)(C)c1cccc(N(c3ccc(-c4ccccc4)cc3)c3ccc4c5ccccc5c5ccccc5c4c3)c1-2]

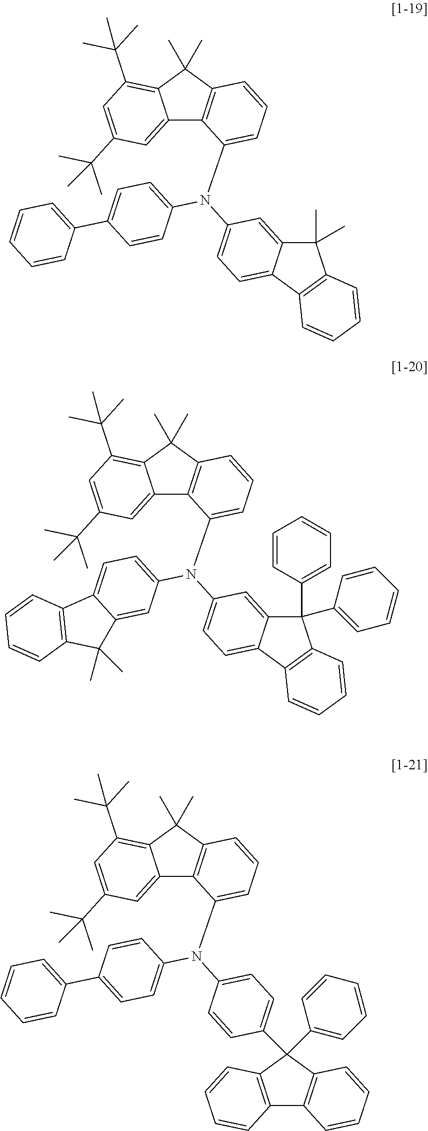 [SMILES: CC(C)(C)c1cc2c(c(C(C)(C)C)c1)C(C)(C)c1cccc(N(c3ccc(-c4ccccc4)cc3)c3ccc4c(c3)C(C)(C)c3ccccc3-4)c1-2 ; CC(C)(C)c1cc2c(c(C(C)(C)C)c1)C(C)(C)c1cccc(N(c3ccc4c(c3)C(C)(C)c3ccccc3-4)c3ccc4c(c3)C(c3ccccc3)(c3ccccc3)c3ccccc3-4)c1-2 ; CC(C)(C)c1cc2c(c(C(C)(C)C)c1)C(C)(C)c1cccc(N(c3ccc(-c4ccccc4)cc3)c3ccc(C4(c5ccccc5)c5ccccc5-c5ccccc54)cc3)c1-2]

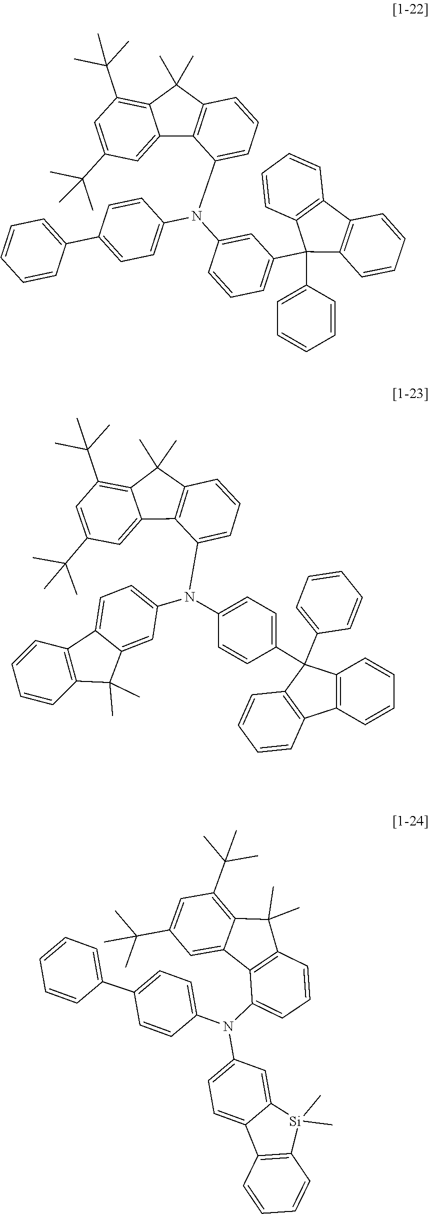 [SMILES: CC(C)(C)c1cc2c(c(C(C)(C)C)c1)C(C)(C)c1cccc(N(c3ccc(-c4ccccc4)cc3)c3cccc(C4(c5ccccc5)c5ccccc5-c5ccccc54)c3)c1-2 ; CC(C)(C)c1cc2c(c(C(C)(C)C)c1)C(C)(C)c1cccc(N(c3ccc(C4(c5ccccc5)c5ccccc5-c5ccccc54)cc3)c3ccc4c(c3)C(C)(C)c3ccccc3-4)c1-2 ; CC(C)(C)c1cc2c(c(C(C)(C)C)c1)C(C)(C)c1cccc(N(c3ccc(-c4ccccc4)cc3)c3ccc4c(c3)[Si](C)(C)c3ccccc3-4)c1-2]

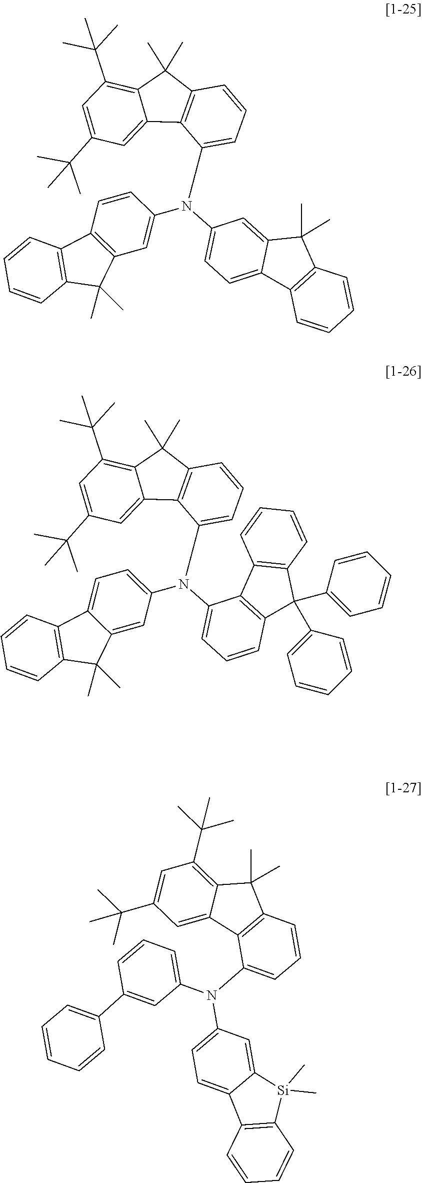 [SMILES: CC(C)(C)c1cc2c(c(C(C)(C)C)c1)C(C)(C)c1cccc(N(c3ccc4c(c3)C(C)(C)c3ccccc3-4)c3ccc4c(c3)C(C)(C)c3ccccc3-4)c1-2 ; CC(C)(C)c1cc2c(c(C(C)(C)C)c1)C(C)(C)c1cccc(N(c3ccc4c(c3)C(C)(C)c3ccccc3-4)c3cccc4c3-c3ccccc3C4(c3ccccc3)c3ccccc3)c1-2 ; CC(C)(C)c1cc2c(c(C(C)(C)C)c1)C(C)(C)c1cccc(N(c3cccc(-c4ccccc4)c3)c3ccc4c(c3)[Si](C)(C)c3ccccc3-4)c1-2]

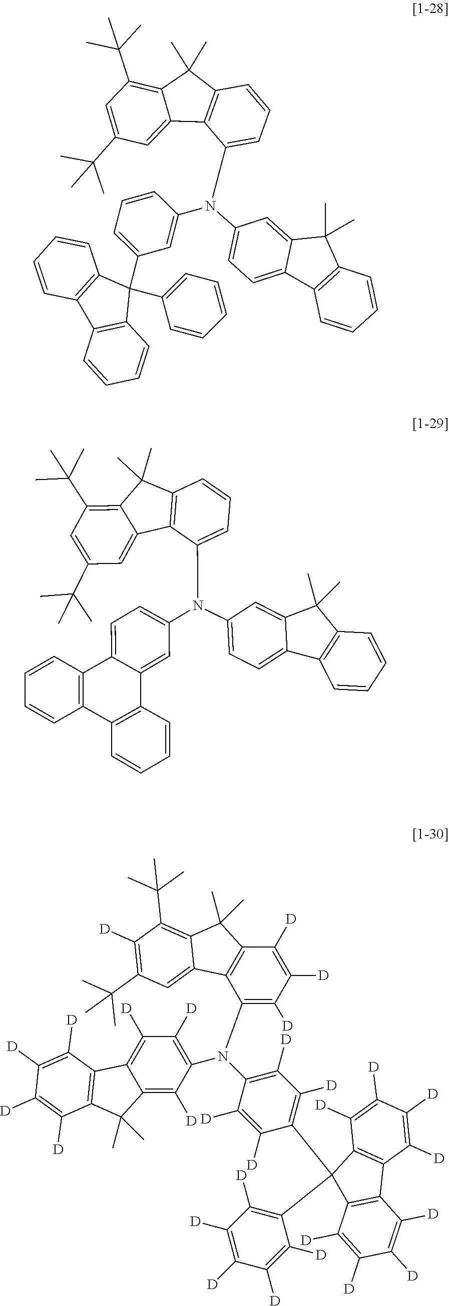

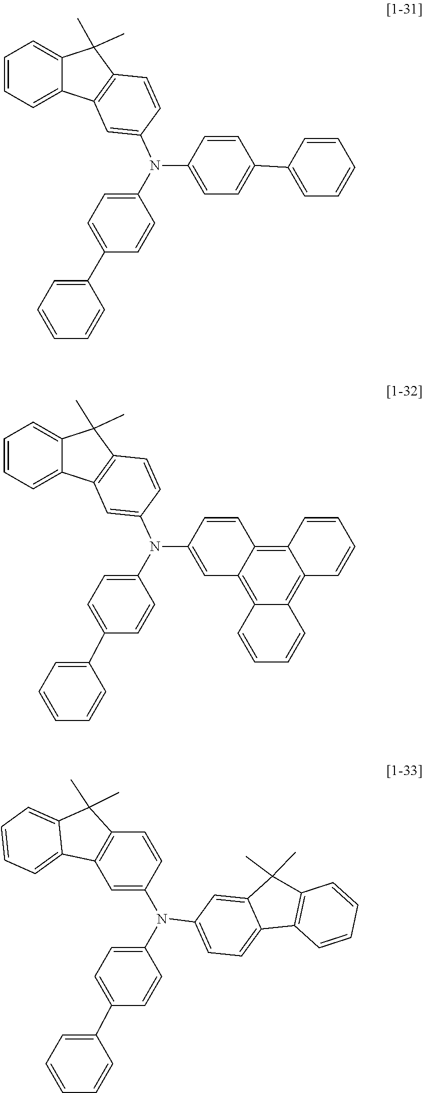 [SMILES: CC1(C)c2ccccc2-c2cc(N(c3ccc(-c4ccccc4)cc3)c3ccc(-c4ccccc4)cc3)ccc21 ; CC1(C)c2ccccc2-c2cc(N(c3ccc(-c4ccccc4)cc3)c3ccc4c5ccccc5c5ccccc5c4c3)ccc21 ; CC1(C)c2ccccc2-c2cc(N(c3ccc(-c4ccccc4)cc3)c3ccc4c(c3)C(C)(C)c3ccccc3-4)ccc21]

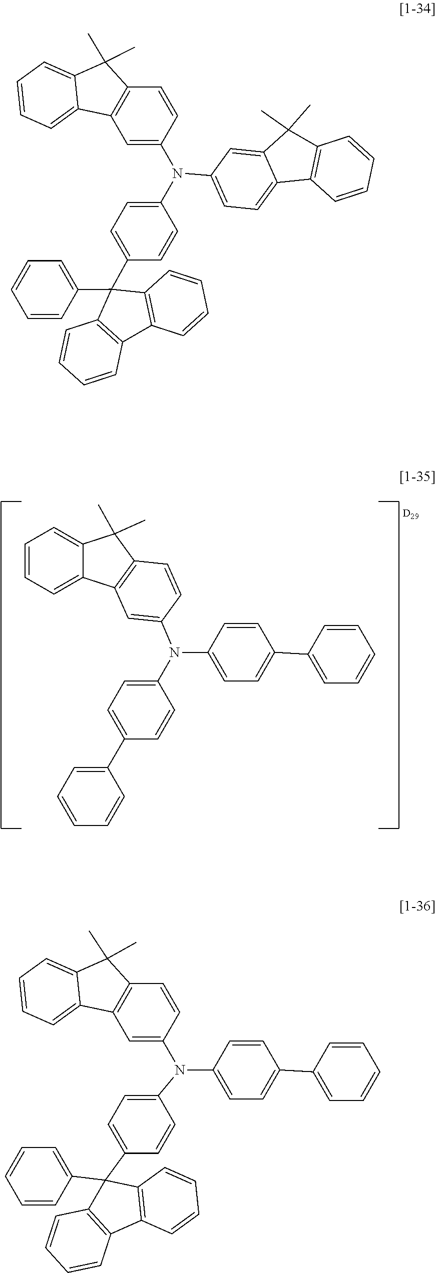

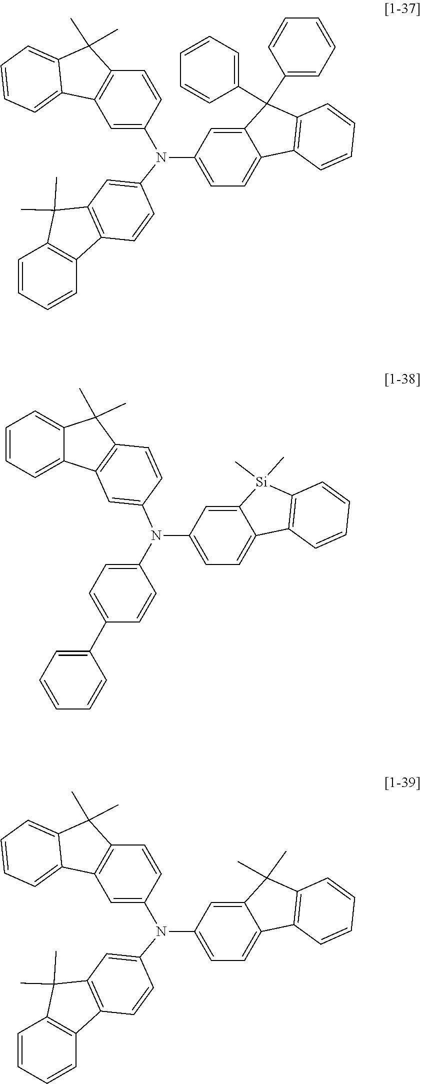 [SMILES: CC1(C)c2ccccc2-c2cc(N(c3ccc4c(c3)C(C)(C)c3ccccc3-4)c3ccc4c(c3)C(c3ccccc3)(c3ccccc3)c3ccccc3-4)ccc21 ; CC1(C)c2ccccc2-c2cc(N(c3ccc(-c4ccccc4)cc3)c3ccc4c(c3)[Si](C)(C)c3ccccc3-4)ccc21 ; CC1(C)c2ccccc2-c2cc(N(c3ccc4c(c3)C(C)(C)c3ccccc3-4)c3ccc4c(c3)C(C)(C)c3ccccc3-4)ccc21]

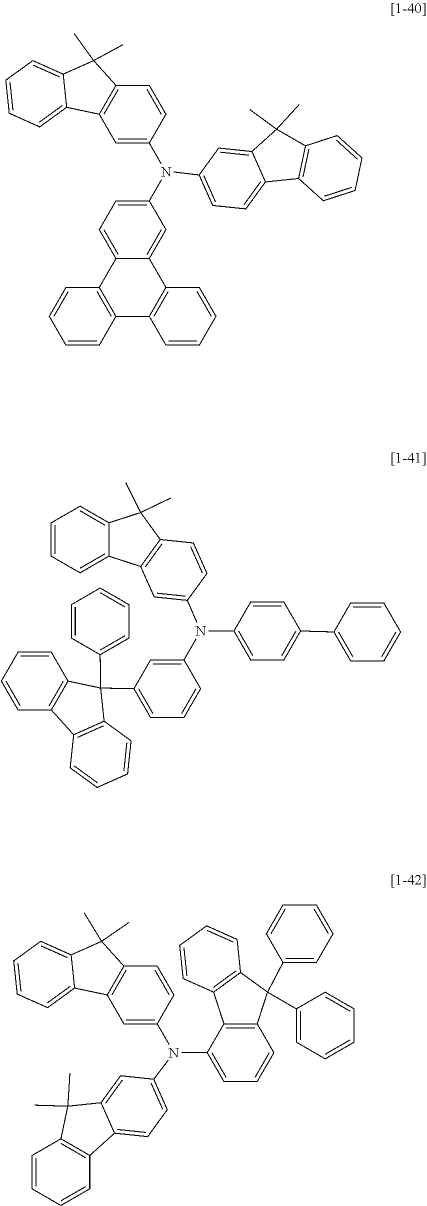 [SMILES: CC1(C)c2ccccc2-c2cc(N(c3ccc4c(c3)C(C)(C)c3ccccc3-4)c3ccc4c5ccccc5c5ccccc5c4c3)ccc21 ; CC1(C)c2ccccc2-c2cc(N(c3ccc(-c4ccccc4)cc3)c3cccc(C4(c5ccccc5)c5ccccc5-c5ccccc54)c3)ccc21 ; CC1(C)c2ccccc2-c2cc(N(c3ccc4c(c3)C(C)(C)c3ccccc3-4)c3cccc4c3-c3ccccc3C4(c3ccccc3)c3ccccc3)ccc21]

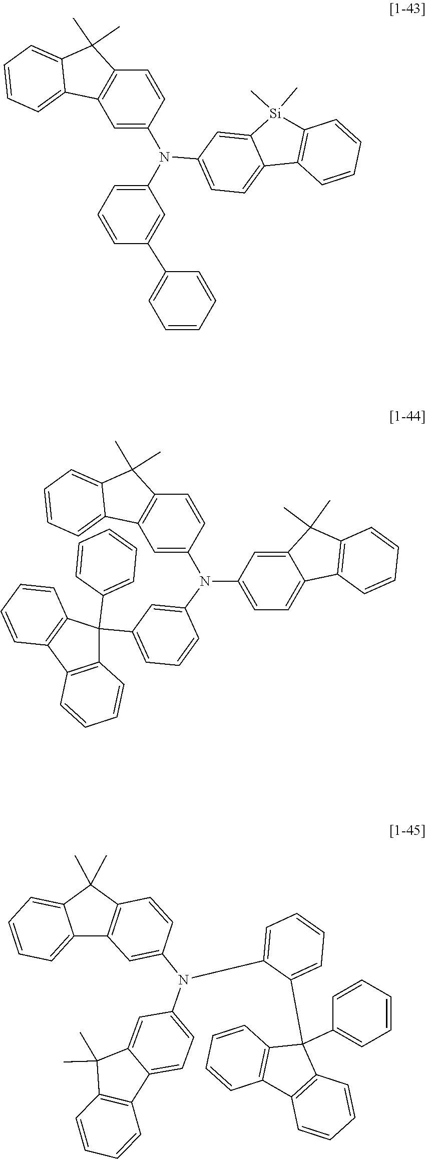 [SMILES: CC1(C)c2ccccc2-c2cc(N(c3cccc(-c4ccccc4)c3)c3ccc4c(c3)[Si](C)(C)c3ccccc3-4)ccc21 ; CC1(C)c2ccccc2-c2cc(N(c3cccc(C4(c5ccccc5)c5ccccc5-c5ccccc54)c3)c3ccc4c(c3)C(C)(C)c3ccccc3-4)ccc21 ; CC1(C)c2ccccc2-c2cc(N(c3ccc4c(c3)C(C)(C)c3ccccc3-4)c3ccccc3C3(c4ccccc4)c4ccccc4-c4ccccc43)ccc21]

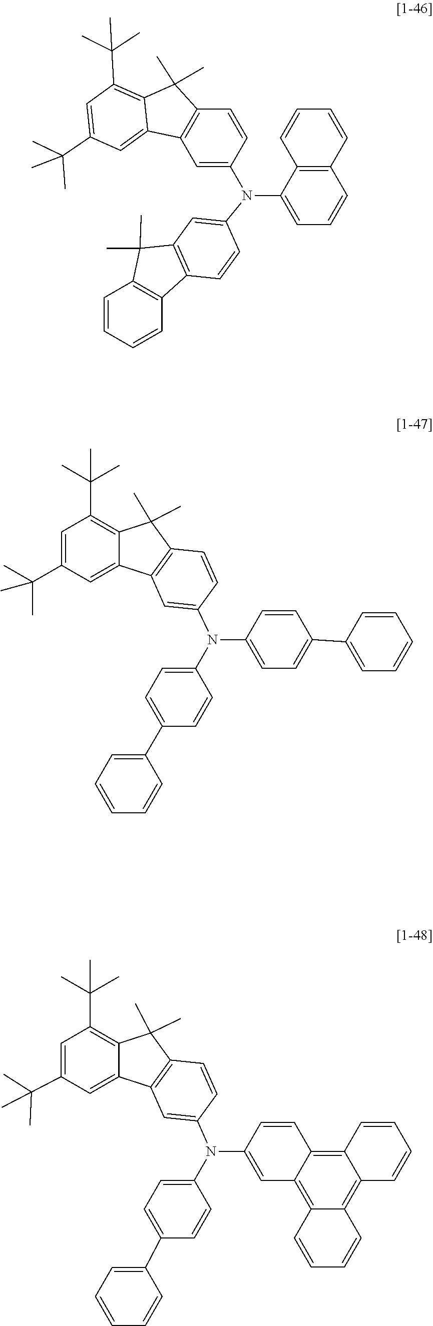 [SMILES: CC(C)(C)c1cc2c(c(C(C)(C)C)c1)C(C)(C)c1ccc(N(c3ccc4c(c3)C(C)(C)c3ccccc3-4)c3cccc4ccccc34)cc1-2 ; CC(C)(C)c1cc2c(c(C(C)(C)C)c1)C(C)(C)c1ccc(N(c3ccc(-c4ccccc4)cc3)c3ccc(-c4ccccc4)cc3)cc1-2 ; CC(C)(C)c1cc2c(c(C(C)(C)C)c1)C(C)(C)c1ccc(N(c3ccc(-c4ccccc4)cc3)c3ccc4c5ccccc5c5ccccc5c4c3)cc1-2]

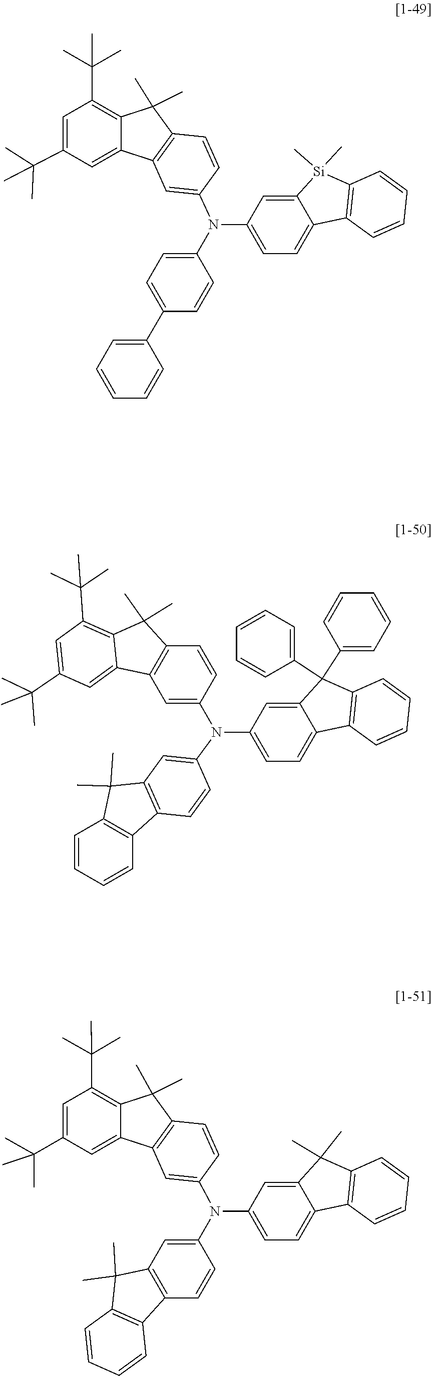 [SMILES: CC(C)(C)c1cc2c(c(C(C)(C)C)c1)C(C)(C)c1ccc(N(c3ccc(-c4ccccc4)cc3)c3ccc4c(c3)[Si](C)(C)c3ccccc3-4)cc1-2 ; CC(C)(C)c1cc2c(c(C(C)(C)C)c1)C(C)(C)c1ccc(N(c3ccc4c(c3)C(C)(C)c3ccccc3-4)c3ccc4c(c3)C(c3ccccc3)(c3ccccc3)c3ccccc3-4)cc1-2 ; CC(C)(C)c1cc2c(c(C(C)(C)C)c1)C(C)(C)c1ccc(N(c3ccc4c(c3)C(C)(C)c3ccccc3-4)c3ccc4c(c3)C(C)(C)c3ccccc3-4)cc1-2]

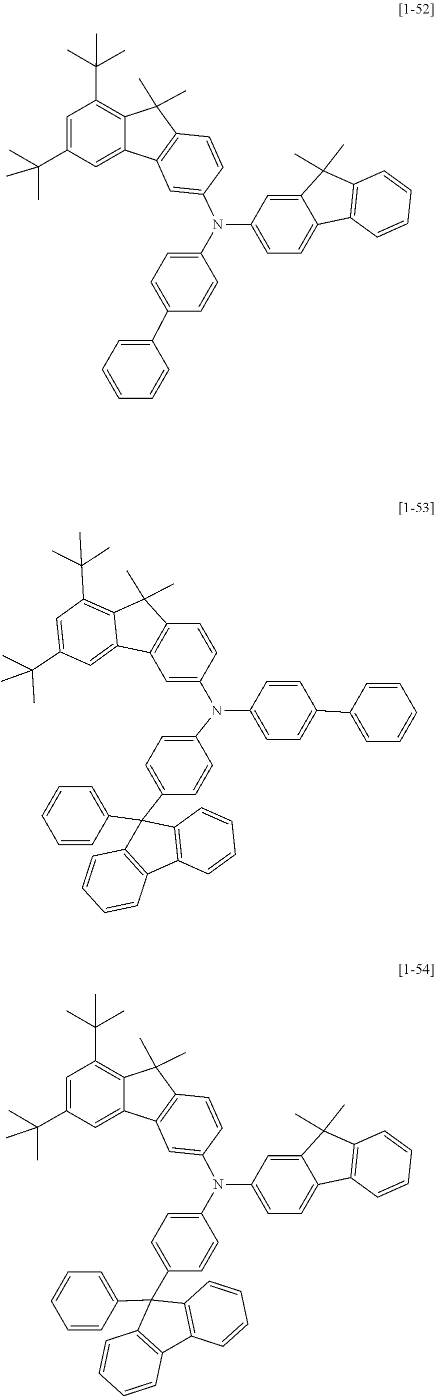 [SMILES: CC(C)(C)c1cc2c(c(C(C)(C)C)c1)C(C)(C)c1ccc(N(c3ccc(-c4ccccc4)cc3)c3ccc4c(c3)C(C)(C)c3ccccc3-4)cc1-2 ; CC(C)(C)c1cc2c(c(C(C)(C)C)c1)C(C)(C)c1ccc(N(c3ccc(-c4ccccc4)cc3)c3ccc(C4(c5ccccc5)c5ccccc5-c5ccccc54)cc3)cc1-2 ; CC(C)(C)c1cc2c(c(C(C)(C)C)c1)C(C)(C)c1ccc(N(c3ccc(C4(c5ccccc5)c5ccccc5-c5ccccc54)cc3)c3ccc4c(c3)C(C)(C)c3ccccc3-4)cc1-2]

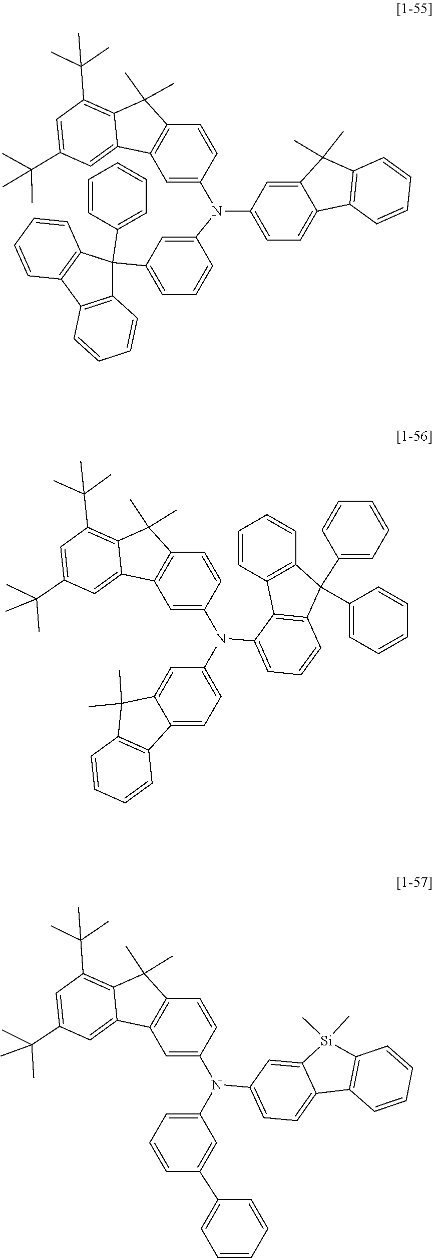 [SMILES: CC(C)(C)c1cc2c(c(C(C)(C)C)c1)C(C)(C)c1ccc(N(c3cccc(C4(c5ccccc5)c5ccccc5-c5ccccc54)c3)c3ccc4c(c3)C(C)(C)c3ccccc3-4)cc1-2 ; CC(C)(C)c1cc2c(c(C(C)(C)C)c1)C(C)(C)c1ccc(N(c3ccc4c(c3)C(C)(C)c3ccccc3-4)c3cccc4c3-c3ccccc3C4(c3ccccc3)c3ccccc3)cc1-2 ; CC(C)(C)c1cc2c(c(C(C)(C)C)c1)C(C)(C)c1ccc(N(c3cccc(-c4ccccc4)c3)c3ccc4c(c3)[Si](C)(C)c3ccccc3-4)cc1-2]

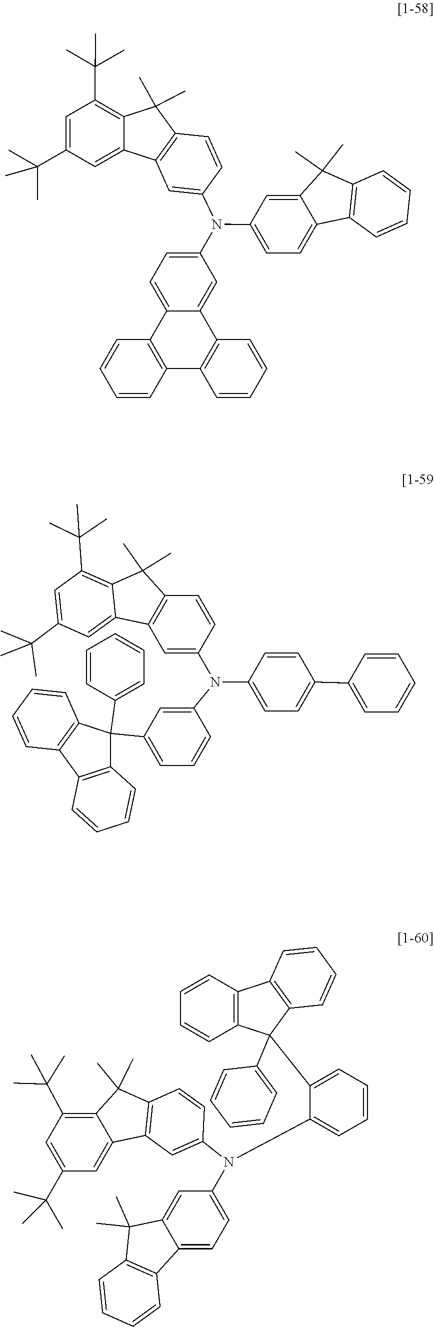 [SMILES: CC(C)(C)c1cc2c(c(C(C)(C)C)c1)C(C)(C)c1ccc(N(c3ccc4c(c3)C(C)(C)c3ccccc3-4)c3ccc4c5ccccc5c5ccccc5c4c3)cc1-2 ; CC(C)(C)c1cc2c(c(C(C)(C)C)c1)C(C)(C)c1ccc(N(c3ccc(-c4ccccc4)cc3)c3cccc(C4(c5ccccc5)c5ccccc5-c5ccccc54)c3)cc1-2 ; CC(C)(C)c1cc2c(c(C(C)(C)C)c1)C(C)(C)c1ccc(N(c3ccc4c(c3)C(C)(C)c3ccccc3-4)c3ccccc3C3(c4ccccc4)c4ccccc4-c4ccccc43)cc1-2]

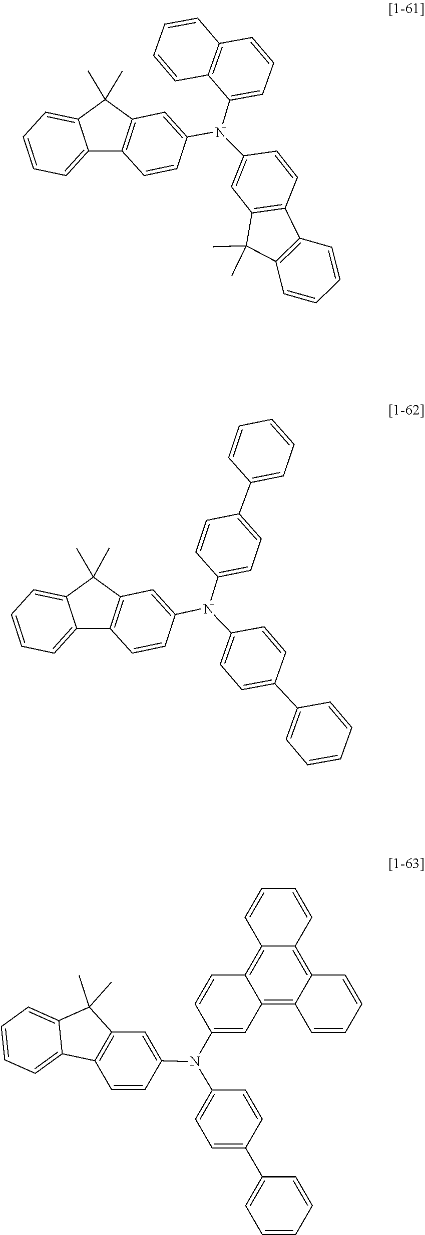 [SMILES: CC1(C)c2ccccc2-c2ccc(N(c3ccc4c(c3)C(C)(C)c3ccccc3-4)c3cccc4ccccc34)cc21 ; CC1(C)c2ccccc2-c2ccc(N(c3ccc(-c4ccccc4)cc3)c3ccc(-c4ccccc4)cc3)cc21 ; CC1(C)c2ccccc2-c2ccc(N(c3ccc(-c4ccccc4)cc3)c3ccc4c5ccccc5c5ccccc5c4c3)cc21]

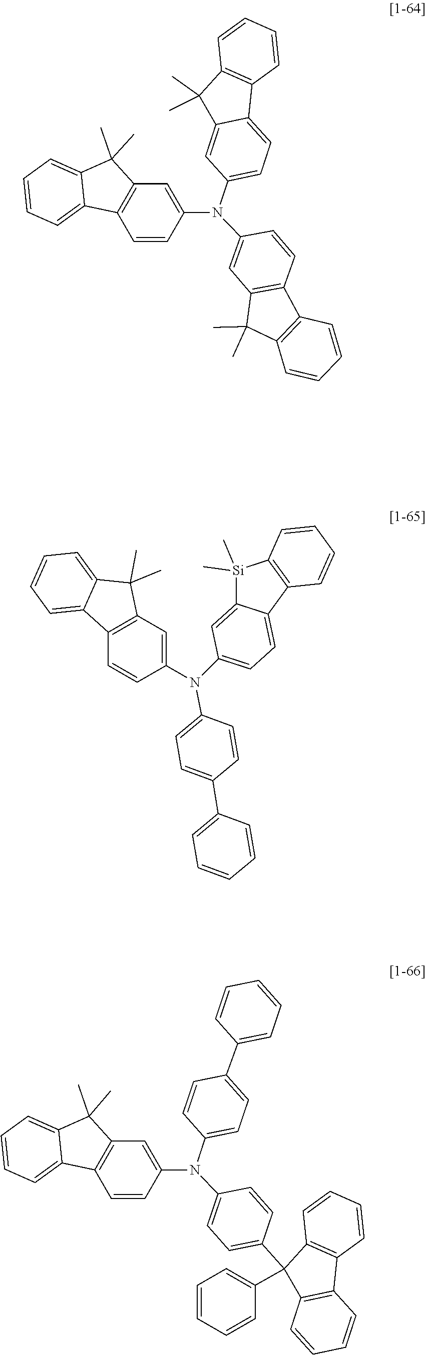 [SMILES: CC1(C)c2ccccc2-c2ccc(N(c3ccc4c(c3)C(C)(C)c3ccccc3-4)c3ccc4c(c3)C(C)(C)c3ccccc3-4)cc21 ; CC1(C)c2ccccc2-c2ccc(N(c3ccc(-c4ccccc4)cc3)c3ccc4c(c3)[Si](C)(C)c3ccccc3-4)cc21 ; CC1(C)c2ccccc2-c2ccc(N(c3ccc(-c4ccccc4)cc3)c3ccc(C4(c5ccccc5)c5ccccc5-c5ccccc54)cc3)cc21]

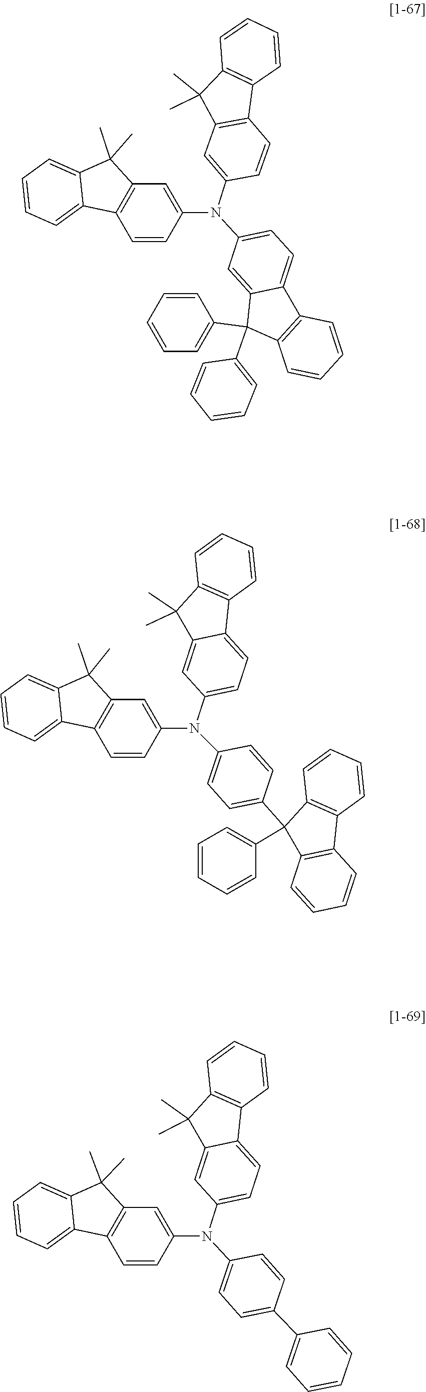 [SMILES: CC1(C)c2ccccc2-c2ccc(N(c3ccc4c(c3)C(C)(C)c3ccccc3-4)c3ccc4c(c3)C(c3ccccc3)(c3ccccc3)c3ccccc3-4)cc21 ; CC1(C)c2ccccc2-c2ccc(N(c3ccc(C4(c5ccccc5)c5ccccc5-c5ccccc54)cc3)c3ccc4c(c3)C(C)(C)c3ccccc3-4)cc21 ; CC1(C)c2ccccc2-c2ccc(N(c3ccc(-c4ccccc4)cc3)c3ccc4c(c3)C(C)(C)c3ccccc3-4)cc21]

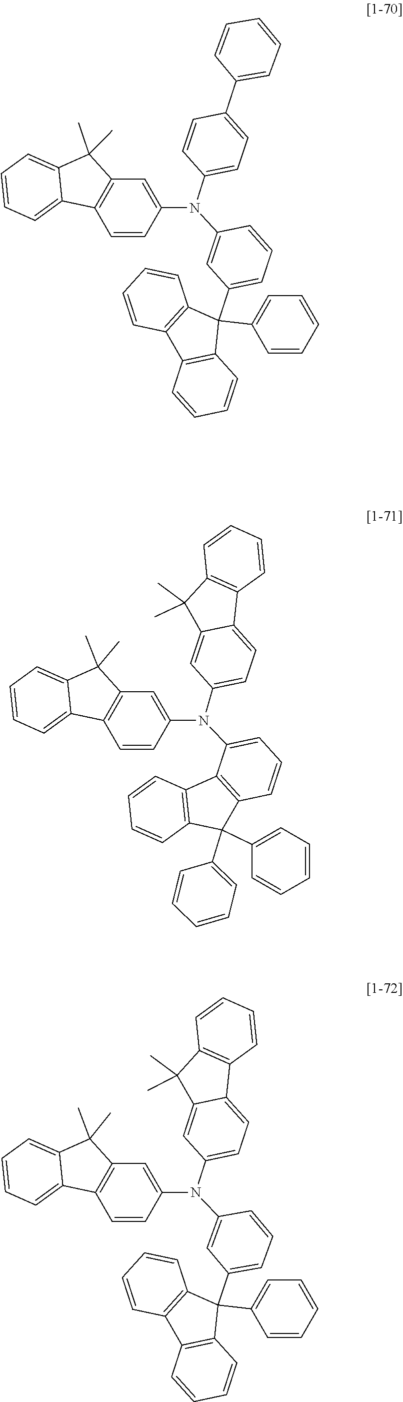 [SMILES: CC1(C)c2ccccc2-c2ccc(N(c3ccc(-c4ccccc4)cc3)c3cccc(C4(c5ccccc5)c5ccccc5-c5ccccc54)c3)cc21 ; CC1(C)c2ccccc2-c2ccc(N(c3ccc4c(c3)C(C)(C)c3ccccc3-4)c3cccc4c3-c3ccccc3C4(c3ccccc3)c3ccccc3)cc21 ; CC1(C)c2ccccc2-c2ccc(N(c3cccc(C4(c5ccccc5)c5ccccc5-c5ccccc54)c3)c3ccc4c(c3)C(C)(C)c3ccccc3-4)cc21]

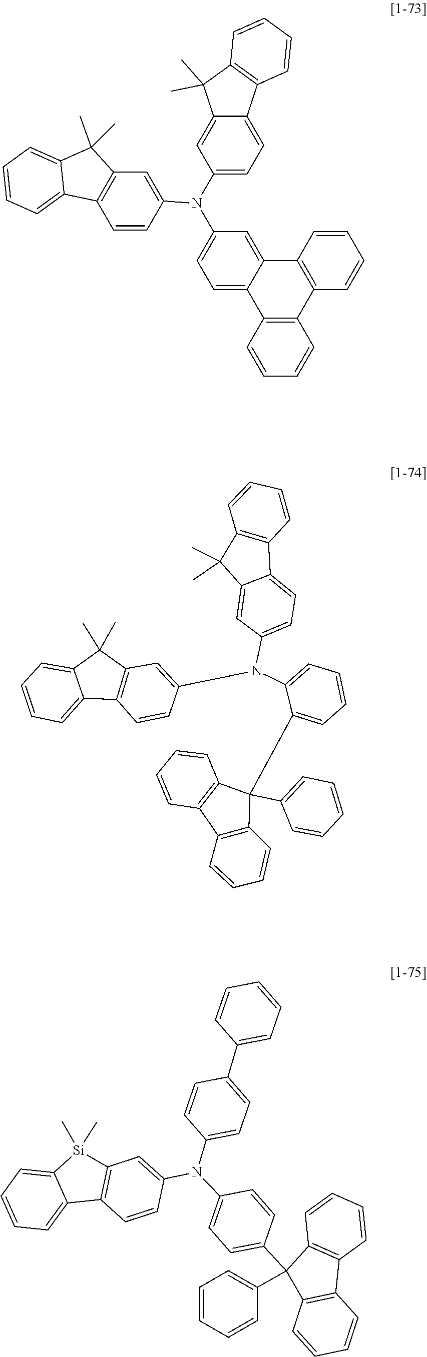 [SMILES: CC1(C)c2ccccc2-c2ccc(N(c3ccc4c(c3)C(C)(C)c3ccccc3-4)c3ccc4c5ccccc5c5ccccc5c4c3)cc21 ; CC1(C)c2ccccc2-c2ccc(N(c3ccc4c(c3)C(C)(C)c3ccccc3-4)c3ccccc3C3(c4ccccc4)c4ccccc4-c4ccccc43)cc21 ; C[Si]1(C)c2ccccc2-c2ccc(N(c3ccc(-c4ccccc4)cc3)c3ccc(C4(c5ccccc5)c5ccccc5-c5ccccc54)cc3)cc21]

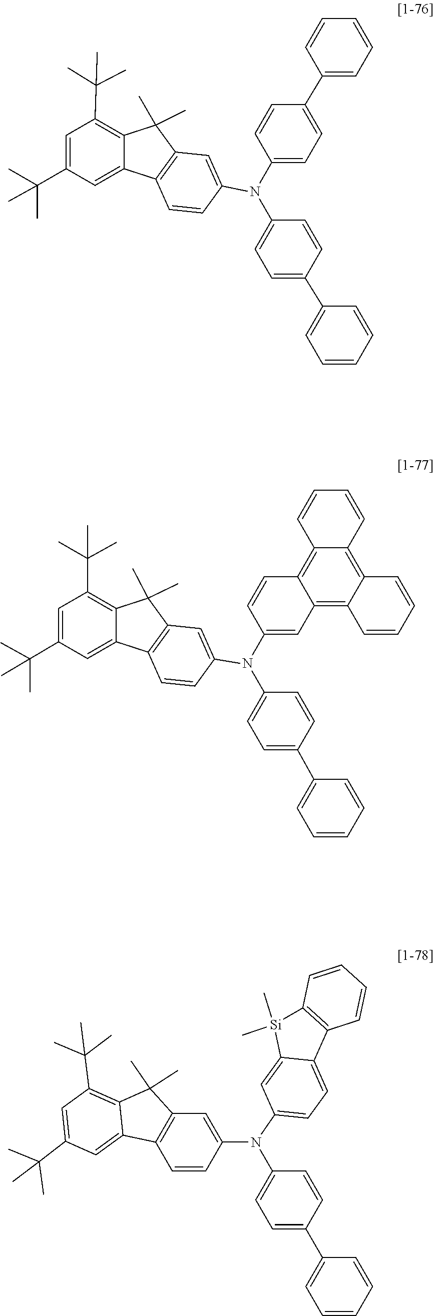 [SMILES: CC(C)(C)c1cc2c(c(C(C)(C)C)c1)C(C)(C)c1cc(N(c3ccc(-c4ccccc4)cc3)c3ccc(-c4ccccc4)cc3)ccc1-2 ; CC(C)(C)c1cc2c(c(C(C)(C)C)c1)C(C)(C)c1cc(N(c3ccc(-c4ccccc4)cc3)c3ccc4c5ccccc5c5ccccc5c4c3)ccc1-2 ; CC(C)(C)c1cc2c(c(C(C)(C)C)c1)C(C)(C)c1cc(N(c3ccc(-c4ccccc4)cc3)c3ccc4c(c3)[Si](C)(C)c3ccccc3-4)ccc1-2]

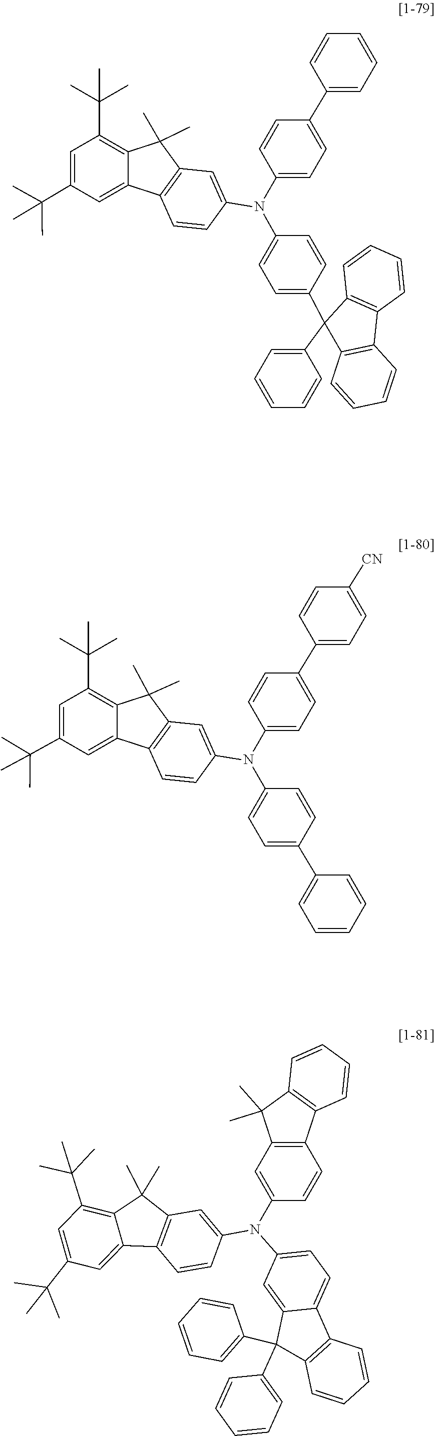 [SMILES: CC(C)(C)c1cc2c(c(C(C)(C)C)c1)C(C)(C)c1cc(N(c3ccc(-c4ccccc4)cc3)c3ccc(C4(c5ccccc5)c5ccccc5-c5ccccc54)cc3)ccc1-2 ; CC(C)(C)c1cc2c(c(C(C)(C)C)c1)C(C)(C)c1cc(N(c3ccc(-c4ccccc4)cc3)c3ccc(-c4ccc(C#N)cc4)cc3)ccc1-2 ; CC(C)(C)c1cc2c(c(C(C)(C)C)c1)C(C)(C)c1cc(N(c3ccc4c(c3)C(C)(C)c3ccccc3-4)c3ccc4c(c3)C(c3ccccc3)(c3ccccc3)c3ccccc3-4)ccc1-2]

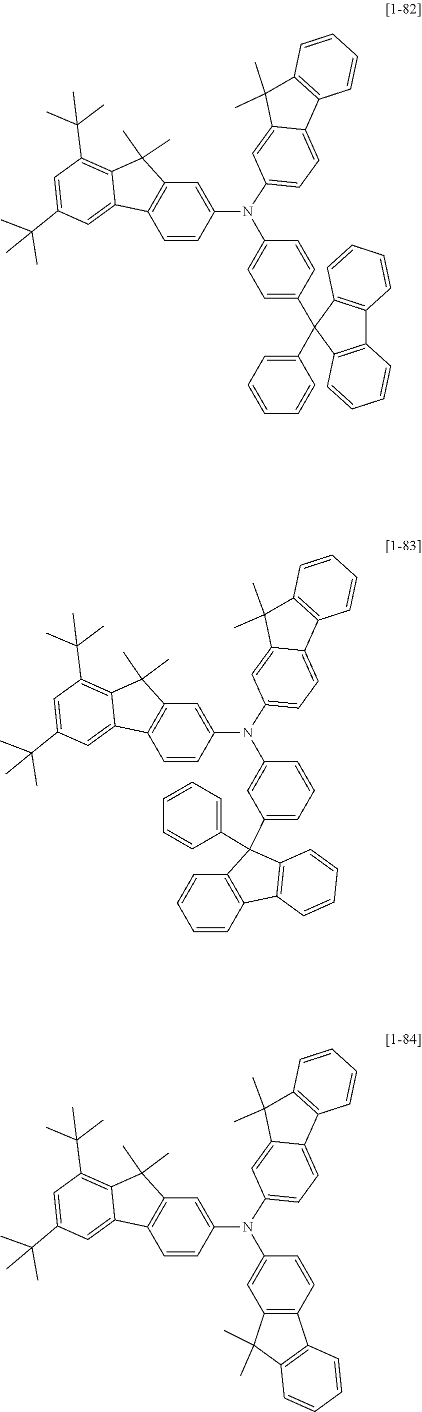 [SMILES: CC(C)(C)c1cc2c(c(C(C)(C)C)c1)C(C)(C)c1cc(N(c3ccc(C4(c5ccccc5)c5ccccc5-c5ccccc54)cc3)c3ccc4c(c3)C(C)(C)c3ccccc3-4)ccc1-2 ; CC(C)(C)c1cc2c(c(C(C)(C)C)c1)C(C)(C)c1cc(N(c3cccc(C4(c5ccccc5)c5ccccc5-c5ccccc54)c3)c3ccc4c(c3)C(C)(C)c3ccccc3-4)ccc1-2 ; CC(C)(C)c1cc2c(c(C(C)(C)C)c1)C(C)(C)c1cc(N(c3ccc4c(c3)C(C)(C)c3ccccc3-4)c3ccc4c(c3)C(C)(C)c3ccccc3-4)ccc1-2]

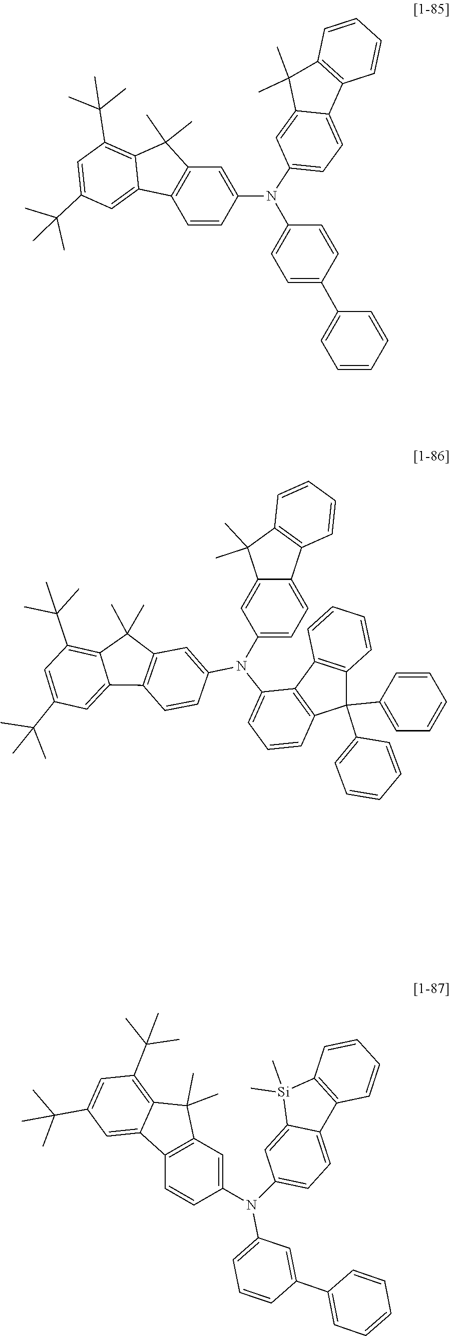 [SMILES: CC(C)(C)c1cc2c(c(C(C)(C)C)c1)C(C)(C)c1cc(N(c3ccc(-c4ccccc4)cc3)c3ccc4c(c3)C(C)(C)c3ccccc3-4)ccc1-2 ; CC(C)(C)c1cc2c(c(C(C)(C)C)c1)C(C)(C)c1cc(N(c3ccc4c(c3)C(C)(C)c3ccccc3-4)c3cccc4c3-c3ccccc3C4(c3ccccc3)c3ccccc3)ccc1-2 ; CC(C)(C)c1cc2c(c(C(C)(C)C)c1)C(C)(C)c1cc(N(c3cccc(-c4ccccc4)c3)c3ccc4c(c3)[Si](C)(C)c3ccccc3-4)ccc1-2]

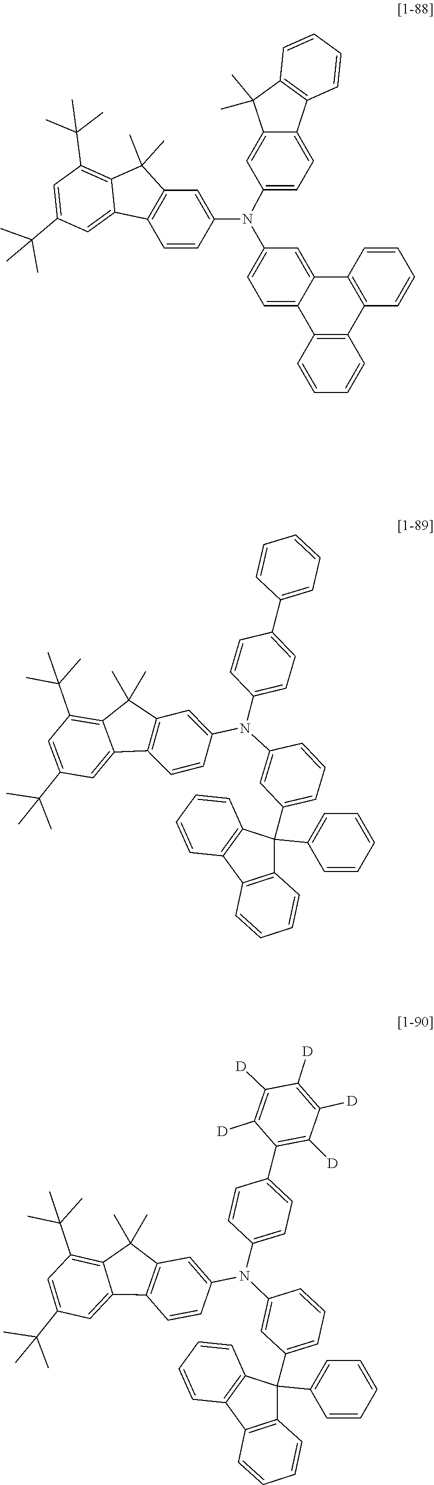 [SMILES: CC(C)(C)c1cc2c(c(C(C)(C)C)c1)C(C)(C)c1cc(N(c3ccc4c(c3)C(C)(C)c3ccccc3-4)c3ccc4c5ccccc5c5ccccc5c4c3)ccc1-2 ; CC(C)(C)c1cc2c(c(C(C)(C)C)c1)C(C)(C)c1cc(N(c3ccc(-c4ccccc4)cc3)c3cccc(C4(c5ccccc5)c5ccccc5-c5ccccc54)c3)ccc1-2 ; [2H]c1c([2H])c([2H])c(-c2ccc(N(c3cccc(C4(c5ccccc5)c5ccccc5-c5ccccc54)c3)c3ccc4c(c3)C(C)(C)c3c-4cc(C(C)(C)C)cc3C(C)(C)C)cc2)c([2H])c1[2H]]

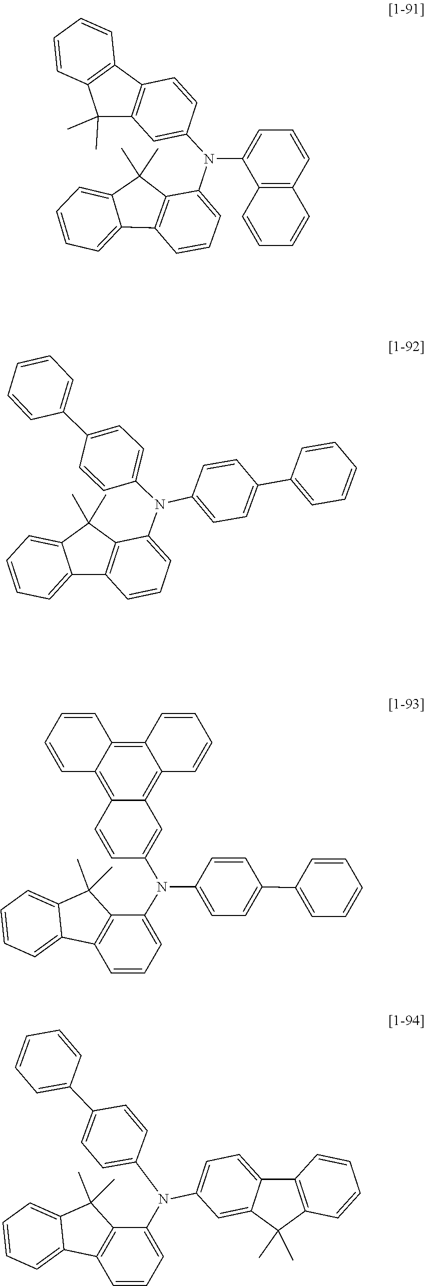 [SMILES: CC1(C)c2ccccc2-c2ccc(N(c3cccc4c3C(C)(C)c3ccccc3-4)c3cccc4ccccc34)cc21 ; CC1(C)c2ccccc2-c2cccc(N(c3ccc(-c4ccccc4)cc3)c3ccc(-c4ccccc4)cc3)c21 ; CC1(C)c2ccccc2-c2cccc(N(c3ccc(-c4ccccc4)cc3)c3ccc4c5ccccc5c5ccccc5c4c3)c21 ; CC1(C)c2ccccc2-c2ccc(N(c3ccc(-c4ccccc4)cc3)c3cccc4c3C(C)(C)c3ccccc3-4)cc21]

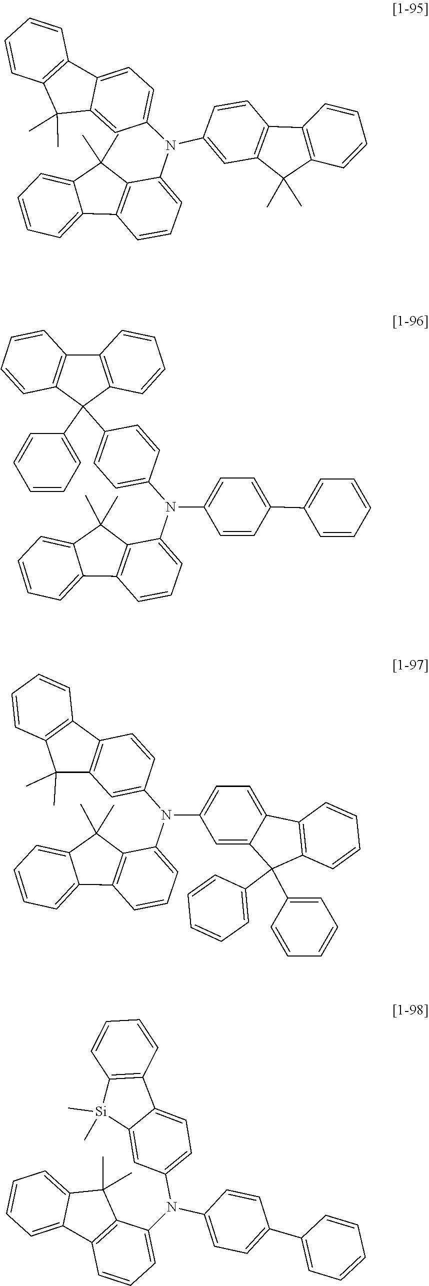 [SMILES: CC1(C)c2ccccc2-c2ccc(N(c3ccc4c(c3)C(C)(C)c3ccccc3-4)c3cccc4c3C(C)(C)c3ccccc3-4)cc21 ; CC1(C)c2ccccc2-c2cccc(N(c3ccc(-c4ccccc4)cc3)c3ccc(C4(c5ccccc5)c5ccccc5-c5ccccc54)cc3)c21 ; CC1(C)c2ccccc2-c2ccc(N(c3ccc4c(c3)C(c3ccccc3)(c3ccccc3)c3ccccc3-4)c3cccc4c3C(C)(C)c3ccccc3-4)cc21 ; CC1(C)c2ccccc2-c2cccc(N(c3ccc(-c4ccccc4)cc3)c3ccc4c(c3)[Si](C)(C)c3ccccc3-4)c21]

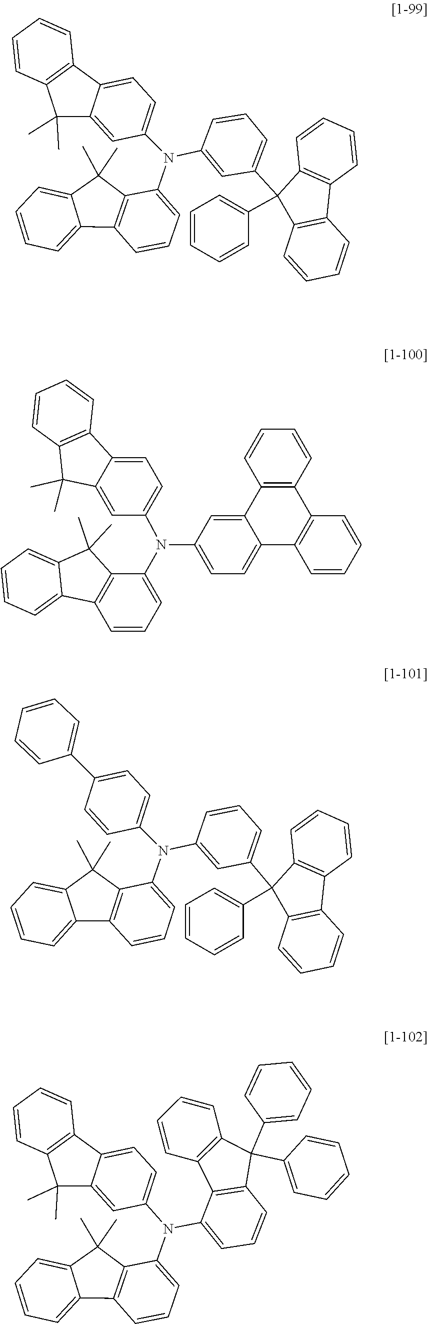 [SMILES: CC1(C)c2ccccc2-c2ccc(N(c3cccc(C4(c5ccccc5)c5ccccc5-c5ccccc54)c3)c3cccc4c3C(C)(C)c3ccccc3-4)cc21 ; CC1(C)c2ccccc2-c2ccc(N(c3ccc4c5ccccc5c5ccccc5c4c3)c3cccc4c3C(C)(C)c3ccccc3-4)cc21 ; CC1(C)c2ccccc2-c2cccc(N(c3ccc(-c4ccccc4)cc3)c3cccc(C4(c5ccccc5)c5ccccc5-c5ccccc54)c3)c21 ; CC1(C)c2ccccc2-c2ccc(N(c3cccc4c3-c3ccccc3C4(c3ccccc3)c3ccccc3)c3cccc4c3C(C)(C)c3ccccc3-4)cc21]

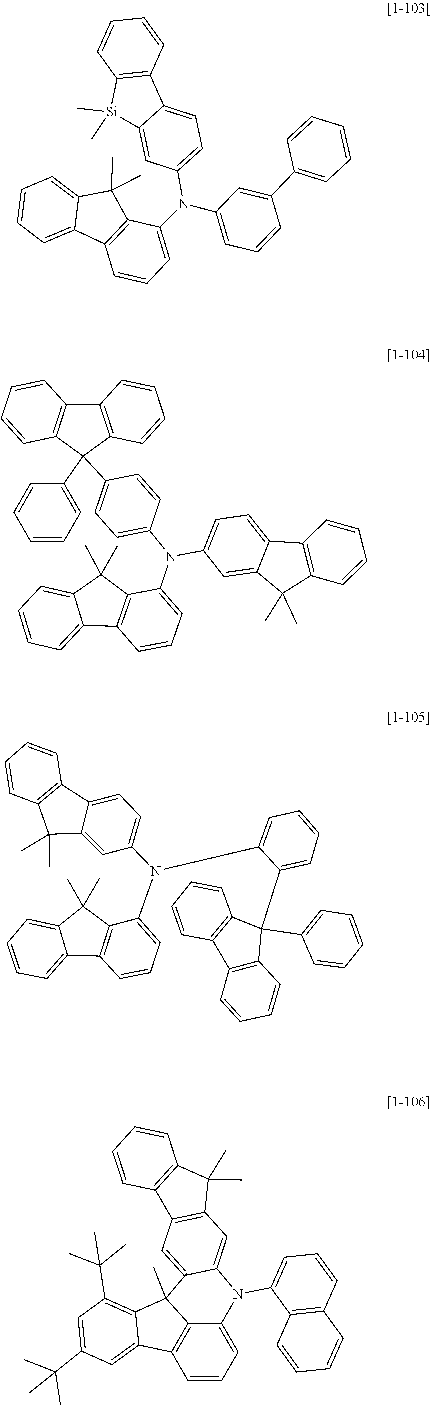

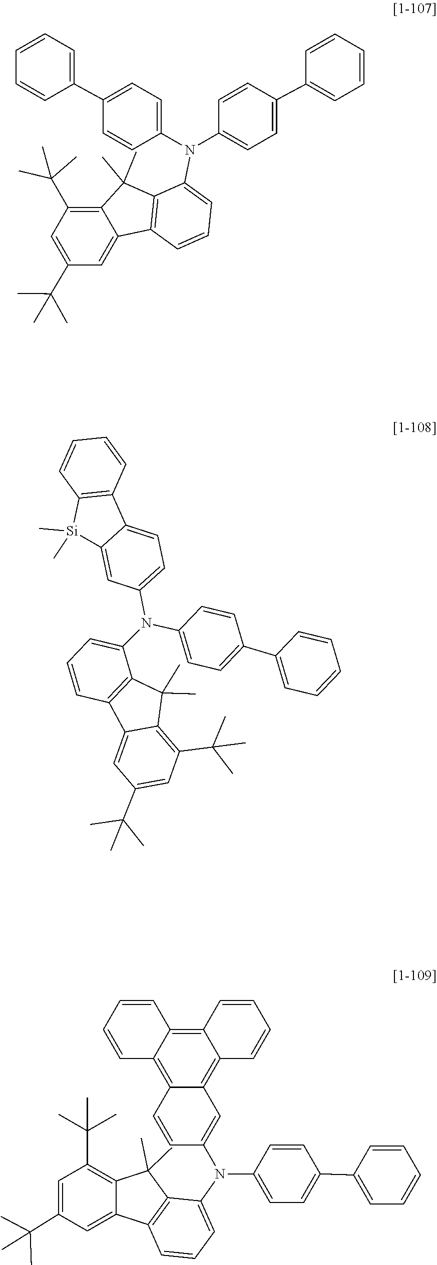 [SMILES: CC(C)(C)c1cc2c(c(C(C)(C)C)c1)C(C)(C)c1c-2cccc1N(c1ccc(-c2ccccc2)cc1)c1ccc(-c2ccccc2)cc1 ; CC(C)(C)c1cc2c(c(C(C)(C)C)c1)C(C)(C)c1c-2cccc1N(c1ccc(-c2ccccc2)cc1)c1ccc2c(c1)[Si](C)(C)c1ccccc1-2 ; CC(C)(C)c1cc2c(c(C(C)(C)C)c1)C(C)(C)c1c-2cccc1N(c1ccc(-c2ccccc2)cc1)c1ccc2c3ccccc3c3ccccc3c2c1]

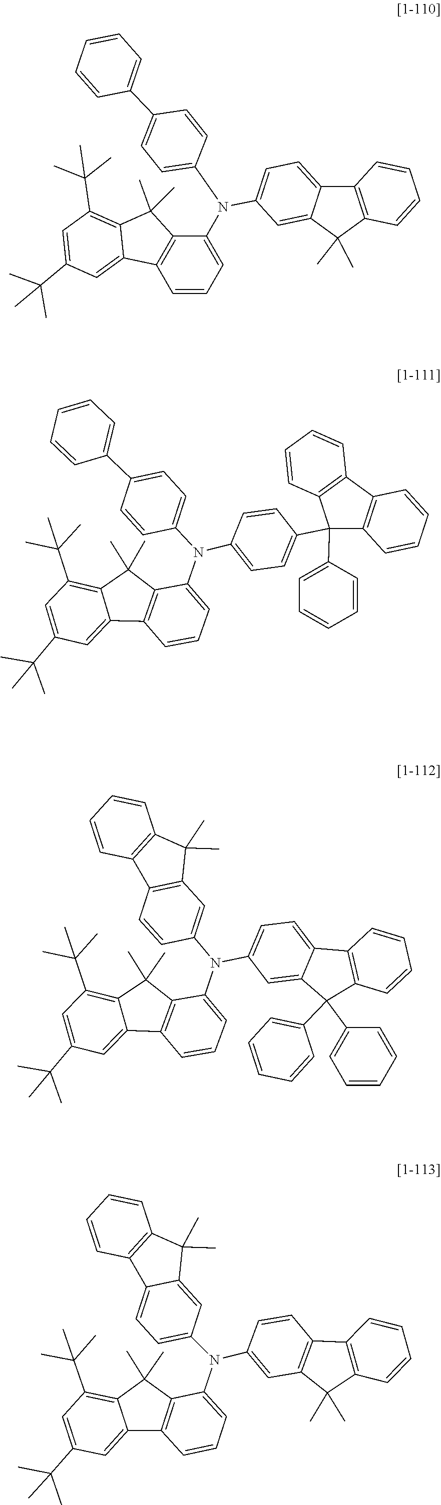 [SMILES: CC(C)(C)c1cc2c(c(C(C)(C)C)c1)C(C)(C)c1c-2cccc1N(c1ccc(-c2ccccc2)cc1)c1ccc2c(c1)C(C)(C)c1ccccc1-2 ; CC(C)(C)c1cc2c(c(C(C)(C)C)c1)C(C)(C)c1c-2cccc1N(c1ccc(-c2ccccc2)cc1)c1ccc(C2(c3ccccc3)c3ccccc3-c3ccccc32)cc1 ; CC(C)(C)c1cc2c(c(C(C)(C)C)c1)C(C)(C)c1c-2cccc1N(c1ccc2c(c1)C(C)(C)c1ccccc1-2)c1ccc2c(c1)C(c1ccccc1)(c1ccccc1)c1ccccc1-2 ; CC(C)(C)c1cc2c(c(C(C)(C)C)c1)C(C)(C)c1c-2cccc1N(c1ccc2c(c1)C(C)(C)c1ccccc1-2)c1ccc2c(c1)C(C)(C)c1ccccc1-2]

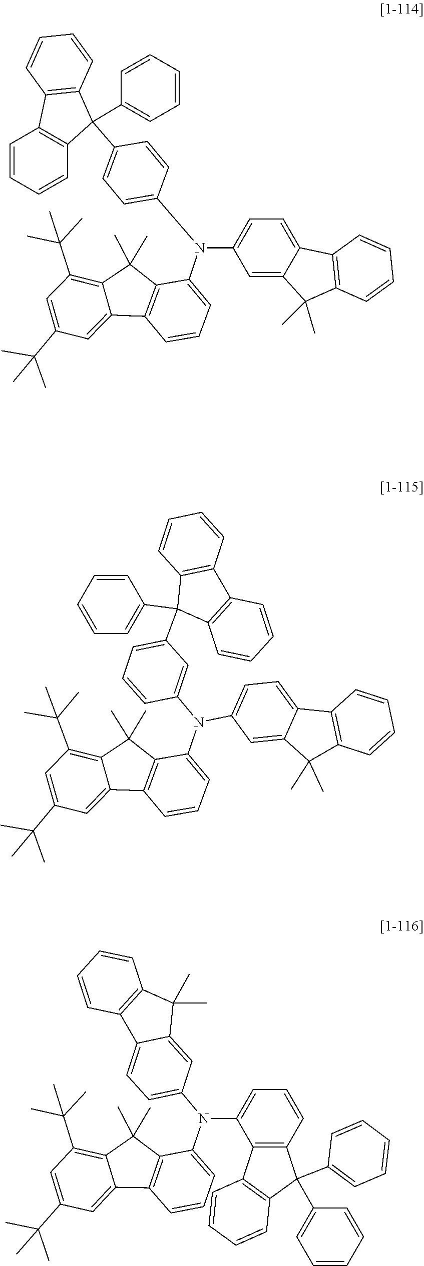 [SMILES: CC(C)(C)c1cc2c(c(C(C)(C)C)c1)C(C)(C)c1c-2cccc1N(c1ccc(C2(c3ccccc3)c3ccccc3-c3ccccc32)cc1)c1ccc2c(c1)C(C)(C)c1ccccc1-2 ; CC(C)(C)c1cc2c(c(C(C)(C)C)c1)C(C)(C)c1c-2cccc1N(c1cccc(C2(c3ccccc3)c3ccccc3-c3ccccc32)c1)c1ccc2c(c1)C(C)(C)c1ccccc1-2 ; CC(C)(C)c1cc2c(c(C(C)(C)C)c1)C(C)(C)c1c-2cccc1N(c1ccc2c(c1)C(C)(C)c1ccccc1-2)c1cccc2c1-c1ccccc1C2(c1ccccc1)c1ccccc1]

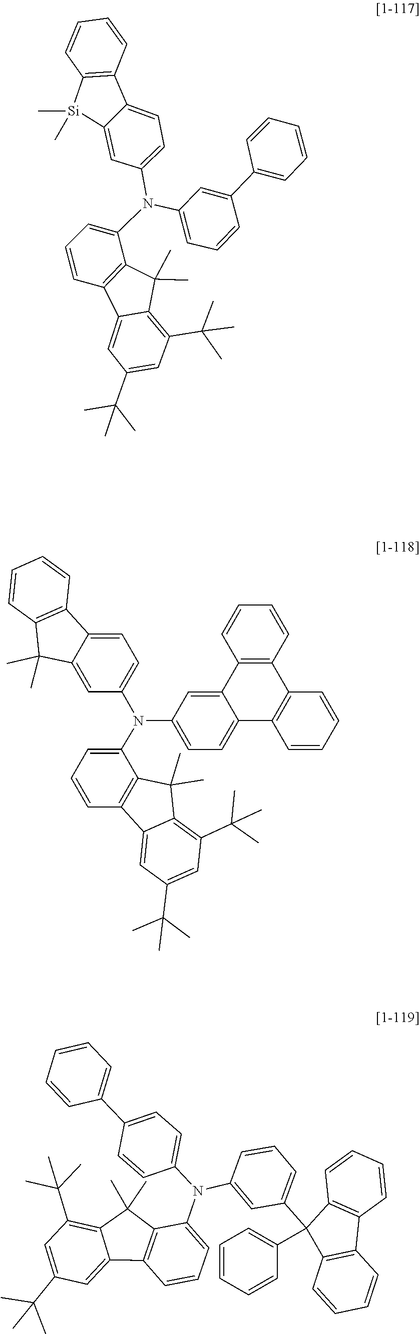 [SMILES: CC(C)(C)c1cc2c(c(C(C)(C)C)c1)C(C)(C)c1c-2cccc1N(c1cccc(-c2ccccc2)c1)c1ccc2c(c1)[Si](C)(C)c1ccccc1-2 ; CC(C)(C)c1cc2c(c(C(C)(C)C)c1)C(C)(C)c1c-2cccc1N(c1ccc2c(c1)C(C)(C)c1ccccc1-2)c1ccc2c3ccccc3c3ccccc3c2c1 ; CC(C)(C)c1cc2c(c(C(C)(C)C)c1)C(C)(C)c1c-2cccc1N(c1ccc(-c2ccccc2)cc1)c1cccc(C2(c3ccccc3)c3ccccc3-c3ccccc32)c1]

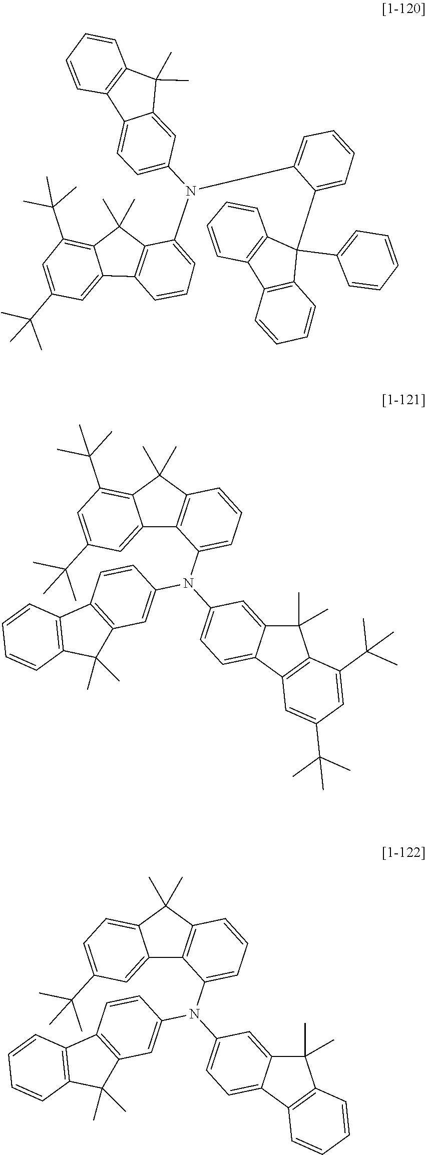 [SMILES: CC(C)(C)c1cc2c(c(C(C)(C)C)c1)C(C)(C)c1c-2cccc1N(c1ccc2c(c1)C(C)(C)c1ccccc1-2)c1ccccc1C1(c2ccccc2)c2ccccc2-c2ccccc21 ; CC(C)(C)c1cc2c(c(C(C)(C)C)c1)C(C)(C)c1cc(N(c3ccc4c(c3)C(C)(C)c3ccccc3-4)c3cccc4c3-c3cc(C(C)(C)C)cc(C(C)(C)C)c3C4(C)C)ccc1-2 ; CC(C)(C)c1ccc2c(c1)-c1c(N(c3ccc4c(c3)C(C)(C)c3ccccc3-4)c3ccc4c(c3)C(C)(C)c3ccccc3-4)cccc1C2(C)C]

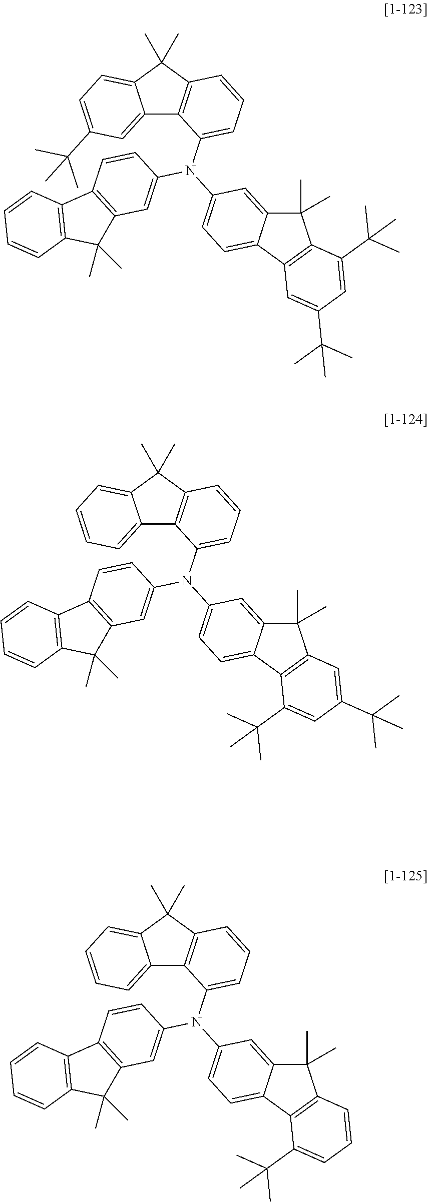 [SMILES: CC(C)(C)c1ccc2c(c1)-c1c(N(c3ccc4c(c3)C(C)(C)c3ccccc3-4)c3ccc4c(c3)C(C)(C)c3c-4cc(C(C)(C)C)cc3C(C)(C)C)cccc1C2(C)C ; CC(C)(C)c1cc(C(C)(C)C)c2c(c1)C(C)(C)c1cc(N(c3ccc4c(c3)C(C)(C)c3ccccc3-4)c3cccc4c3-c3ccccc3C4(C)C)ccc1-2 ; CC(C)(C)c1cccc2c1-c1ccc(N(c3ccc4c(c3)C(C)(C)c3ccccc3-4)c3cccc4c3-c3ccccc3C4(C)C)cc1C2(C)C]

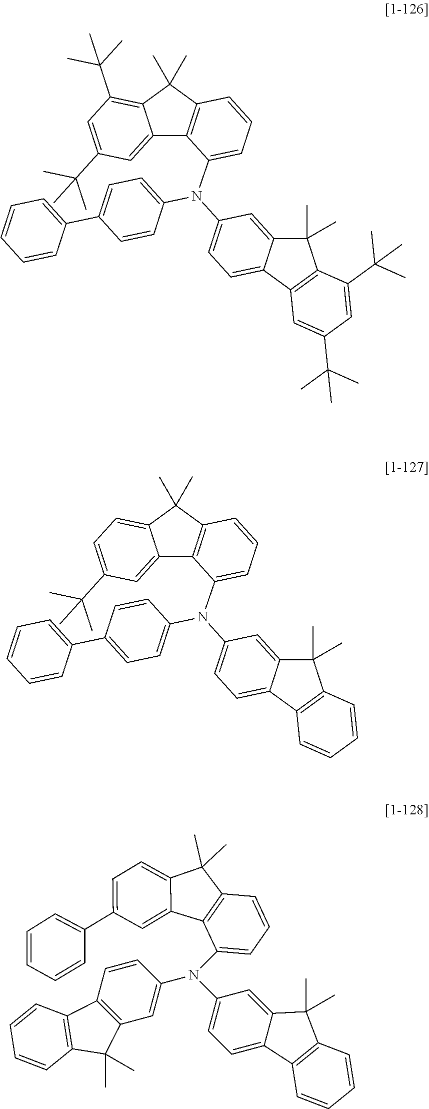 [SMILES: CC(C)(C)c1cc2c(c(C(C)(C)C)c1)C(C)(C)c1cc(N(c3ccc(-c4ccccc4)cc3)c3cccc4c3-c3cc(C(C)(C)C)cc(C(C)(C)C)c3C4(C)C)ccc1-2 ; CC(C)(C)c1ccc2c(c1)-c1c(N(c3ccc(-c4ccccc4)cc3)c3ccc4c(c3)C(C)(C)c3ccccc3-4)cccc1C2(C)C ; CC1(C)c2ccccc2-c2ccc(N(c3ccc4c(c3)C(C)(C)c3ccccc3-4)c3cccc4c3-c3cc(-c5ccccc5)ccc3C4(C)C)cc21]

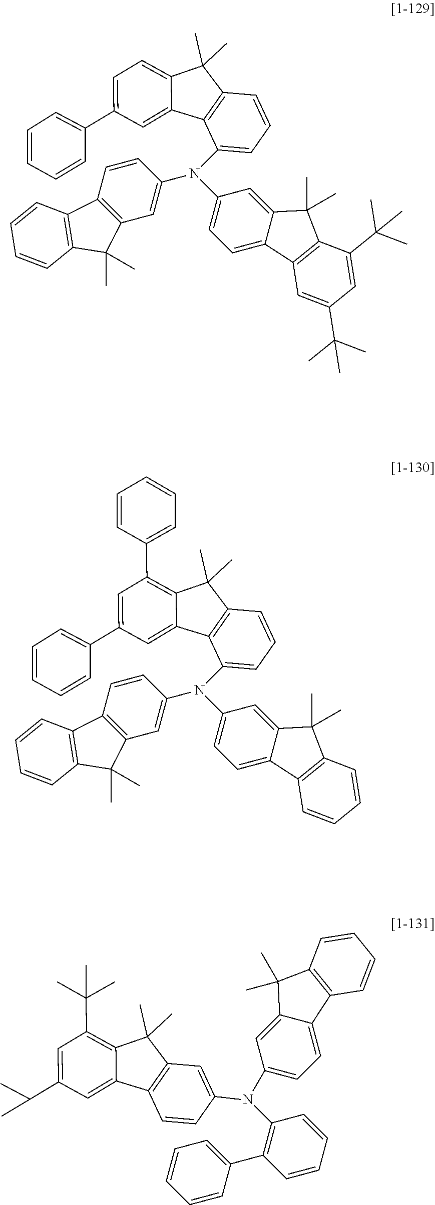

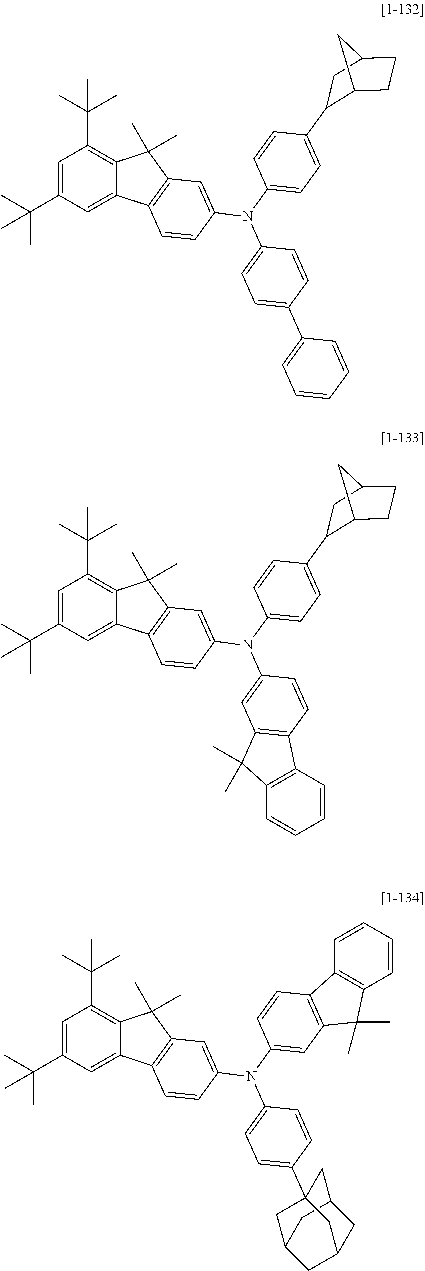 [SMILES: CC(C)(C)c1cc2c(c(C(C)(C)C)c1)C(C)(C)c1cc(N(c3ccc(-c4ccccc4)cc3)c3ccc(C4CC5CCC4C5)cc3)ccc1-2 ; CC(C)(C)c1cc2c(c(C(C)(C)C)c1)C(C)(C)c1cc(N(c3ccc(C4CC5CCC4C5)cc3)c3ccc4c(c3)C(C)(C)c3ccccc3-4)ccc1-2 ; CC(C)(C)c1cc2c(c(C(C)(C)C)c1)C(C)(C)c1cc(N(c3ccc(C45CC6CC(CC(C6)C4)C5)cc3)c3ccc4c(c3)C(C)(C)c3ccccc3-4)ccc1-2]

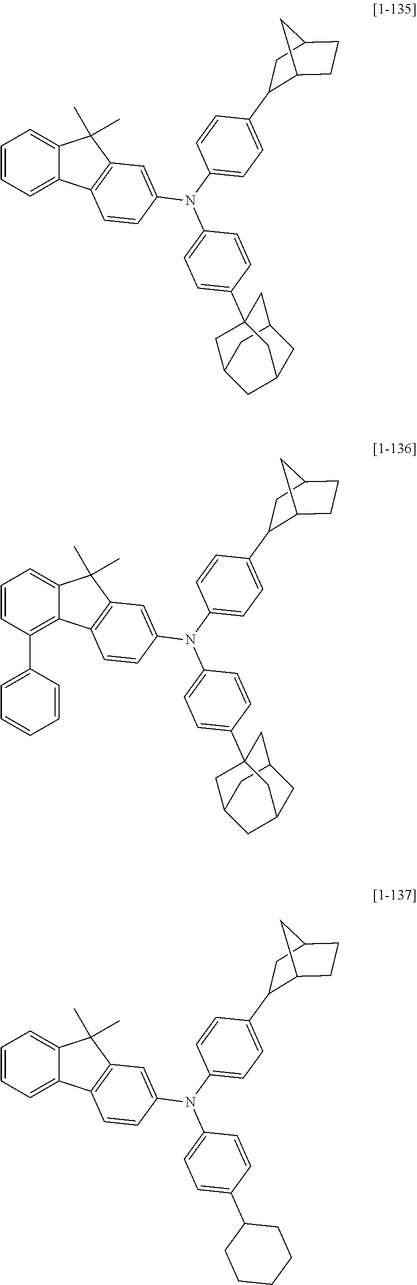 [SMILES: CC1(C)c2ccccc2-c2ccc(N(c3ccc(C4CC5CCC4C5)cc3)c3ccc(C45CC6CC(CC(C6)C4)C5)cc3)cc21 ; CC1(C)c2cc(N(c3ccc(C4CC5CCC4C5)cc3)c3ccc(C45CC6CC(CC(C6)C4)C5)cc3)ccc2-c2c(-c3ccccc3)cccc21 ; CC1(C)c2ccccc2-c2ccc(N(c3ccc(C4CCCCC4)cc3)c3ccc(C4CC5CCC4C5)cc3)cc21]

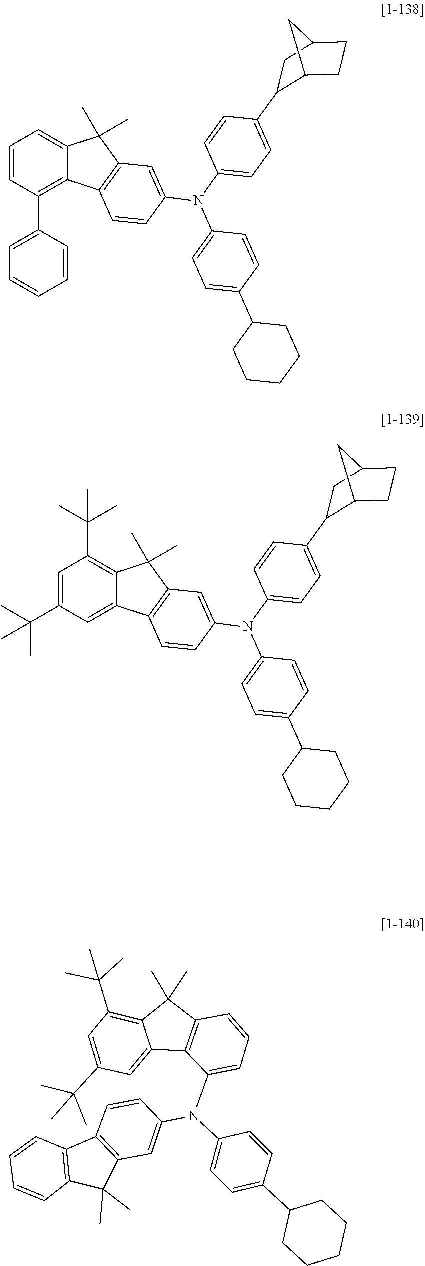 [SMILES: CC1(C)c2cc(N(c3ccc(C4CCCCC4)cc3)c3ccc(C4CC5CCC4C5)cc3)ccc2-c2c(-c3ccccc3)cccc21 ; CC(C)(C)c1cc2c(c(C(C)(C)C)c1)C(C)(C)c1cc(N(c3ccc(C4CCCCC4)cc3)c3ccc(C4CC5CCC4C5)cc3)ccc1-2 ; CC(C)(C)c1cc2c(c(C(C)(C)C)c1)C(C)(C)c1cccc(N(c3ccc(C4CCCCC4)cc3)c3ccc4c(c3)C(C)(C)c3ccccc3-4)c1-2]

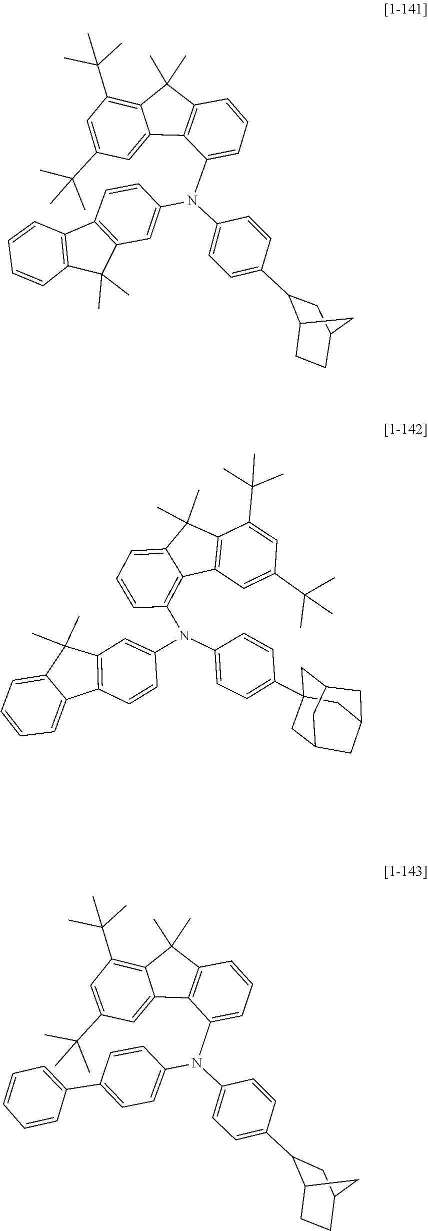 [SMILES: CC(C)(C)c1cc2c(c(C(C)(C)C)c1)C(C)(C)c1cccc(N(c3ccc(C4CC5CCC4C5)cc3)c3ccc4c(c3)C(C)(C)c3ccccc3-4)c1-2 ; CC(C)(C)c1cc2c(c(C(C)(C)C)c1)C(C)(C)c1cccc(N(c3ccc(C45CC6CC(CC(C6)C4)C5)cc3)c3ccc4c(c3)C(C)(C)c3ccccc3-4)c1-2 ; CC(C)(C)c1cc2c(c(C(C)(C)C)c1)C(C)(C)c1cccc(N(c3ccc(-c4ccccc4)cc3)c3ccc(C4CC5CCC4C5)cc3)c1-2]

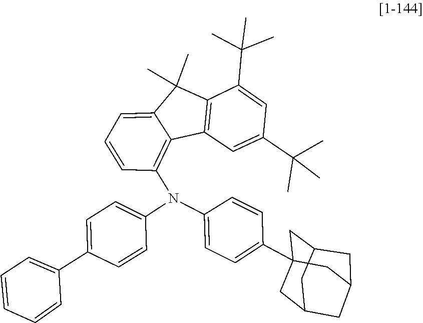 [SMILES: CC(C)(C)c1cc2c(c(C(C)(C)C)c1)C(C)(C)c1cccc(N(c3ccc(-c4ccccc4)cc3)c3ccc(C45CC6CC(CC(C6)C4)C5)cc3)c1-2]

In an implementation, m3 may be, e.g., 2, 3, or 4 and each R³ may be the same or different from each other.

In an implementation, m4 may be, e.g., 2 or 3 and each R⁴ may be the same or different from each other.

In an implementation, Ar³ and Ar⁴ in Chemical Formula 2 may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or an unsubstituted anthracenyl group, a substituted or unsubstituted fluorenyl group, a substituted or unsubstituted spirofluorenyl group, a substituted or unsubstituted triphenylene group, a substituted or unsubstituted dibenzofuranyl group, a substituted or unsubstituted dibenzothiophenyl group, or a substituted or unsubstituted dibenzosilolyl group.

In an implementation, at least one of Ar³ and Ar⁴ may be, e.g., a substituted or unsubstituted 9-fluorenyl group.

In an implementation, Ar³ and Ar⁴ in Chemical Formula 2 may each independently be, e.g., a substituted or unsubstituted phenyl group, a substituted or unsubstituted biphenyl group, a substituted or unsubstituted fluorenyl group, a substituted or unsubstituted dibenzofuranyl group, or a substituted or unsubstituted dibenzothiophenyl group, and at least one of Ar³ and Ar⁴ may be a substituted or unsubstituted 9-fluorenyl group.

In an implementation, L⁴ to L⁶ may each independently be, e.g., a single bond, a substituted or unsubstituted phenylene group, or a substituted or unsubstituted biphenylene group.

In an implementation, L⁴ may be a single bond, and at least one of L⁵ and L⁶ may be, e.g., a substituted or unsubstituted phenylene group.

In an implementation, R³ and R⁴ may each independently be, e.g., hydrogen, deuterium, a substituted or unsubstituted C1 to C10 alkyl group, or a substituted or unsubstituted C6 to C12 aryl group.

In an implementation, moieties -L⁵-Ar³ and -L⁶-Ar⁴ may each independently be a moiety of Group II, and at least one of Ar³ and Ar⁴ may be a substituted or unsubstituted 9-fluorenyl group.

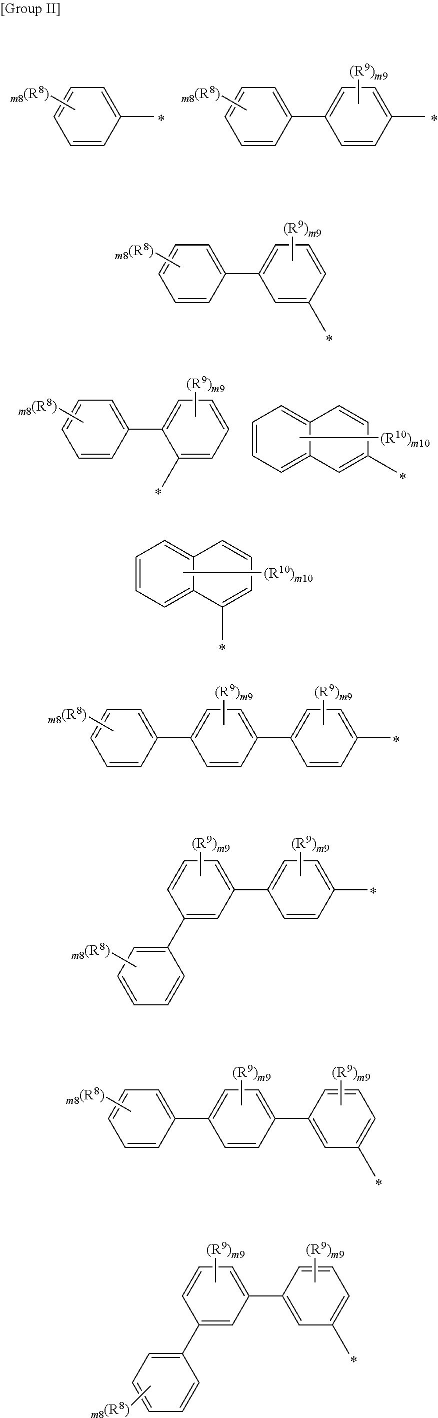

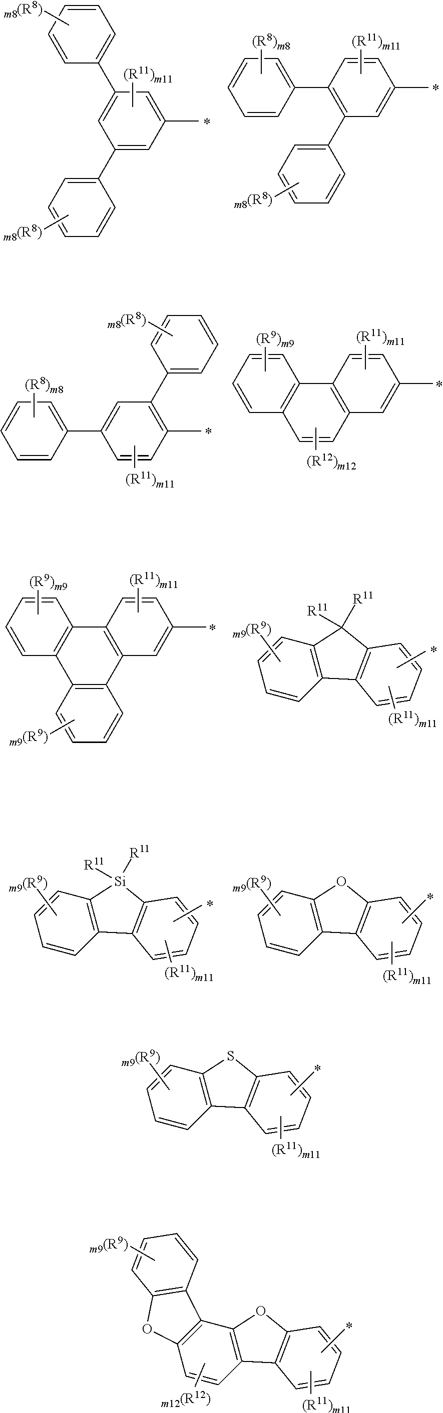

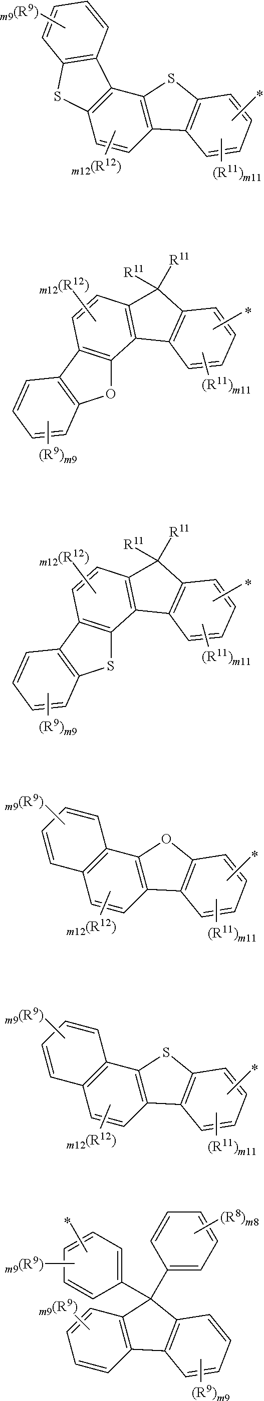

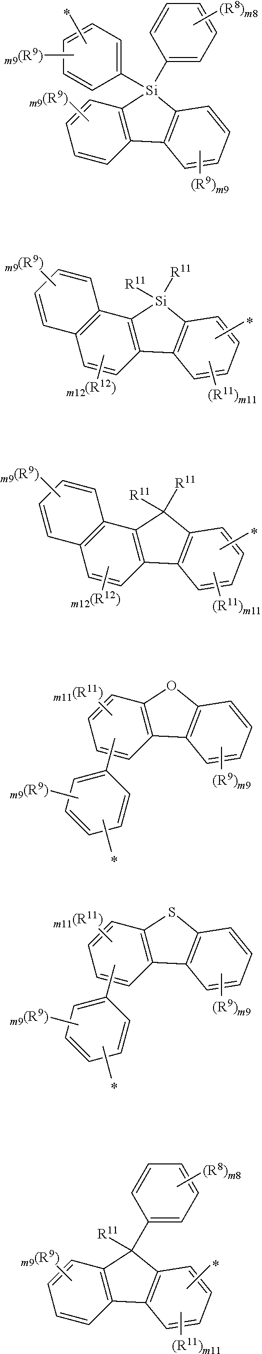

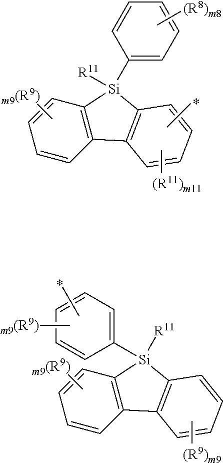

In Group II, R⁸ to R¹² may each independently be, e.g., hydrogen, deuterium, a cyano group, a substituted or unsubstituted C1 to C10 alkyl group, or a substituted or unsubstituted C6 to C12 aryl group.

m8 may be, e.g., an integer of 1 to 5.

m9 may be, e.g., an integer of 1 to 4.

m10 may be, e.g., an integer of 1 to 7.

m11 may be, e.g., an integer of 1 to 3.

m12 may be, e.g., an integer of 1 or 2.

* is a linking point.

In an implementation, at least one of Ar³ and Ar⁴ may be represented by Chemical Formula II-1.

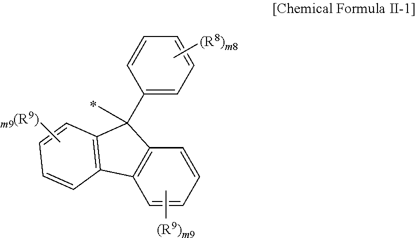

In Chemical Formula II-1, R⁸, R⁹, m8, and m9 may be defined the same as those described above.

In an implementation, the second compound may be, e.g., a compound of Group 2.

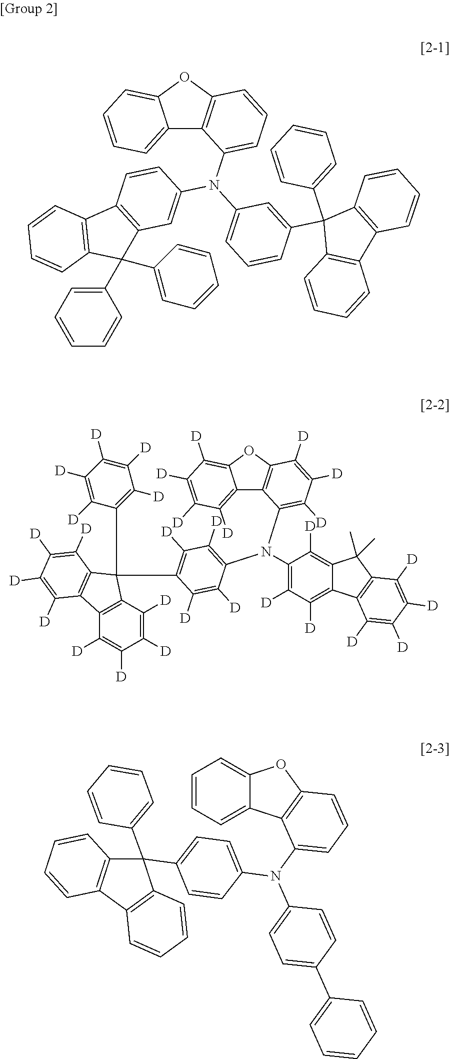 [SMILES: c1ccc(C2(c3cccc(N(c4ccc5c(c4)C(c4ccccc4)(c4ccccc4)c4ccccc4-5)c4cccc5oc6ccccc6c45)c3)c3ccccc3-c3ccccc32)cc1 ; [2H]c1c([2H])c([2H])c(C2(c3c([2H])c([2H])c(N(c4c([2H])c([2H])c5c(c4[2H])C(C)(C)c4c([2H])c([2H])c([2H])c([2H])c4-5)c4c([2H])c([2H])c([2H])c5oc6c([2H])c([2H])c([2H])c([2H])c6c45)c([2H])c3[2H])c3c([2H])c([2H])c([2H])c([2H])c3-c3c([2H])c([2H])c([2H])c([2H])c32)c([2H])c1[2H] ; c1ccc(-c2ccc(N(c3ccc(C4(c5ccccc5)c5ccccc5-c5ccccc54)cc3)c3cccc4oc5ccccc5c34)cc2)cc1]

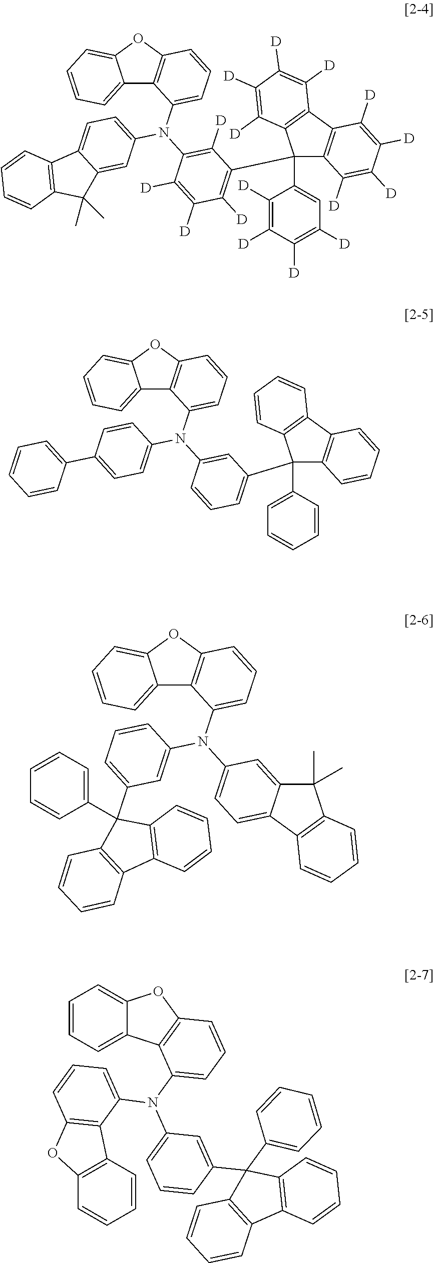 [SMILES: [2H]c1cc(C2(c3c([2H])c([2H])c([2H])c(N(c4ccc5c(c4)C(C)(C)c4ccccc4-5)c4cccc5oc6ccccc6c45)c3[2H])c3c([2H])c([2H])c([2H])c([2H])c3-c3c([2H])c([2H])c([2H])c([2H])c32)c([2H])c([2H])c1[2H] ; c1ccc(-c2ccc(N(c3cccc(C4(c5ccccc5)c5ccccc5-c5ccccc54)c3)c3cccc4oc5ccccc5c34)cc2)cc1 ; CC1(C)c2ccccc2-c2ccc(N(c3cccc(C4(c5ccccc5)c5ccccc5-c5ccccc54)c3)c3cccc4oc5ccccc5c34)cc21 ; c1ccc(C2(c3cccc(N(c4cccc5oc6ccccc6c45)c4cccc5oc6ccccc6c45)c3)c3ccccc3-c3ccccc32)cc1]

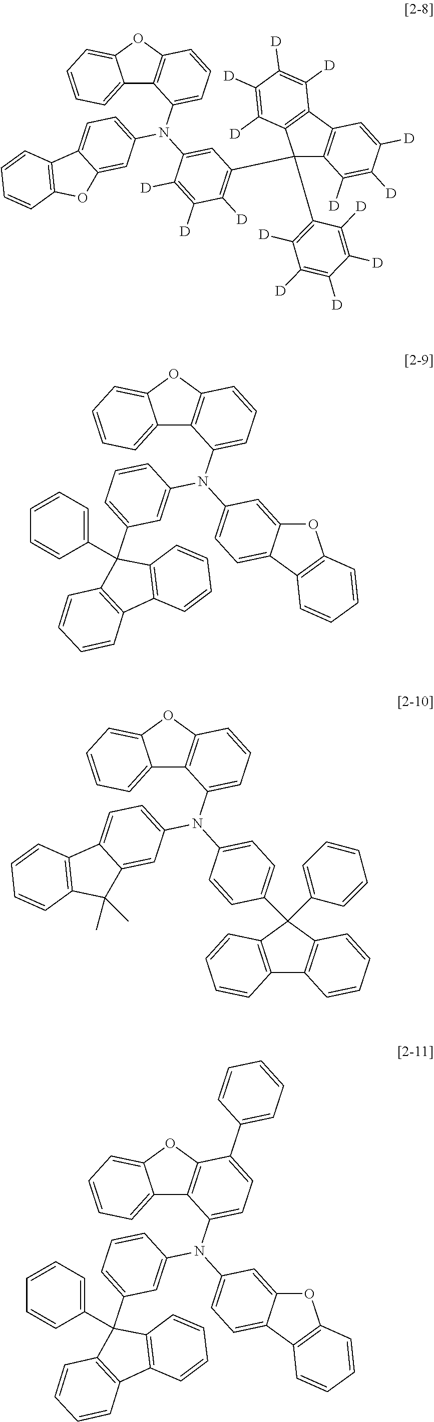 [SMILES: [2H]c1cc2c(c([2H])c1[2H])C(c1cc(N(c3ccc4c(c3)oc3ccccc34)c3cccc4oc5ccccc5c34)c([2H])c([2H])c1[2H])(c1c([2H])c([2H])c([2H])c([2H])c1[2H])c1c([2H])c([2H])c([2H])c([2H])c1-2 ; c1ccc(C2(c3cccc(N(c4ccc5c(c4)oc4ccccc45)c4cccc5oc6ccccc6c45)c3)c3ccccc3-c3ccccc32)cc1 ; CC1(C)c2ccccc2-c2ccc(N(c3ccc(C4(c5ccccc5)c5ccccc5-c5ccccc54)cc3)c3cccc4oc5ccccc5c34)cc21 ; c1ccc(-c2ccc(N(c3cccc(C4(c5ccccc5)c5ccccc5-c5ccccc54)c3)c3ccc4c(c3)oc3ccccc34)c3c2oc2ccccc23)cc1]

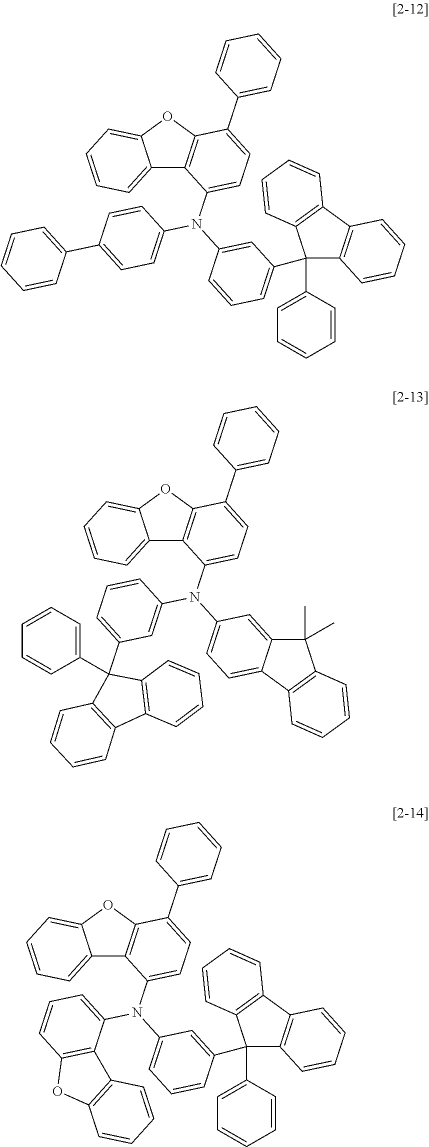 [SMILES: c1ccc(-c2ccc(N(c3cccc(C4(c5ccccc5)c5ccccc5-c5ccccc54)c3)c3ccc(-c4ccccc4)c4oc5ccccc5c34)cc2)cc1 ; CC1(C)c2ccccc2-c2ccc(N(c3cccc(C4(c5ccccc5)c5ccccc5-c5ccccc54)c3)c3ccc(-c4ccccc4)c4oc5ccccc5c34)cc21 ; c1ccc(-c2ccc(N(c3cccc(C4(c5ccccc5)c5ccccc5-c5ccccc54)c3)c3cccc4oc5ccccc5c34)c3c2oc2ccccc23)cc1]

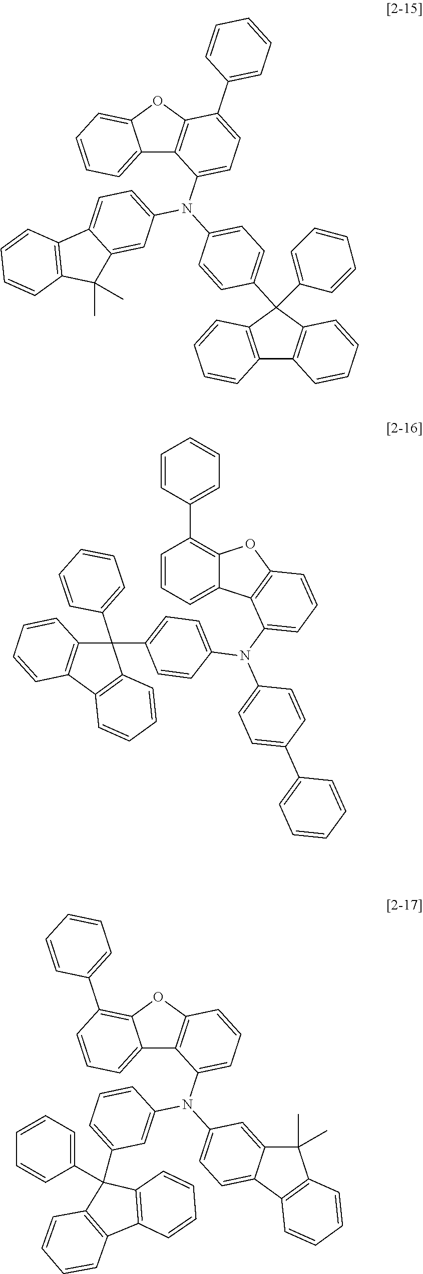 [SMILES: CC1(C)c2ccccc2-c2ccc(N(c3ccc(C4(c5ccccc5)c5ccccc5-c5ccccc54)cc3)c3ccc(-c4ccccc4)c4oc5ccccc5c34)cc21 ; c1ccc(-c2ccc(N(c3ccc(C4(c5ccccc5)c5ccccc5-c5ccccc54)cc3)c3cccc4oc5c(-c6ccccc6)cccc5c34)cc2)cc1 ; CC1(C)c2ccccc2-c2ccc(N(c3cccc(C4(c5ccccc5)c5ccccc5-c5ccccc54)c3)c3cccc4oc5c(-c6ccccc6)cccc5c34)cc21]

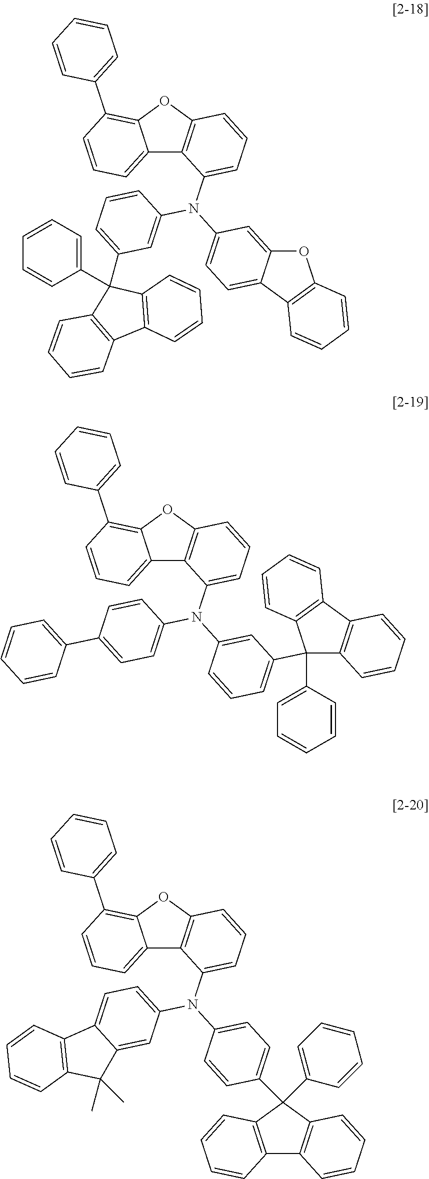 [SMILES: c1ccc(-c2cccc3c2oc2cccc(N(c4cccc(C5(c6ccccc6)c6ccccc6-c6ccccc65)c4)c4ccc5c(c4)oc4ccccc45)c23)cc1 ; c1ccc(-c2ccc(N(c3cccc(C4(c5ccccc5)c5ccccc5-c5ccccc54)c3)c3cccc4oc5c(-c6ccccc6)cccc5c34)cc2)cc1 ; CC1(C)c2ccccc2-c2ccc(N(c3ccc(C4(c5ccccc5)c5ccccc5-c5ccccc54)cc3)c3cccc4oc5c(-c6ccccc6)cccc5c34)cc21]

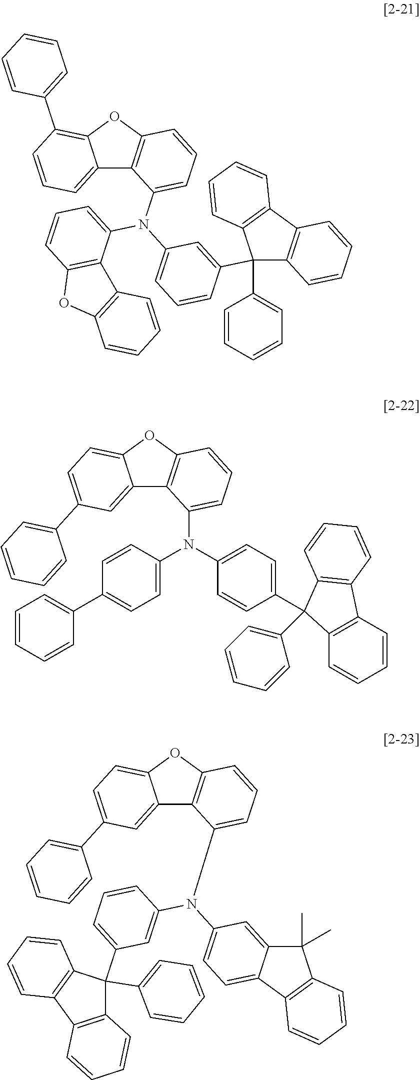 [SMILES: c1ccc(-c2cccc3c2oc2cccc(N(c4cccc(C5(c6ccccc6)c6ccccc6-c6ccccc65)c4)c4cccc5oc6ccccc6c45)c23)cc1 ; c1ccc(-c2ccc(N(c3ccc(C4(c5ccccc5)c5ccccc5-c5ccccc54)cc3)c3cccc4oc5ccc(-c6ccccc6)cc5c34)cc2)cc1 ; CC1(C)c2ccccc2-c2ccc(N(c3cccc(C4(c5ccccc5)c5ccccc5-c5ccccc54)c3)c3cccc4oc5ccc(-c6ccccc6)cc5c34)cc21]

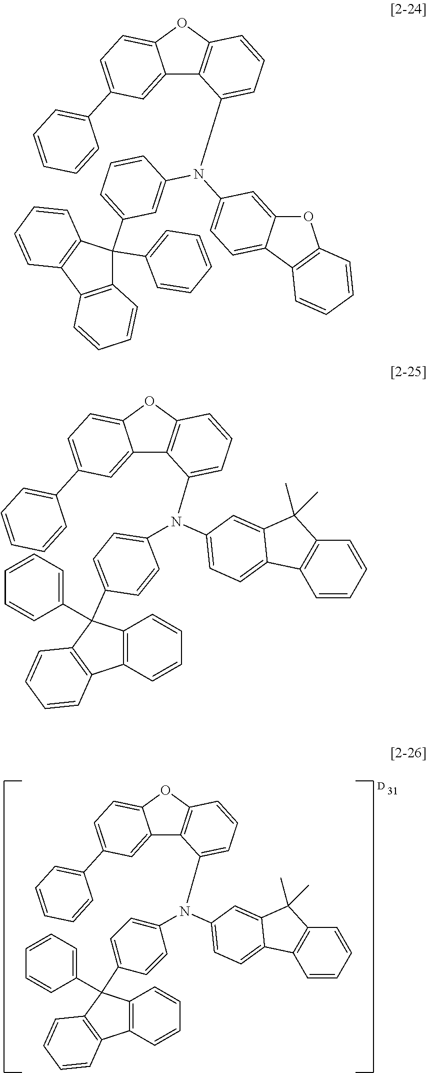

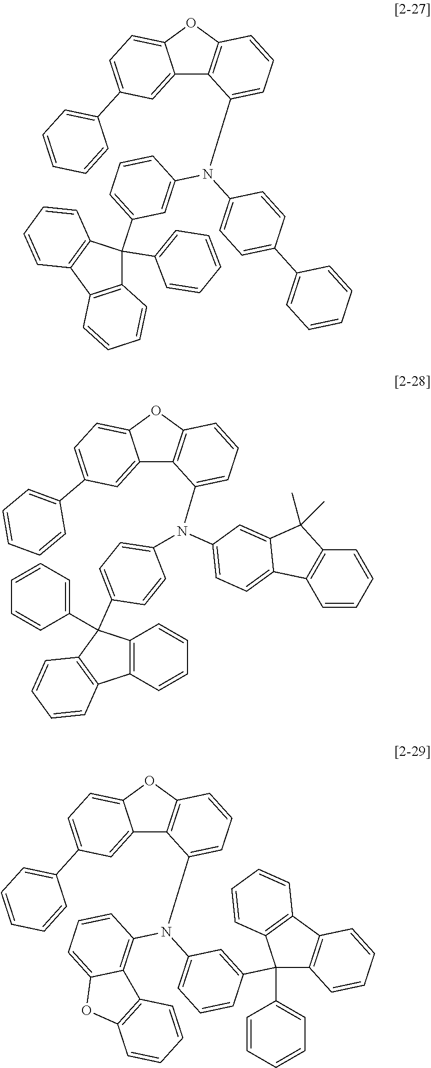 [SMILES: c1ccc(-c2ccc(N(c3cccc(C4(c5ccccc5)c5ccccc5-c5ccccc54)c3)c3cccc4oc5ccc(-c6ccccc6)cc5c34)cc2)cc1 ; CC1(C)c2ccccc2-c2ccc(N(c3ccc(C4(c5ccccc5)c5ccccc5-c5ccccc54)cc3)c3cccc4oc5ccc(-c6ccccc6)cc5c34)cc21 ; c1ccc(-c2ccc3oc4cccc(N(c5cccc(C6(c7ccccc7)c7ccccc7-c7ccccc76)c5)c5cccc6oc7ccccc7c56)c4c3c2)cc1]

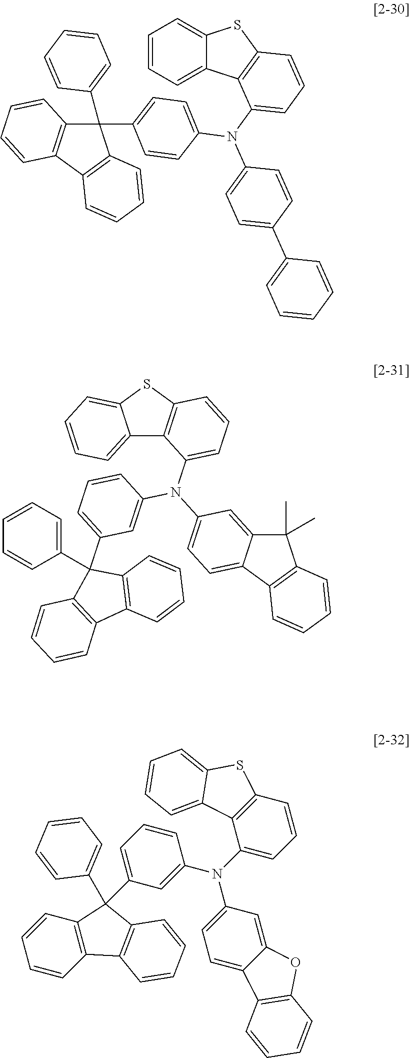 [SMILES: c1ccc(-c2ccc(N(c3ccc(C4(c5ccccc5)c5ccccc5-c5ccccc54)cc3)c3cccc4sc5ccccc5c34)cc2)cc1 ; CC1(C)c2ccccc2-c2ccc(N(c3cccc(C4(c5ccccc5)c5ccccc5-c5ccccc54)c3)c3cccc4sc5ccccc5c34)cc21 ; c1ccc(C2(c3cccc(N(c4ccc5c(c4)oc4ccccc45)c4cccc5sc6ccccc6c45)c3)c3ccccc3-c3ccccc32)cc1]

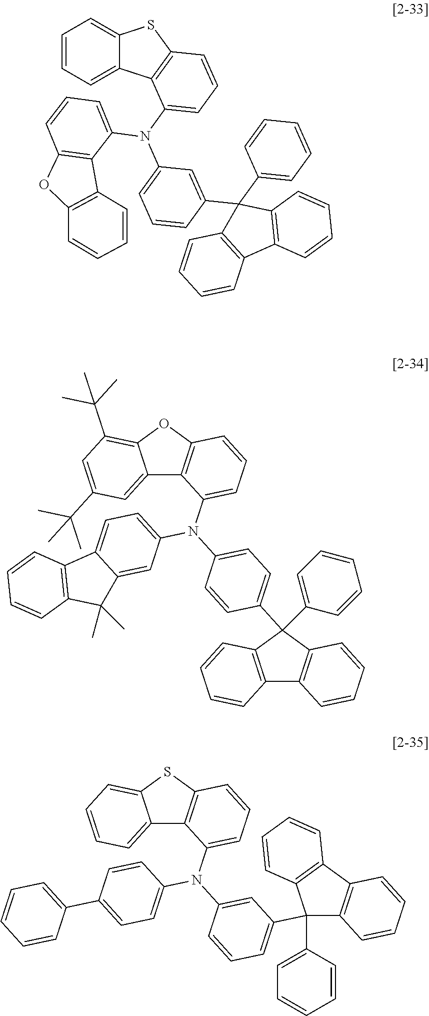 [SMILES: c1ccc(C2(c3cccc(N(c4cccc5oc6ccccc6c45)c4cccc5sc6ccccc6c45)c3)c3ccccc3-c3ccccc32)cc1 ; CC(C)(C)c1cc(C(C)(C)C)c2oc3cccc(N(c4ccc(C5(c6ccccc6)c6ccccc6-c6ccccc65)cc4)c4ccc5c(c4)C(C)(C)c4ccccc4-5)c3c2c1 ; c1ccc(-c2ccc(N(c3cccc(C4(c5ccccc5)c5ccccc5-c5ccccc54)c3)c3cccc4sc5ccccc5c34)cc2)cc1]

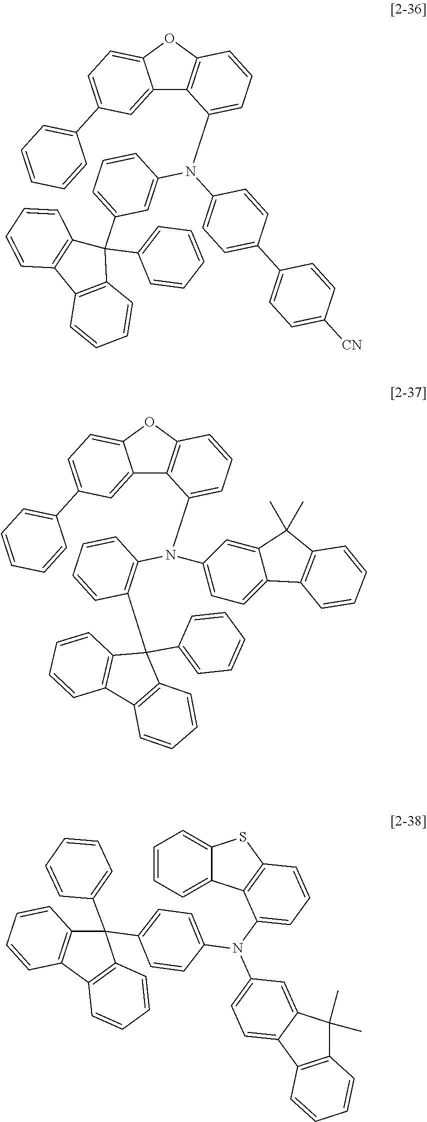 [SMILES: N#Cc1ccc(-c2ccc(N(c3cccc(C4(c5ccccc5)c5ccccc5-c5ccccc54)c3)c3cccc4oc5ccc(-c6ccccc6)cc5c34)cc2)cc1 ; CC1(C)c2ccccc2-c2ccc(N(c3ccccc3C3(c4ccccc4)c4ccccc4-c4ccccc43)c3cccc4oc5ccc(-c6ccccc6)cc5c34)cc21 ; CC1(C)c2ccccc2-c2ccc(N(c3ccc(C4(c5ccccc5)c5ccccc5-c5ccccc54)cc3)c3cccc4sc5ccccc5c34)cc21]

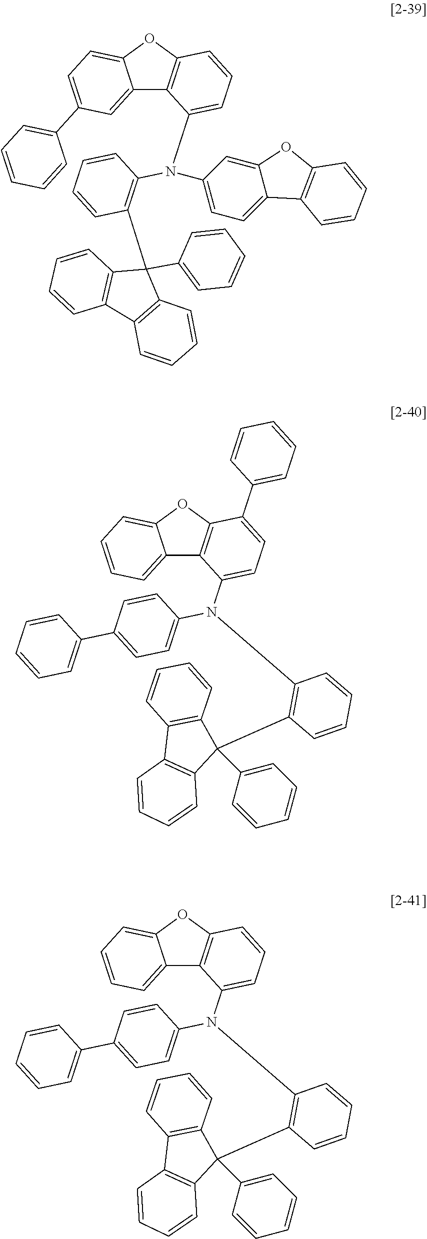 [SMILES: c1ccc(-c2ccc3oc4cccc(N(c5ccc6c(c5)oc5ccccc56)c5ccccc5C5(c6ccccc6)c6ccccc6-c6ccccc65)c4c3c2)cc1 ; c1ccc(-c2ccc(N(c3ccccc3C3(c4ccccc4)c4ccccc4-c4ccccc43)c3ccc(-c4ccccc4)c4oc5ccccc5c34)cc2)cc1 ; c1ccc(-c2ccc(N(c3ccccc3C3(c4ccccc4)c4ccccc4-c4ccccc43)c3cccc4oc5ccccc5c34)cc2)cc1]

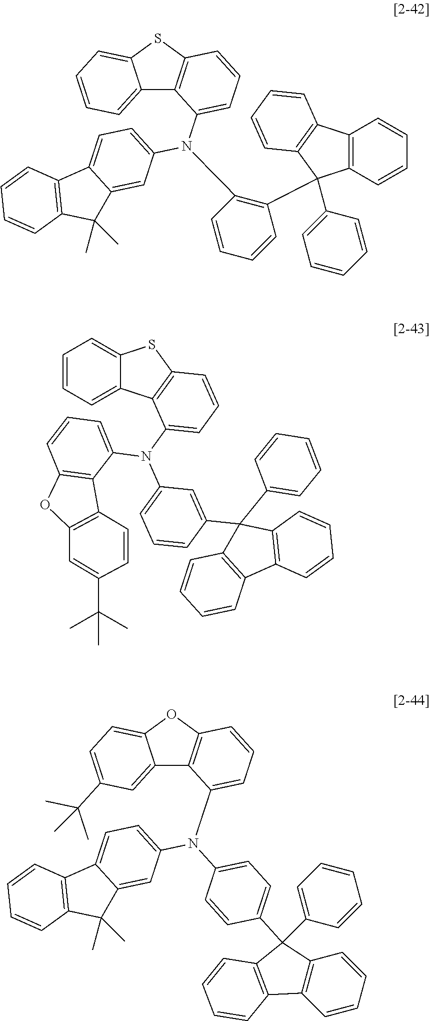 [SMILES: CC1(C)c2ccccc2-c2ccc(N(c3ccccc3C3(c4ccccc4)c4ccccc4-c4ccccc43)c3cccc4sc5ccccc5c34)cc21 ; CC(C)(C)c1ccc2c(c1)oc1cccc(N(c3cccc(C4(c5ccccc5)c5ccccc5-c5ccccc54)c3)c3cccc4sc5ccccc5c34)c12 ; CC(C)(C)c1ccc2oc3cccc(N(c4ccc(C5(c6ccccc6)c6ccccc6-c6ccccc65)cc4)c4ccc5c(c4)C(C)(C)c4ccccc4-5)c3c2c1]

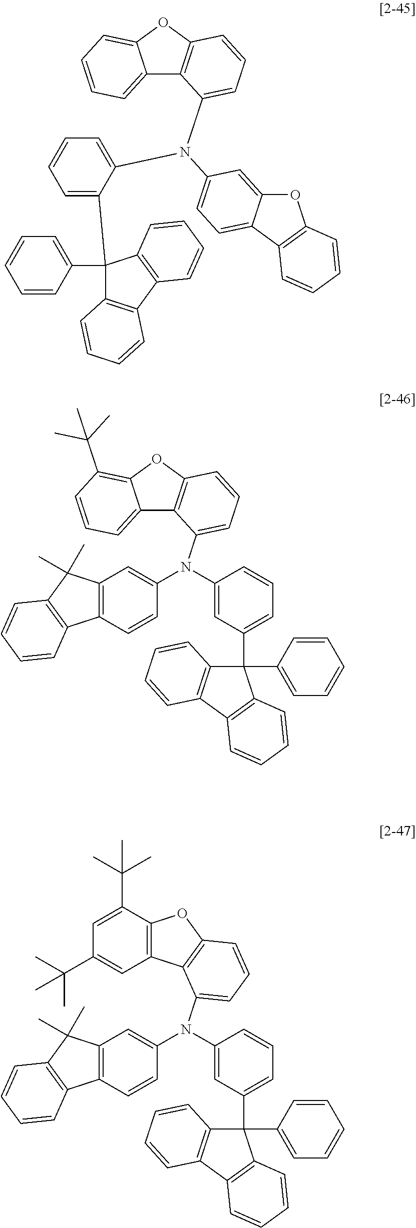 [SMILES: c1ccc(C2(c3ccccc3N(c3ccc4c(c3)oc3ccccc34)c3cccc4oc5ccccc5c34)c3ccccc3-c3ccccc32)cc1 ; CC(C)(C)c1cccc2c1oc1cccc(N(c3cccc(C4(c5ccccc5)c5ccccc5-c5ccccc54)c3)c3ccc4c(c3)C(C)(C)c3ccccc3-4)c12 ; CC(C)(C)c1cc(C(C)(C)C)c2oc3cccc(N(c4cccc(C5(c6ccccc6)c6ccccc6-c6ccccc65)c4)c4ccc5c(c4)C(C)(C)c4ccccc4-5)c3c2c1]

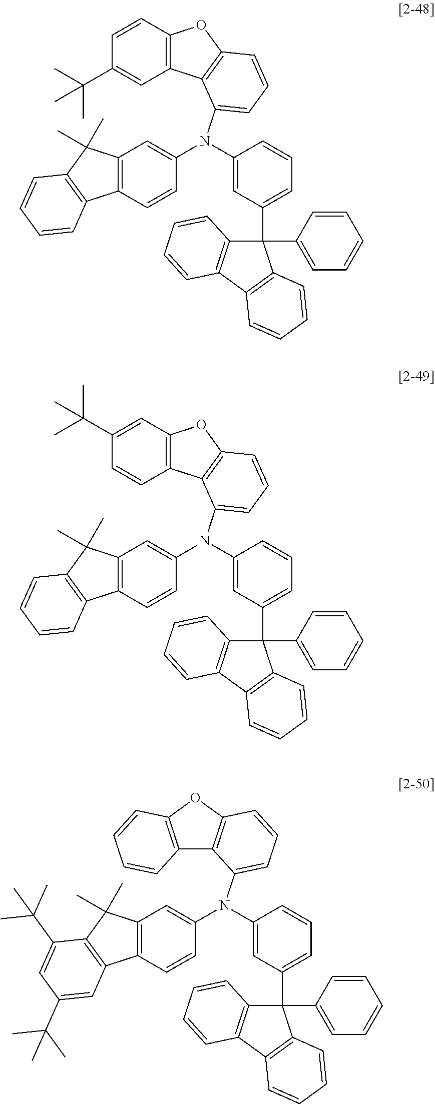 [SMILES: CC(C)(C)c1ccc2oc3cccc(N(c4cccc(C5(c6ccccc6)c6ccccc6-c6ccccc65)c4)c4ccc5c(c4)C(C)(C)c4ccccc4-5)c3c2c1 ; CC(C)(C)c1ccc2c(c1)oc1cccc(N(c3cccc(C4(c5ccccc5)c5ccccc5-c5ccccc54)c3)c3ccc4c(c3)C(C)(C)c3ccccc3-4)c12 ; CC(C)(C)c1cc2c(c(C(C)(C)C)c1)C(C)(C)c1cc(N(c3cccc(C4(c5ccccc5)c5ccccc5-c5ccccc54)c3)c3cccc4oc5ccccc5c34)ccc1-2]

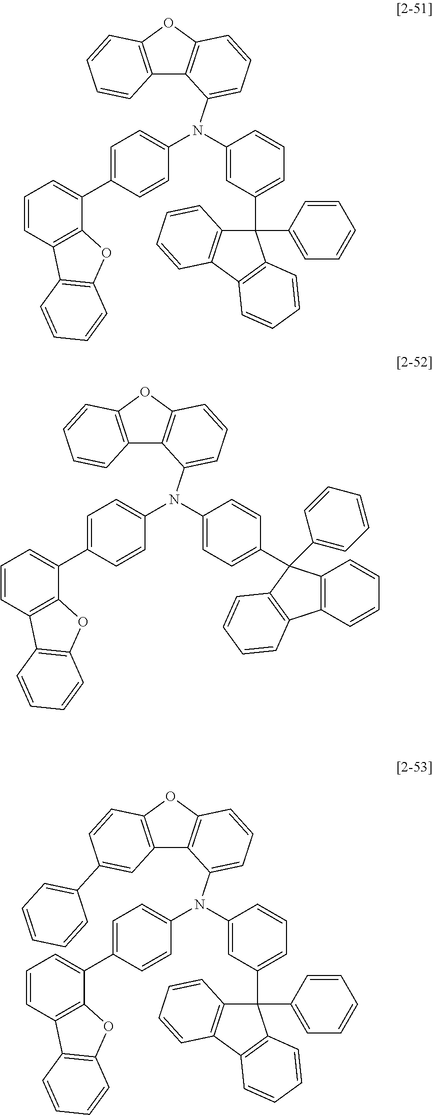 [SMILES: c1ccc(C2(c3cccc(N(c4ccc(-c5cccc6c5oc5ccccc56)cc4)c4cccc5oc6ccccc6c45)c3)c3ccccc3-c3ccccc32)cc1 ; c1ccc(C2(c3ccc(N(c4ccc(-c5cccc6c5oc5ccccc56)cc4)c4cccc5oc6ccccc6c45)cc3)c3ccccc3-c3ccccc32)cc1 ; c1ccc(-c2ccc3oc4cccc(N(c5ccc(-c6cccc7c6oc6ccccc67)cc5)c5cccc(C6(c7ccccc7)c7ccccc7-c7ccccc76)c5)c4c3c2)cc1]

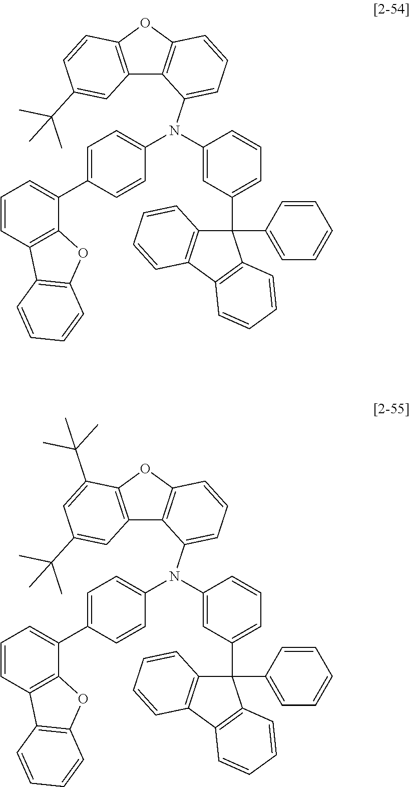 [SMILES: CC(C)(C)c1ccc2oc3cccc(N(c4ccc(-c5cccc6c5oc5ccccc56)cc4)c4cccc(C5(c6ccccc6)c6ccccc6-c6ccccc65)c4)c3c2c1 ; CC(C)(C)c1cc(C(C)(C)C)c2oc3cccc(N(c4ccc(-c5cccc6c5oc5ccccc56)cc4)c4cccc(C5(c6ccccc6)c6ccccc6-c6ccccc65)c4)c3c2c1]

The organic layer 30 may include an electron transport region. The electron transport region may help further increase electron injection or electron mobility and block holes between the cathode 20 and the light emitting layer 31.

In an implementation, the electron transport region may include an electron transport layer 35 between the cathode 20 and the light emitting layer 31, and an electron transport auxiliary layer between the light emitting layer 31 and the electron transport layer 35 and a compound of Group C may be included in at least one layer of the electron transport layer 35 and the electron transport auxiliary layer.

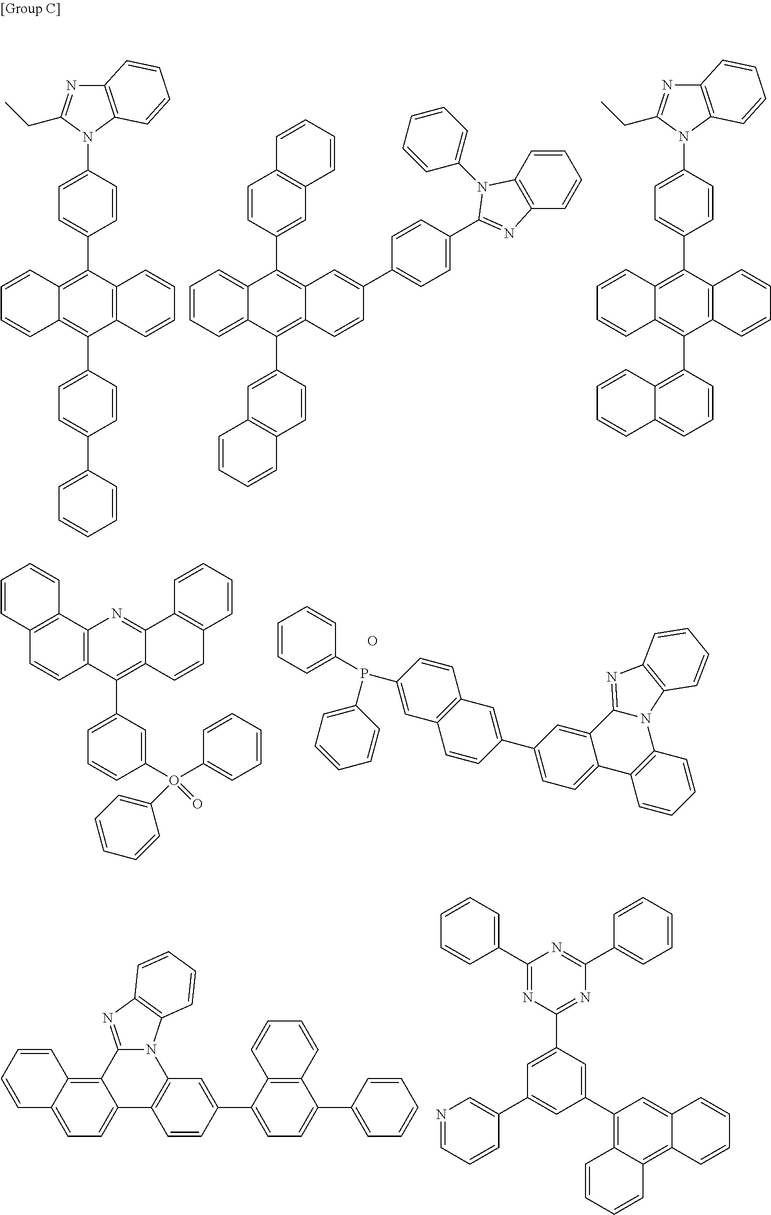

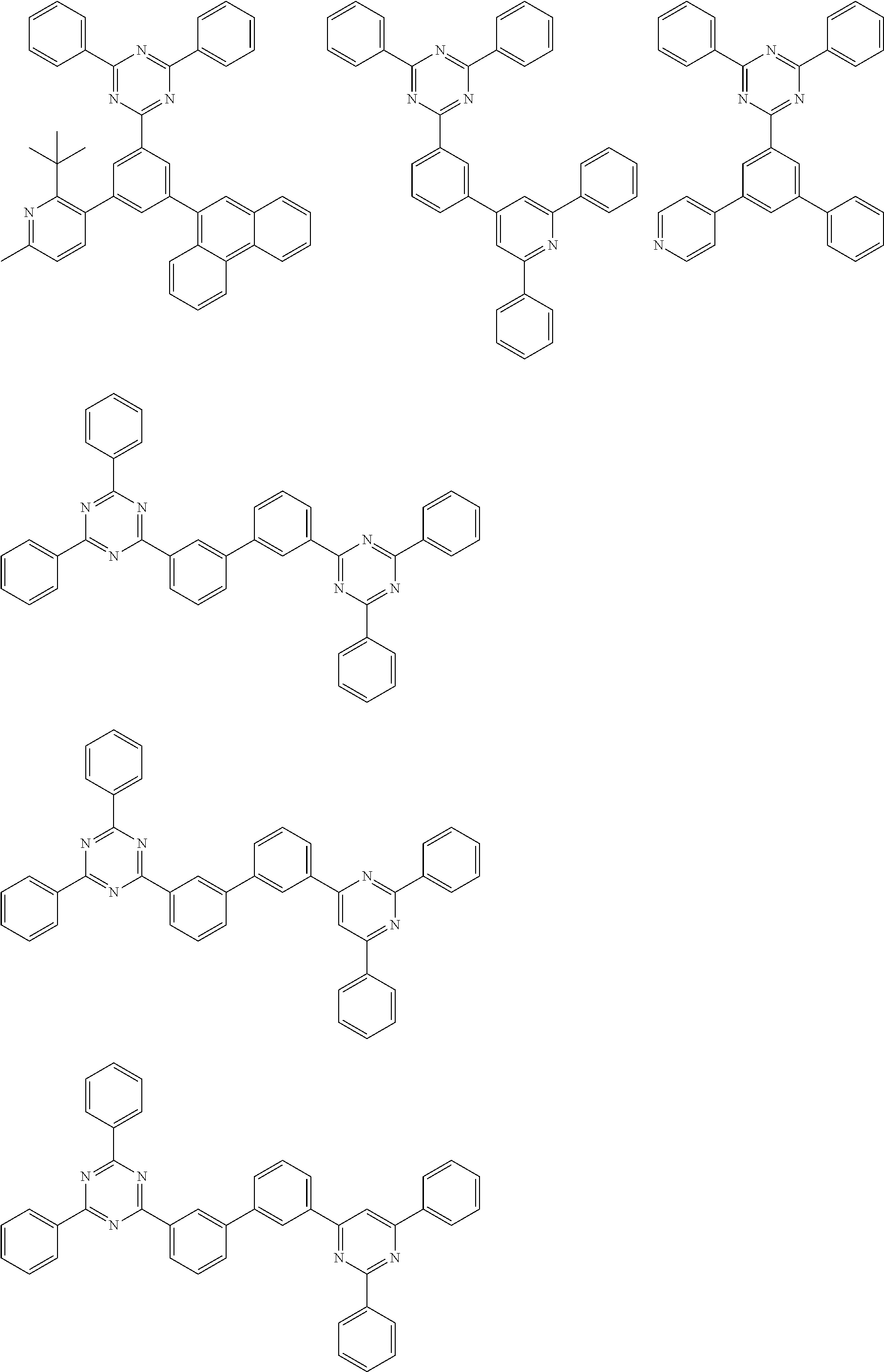

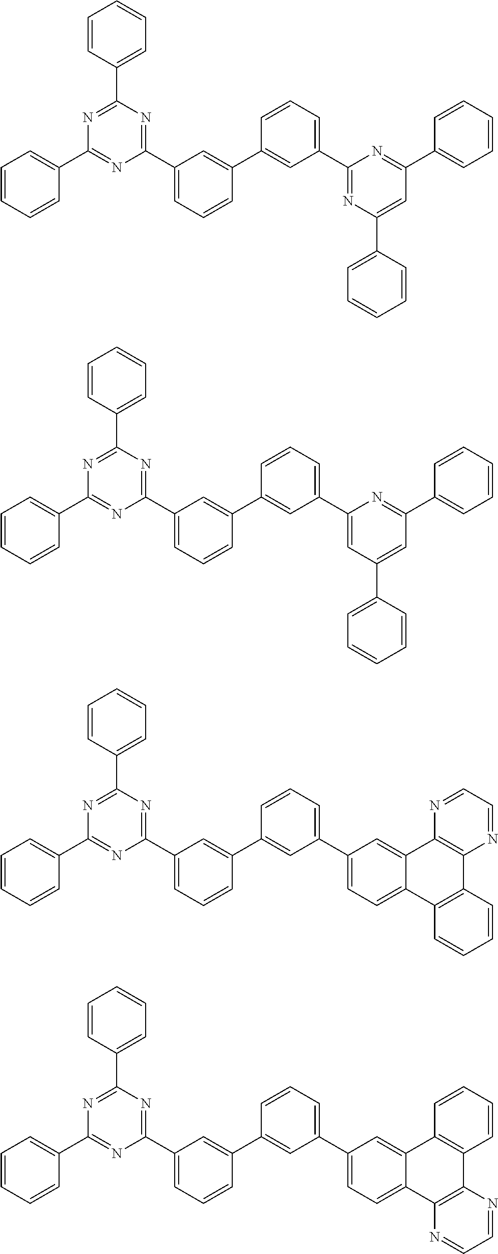 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

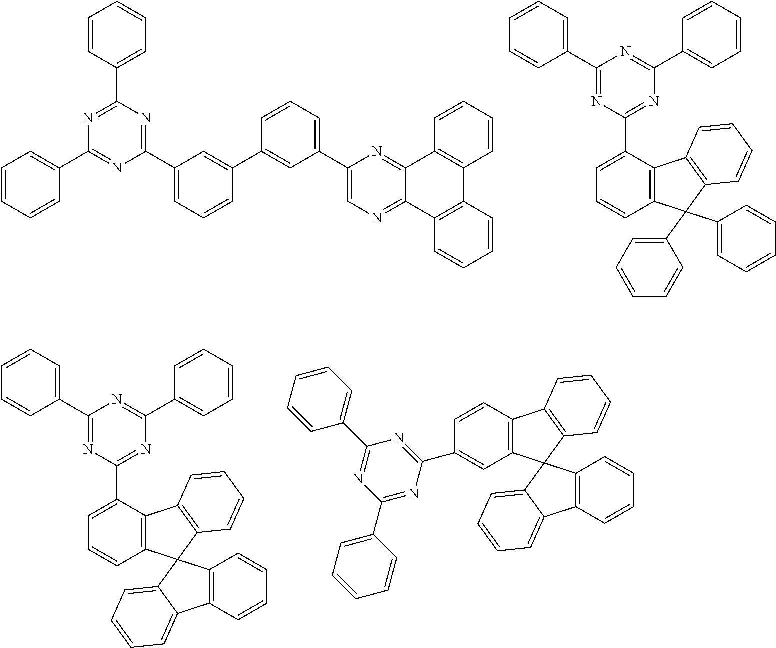 [SMILES: c1ccc(-c2nc(-c3ccccc3)nc(-c3cccc(-c4cccc(-c5cnc6c7ccccc7c7ccccc7c6n5)c4)c3)n2)cc1 ; c1ccc(-c2nc(-c3ccccc3)nc(-c3cccc4c3-c3ccccc3C4(c3ccccc3)c3ccccc3)n2)cc1 ; c1ccc(-c2nc(-c3ccccc3)nc(-c3cccc4c3-c3ccccc3C43c4ccccc4-c4ccccc43)n2)cc1 ; c1ccc(-c2nc(-c3ccccc3)nc(-c3ccc4c(c3)C3(c5ccccc5-c5ccccc53)c3ccccc3-4)n2)cc1]

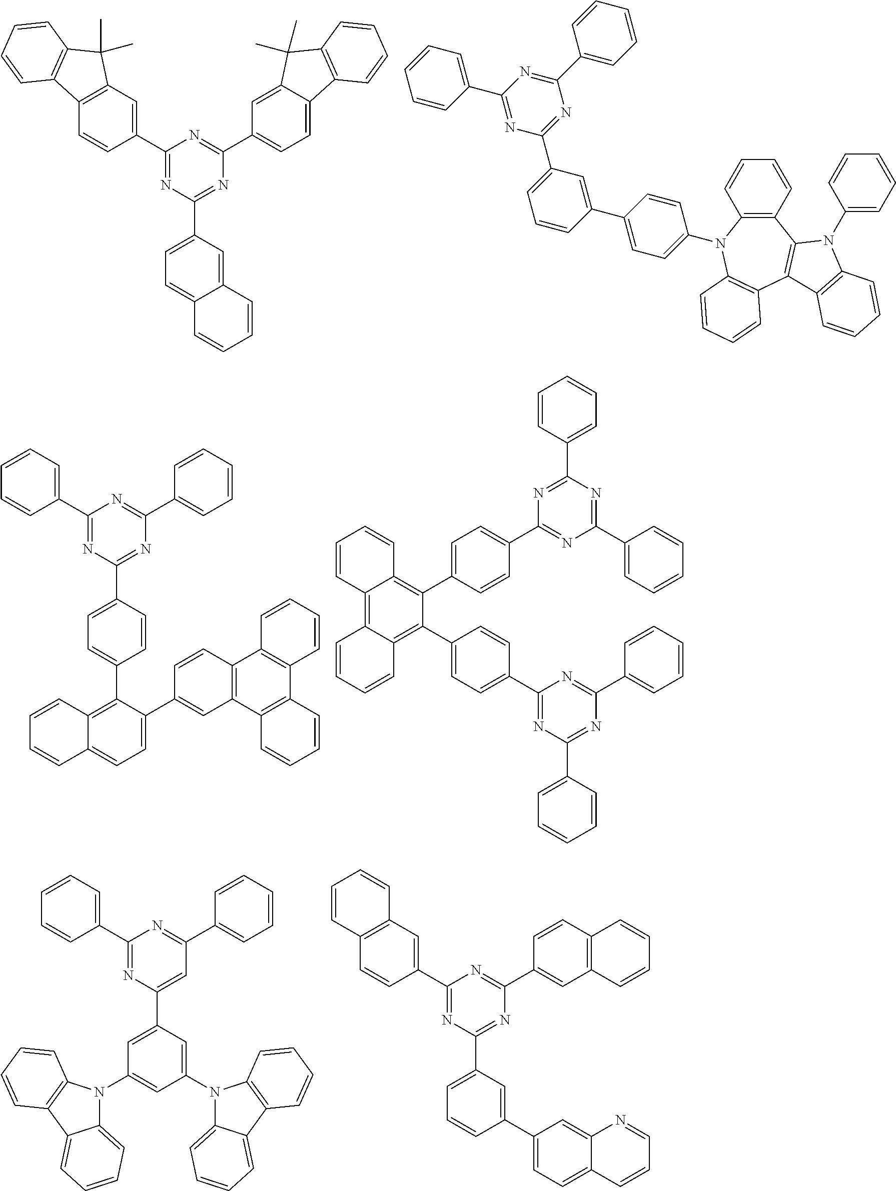

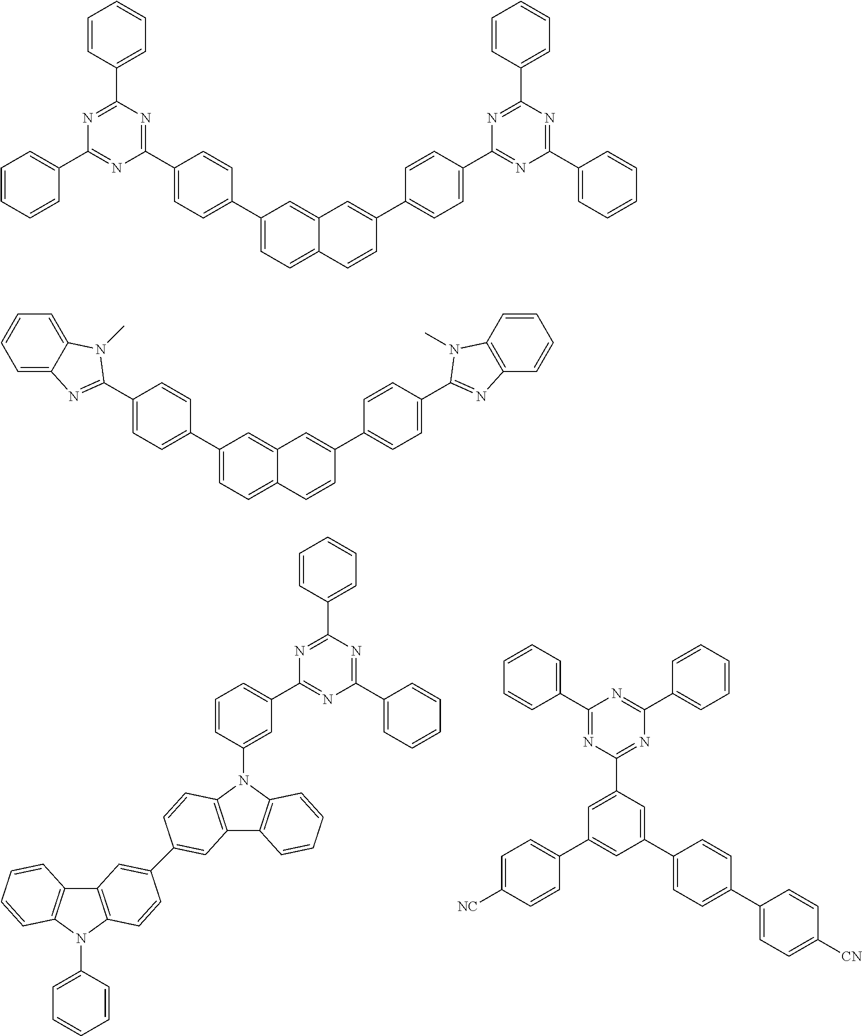

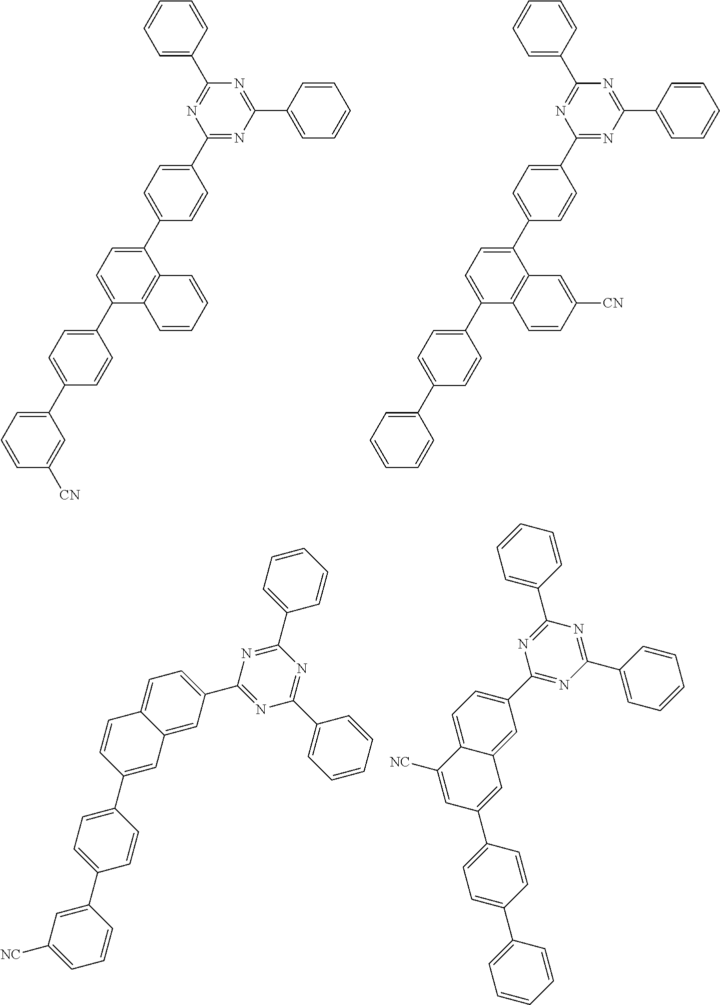

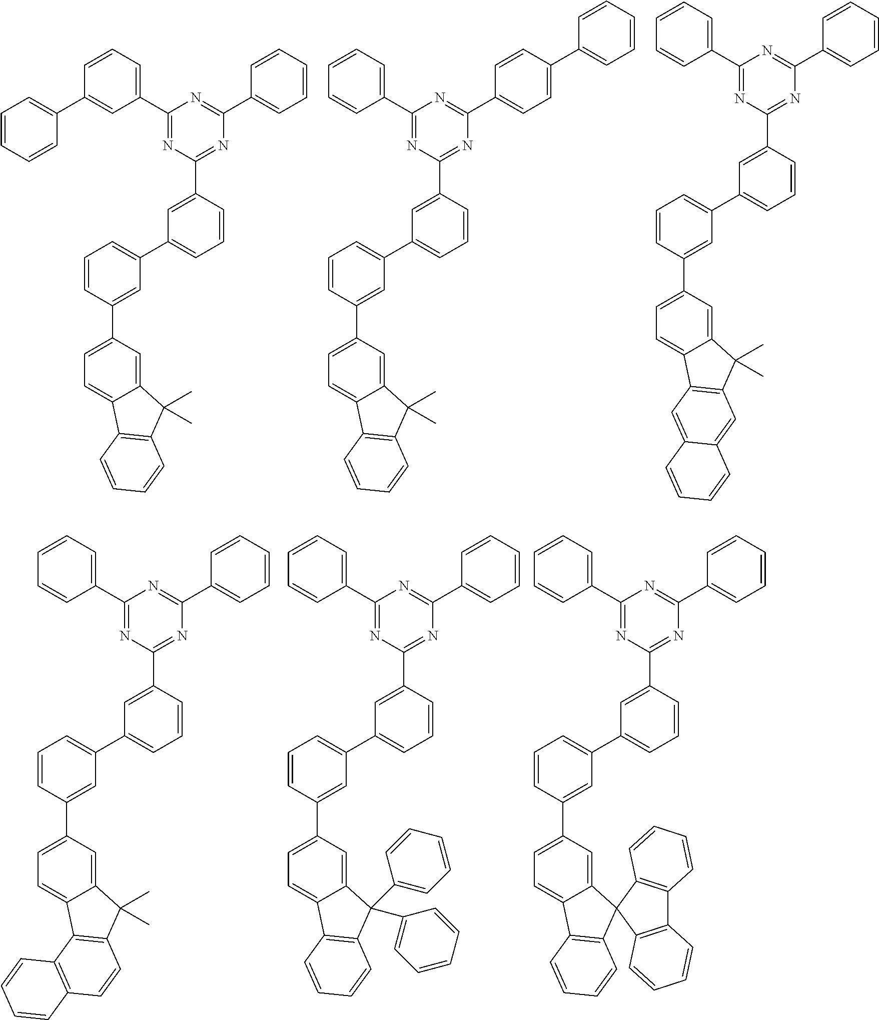

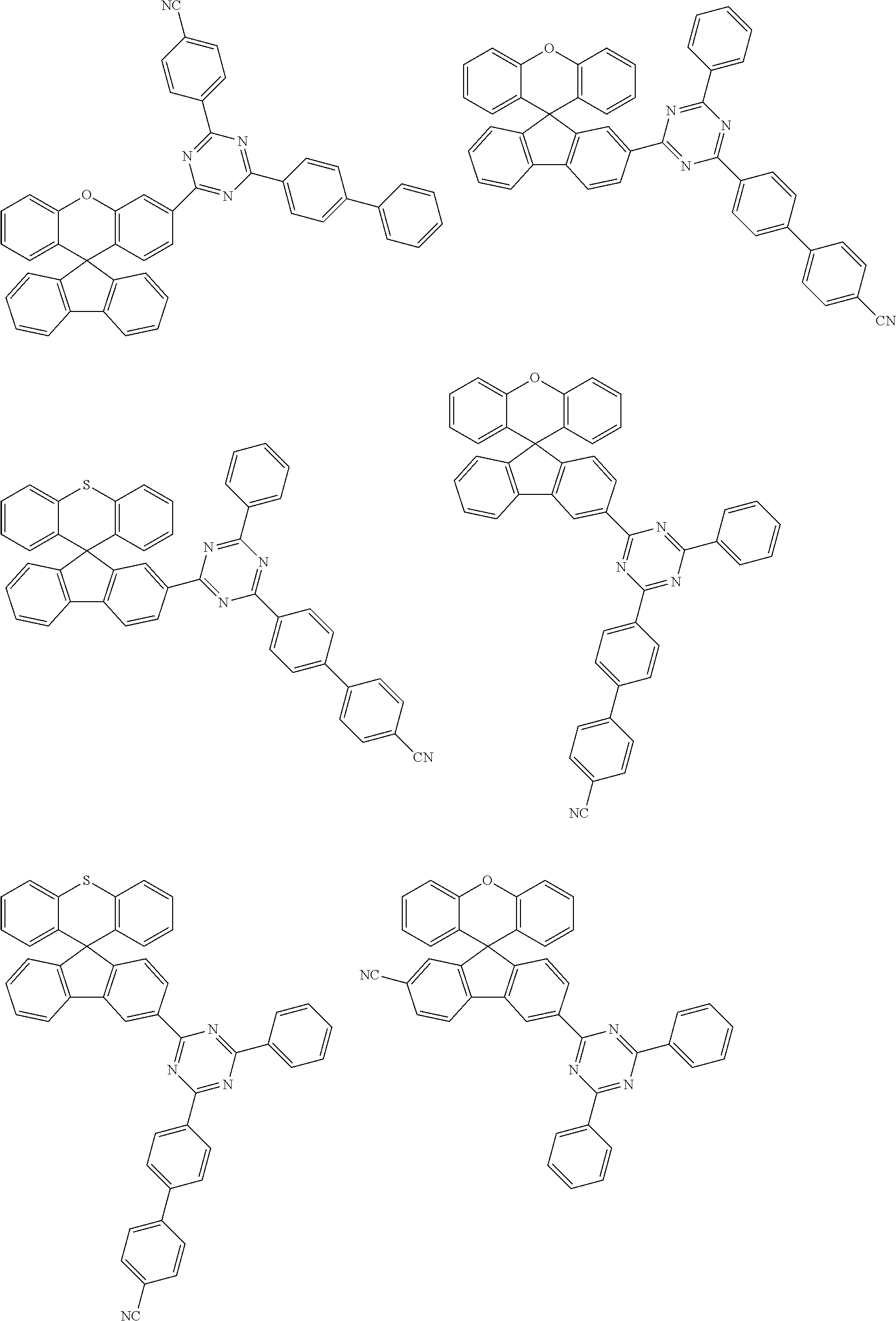

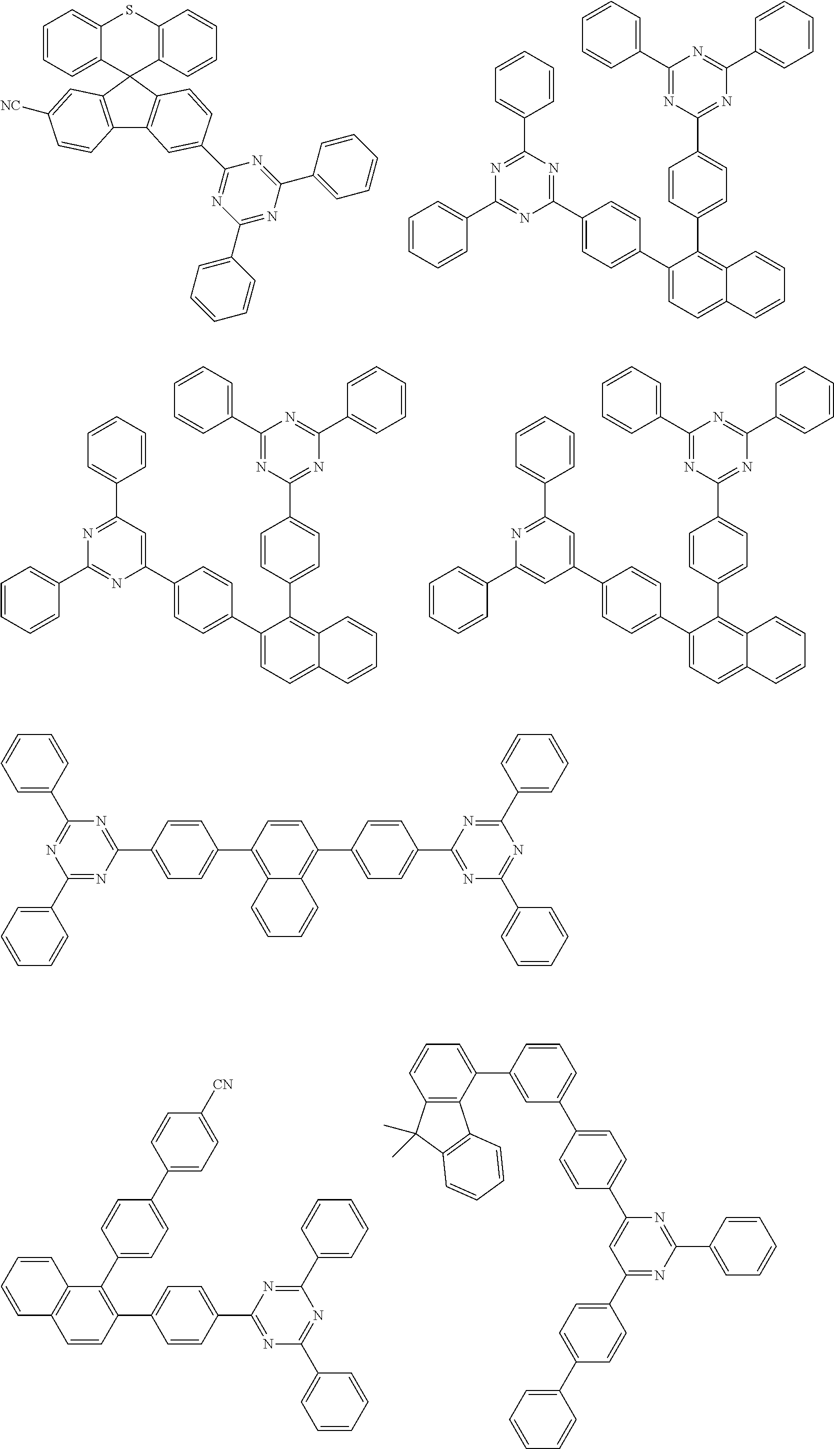

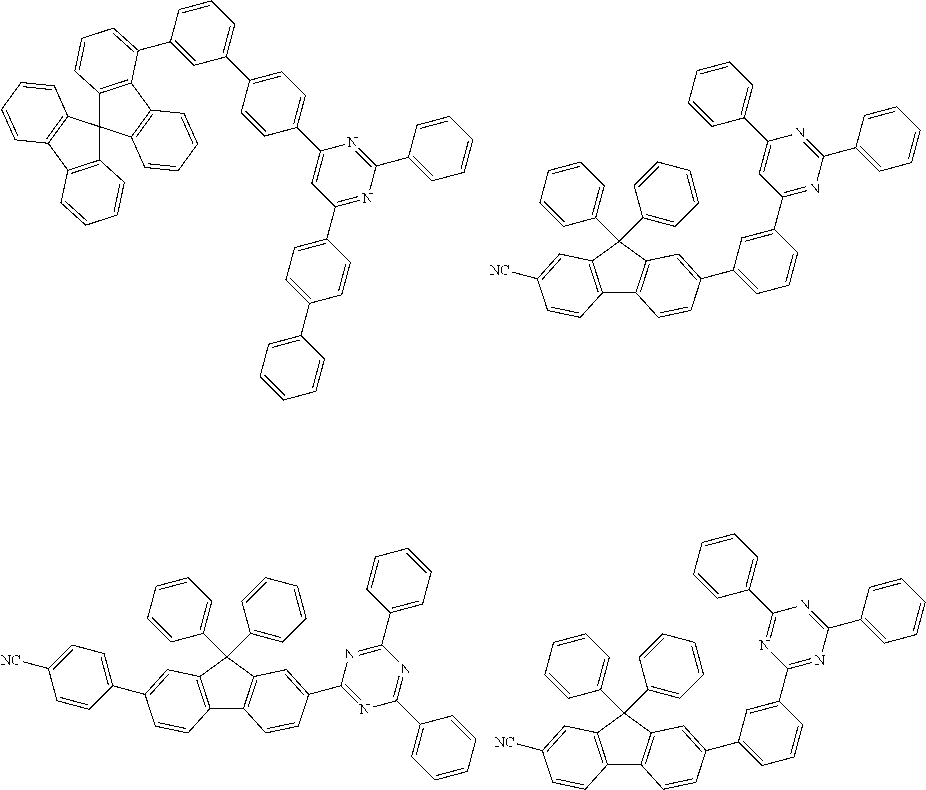

In some embodiments, an organic light emitting diode may include a light emitting layer 31, a hole transport layer 32, a first hole transport auxiliary layer 33a, and a second hole transport auxiliary layer 33b as an organic layer.

In some embodiments, an organic light emitting diode may additionally include a hole injection layer 34 as an organic layer.

In some embodiments, an organic light emitting diode may additionally include an electron transport region as an organic layer.

In an implementation, the organic light emitting diode may further include an electron injection layer in addition to the light emitting layer as the aforementioned organic layer.

The organic light emitting diode may be manufactured by forming an anode or cathode on a substrate, then forming an organic layer by dry film method such as evaporation, sputtering, plasma plating, or ion plating, and then forming a cathode or anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in the Examples and Synthesis Examples were purchased from Sigma-Aldrich Co. Ltd., TCI Inc., Tokyo Chemical Industry, or P&H Tech as far as there in no particular comment or were synthesized by suitable methods.

### (Preparation of First Compound)

### Synthesis Example 1: Synthesis of Compound 1-21

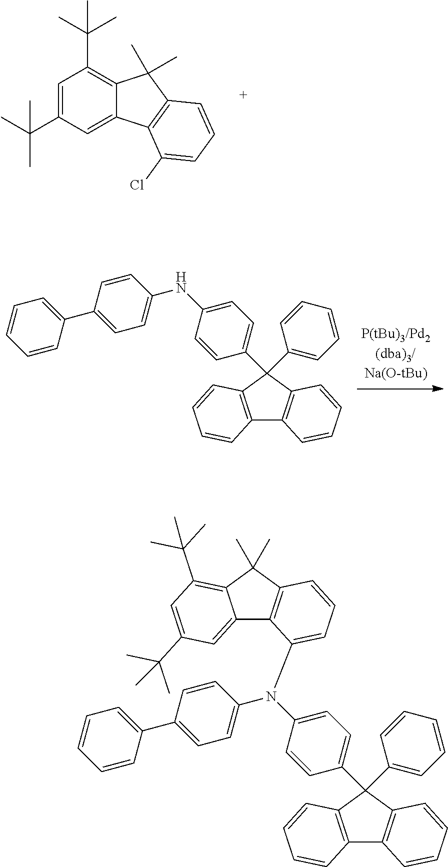 [SMILES: CC(C)(C)c1cc2c(c(C(C)(C)C)c1)C(C)(C)c1cccc(Cl)c1-2 ; c1ccc(-c2ccc(Nc3ccc(C4(c5ccccc5)c5ccccc5-c5ccccc54)cc3)cc2)cc1 ; CC(C)(C)c1cc2c(c(C(C)(C)C)c1)C(C)(C)c1cccc(N(c3ccc(-c4ccccc4)cc3)c3ccc(C4(c5ccccc5)c5ccccc5-c5ccccc54)cc3)c1-2]

10.0 g (29.33 mmol) of an intermediate of 1,3-di-tert-butyl-5-chloro-9,9-dimethyl-9H-fluorene, 15.67 g (32.26 mmol) of an intermediate of N-(4-(9-phenyl-9H-fluoren-9-yl)phenyl)-[1,1′-biphenyl]-4-amine), 5.64 g (58.66 mmol) of sodium t-butoxide, and 0.7 g (1.76 mmol) of tri-tert-butylphosphine were dissolved in 293 m1 of toluene, and 0.8 g (0.88 mmol) of Pd₂(dba)₃ was added thereto and then, stirred under reflux for 12 hours under a nitrogen atmosphere. After a reaction was completed, an organic layer, which was extracted therefrom with ethyl acetate and distilled water, was dried with anhydrous magnesium sulfate and filtered, and a filtrate therefrom was concentrated under a reduced pressure. A product therefrom was purified through silica gel column chromatography with normal hexane/dichloromethane (in a volume ratio of 3:1) to obtain 17.9 g (a yield: 77%) of Compound 1-21 as a white solid.

Calculation value: C, 91.21; H, 7.02; N, 1.77.

Analysis value: C, 91.21; H, 7.02; N, 1.77.

### Synthesis Example 2: Synthesis of Compound 1-25

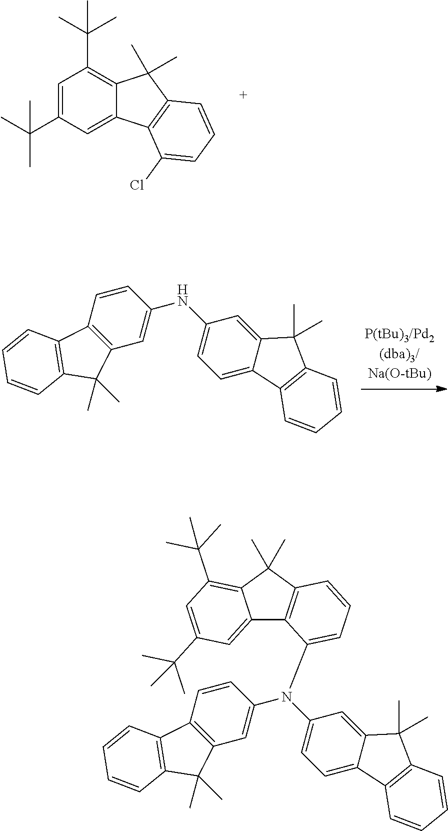 [SMILES: CC(C)(C)c1cc2c(c(C(C)(C)C)c1)C(C)(C)c1cccc(Cl)c1-2 ; CC1(C)c2ccccc2-c2ccc(Nc3ccc4c(c3)C(C)(C)c3ccccc3-4)cc21 ; CC(C)(C)c1cc2c(c(C(C)(C)C)c1)C(C)(C)c1cccc(N(c3ccc4c(c3)C(C)(C)c3ccccc3-4)c3ccc4c(c3)C(C)(C)c3ccccc3-4)c1-2]

Compound 1-25 (16.8 g, a yield: 81%) was synthesized in the same manner as in Synthesis Example 1 except that 10 g of the intermediate of 1,3-di-tert-butyl-5-chloro-9,9-dimethyl-9H-fluorene and 12.96 g of the intermediate of bis(9,9-dimethyl-9H-fluoren-2-yl)amine were used in an equivalent ratio of 1:1.1.

Calculation value: C, 90.16; H, 7.85; N, 1.98.

Analysis value: C, 90.16; H, 7.85; N, 1.98.

### Synthesis Example 3: Synthesis of Compound 1-28

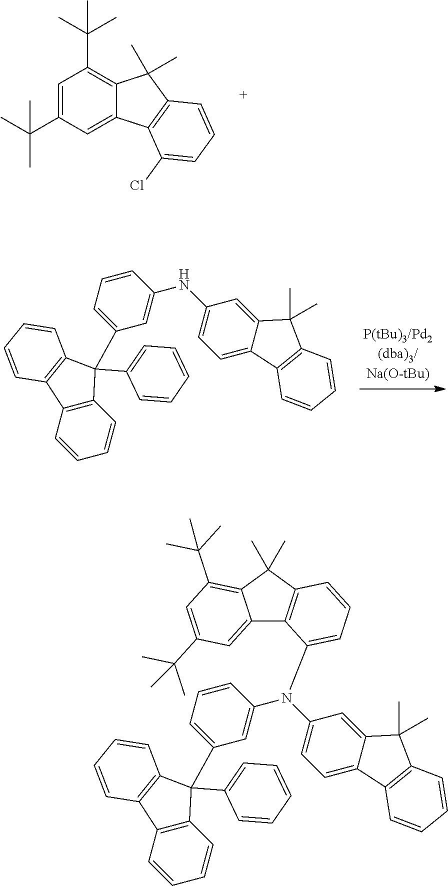 [SMILES: CC(C)(C)c1cc2c(c(C(C)(C)C)c1)C(C)(C)c1cccc(Cl)c1-2 ; CC1(C)c2ccccc2-c2ccc(Nc3cccc(C4(c5ccccc5)c5ccccc5-c5ccccc54)c3)cc21 ; CC(C)(C)c1cc2c(c(C(C)(C)C)c1)C(C)(C)c1cccc(N(c3cccc(C4(c5ccccc5)c5ccccc5-c5ccccc54)c3)c3ccc4c(c3)C(C)(C)c3ccccc3-4)c1-2]

Compound 1-28 (17.5 g, a yield: 72%) was synthesized in the same manner as in Synthesis Example 1 except that 10 g of the intermediate of 1,3-di-tert-butyl-5-chloro-9,9-dimethyl-9H-fluorene and 16.96 g of the intermediate of 9,9-dimethyl-N-(3-(9-phenyl-9H-fluoren-9-yl)phenyl)-9H-fluoren-2-amine were used in an equivalent ratio of 1:1.1.

Calculation value: C, 91.15; H, 7.16; N, 1.69.

Analysis value: C, 91.15; H, 7.16; N, 1.69.

### Synthesis Example 4: Synthesis of Compound 1-63

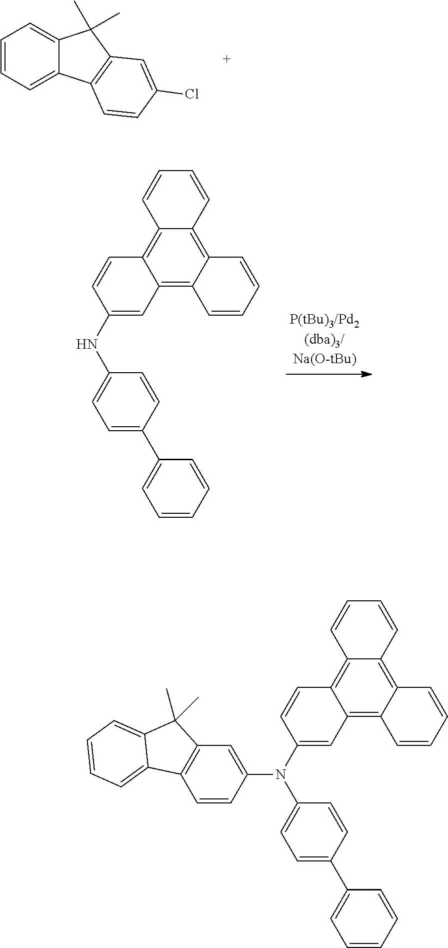 [SMILES: CC1(C)c2ccccc2-c2ccc(Cl)cc21 ; c1ccc(-c2ccc(Nc3ccc4c5ccccc5c5ccccc5c4c3)cc2)cc1 ; CC1(C)c2ccccc2-c2ccc(N(c3ccc(-c4ccccc4)cc3)c3ccc4c5ccccc5c5ccccc5c4c3)cc21]

Compound 1-63 (17.0 g, a yield: 83%) was synthesized in the same manner as in Synthesis Example 1 except that 8.0 g of the intermediate of 2-chloro-9,9-dimethyl-9H-fluorene and 15.22 g of the intermediate of N-([1,1′-biphenyl]-4-yl)triphenylene-2-amine were used in an equivalent ratio of 1:1.1.

Calculation value: C, 91.96; H, 5.66; N, 2.38.

Analysis value: C, 91.96; H, 5.66; N, 2.38.

### Synthesis Example 5: Synthesis of Compound 1-66

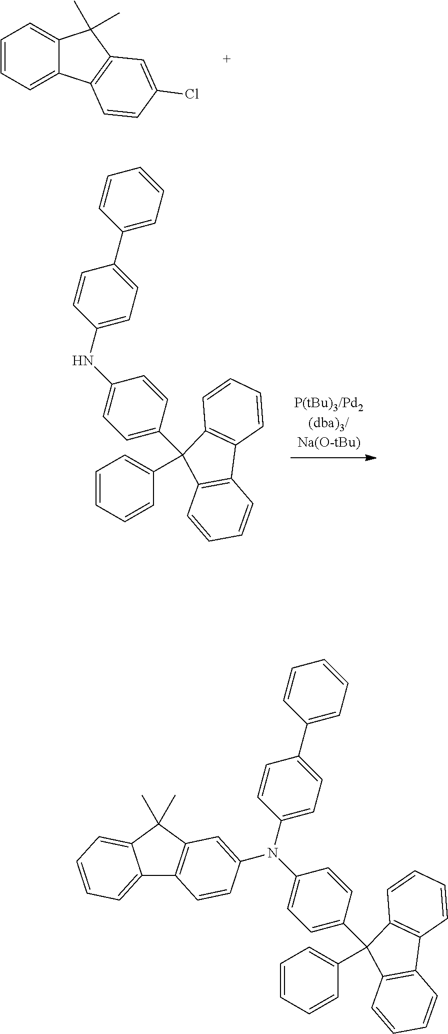 [SMILES: CC1(C)c2ccccc2-c2ccc(Cl)cc21 ; c1ccc(-c2ccc(Nc3ccc(C4(c5ccccc5)c5ccccc5-c5ccccc54)cc3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21]

Compound 1-66 (21.8 g, a yield: 82%) was synthesized in the same manner as in Synthesis Example 1 except that 9.0 g of the intermediate of 2-chloro-9,9-dimethyl-9H-fluorene and 21.0 g of the intermediate of N-(4-(9-phenyl-9H-fluoren-9-yl)phenyl)-[1,1′-biphenyl]-4-amine were used in an equivalent ratio of 1:1.1.

Calculation value: C, 92.13; H, 5.80; N, 2.07.

Analysis value: C, 92.13; H, 5.80; N, 2.07

### Synthesis Example 6: Synthesis of Compound 1-70

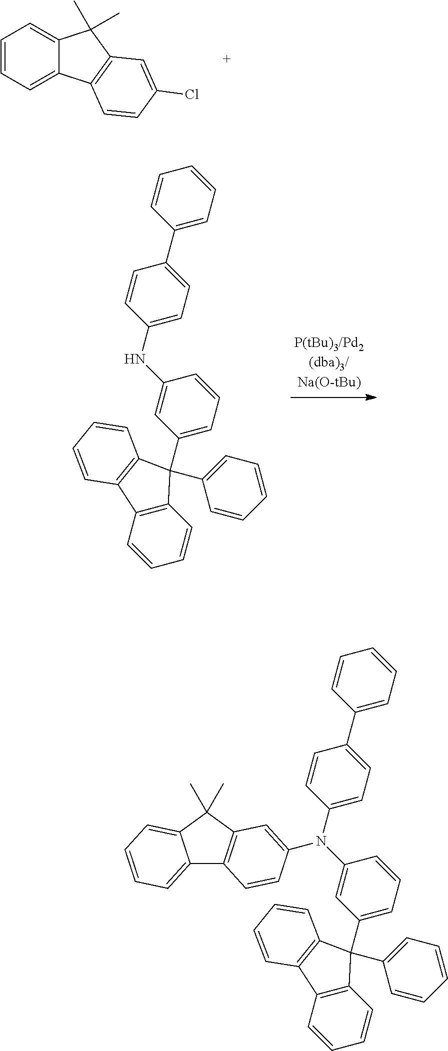 [SMILES: CC1(C)c2ccccc2-c2ccc(Cl)cc21 ; c1ccc(-c2ccc(Nc3cccc(C4(c5ccccc5)c5ccccc5-c5ccccc54)c3)cc2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

Compound 1-70 (15.8 g, a yield: 76%) was synthesized in the same manner as in Synthesis Example 1 except that 7.0 g of the intermediate of 2-chloro-9,9-dimethyl-9H-fluorene and 16.35 g of the intermediate of N-(3-(9-phenyl-9H-fluoren-9-yl)phenyl)-[1,1′-biphenyl]-4-amine were used in an equivalent ratio of 1:1.1.

Calculation value: C, 92.13; H, 5.80; N, 2.07

Analysis value: C, 92.13; H, 5.80; N, 2.07.

### Synthesis Example 7: Synthesis of Compound 1-75

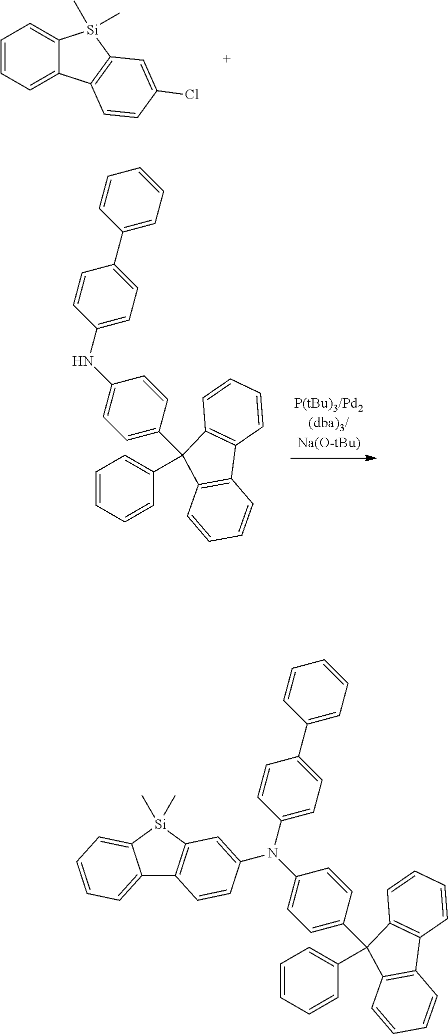 [SMILES: C[Si]1(C)c2ccccc2-c2ccc(Cl)cc21 ; c1ccc(-c2ccc(Nc3ccc(C4(c5ccccc5)c5ccccc5-c5ccccc54)cc3)cc2)cc1 ; C[Si]1(C)c2ccccc2-c2ccc(N(c3ccc(-c4ccccc4)cc3)c3ccc(C4(c5ccccc5)c5ccccc5-c5ccccc54)cc3)cc21]

Compound 1-75 (17.7 g, a yield: 62%) was synthesized in the same manner as in Synthesis Example 1 except that 10.0 g of the intermediate of 3-chloro-5,5-dimethyl-5H-dibenzo[b,d]silole and 21.82 g of the intermediate of N-(4-(9-phenyl-9H-fluoren-9-yl)phenyl)-[1,1′-biphenyl]-4-amine were used in an equivalent ratio of 1:1.1.

Calculation Value: C, 88.27; H, 5.66; N, 2.02; Si, 4.05.

Analysis Value: C, 88.27; H, 5.66; N, 2.02; Si, 4.04.

### (Preparation of Second Compound)

### Synthesis Example 8: Synthesis of Compound 2-6

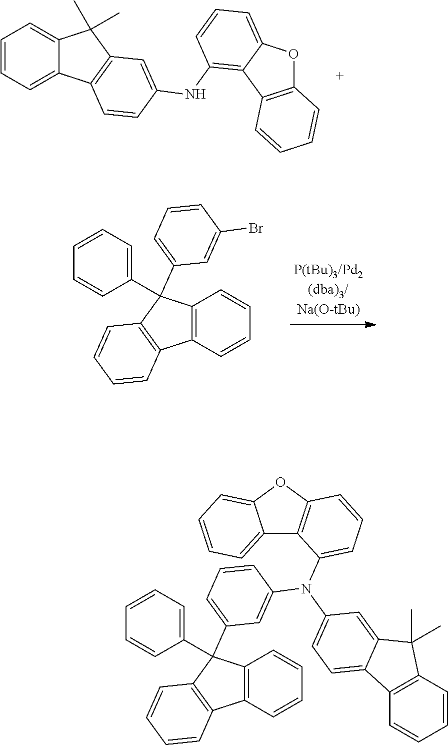 [SMILES: CC1(C)c2ccccc2-c2ccc(Nc3cccc4oc5ccccc5c34)cc21 ; Brc1cccc(C2(c3ccccc3)c3ccccc3-c3ccccc32)c1 ; CC1(C)c2ccccc2-c2ccc(N(c3cccc(C4(c5ccccc5)c5ccccc5-c5ccccc54)c3)c3cccc4oc5ccccc5c34)cc21]

Compound 2-6 (15.3 g, a yield 83%) was synthesized in the same manner as in Synthesis Example 1 except that 10 g of the intermediate of N-(9,9-dimethyl-9H-fluoren-2-yl)dibenzo[b,d]furan-1-amine and 11.64 g of the intermediate of 9-(3-bromophenyl)-9-phenyl-9H-fluorene were used in an equivalent ratio of 1:1.1.

Calculation value: C, 90.27; H, 5.39; N, 2.02; O, 2.31.

Analysis value: C, 90.27; H, 5.39; N, 2.02; O, 2.31.

### Synthesis Example 9: Synthesis of Compound 2-10

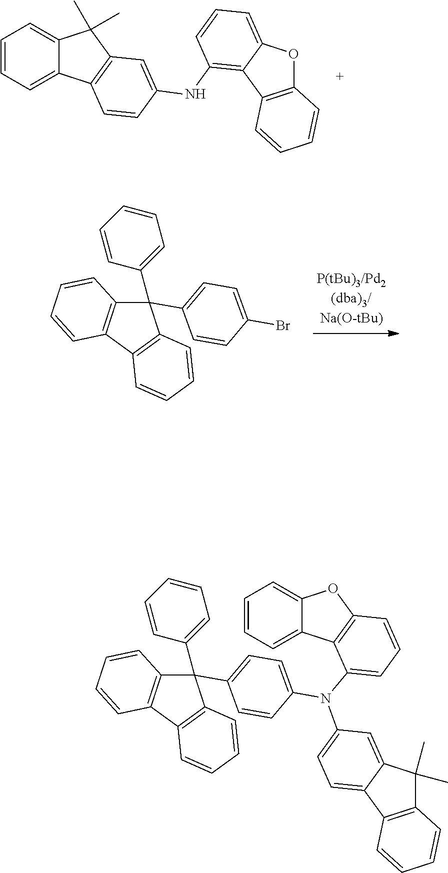 [SMILES: CC1(C)c2ccccc2-c2ccc(Nc3cccc4oc5ccccc5c34)cc21 ; Brc1ccc(C2(c3ccccc3)c3ccccc3-c3ccccc32)cc1 ; CC1(C)c2ccccc2-c2ccc(N(c3ccc(C4(c5ccccc5)c5ccccc5-c5ccccc54)cc3)c3cccc4oc5ccccc5c34)cc21]

Compound 2-10 (22.4 g, a yield: 81%) was synthesized in the same manner as in Synthesis Example 1 except that 15 g of the intermediate of N-(9,9-dimethyl-9H-fluoren-2-yl)dibenzo[b,d]furan-1-amine and 19.05 g of the intermediate of 9-(4-bromophenyl)-9-phenyl-9H-fluorene were used in an equivalent ratio of 1:1.2.

Calculation value: C, 90.27; H, 5.39; N, 2.02; O, 2.31.

Analysis value: C, 90.27; H, 5.39; N, 2.02; O, 2.31.

### Synthesis Example 10: Synthesis of Compound 2-13

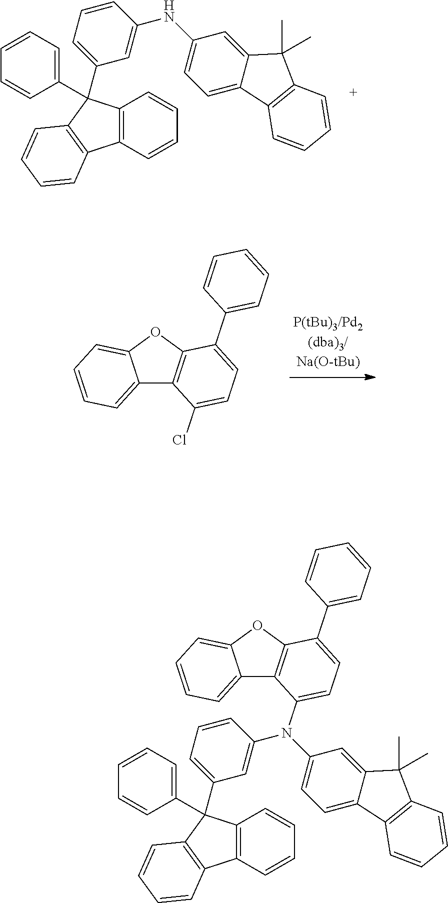 [SMILES: CC1(C)c2ccccc2-c2ccc(Nc3cccc(C4(c5ccccc5)c5ccccc5-c5ccccc54)c3)cc21 ; Clc1ccc(-c2ccccc2)c2oc3ccccc3c12 ; CC1(C)c2ccccc2-c2ccc(N(c3cccc(C4(c5ccccc5)c5ccccc5-c5ccccc54)c3)c3ccc(-c4ccccc4)c4oc5ccccc5c34)cc21]

Compound 2-13 (17.9 g, a yield: 82%) was synthesized in the same manner as in Synthesis Example 1 except that 15 g of the intermediate of 9,9-dimethyl-N-(3-(9-phenyl-9H-fluoren-9-yl)phenyl)-9H-fluoren-2-amine and 9.54 g of the intermediate of 1-chloro-4-phenyldibenzo[b,d]furan were used in an equivalent ratio of 1:1.2.

Calculation value: C, 90.71; H, 5.38; N, 1.82; O, 2.08.

Analysis value: C, 90.71; H, 5.38; N, 1.82; O, 2.07.

### Synthesis Example 11: Synthesis of Compound 2-30

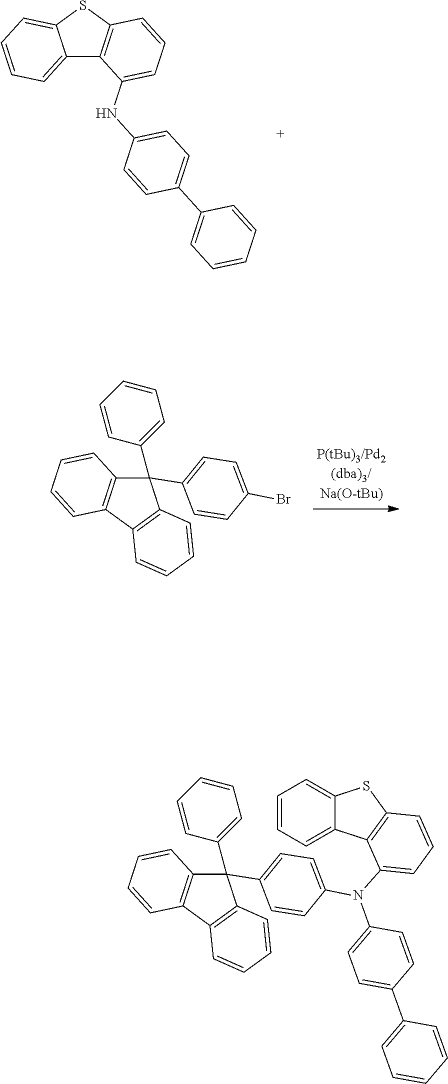 [SMILES: c1ccc(-c2ccc(Nc3cccc4sc5ccccc5c34)cc2)cc1 ; Brc1ccc(C2(c3ccccc3)c3ccccc3-c3ccccc32)cc1 ; c1ccc(-c2ccc(N(c3ccc(C4(c5ccccc5)c5ccccc5-c5ccccc54)cc3)c3cccc4sc5ccccc5c34)cc2)cc1]

Compound 2-30 (23.3 g, a yield: 77%) was synthesized in the same manner as in Synthesis Example 1 except that 16 g of the intermediate of N-([1,1′-biphenyl]-4-yl)dibenzo[b,d]thiophen-1-amine and 21.71 g of the intermediate of 9-(4-bromophenyl)-9-phenyl-9H-fluorene were used in an equivalent ratio of 1:1.2.

Calculation value: C, 88.12; H, 4.98; N, 2.10; S, 4.80.

Analysis value: C, 88.12; H, 4.98; N, 2.10; S, 4.80.

### Comparative Synthesis Example 1: Synthesis of Compound F-1

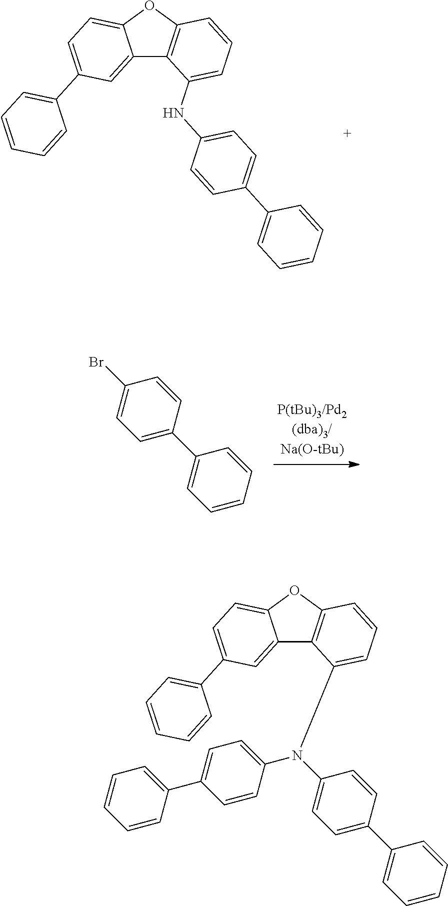 [SMILES: c1ccc(-c2ccc(Nc3cccc4oc5ccc(-c6ccccc6)cc5c34)cc2)cc1 ; Brc1ccc(-c2ccccc2)cc1 ; c1ccc(-c2ccc(N(c3ccc(-c4ccccc4)cc3)c3cccc4oc5ccc(-c6ccccc6)cc5c34)cc2)cc1]

Compound F-1 (16.5 g, a yield: 80%) was synthesized in the same manner as in Synthesis Example 1 except that 15 g of the intermediate of N-([1,1′-biphenyl]-4-yl)-8-phenyldibenzo[b,d]furan-1-amine and 10.2 g of the intermediate of 4-bromo-1,1′-biphenyl were used in an equivalent ratio of 1:1.2.

Calculation value: C, 90.27; H, 5.39; N, 2.02; O, 2.31.

Analysis value: C, 90.27; H, 5.39; N, 2.02; O, 2.31.

### Example 1

A glass substrate coated with a thin film of ITO/Ag/ITO was washed ultrasonically with distilled water. After washing with the distilled water, the glass substrate was ultrasonically washed with acetone or isopropyl alcohol, and dried and then, moved to a plasma cleaner, cleaned by using oxygen plasma for 10 minutes, and moved to a vacuum depositor. This prepared ITO/Ag/ITO (reflecting electrode) was used as an anode, Compound A doped with 3% NDP-9 (Novaled GmbH) was vacuum-deposited on the ITO/Ag/ITO substrate to form a 100 Å-thick hole injection layer, and Compound A was deposited on the hole injection layer to a thickness of 1,350 Å to form a hole transport layer. Compound 1-21 obtained in Synthesis Example 1 was deposited on the hole transport layer to a thickness of 285 Å to form a first hole transport auxiliary layer, and Compound 2-6 obtained in Synthesis Example 8 was deposited on the first hole transport auxiliary layer to a thickness of 50 Å to form a second hole transport auxiliary layer. On the second hole transport auxiliary layer, 85 wt % of Host H1 (40%) and Host H2 (60%) were used as a host, and 15 wt % of PtGD was doped as a dopant to form a 380 Å-thick light emitting layer by vacuum deposition. Subsequently, Compound C was deposited to form a 50 Å-thick electron transport auxiliary layer on the light emitting layer, and Compound D and Liq were simultaneously vacuum-deposited in a weight ratio of 1:1 to form a 310 Å-thick electron transport layer. On the electron transport layer, Yb and AgMg were sequentially vacuum-deposited, manufacturing an organic light emitting diode.

The structure was ITO/Ag/ITO/hole injection layer (Compound A doped with 3% NDP-9, 100 Å)/hole transport layer (Compound A, 1,350 Å)/first hole transport auxiliary layer (Compound 1-21, 285 Å)/second hole transport auxiliary layer (Compound 2-6, 50 Å)/light emitting layer [Host (Host H1, Host H2): PtGD=85 wt %:15 wt %] (380 Å)/electron transport auxiliary layer (Compound C, 50 Å)/electron transport layer (Compound D: Liq, 310 Å)/Yb/AgMg.
Compound A: N-(9,9-diphenyl-9H-fluoren-2-yl)-N,9-diphenyl-9H-carbazol-2-amine
Compound C: 4-{4-[3-(9,9-dimethyl-9H-fluoren-4-yl)phenyl]phenyl}-2-phenyl-6-(4-phenylphenyl)pyrimidine
Compound D: 2-(4-{2-[4-(diphenyl-1,3,5-triazin-2-yl)phenyl]naphthalen-1-yl}phenyl)-4,6-diphenyl-1,3,5-triazine

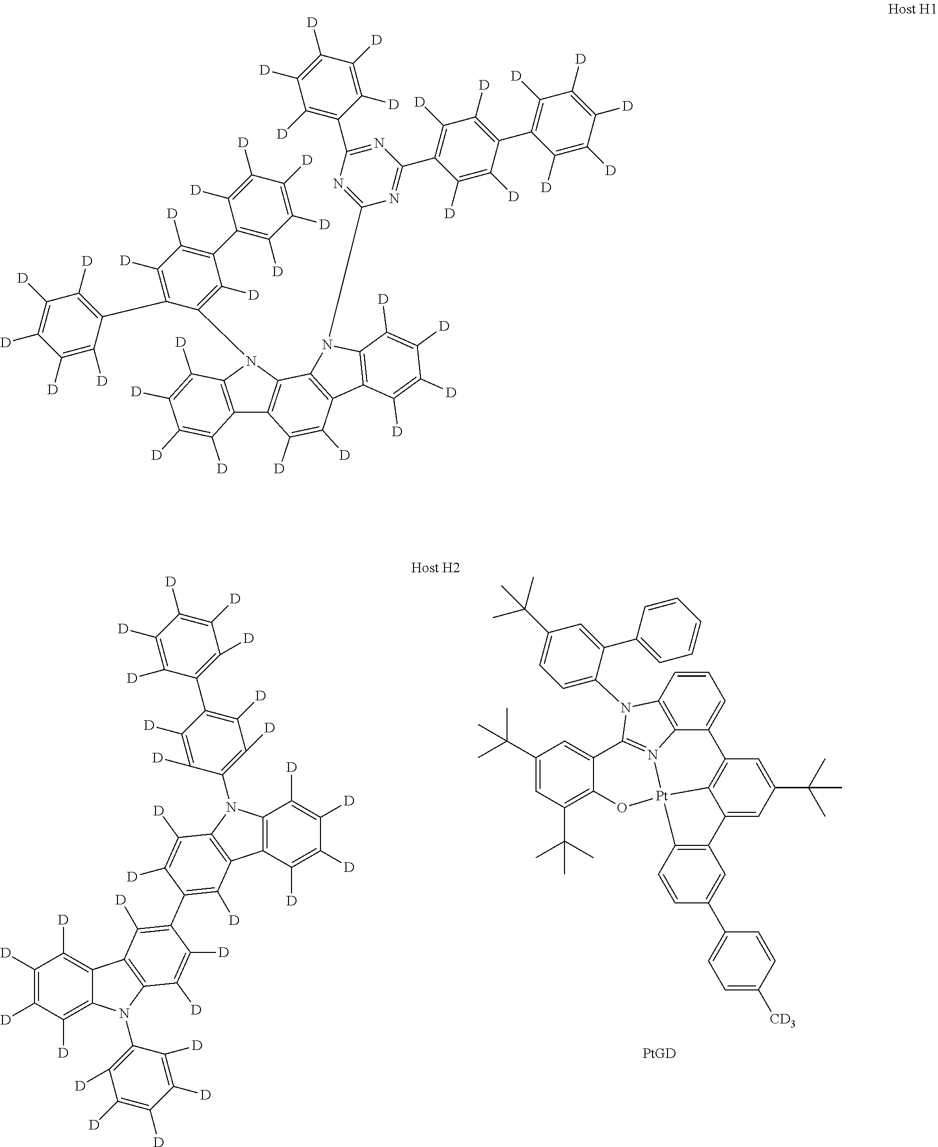

### Examples 2 to 17 and Comparative Examples 1 to 5

Each organic light emitting diode was manufactured in the same manner as in Example 1, except that the composition was changed to the one shown in Table 1.

### Evaluation

The driving voltage, luminous efficiency, and life-span characteristics of the organic light emitting diodes according to Examples 1 to 17 and Comparative Examples 1 to 5 were evaluated.

The specific measurement method was as follows, and the results are shown in Table 1.

### (1) Measurement of Current Density Change Depending on Voltage Change

The obtained organic light emitting diodes were measured regarding a current value flowing in the unit diode, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta CS-1000A), while the voltage of the organic light emitting diodes was increased from 0 V to 10 V.

### (3) Measurement of Luminous Efficiency

Using the luminance and current density measured from (1) and (2) above and a voltage, the current efficiency (cd/A) at the same current density (10 mA/cm²) was calculated.

The luminous efficiency values of Examples 1 to 17 and Comparative Examples 1 to 5 were calculated as relative values based on Comparative Example 1 and are listed in Table 1.

### (4) Measurement of Life-Span

The results were obtained by maintaining the luminance (cd/m²) at 24000 cd/m² and measuring the time for the current efficiency (cd/A) to decrease to 97%.

The life-span measurements of Examples 1 to 17 and Comparative Examples 1 to 5 were calculated as relative values based on Comparative Example 1 and are listed in Table 1.

### (5) Measurement of Driving Voltage

The results were obtained by measuring the driving voltage of each device at 15 mA/cm² using a current-voltmeter (Keithley 2400).

The driving voltages of Examples 1 to 17 and Comparative Examples 1 to 5 were calculated as relative values based on Comparative Example 1 and are listed in Table 1.

TABLE 1

| | | | Driving | | Life- |
|---|---|---|---|---|---|
| | First | Second | voltage | Efficiency | span |
| Nos. | compound | compound | (%) | (%) | (%) |
| Example 1 | 1-21 | 2-6 | 97 | 105 | 128 |
| Example 2 | 1-21 | 2-13 | 96 | 104 | 126 |
| Example 3 | 1-25 | 2-6 | 97 | 105 | 130 |
| Example 4 | 1-25 | 2-10 | 96 | 104 | 129 |
| Example 5 | 1-25 | 2-13 | 95 | 103 | 126 |
| Example 6 | 1-28 | 2-10 | 96 | 103 | 125 |
| Example 7 | 1-28 | 2-30 | 96 | 103 | 129 |
| Example 8 | 1-63 | 2-6 | 95 | 104 | 125 |
| Example 9 | 1-63 | 2-10 | 96 | 104 | 126 |
| Example 10 | 1-63 | 2-13 | 96 | 103 | 126 |
| Example 11 | 1-66 | 2-6 | 96 | 105 | 128 |
| Example 12 | 1-66 | 2-10 | 96 | 103 | 130 |
| Example 13 | 1-70 | 2-6 | 96 | 103 | 130 |
| Example 14 | 1-70 | 2-13 | 97 | 104 | 128 |
| Example 15 | 1-75 | 2-6 | 96 | 105 | 129 |
| Example 16 | 1-75 | 2-10 | 95 | 103 | 127 |
| Example 17 | 1-75 | 2-30 | 96 | 104 | 125 |
| Comparative | 1-21 | F-1 | 100 | 100 | 100 |
| Example 1 | | | | | |
| Comparative | 1-63 | F-1 | 98 | 95 | 80 |
| Example 2 | | | | | |
| Comparative | 1-66 | F-1 | 99 | 97 | 85 |
| Example 3 | | | | | |
| Comparative | F-1 | — | 107 | 100 | 95 |
| Example 4 | | | | | |
| Comparative | — | 2-13 | 104 | 99 | 105 |
| Example 5 | | | | | |

Referring to Table 1, the driving voltage, luminous efficiency, and life-span characteristics of the organic light emitting diodes according to Examples 1 to 17 were significantly improved compared to the organic light emitting diodes according to Comparative Examples 1 to 5.

One or more embodiments may provide a high efficiency, low driving voltage, and long life-span organic optoelectronic device.

One or more embodiments may provide a display device including an organic optoelectronic device.

An organic optoelectronic device having high efficiency, low driving voltage, and long life-span may be realized.

Example embodiments have been disclosed herein, and although specific terms are employed, they are used and are to be interpreted in a generic and descriptive sense only and not for purposes of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. An organic optoelectronic device, comprising:
an anode and a cathode facing each other;
a light emitting layer between the anode and the cathode;
a hole transport layer between the anode and the light emitting layer; and
a hole transport auxiliary layer between the hole transport layer and the light emitting layer,
wherein:
the hole transport auxiliary layer includes a first hole transport auxiliary layer adjacent to the hole transport layer and a second hole transport auxiliary layer adjacent to the light emitting layer,
the first hole transport auxiliary layer includes a first compound represented by Chemical Formula 1, and
the second hole transport auxiliary layer includes a second compound represented by Chemical Formula 2, 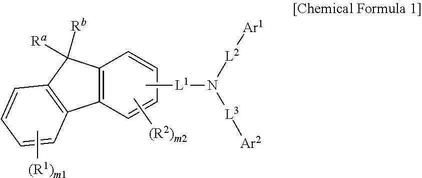
in Chemical Formula 1,
R^{a} and R^{b} are each independently a substituted or unsubstituted C1 to C30 alkyl group or a substituted or unsubstituted C6 to C30 aryl group,
L¹ to L³ are each independently a single bond, a substituted or unsubstituted C6 to C20 arylene group, or a substituted or unsubstituted C2 to C30 heterocyclic group,
R¹ and R² are each independently hydrogen, deuterium, a cyano group, a substituted or unsubstituted C1 to C30 alkyl group, a substituted or unsubstituted C6 to C30 aryl group, or a substituted or unsubstituted C2 to C30 heterocyclic group,
Ar¹ and Ar² are each independently a substituted or unsubstituted C3 to C40 cycloalkyl group or a substituted or unsubstituted C6 to C20 aryl group,
m1 is an integer of 1 to 4, and
m2 is an integer of 1 to 3; 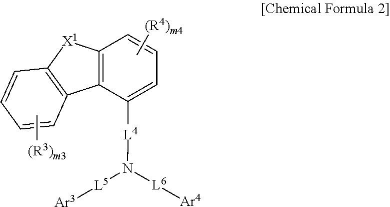
in Chemical Formula 2,
X¹ is O or S,
L⁴ to L⁶ are each independently a single bond, a substituted or unsubstituted C6 to C20 arylene group, or a substituted or unsubstituted C2 to C30 heterocyclic group,
R³ and R⁴ are each independently hydrogen, deuterium, a cyano group, a substituted or unsubstituted C1 to C30 alkyl group, a substituted or unsubstituted C6 to C30 aryl group, or a substituted or unsubstituted C2 to C30 heterocyclic group,
Ar³ and Ar⁴ are each independently a substituted or unsubstituted C6 to C20 aryl group or a substituted or unsubstituted C2 to C30 heterocyclic group,
at least one of Ar³ and Ar⁴ is a substituted or unsubstituted 9-fluorenyl group,
m3 is an integer of 1 to 4, and
m4 is an integer of 1 to 3.

2. The organic optoelectronic device as claimed in claim 1, wherein:
Chemical Formula 1 is represented by one of Chemical Formula 1-1 to Chemical Formula 1-4, and 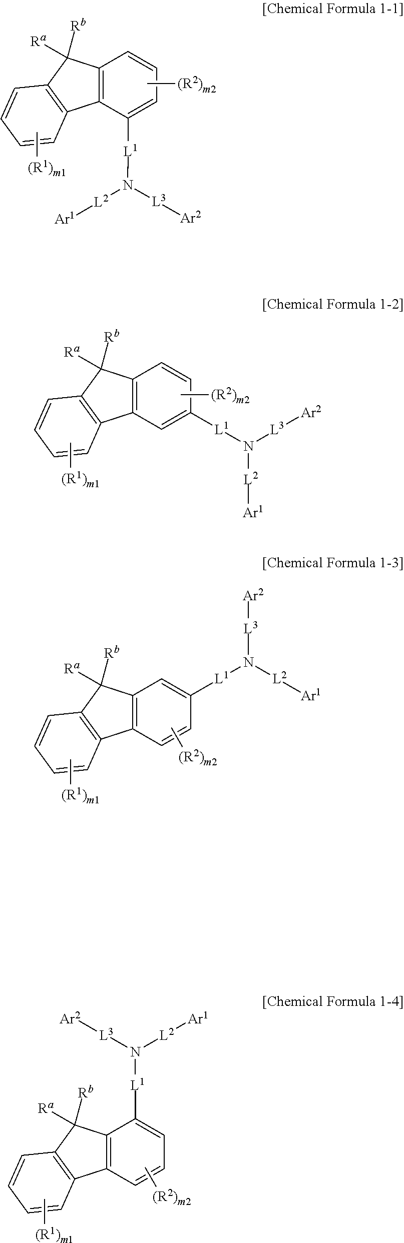
in Chemical Formula 1-1 to Chemical Formula 1-4, L¹ to L³, R^{a}, R^{b}, R¹, R², Ar¹, Ar², m1, and m2 are defined the same as those of Chemical Formula 1.

3. The organic optoelectronic device as claimed in claim 1, wherein Ar¹ and Ar² are each independently a substituted or unsubstituted cyclopentyl group, a substituted or unsubstituted cyclohexyl group, a substituted or unsubstituted bicyclo[2,2,1]heptyl group, a substituted or unsubstituted adamantyl group, a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, or a substituted or unsubstituted phenanthrenyl group, a substituted or unsubstituted triphenylene group, a substituted or unsubstituted fluorenyl group, or a substituted or unsubstituted dibenzosilolyl group.

4. The organic optoelectronic device as claimed in claim 1, wherein:
moieties L²-Ar¹ and L³-Ar² are each independently a moiety of Group I, 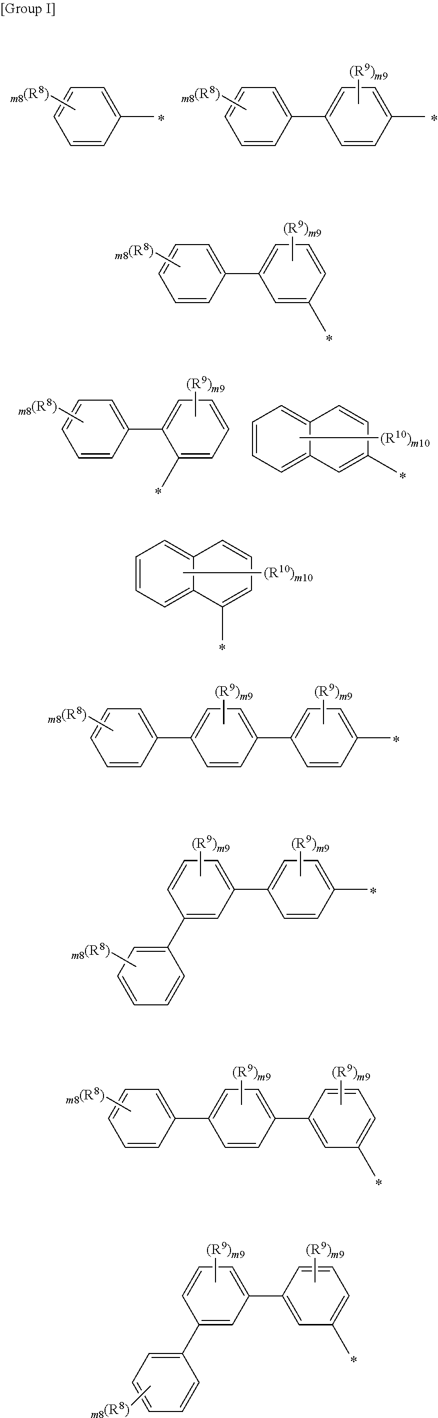 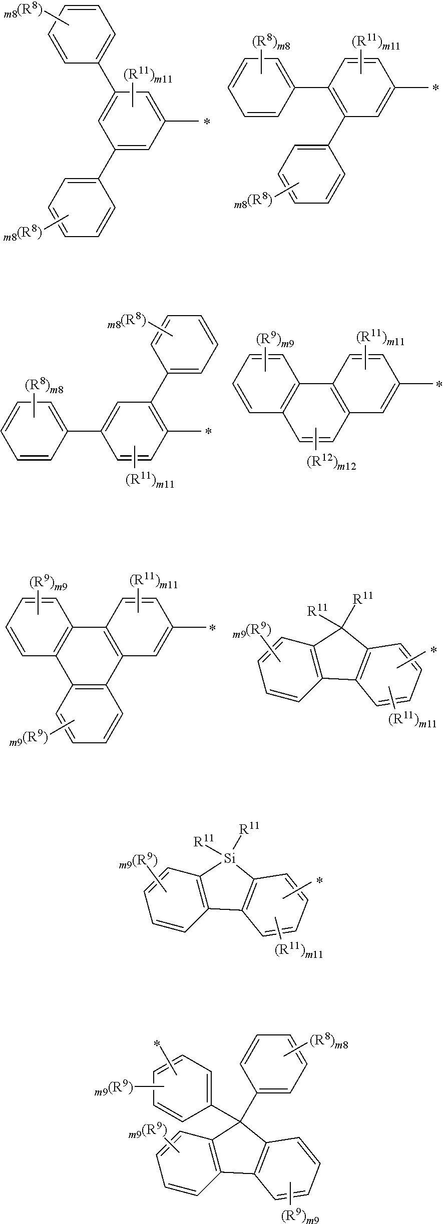 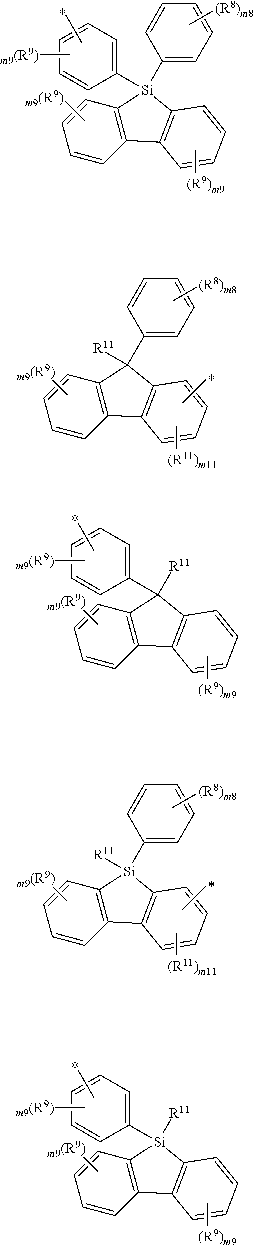
in Group I,
R⁸ to R¹² are each independently hydrogen, deuterium, a cyano group, a substituted or unsubstituted C1 to C10 alkyl group, or a substituted or unsubstituted C6 to C12 aryl group,
m8 is an integer of 1 to 5,
m9 is an integer of 1 to 4,
m10 is an integer of 1 to 7,
m11 is an integer of 1 to 3,
m12 is an integer of 1 or 2, and
* is a linking point.

5. The organic optoelectronic device as claimed in claim 1, wherein the first compound is a compound of Group 1:

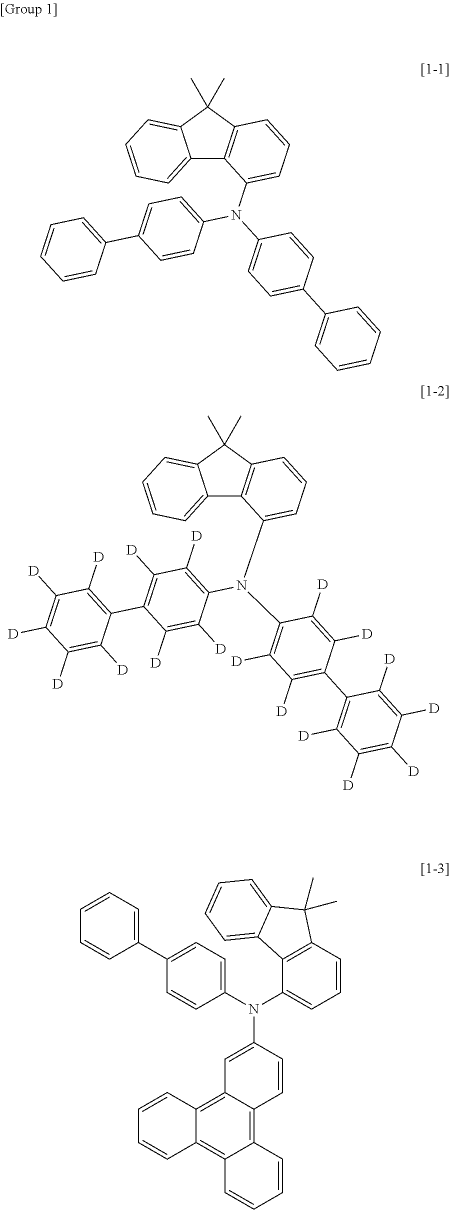 [SMILES: CC1(C)c2ccccc2-c2c(N(c3ccc(-c4ccccc4)cc3)c3ccc(-c4ccccc4)cc3)cccc21 ; [2H]c1c([2H])c([2H])c(-c2c([2H])c([2H])c(N(c3cccc4c3-c3ccccc3C4(C)C)c3c([2H])c([2H])c(-c4c([2H])c([2H])c([2H])c([2H])c4[2H])c([2H])c3[2H])c([2H])c2[2H])c([2H])c1[2H] ; CC1(C)c2ccccc2-c2c(N(c3ccc(-c4ccccc4)cc3)c3ccc4c5ccccc5c5ccccc5c4c3)cccc21]

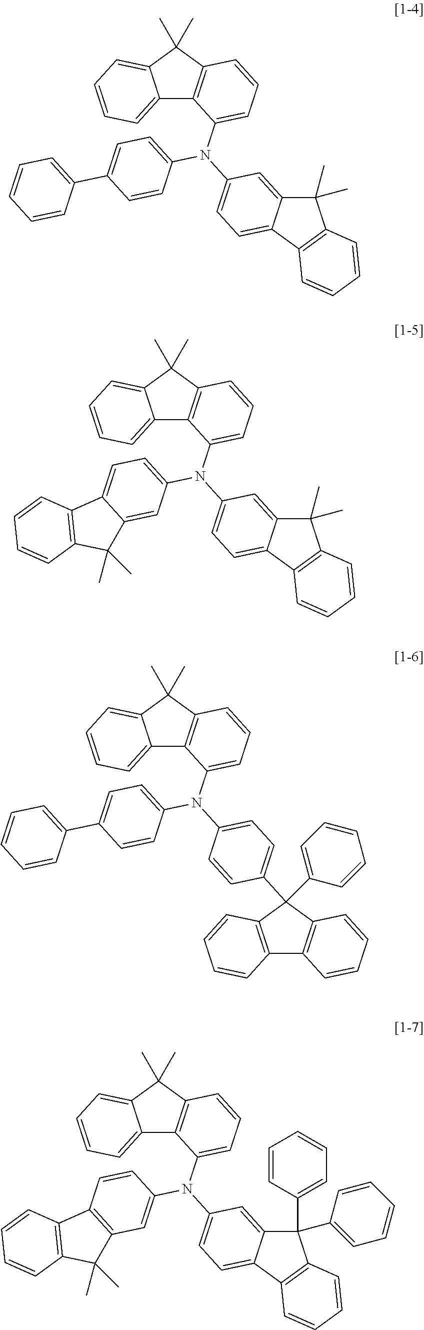 [SMILES: CC1(C)c2ccccc2-c2ccc(N(c3ccc(-c4ccccc4)cc3)c3cccc4c3-c3ccccc3C4(C)C)cc21 ; CC1(C)c2ccccc2-c2ccc(N(c3ccc4c(c3)C(C)(C)c3ccccc3-4)c3cccc4c3-c3ccccc3C4(C)C)cc21 ; CC1(C)c2ccccc2-c2c(N(c3ccc(-c4ccccc4)cc3)c3ccc(C4(c5ccccc5)c5ccccc5-c5ccccc54)cc3)cccc21 ; CC1(C)c2ccccc2-c2ccc(N(c3ccc4c(c3)C(c3ccccc3)(c3ccccc3)c3ccccc3-4)c3cccc4c3-c3ccccc3C4(C)C)cc21]

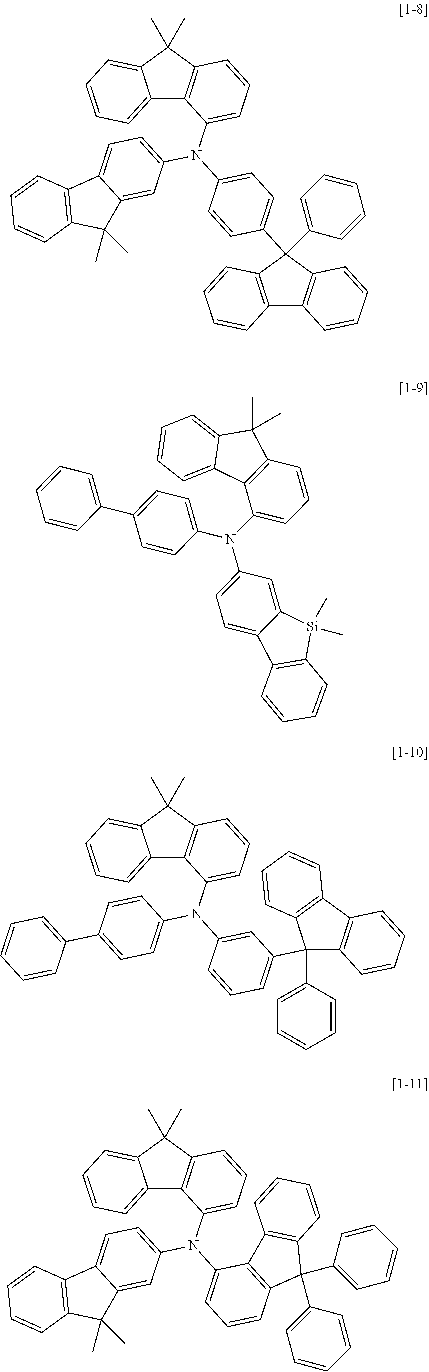 [SMILES: CC1(C)c2ccccc2-c2ccc(N(c3ccc(C4(c5ccccc5)c5ccccc5-c5ccccc54)cc3)c3cccc4c3-c3ccccc3C4(C)C)cc21 ; CC1(C)c2ccccc2-c2c(N(c3ccc(-c4ccccc4)cc3)c3ccc4c(c3)[Si](C)(C)c3ccccc3-4)cccc21 ; CC1(C)c2ccccc2-c2c(N(c3ccc(-c4ccccc4)cc3)c3cccc(C4(c5ccccc5)c5ccccc5-c5ccccc54)c3)cccc21 ; CC1(C)c2ccccc2-c2ccc(N(c3cccc4c3-c3ccccc3C4(C)C)c3cccc4c3-c3ccccc3C4(c3ccccc3)c3ccccc3)cc21]

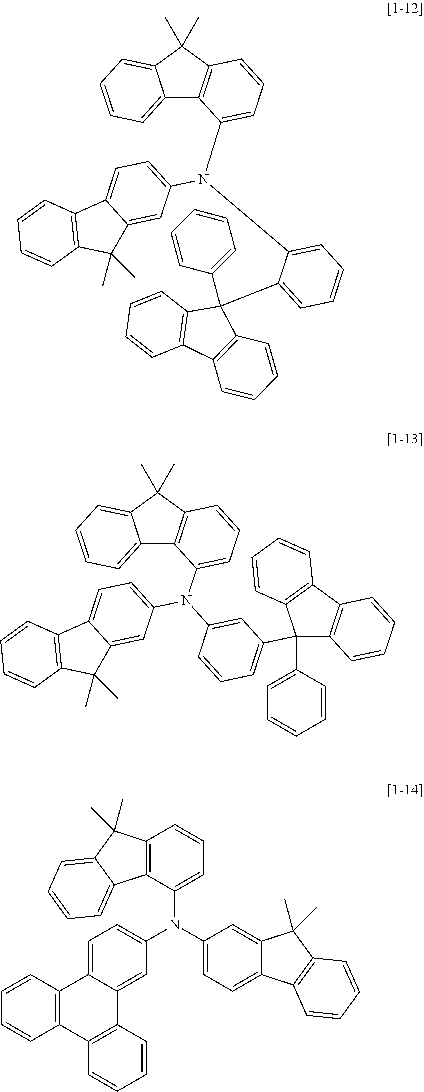 [SMILES: CC1(C)c2ccccc2-c2ccc(N(c3ccccc3C3(c4ccccc4)c4ccccc4-c4ccccc43)c3cccc4c3-c3ccccc3C4(C)C)cc21 ; CC1(C)c2ccccc2-c2ccc(N(c3cccc(C4(c5ccccc5)c5ccccc5-c5ccccc54)c3)c3cccc4c3-c3ccccc3C4(C)C)cc21 ; CC1(C)c2ccccc2-c2ccc(N(c3ccc4c5ccccc5c5ccccc5c4c3)c3cccc4c3-c3ccccc3C4(C)C)cc21]

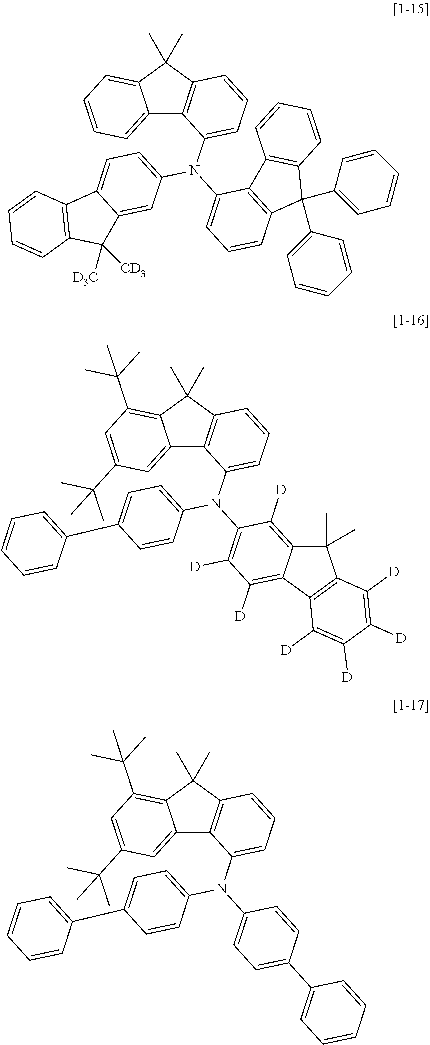 [SMILES: [2H]C([2H])([2H])C1(C([2H])([2H])[2H])c2ccccc2-c2ccc(N(c3cccc4c3-c3ccccc3C4(C)C)c3cccc4c3-c3ccccc3C4(c3ccccc3)c3ccccc3)cc21 ; [2H]c1c([2H])c([2H])c2c(c1[2H])-c1c([2H])c([2H])c(N(c3ccc(-c4ccccc4)cc3)c3cccc4c3-c3cc(C(C)(C)C)cc(C(C)(C)C)c3C4(C)C)c([2H])c1C2(C)C ; CC(C)(C)c1cc2c(c(C(C)(C)C)c1)C(C)(C)c1cccc(N(c3ccc(-c4ccccc4)cc3)c3ccc(-c4ccccc4)cc3)c1-2]

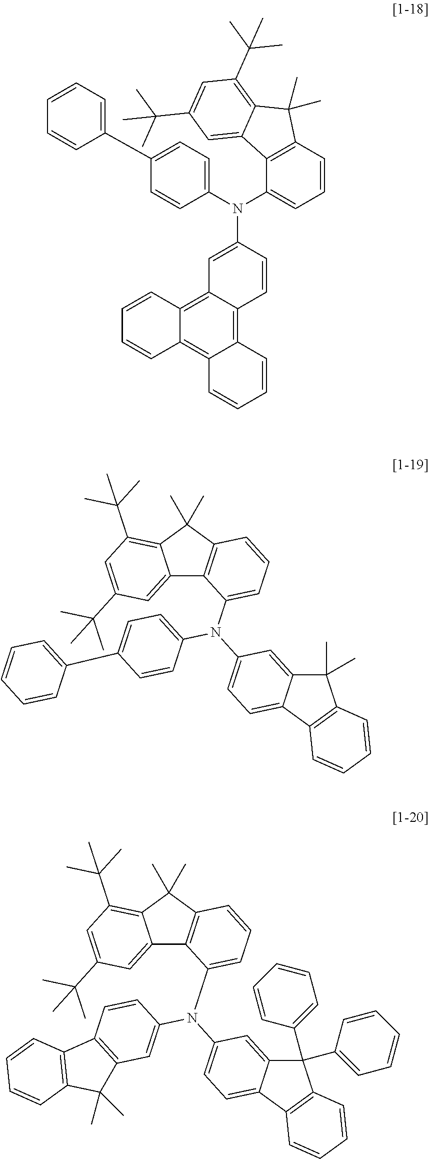 [SMILES: CC(C)(C)c1cc2c(c(C(C)(C)C)c1)C(C)(C)c1cccc(N(c3ccc(-c4ccccc4)cc3)c3ccc4c5ccccc5c5ccccc5c4c3)c1-2 ; CC(C)(C)c1cc2c(c(C(C)(C)C)c1)C(C)(C)c1cccc(N(c3ccc(-c4ccccc4)cc3)c3ccc4c(c3)C(C)(C)c3ccccc3-4)c1-2 ; CC(C)(C)c1cc2c(c(C(C)(C)C)c1)C(C)(C)c1cccc(N(c3ccc4c(c3)C(C)(C)c3ccccc3-4)c3ccc4c(c3)C(c3ccccc3)(c3ccccc3)c3ccccc3-4)c1-2]

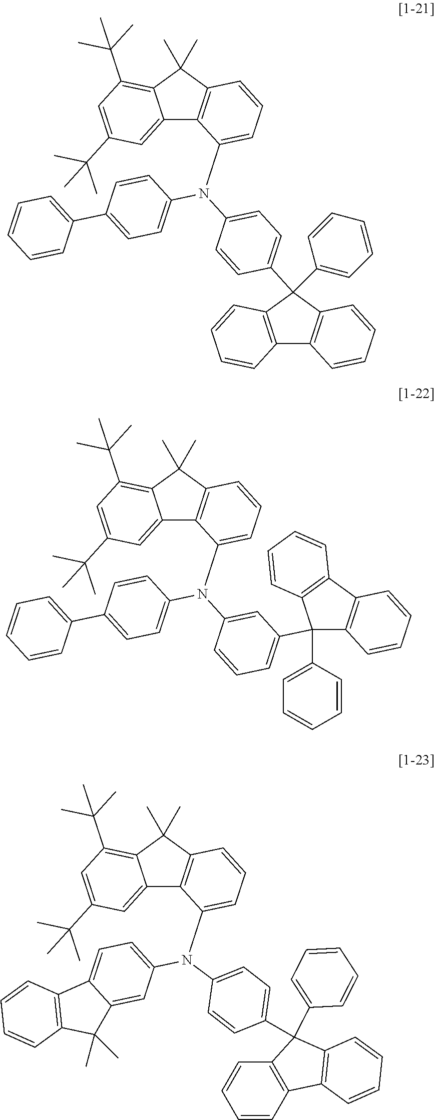 [SMILES: CC(C)(C)c1cc2c(c(C(C)(C)C)c1)C(C)(C)c1cccc(N(c3ccc(-c4ccccc4)cc3)c3ccc(C4(c5ccccc5)c5ccccc5-c5ccccc54)cc3)c1-2 ; CC(C)(C)c1cc2c(c(C(C)(C)C)c1)C(C)(C)c1cccc(N(c3ccc(-c4ccccc4)cc3)c3cccc(C4(c5ccccc5)c5ccccc5-c5ccccc54)c3)c1-2 ; CC(C)(C)c1cc2c(c(C(C)(C)C)c1)C(C)(C)c1cccc(N(c3ccc(C4(c5ccccc5)c5ccccc5-c5ccccc54)cc3)c3ccc4c(c3)C(C)(C)c3ccccc3-4)c1-2]

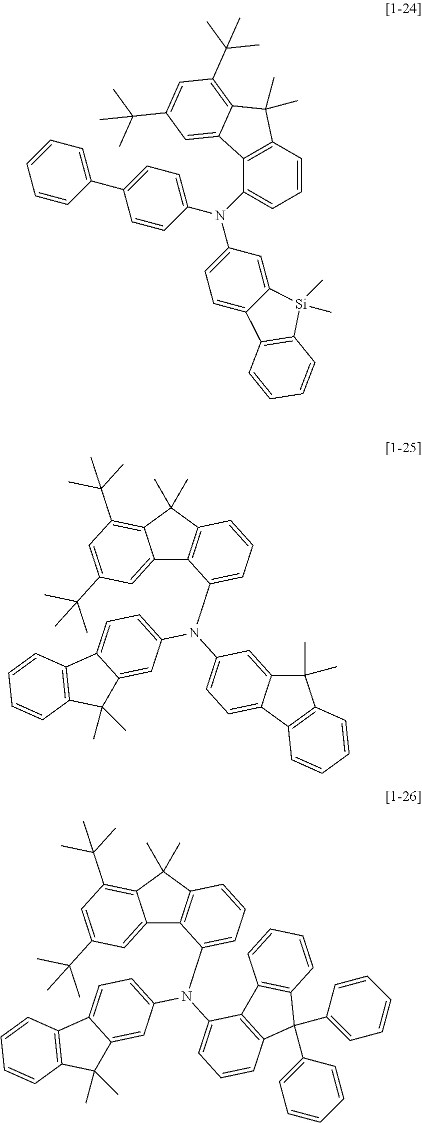 [SMILES: CC(C)(C)c1cc2c(c(C(C)(C)C)c1)C(C)(C)c1cccc(N(c3ccc(-c4ccccc4)cc3)c3ccc4c(c3)[Si](C)(C)c3ccccc3-4)c1-2 ; CC(C)(C)c1cc2c(c(C(C)(C)C)c1)C(C)(C)c1cccc(N(c3ccc4c(c3)C(C)(C)c3ccccc3-4)c3ccc4c(c3)C(C)(C)c3ccccc3-4)c1-2 ; CC(C)(C)c1cc2c(c(C(C)(C)C)c1)C(C)(C)c1cccc(N(c3ccc4c(c3)C(C)(C)c3ccccc3-4)c3cccc4c3-c3ccccc3C4(c3ccccc3)c3ccccc3)c1-2]

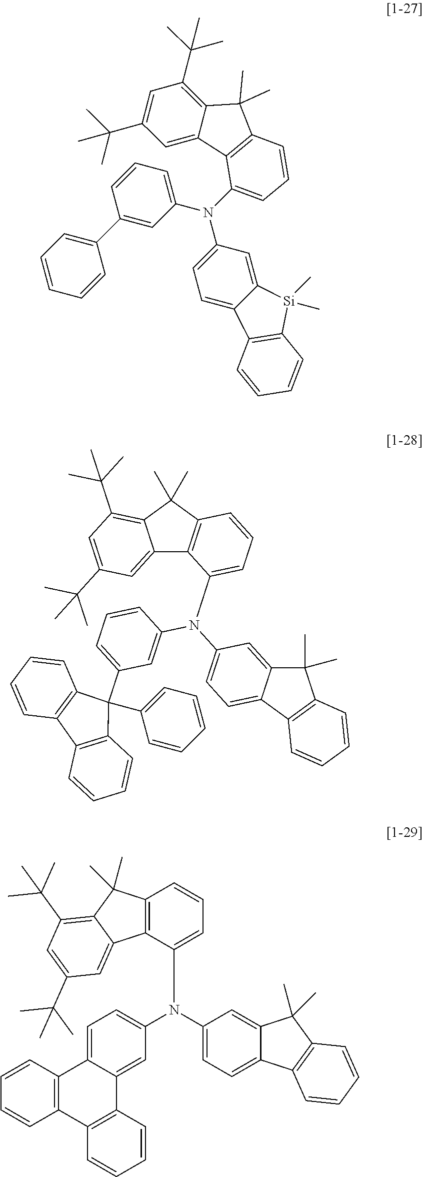 [SMILES: CC(C)(C)c1cc2c(c(C(C)(C)C)c1)C(C)(C)c1cccc(N(c3cccc(-c4ccccc4)c3)c3ccc4c(c3)[Si](C)(C)c3ccccc3-4)c1-2 ; CC(C)(C)c1cc2c(c(C(C)(C)C)c1)C(C)(C)c1cccc(N(c3cccc(C4(c5ccccc5)c5ccccc5-c5ccccc54)c3)c3ccc4c(c3)C(C)(C)c3ccccc3-4)c1-2 ; CC(C)(C)c1cc2c(c(C(C)(C)C)c1)C(C)(C)c1cccc(N(c3ccc4c(c3)C(C)(C)c3ccccc3-4)c3ccc4c5ccccc5c5ccccc5c4c3)c1-2]

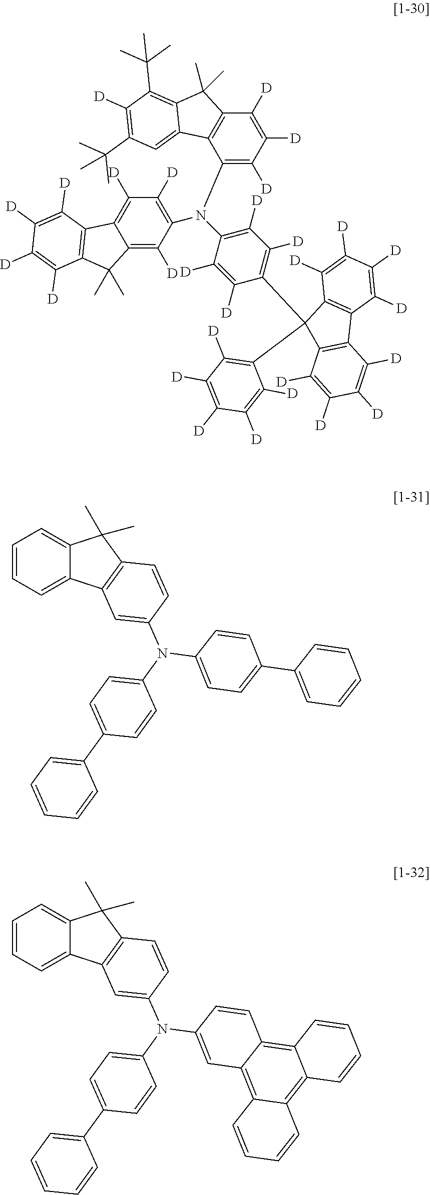 [SMILES: [2H]c1c([2H])c([2H])c(C2(c3c([2H])c([2H])c(N(c4c([2H])c([2H])c5c(c4[2H])C(C)(C)c4c([2H])c([2H])c([2H])c([2H])c4-5)c4c([2H])c([2H])c([2H])c5c4-c4cc(C(C)(C)C)c([2H])c(C(C)(C)C)c4C5(C)C)c([2H])c3[2H])c3c([2H])c([2H])c([2H])c([2H])c3-c3c([2H])c([2H])c([2H])c([2H])c32)c([2H])c1[2H] ; CC1(C)c2ccccc2-c2cc(N(c3ccc(-c4ccccc4)cc3)c3ccc(-c4ccccc4)cc3)ccc21 ; CC1(C)c2ccccc2-c2cc(N(c3ccc(-c4ccccc4)cc3)c3ccc4c5ccccc5c5ccccc5c4c3)ccc21]

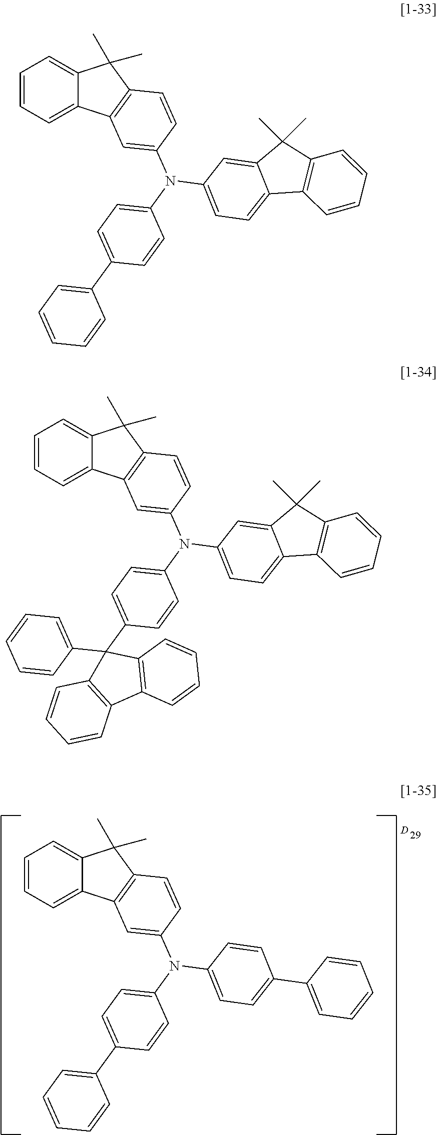

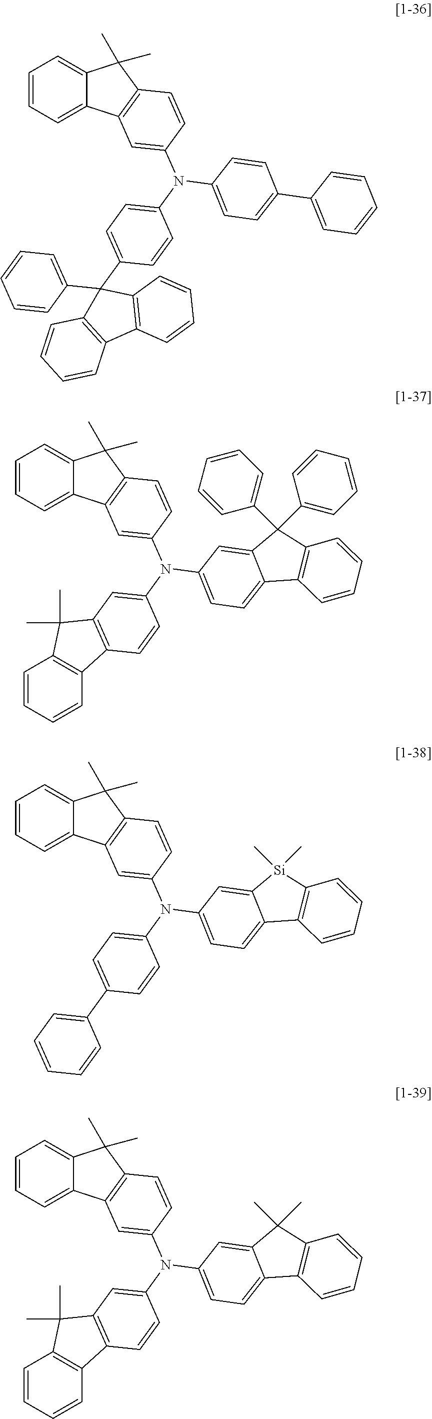

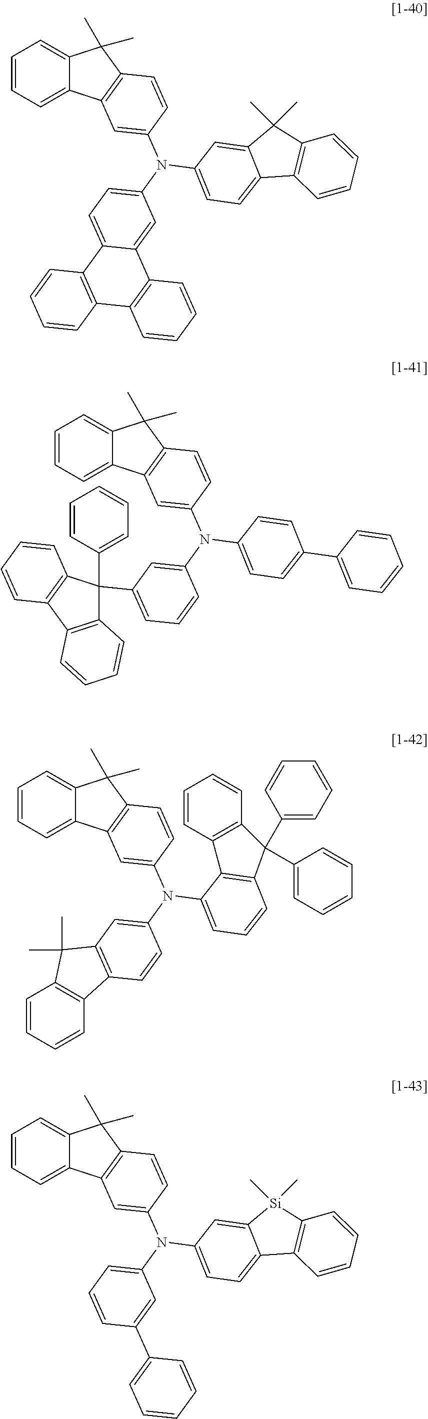 [SMILES: CC1(C)c2ccccc2-c2cc(N(c3ccc4c(c3)C(C)(C)c3ccccc3-4)c3ccc4c5ccccc5c5ccccc5c4c3)ccc21 ; CC1(C)c2ccccc2-c2cc(N(c3ccc(-c4ccccc4)cc3)c3cccc(C4(c5ccccc5)c5ccccc5-c5ccccc54)c3)ccc21 ; CC1(C)c2ccccc2-c2cc(N(c3ccc4c(c3)C(C)(C)c3ccccc3-4)c3cccc4c3-c3ccccc3C4(c3ccccc3)c3ccccc3)ccc21 ; CC1(C)c2ccccc2-c2cc(N(c3cccc(-c4ccccc4)c3)c3ccc4c(c3)[Si](C)(C)c3ccccc3-4)ccc21]

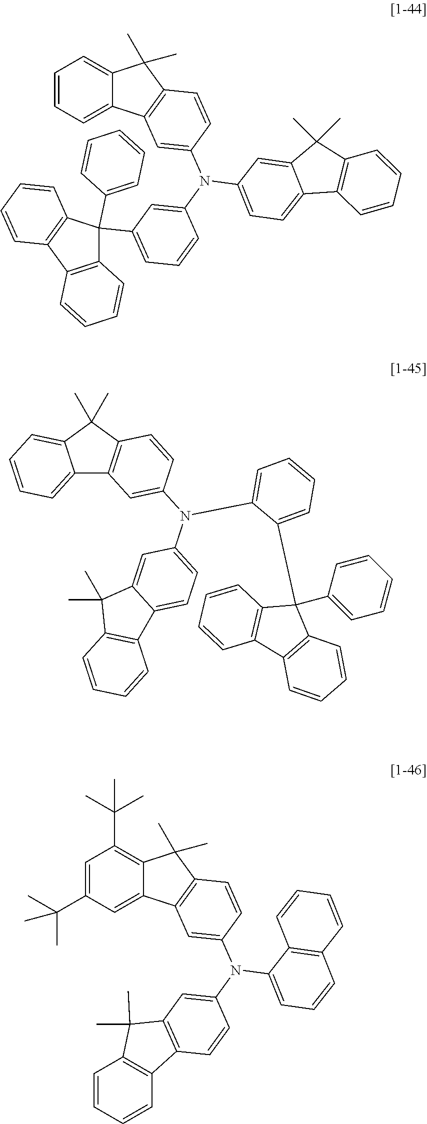 [SMILES: CC1(C)c2ccccc2-c2cc(N(c3cccc(C4(c5ccccc5)c5ccccc5-c5ccccc54)c3)c3ccc4c(c3)C(C)(C)c3ccccc3-4)ccc21 ; CC1(C)c2ccccc2-c2cc(N(c3ccc4c(c3)C(C)(C)c3ccccc3-4)c3ccccc3C3(c4ccccc4)c4ccccc4-c4ccccc43)ccc21 ; CC(C)(C)c1cc2c(c(C(C)(C)C)c1)C(C)(C)c1ccc(N(c3ccc4c(c3)C(C)(C)c3ccccc3-4)c3cccc4ccccc34)cc1-2]

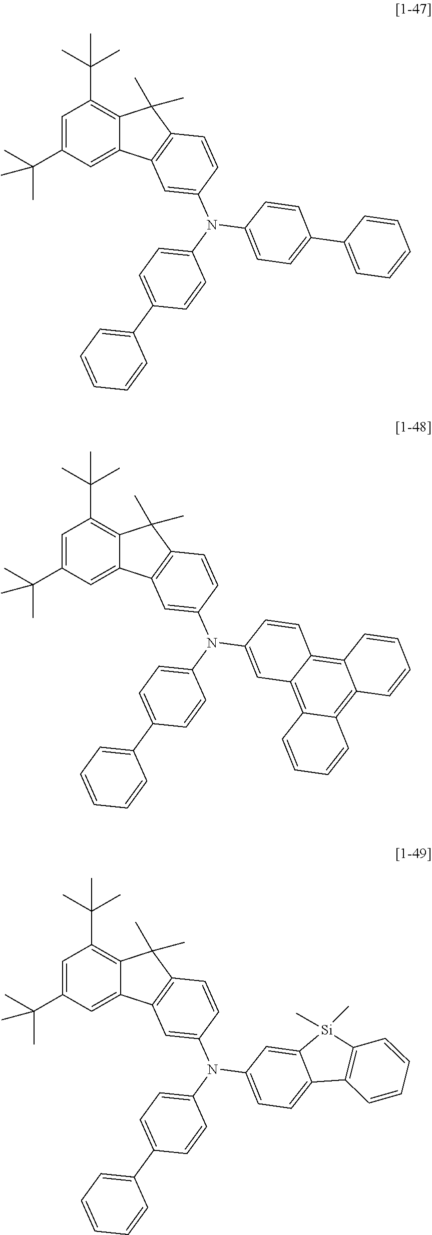 [SMILES: CC(C)(C)c1cc2c(c(C(C)(C)C)c1)C(C)(C)c1ccc(N(c3ccc(-c4ccccc4)cc3)c3ccc(-c4ccccc4)cc3)cc1-2 ; CC(C)(C)c1cc2c(c(C(C)(C)C)c1)C(C)(C)c1ccc(N(c3ccc(-c4ccccc4)cc3)c3ccc4c5ccccc5c5ccccc5c4c3)cc1-2 ; CC(C)(C)c1cc2c(c(C(C)(C)C)c1)C(C)(C)c1ccc(N(c3ccc(-c4ccccc4)cc3)c3ccc4c(c3)[Si](C)(C)c3ccccc3-4)cc1-2]

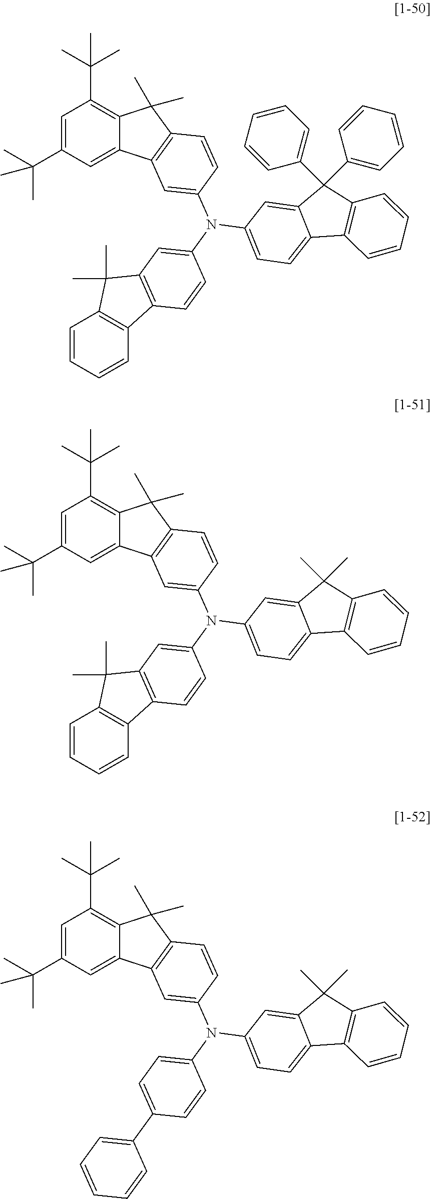 [SMILES: CC(C)(C)c1cc2c(c(C(C)(C)C)c1)C(C)(C)c1ccc(N(c3ccc4c(c3)C(C)(C)c3ccccc3-4)c3ccc4c(c3)C(c3ccccc3)(c3ccccc3)c3ccccc3-4)cc1-2 ; CC(C)(C)c1cc2c(c(C(C)(C)C)c1)C(C)(C)c1ccc(N(c3ccc4c(c3)C(C)(C)c3ccccc3-4)c3ccc4c(c3)C(C)(C)c3ccccc3-4)cc1-2 ; CC(C)(C)c1cc2c(c(C(C)(C)C)c1)C(C)(C)c1ccc(N(c3ccc(-c4ccccc4)cc3)c3ccc4c(c3)C(C)(C)c3ccccc3-4)cc1-2]

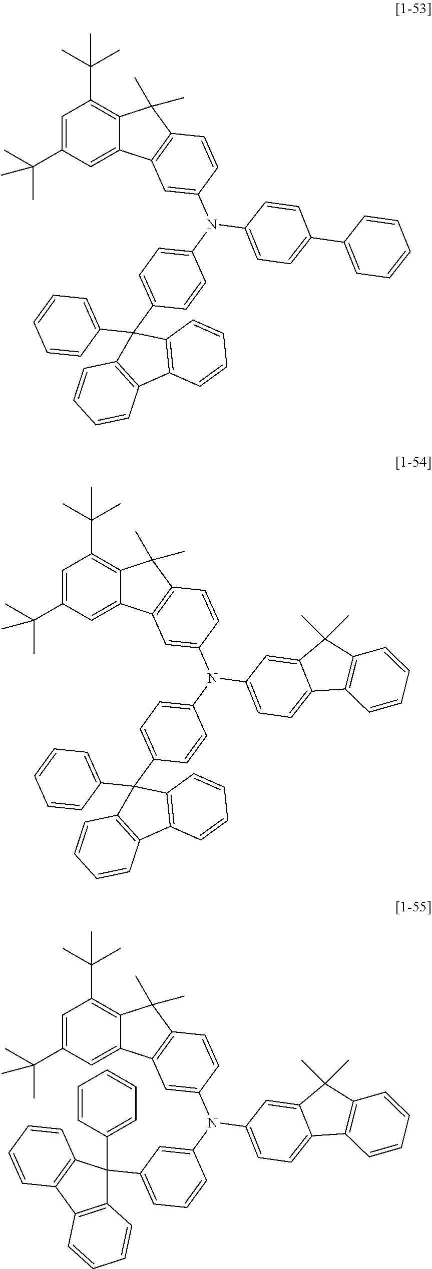 [SMILES: CC(C)(C)c1cc2c(c(C(C)(C)C)c1)C(C)(C)c1ccc(N(c3ccc(-c4ccccc4)cc3)c3ccc(C4(c5ccccc5)c5ccccc5-c5ccccc54)cc3)cc1-2 ; CC(C)(C)c1cc2c(c(C(C)(C)C)c1)C(C)(C)c1ccc(N(c3ccc(C4(c5ccccc5)c5ccccc5-c5ccccc54)cc3)c3ccc4c(c3)C(C)(C)c3ccccc3-4)cc1-2 ; CC(C)(C)c1cc2c(c(C(C)(C)C)c1)C(C)(C)c1ccc(N(c3cccc(C4(c5ccccc5)c5ccccc5-c5ccccc54)c3)c3ccc4c(c3)C(C)(C)c3ccccc3-4)cc1-2]

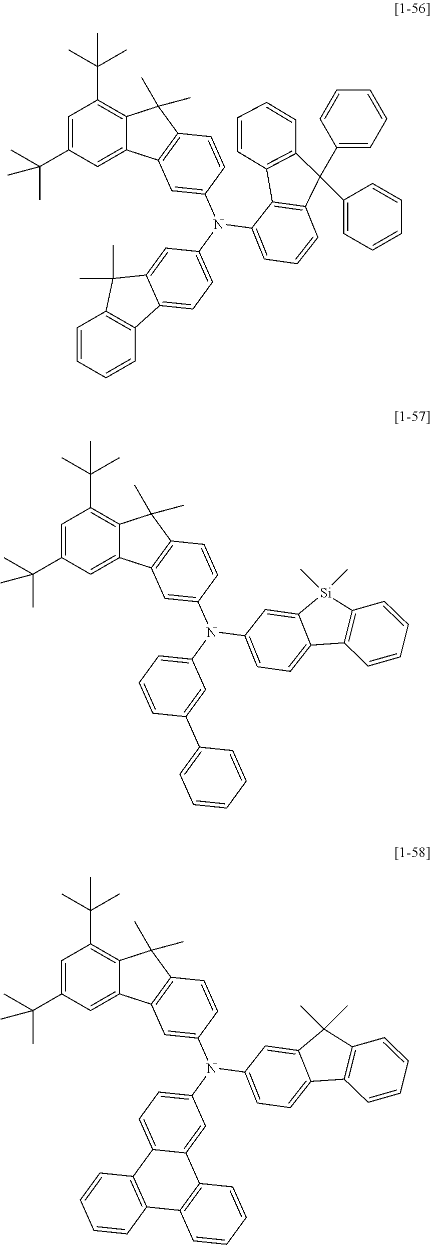 [SMILES: CC(C)(C)c1cc2c(c(C(C)(C)C)c1)C(C)(C)c1ccc(N(c3ccc4c(c3)C(C)(C)c3ccccc3-4)c3cccc4c3-c3ccccc3C4(c3ccccc3)c3ccccc3)cc1-2 ; CC(C)(C)c1cc2c(c(C(C)(C)C)c1)C(C)(C)c1ccc(N(c3cccc(-c4ccccc4)c3)c3ccc4c(c3)[Si](C)(C)c3ccccc3-4)cc1-2 ; CC(C)(C)c1cc2c(c(C(C)(C)C)c1)C(C)(C)c1ccc(N(c3ccc4c(c3)C(C)(C)c3ccccc3-4)c3ccc4c5ccccc5c5ccccc5c4c3)cc1-2]

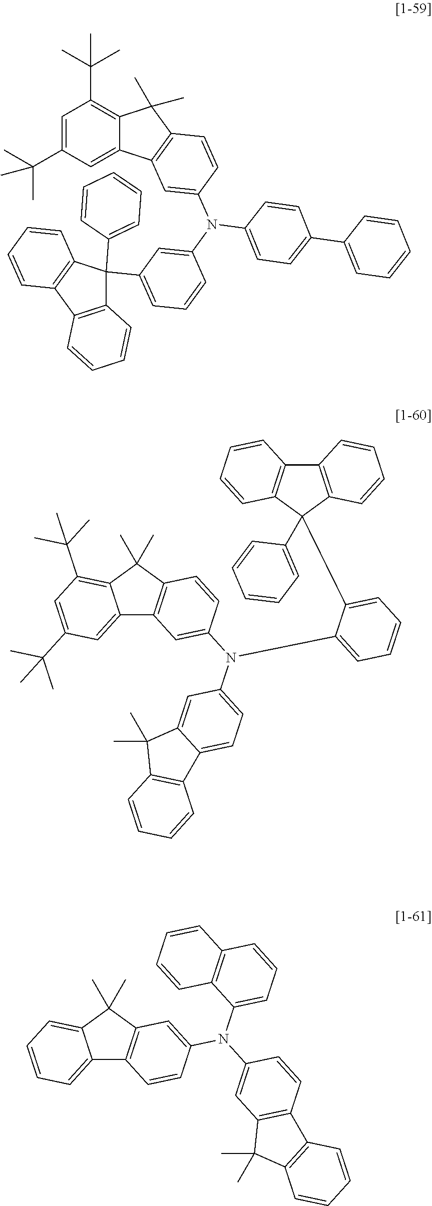 [SMILES: CC(C)(C)c1cc2c(c(C(C)(C)C)c1)C(C)(C)c1ccc(N(c3ccc(-c4ccccc4)cc3)c3cccc(C4(c5ccccc5)c5ccccc5-c5ccccc54)c3)cc1-2 ; CC(C)(C)c1cc2c(c(C(C)(C)C)c1)C(C)(C)c1ccc(N(c3ccc4c(c3)C(C)(C)c3ccccc3-4)c3ccccc3C3(c4ccccc4)c4ccccc4-c4ccccc43)cc1-2 ; CC1(C)c2ccccc2-c2ccc(N(c3ccc4c(c3)C(C)(C)c3ccccc3-4)c3cccc4ccccc34)cc21]

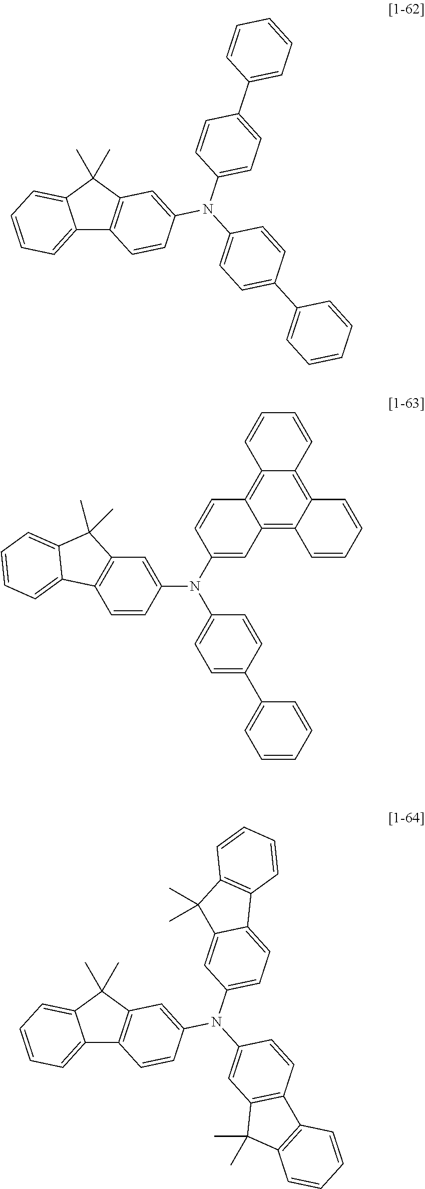 [SMILES: CC1(C)c2ccccc2-c2ccc(N(c3ccc(-c4ccccc4)cc3)c3ccc(-c4ccccc4)cc3)cc21 ; CC1(C)c2ccccc2-c2ccc(N(c3ccc(-c4ccccc4)cc3)c3ccc4c5ccccc5c5ccccc5c4c3)cc21 ; CC1(C)c2ccccc2-c2ccc(N(c3ccc4c(c3)C(C)(C)c3ccccc3-4)c3ccc4c(c3)C(C)(C)c3ccccc3-4)cc21]

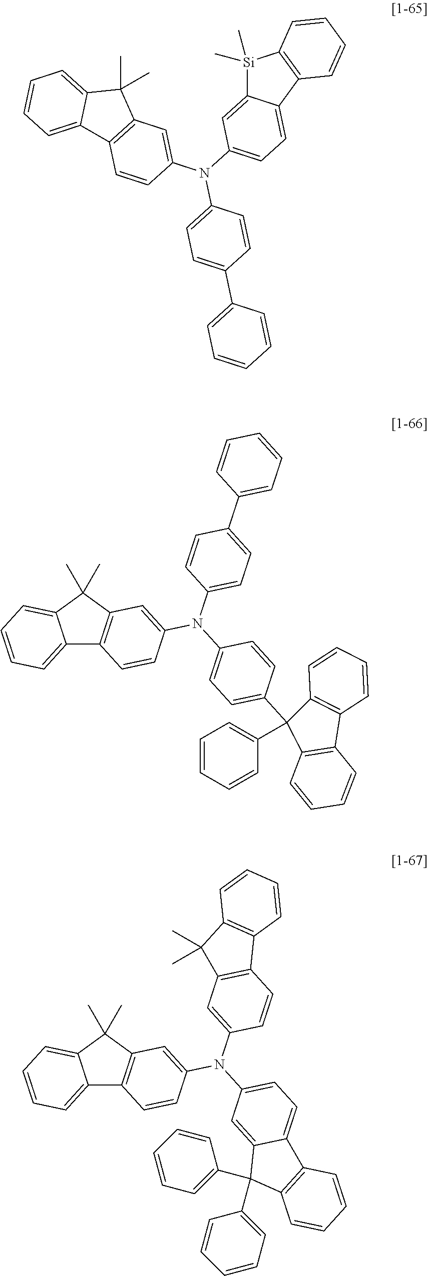 [SMILES: CC1(C)c2ccccc2-c2ccc(N(c3ccc(-c4ccccc4)cc3)c3ccc4c(c3)[Si](C)(C)c3ccccc3-4)cc21 ; CC1(C)c2ccccc2-c2ccc(N(c3ccc(-c4ccccc4)cc3)c3ccc(C4(c5ccccc5)c5ccccc5-c5ccccc54)cc3)cc21 ; CC1(C)c2ccccc2-c2ccc(N(c3ccc4c(c3)C(C)(C)c3ccccc3-4)c3ccc4c(c3)C(c3ccccc3)(c3ccccc3)c3ccccc3-4)cc21]

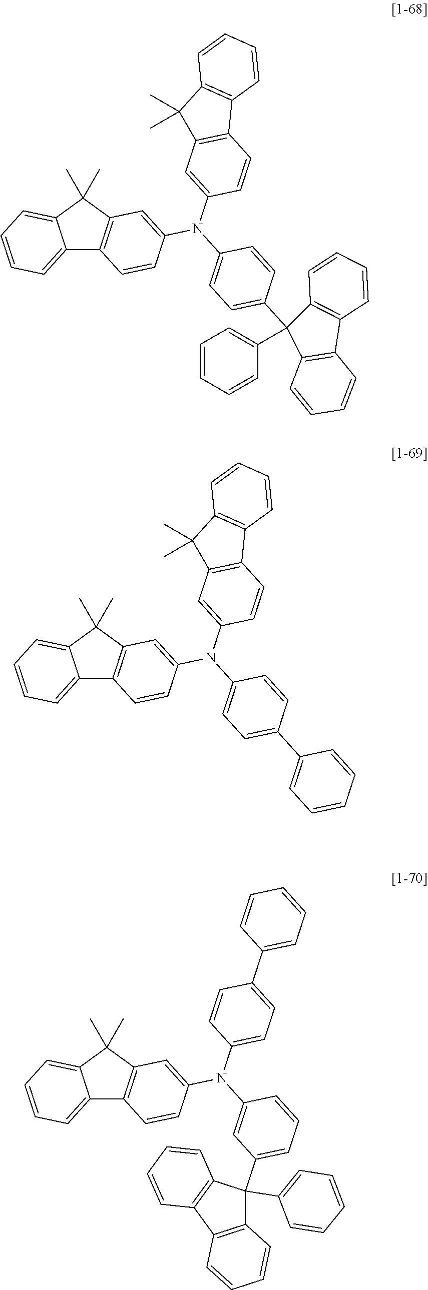 [SMILES: CC1(C)c2ccccc2-c2ccc(N(c3ccc(C4(c5ccccc5)c5ccccc5-c5ccccc54)cc3)c3ccc4c(c3)C(C)(C)c3ccccc3-4)cc21 ; CC1(C)c2ccccc2-c2ccc(N(c3ccc(-c4ccccc4)cc3)c3ccc4c(c3)C(C)(C)c3ccccc3-4)cc21 ; CC1(C)c2ccccc2-c2ccc(N(c3ccc(-c4ccccc4)cc3)c3cccc(C4(c5ccccc5)c5ccccc5-c5ccccc54)c3)cc21]

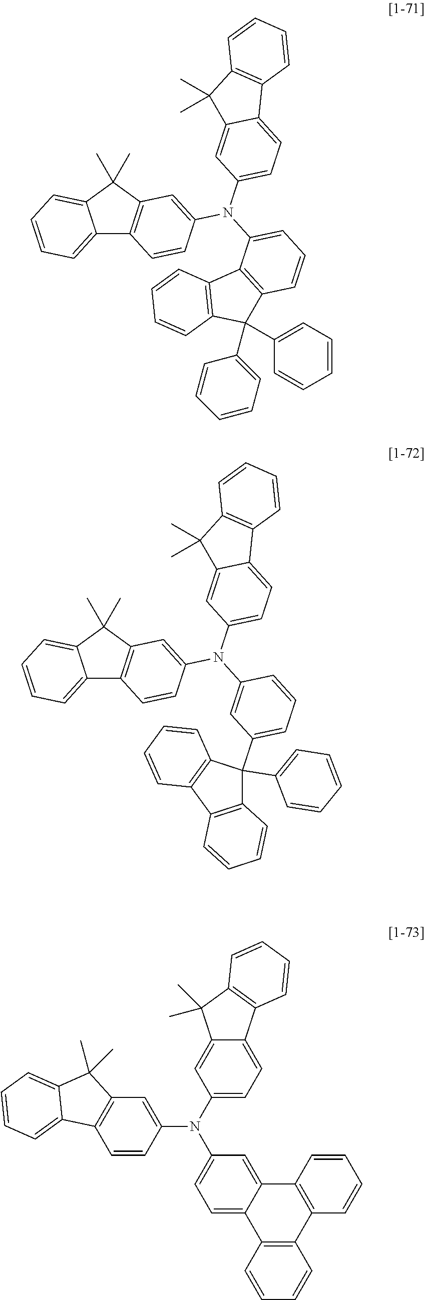 [SMILES: CC1(C)c2ccccc2-c2ccc(N(c3ccc4c(c3)C(C)(C)c3ccccc3-4)c3cccc4c3-c3ccccc3C4(c3ccccc3)c3ccccc3)cc21 ; CC1(C)c2ccccc2-c2ccc(N(c3cccc(C4(c5ccccc5)c5ccccc5-c5ccccc54)c3)c3ccc4c(c3)C(C)(C)c3ccccc3-4)cc21 ; CC1(C)c2ccccc2-c2ccc(N(c3ccc4c(c3)C(C)(C)c3ccccc3-4)c3ccc4c5ccccc5c5ccccc5c4c3)cc21]

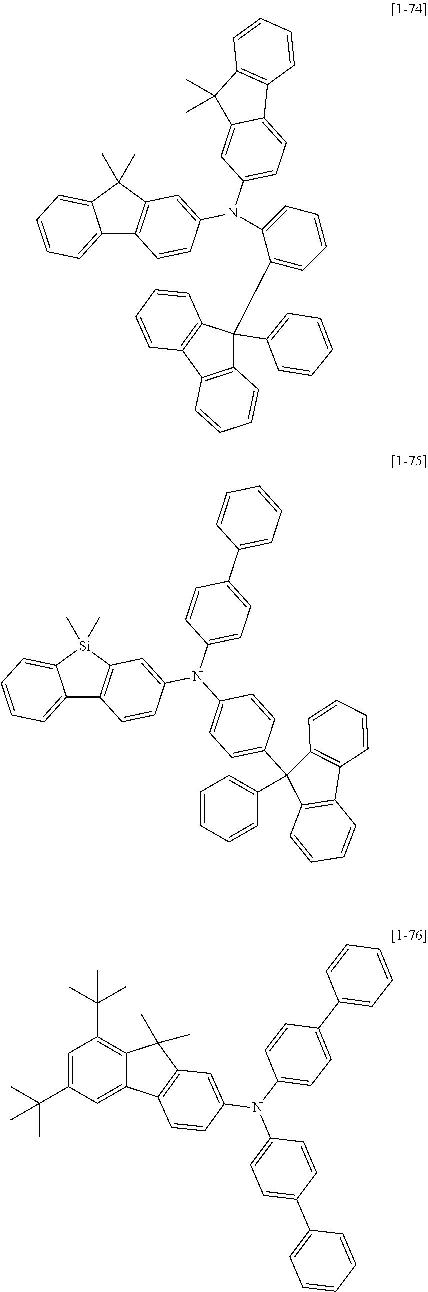 [SMILES: CC1(C)c2ccccc2-c2ccc(N(c3ccc4c(c3)C(C)(C)c3ccccc3-4)c3ccccc3C3(c4ccccc4)c4ccccc4-c4ccccc43)cc21 ; C[Si]1(C)c2ccccc2-c2ccc(N(c3ccc(-c4ccccc4)cc3)c3ccc(C4(c5ccccc5)c5ccccc5-c5ccccc54)cc3)cc21 ; CC(C)(C)c1cc2c(c(C(C)(C)C)c1)C(C)(C)c1cc(N(c3ccc(-c4ccccc4)cc3)c3ccc(-c4ccccc4)cc3)ccc1-2]

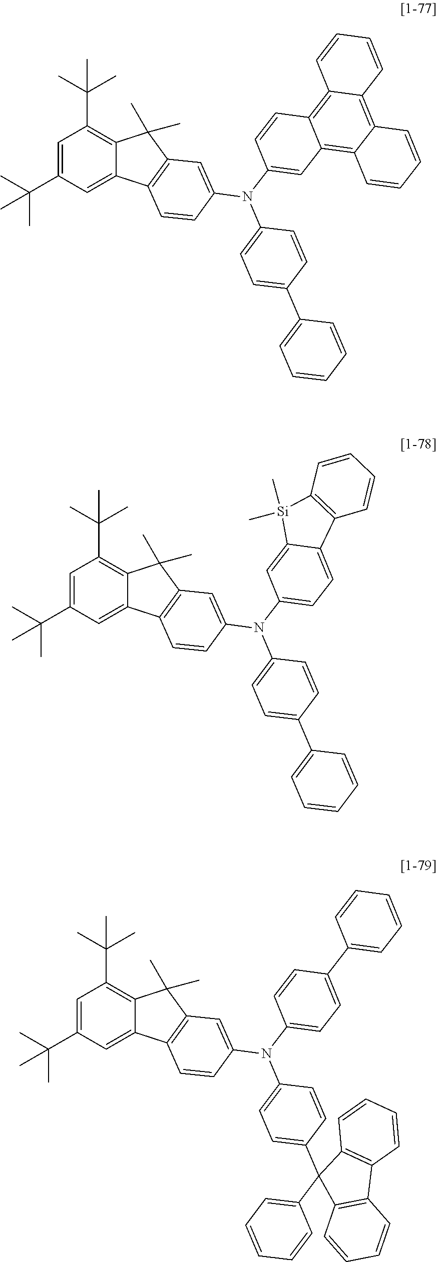 [SMILES: CC(C)(C)c1cc2c(c(C(C)(C)C)c1)C(C)(C)c1cc(N(c3ccc(-c4ccccc4)cc3)c3ccc4c5ccccc5c5ccccc5c4c3)ccc1-2 ; CC(C)(C)c1cc2c(c(C(C)(C)C)c1)C(C)(C)c1cc(N(c3ccc(-c4ccccc4)cc3)c3ccc4c(c3)[Si](C)(C)c3ccccc3-4)ccc1-2 ; CC(C)(C)c1cc2c(c(C(C)(C)C)c1)C(C)(C)c1cc(N(c3ccc(-c4ccccc4)cc3)c3ccc(C4(c5ccccc5)c5ccccc5-c5ccccc54)cc3)ccc1-2]

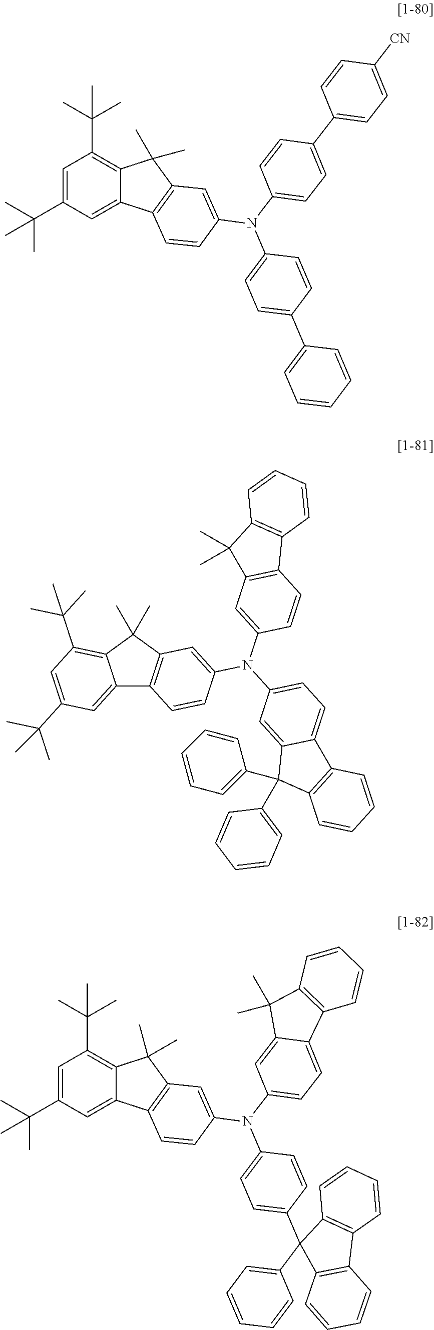 [SMILES: CC(C)(C)c1cc2c(c(C(C)(C)C)c1)C(C)(C)c1cc(N(c3ccc(-c4ccccc4)cc3)c3ccc(-c4ccc(C#N)cc4)cc3)ccc1-2 ; CC(C)(C)c1cc2c(c(C(C)(C)C)c1)C(C)(C)c1cc(N(c3ccc4c(c3)C(C)(C)c3ccccc3-4)c3ccc4c(c3)C(c3ccccc3)(c3ccccc3)c3ccccc3-4)ccc1-2 ; CC(C)(C)c1cc2c(c(C(C)(C)C)c1)C(C)(C)c1cc(N(c3ccc(C4(c5ccccc5)c5ccccc5-c5ccccc54)cc3)c3ccc4c(c3)C(C)(C)c3ccccc3-4)ccc1-2]

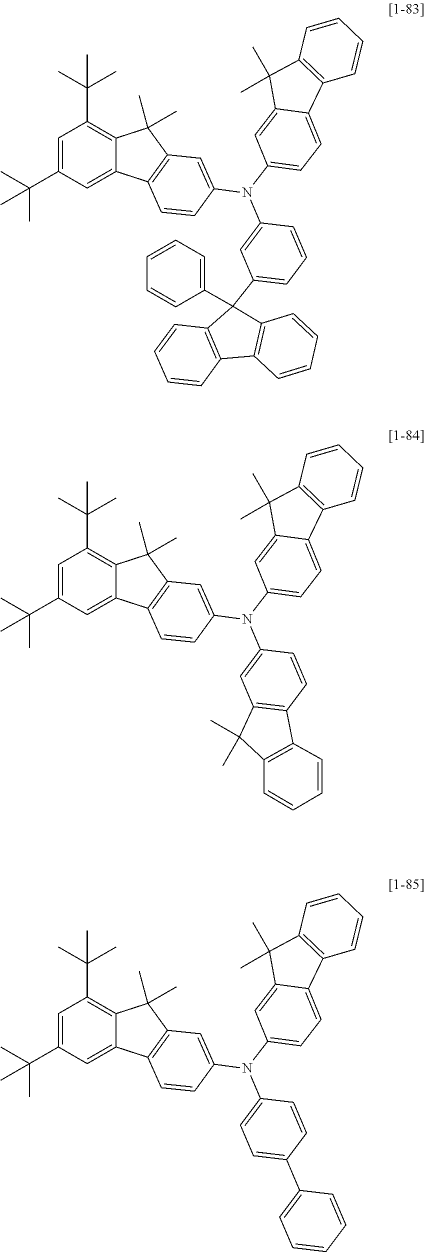 [SMILES: CC(C)(C)c1cc2c(c(C(C)(C)C)c1)C(C)(C)c1cc(N(c3cccc(C4(c5ccccc5)c5ccccc5-c5ccccc54)c3)c3ccc4c(c3)C(C)(C)c3ccccc3-4)ccc1-2 ; CC(C)(C)c1cc2c(c(C(C)(C)C)c1)C(C)(C)c1cc(N(c3ccc4c(c3)C(C)(C)c3ccccc3-4)c3ccc4c(c3)C(C)(C)c3ccccc3-4)ccc1-2 ; CC(C)(C)c1cc2c(c(C(C)(C)C)c1)C(C)(C)c1cc(N(c3ccc(-c4ccccc4)cc3)c3ccc4c(c3)C(C)(C)c3ccccc3-4)ccc1-2]

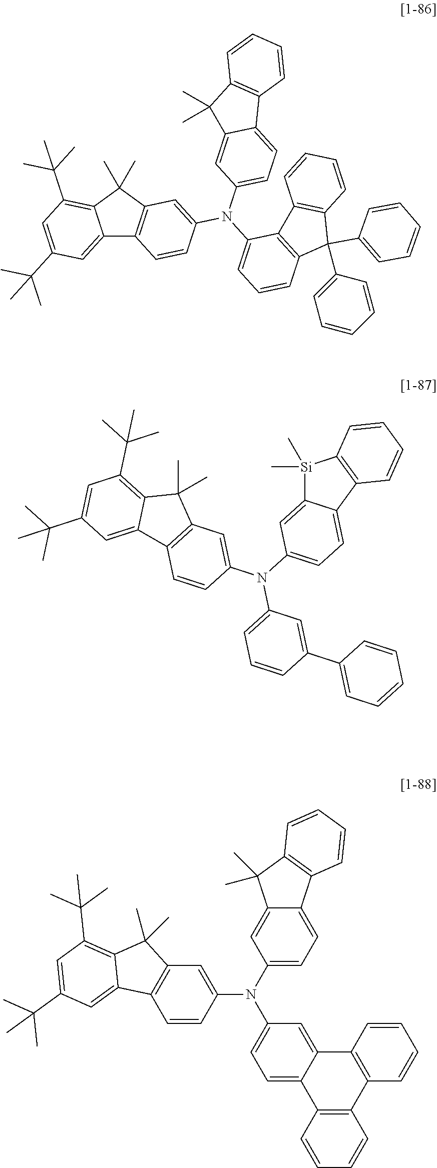 [SMILES: CC(C)(C)c1cc2c(c(C(C)(C)C)c1)C(C)(C)c1cc(N(c3ccc4c(c3)C(C)(C)c3ccccc3-4)c3cccc4c3-c3ccccc3C4(c3ccccc3)c3ccccc3)ccc1-2 ; CC(C)(C)c1cc2c(c(C(C)(C)C)c1)C(C)(C)c1cc(N(c3cccc(-c4ccccc4)c3)c3ccc4c(c3)[Si](C)(C)c3ccccc3-4)ccc1-2 ; CC(C)(C)c1cc2c(c(C(C)(C)C)c1)C(C)(C)c1cc(N(c3ccc4c(c3)C(C)(C)c3ccccc3-4)c3ccc4c5ccccc5c5ccccc5c4c3)ccc1-2]

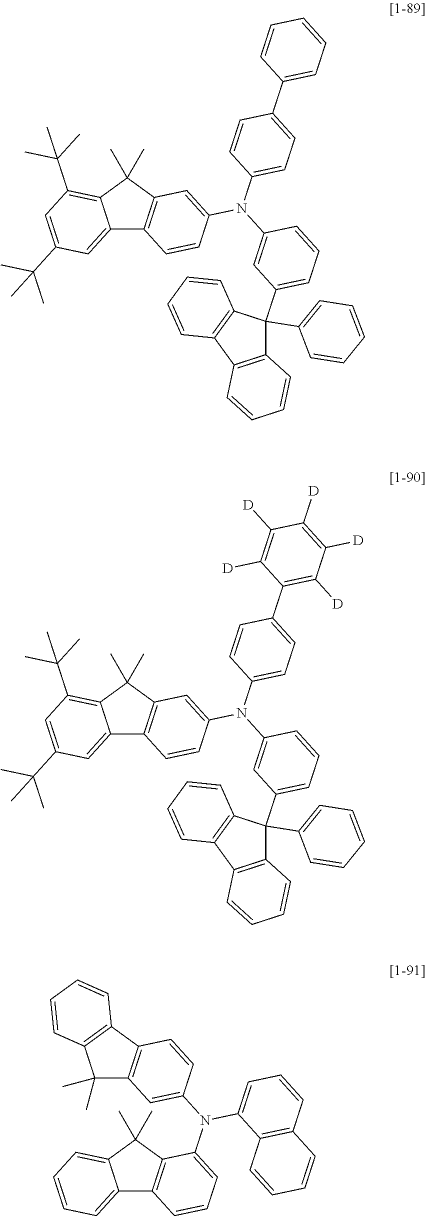 [SMILES: CC(C)(C)c1cc2c(c(C(C)(C)C)c1)C(C)(C)c1cc(N(c3ccc(-c4ccccc4)cc3)c3cccc(C4(c5ccccc5)c5ccccc5-c5ccccc54)c3)ccc1-2 ; [2H]c1c([2H])c([2H])c(-c2ccc(N(c3cccc(C4(c5ccccc5)c5ccccc5-c5ccccc54)c3)c3ccc4c(c3)C(C)(C)c3c-4cc(C(C)(C)C)cc3C(C)(C)C)cc2)c([2H])c1[2H] ; CC1(C)c2ccccc2-c2ccc(N(c3cccc4c3C(C)(C)c3ccccc3-4)c3cccc4ccccc34)cc21]

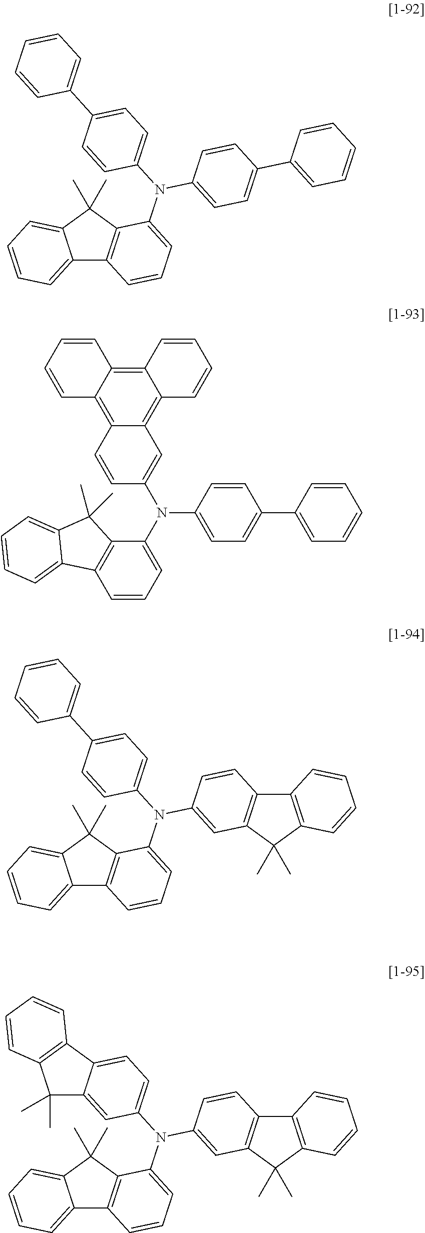 [SMILES: CC1(C)c2ccccc2-c2cccc(N(c3ccc(-c4ccccc4)cc3)c3ccc(-c4ccccc4)cc3)c21 ; CC1(C)c2ccccc2-c2cccc(N(c3ccc(-c4ccccc4)cc3)c3ccc4c5ccccc5c5ccccc5c4c3)c21 ; CC1(C)c2ccccc2-c2ccc(N(c3ccc(-c4ccccc4)cc3)c3cccc4c3C(C)(C)c3ccccc3-4)cc21 ; CC1(C)c2ccccc2-c2ccc(N(c3ccc4c(c3)C(C)(C)c3ccccc3-4)c3cccc4c3C(C)(C)c3ccccc3-4)cc21]

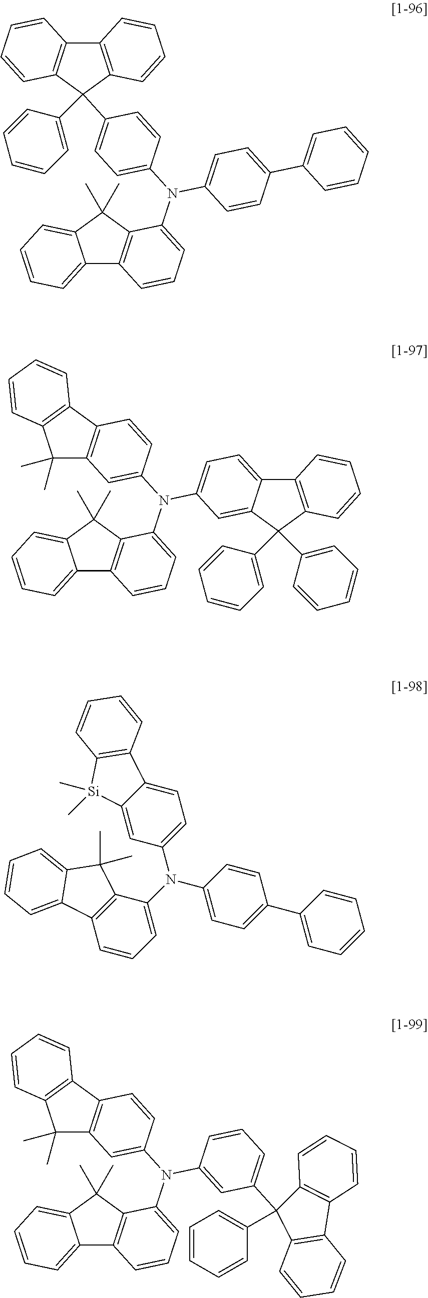 [SMILES: CC1(C)c2ccccc2-c2cccc(N(c3ccc(-c4ccccc4)cc3)c3ccc(C4(c5ccccc5)c5ccccc5-c5ccccc54)cc3)c21 ; CC1(C)c2ccccc2-c2ccc(N(c3ccc4c(c3)C(c3ccccc3)(c3ccccc3)c3ccccc3-4)c3cccc4c3C(C)(C)c3ccccc3-4)cc21 ; CC1(C)c2ccccc2-c2cccc(N(c3ccc(-c4ccccc4)cc3)c3ccc4c(c3)[Si](C)(C)c3ccccc3-4)c21 ; CC1(C)c2ccccc2-c2ccc(N(c3cccc(C4(c5ccccc5)c5ccccc5-c5ccccc54)c3)c3cccc4c3C(C)(C)c3ccccc3-4)cc21]

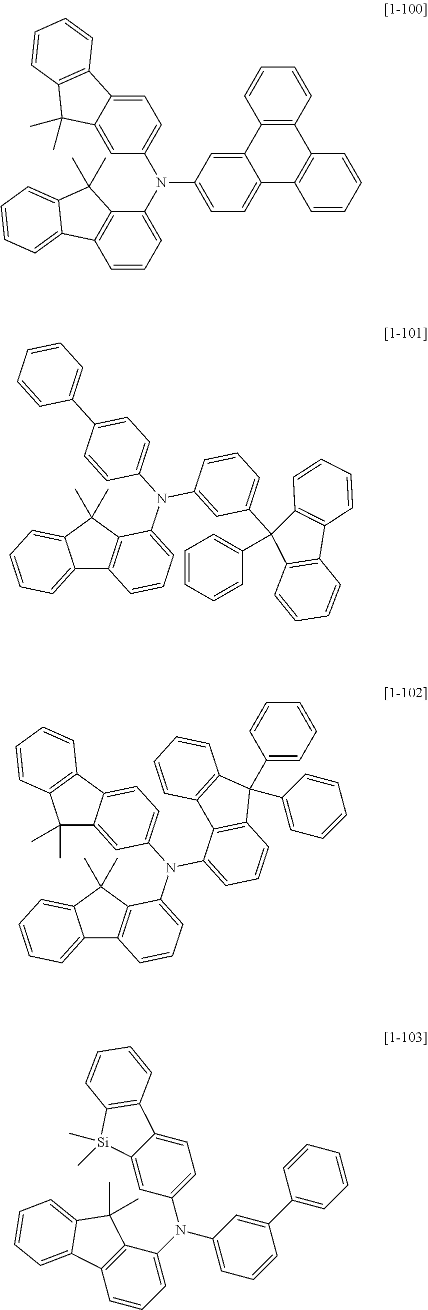 [SMILES: CC1(C)c2ccccc2-c2ccc(N(c3ccc4c5ccccc5c5ccccc5c4c3)c3cccc4c3C(C)(C)c3ccccc3-4)cc21 ; CC1(C)c2ccccc2-c2cccc(N(c3ccc(-c4ccccc4)cc3)c3cccc(C4(c5ccccc5)c5ccccc5-c5ccccc54)c3)c21 ; CC1(C)c2ccccc2-c2ccc(N(c3cccc4c3-c3ccccc3C4(c3ccccc3)c3ccccc3)c3cccc4c3C(C)(C)c3ccccc3-4)cc21 ; CC1(C)c2ccccc2-c2cccc(N(c3cccc(-c4ccccc4)c3)c3ccc4c(c3)[Si](C)(C)c3ccccc3-4)c21]

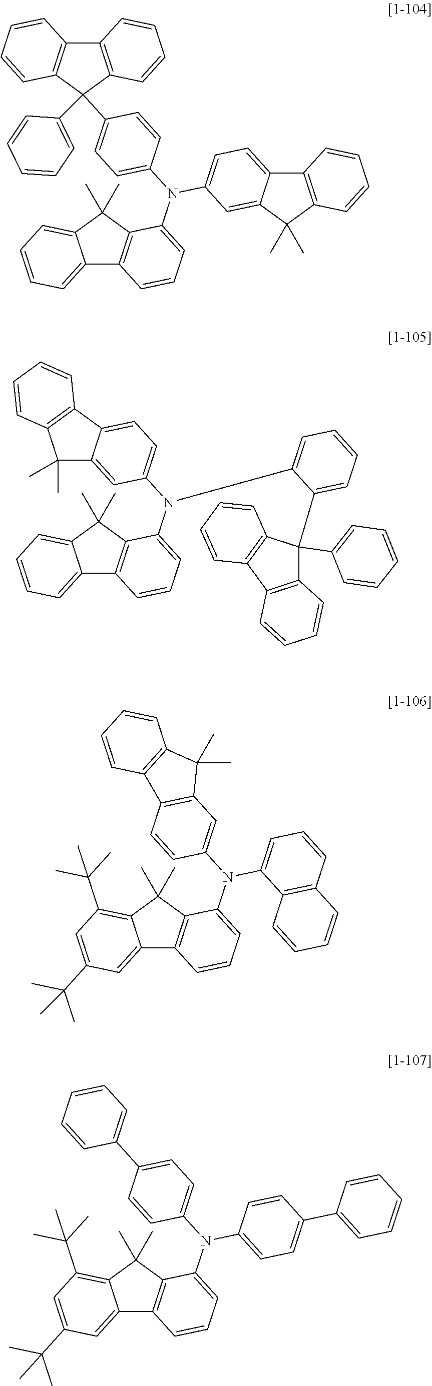 [SMILES: CC1(C)c2ccccc2-c2ccc(N(c3ccc(C4(c5ccccc5)c5ccccc5-c5ccccc54)cc3)c3cccc4c3C(C)(C)c3ccccc3-4)cc21 ; CC1(C)c2ccccc2-c2ccc(N(c3ccccc3C3(c4ccccc4)c4ccccc4-c4ccccc43)c3cccc4c3C(C)(C)c3ccccc3-4)cc21 ; CC(C)(C)c1cc2c(c(C(C)(C)C)c1)C(C)(C)c1c-2cccc1N(c1ccc2c(c1)C(C)(C)c1ccccc1-2)c1cccc2ccccc12 ; CC(C)(C)c1cc2c(c(C(C)(C)C)c1)C(C)(C)c1c-2cccc1N(c1ccc(-c2ccccc2)cc1)c1ccc(-c2ccccc2)cc1]

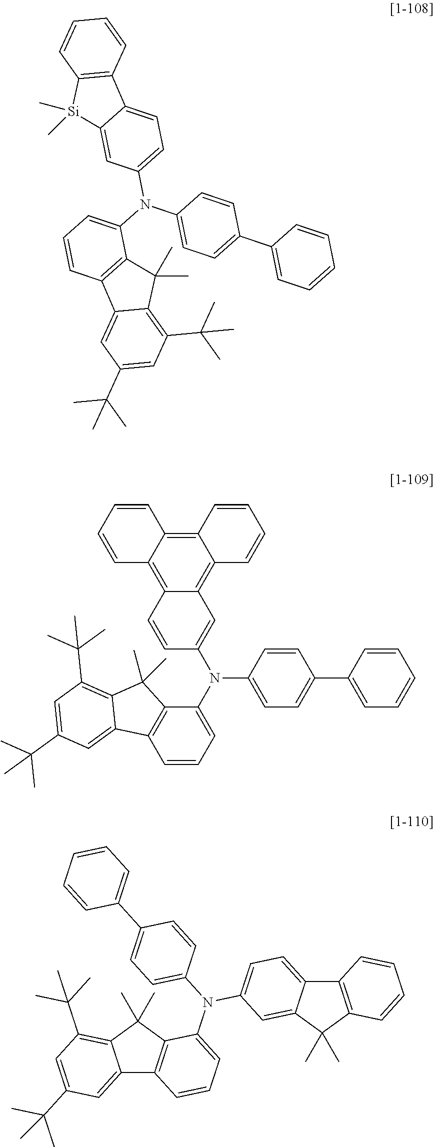 [SMILES: CC(C)(C)c1cc2c(c(C(C)(C)C)c1)C(C)(C)c1c-2cccc1N(c1ccc(-c2ccccc2)cc1)c1ccc2c(c1)[Si](C)(C)c1ccccc1-2 ; CC(C)(C)c1cc2c(c(C(C)(C)C)c1)C(C)(C)c1c-2cccc1N(c1ccc(-c2ccccc2)cc1)c1ccc2c3ccccc3c3ccccc3c2c1 ; CC(C)(C)c1cc2c(c(C(C)(C)C)c1)C(C)(C)c1c-2cccc1N(c1ccc(-c2ccccc2)cc1)c1ccc2c(c1)C(C)(C)c1ccccc1-2]

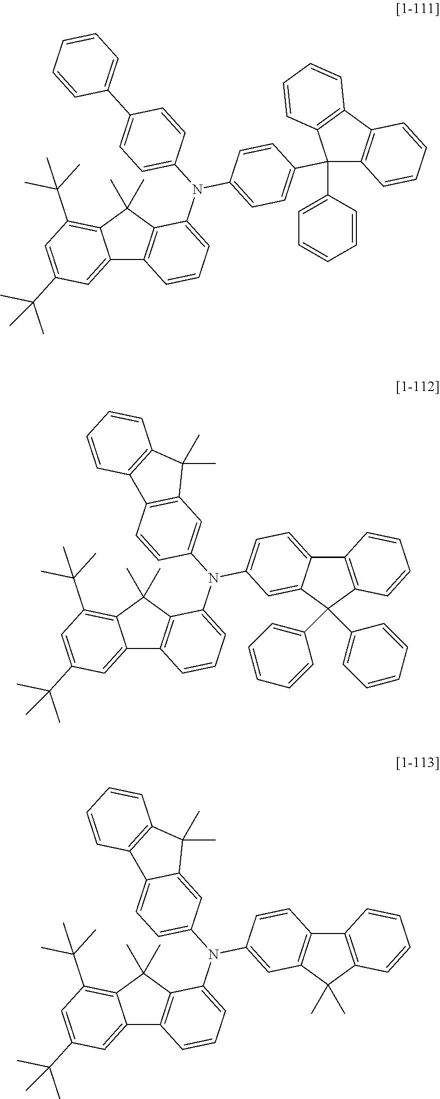 [SMILES: CC(C)(C)c1cc2c(c(C(C)(C)C)c1)C(C)(C)c1c-2cccc1N(c1ccc(-c2ccccc2)cc1)c1ccc(C2(c3ccccc3)c3ccccc3-c3ccccc32)cc1 ; CC(C)(C)c1cc2c(c(C(C)(C)C)c1)C(C)(C)c1c-2cccc1N(c1ccc2c(c1)C(C)(C)c1ccccc1-2)c1ccc2c(c1)C(c1ccccc1)(c1ccccc1)c1ccccc1-2 ; CC(C)(C)c1cc2c(c(C(C)(C)C)c1)C(C)(C)c1c-2cccc1N(c1ccc2c(c1)C(C)(C)c1ccccc1-2)c1ccc2c(c1)C(C)(C)c1ccccc1-2]

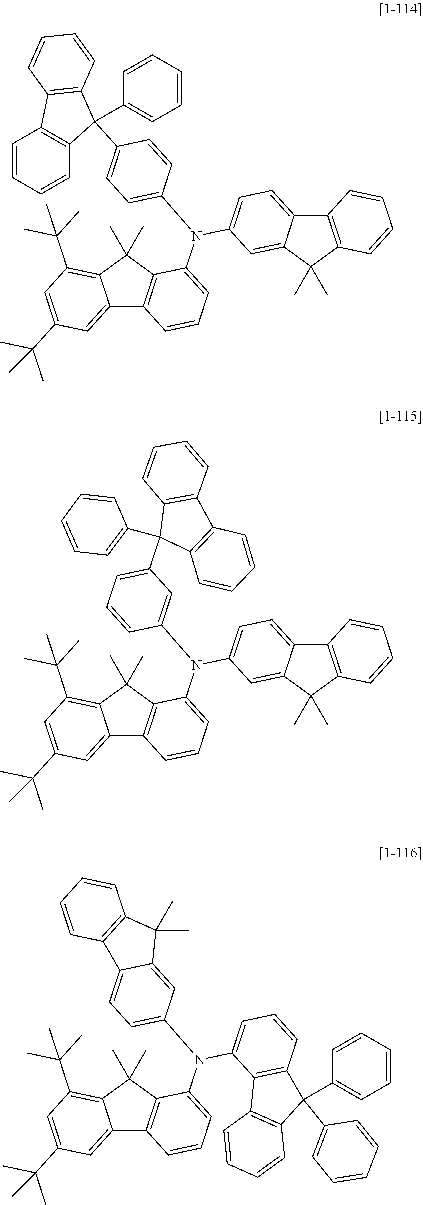 [SMILES: CC(C)(C)c1cc2c(c(C(C)(C)C)c1)C(C)(C)c1c-2cccc1N(c1ccc(C2(c3ccccc3)c3ccccc3-c3ccccc32)cc1)c1ccc2c(c1)C(C)(C)c1ccccc1-2 ; CC(C)(C)c1cc2c(c(C(C)(C)C)c1)C(C)(C)c1c-2cccc1N(c1cccc(C2(c3ccccc3)c3ccccc3-c3ccccc32)c1)c1ccc2c(c1)C(C)(C)c1ccccc1-2 ; CC(C)(C)c1cc2c(c(C(C)(C)C)c1)C(C)(C)c1c-2cccc1N(c1ccc2c(c1)C(C)(C)c1ccccc1-2)c1cccc2c1-c1ccccc1C2(c1ccccc1)c1ccccc1]

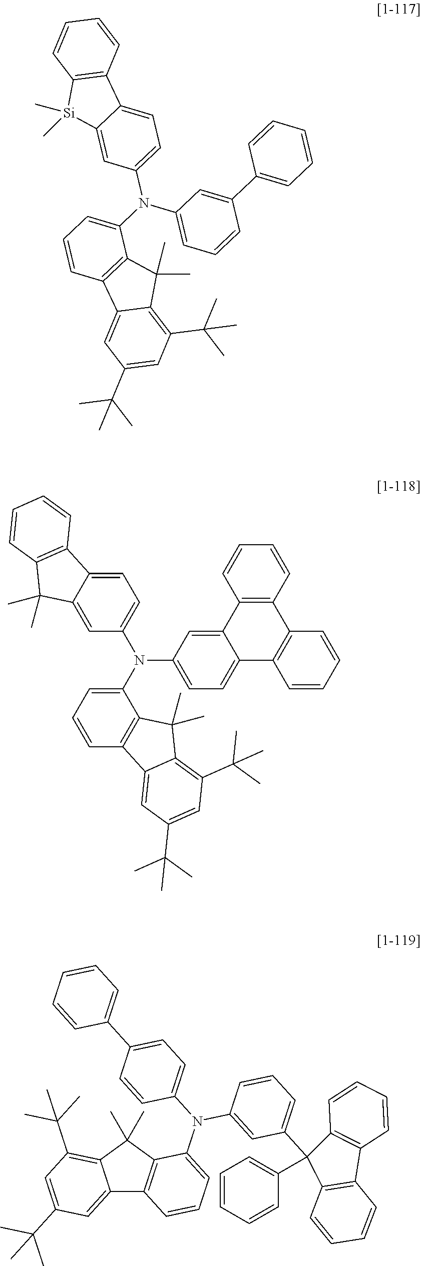 [SMILES: CC(C)(C)c1cc2c(c(C(C)(C)C)c1)C(C)(C)c1c-2cccc1N(c1cccc(-c2ccccc2)c1)c1ccc2c(c1)[Si](C)(C)c1ccccc1-2 ; CC(C)(C)c1cc2c(c(C(C)(C)C)c1)C(C)(C)c1c-2cccc1N(c1ccc2c(c1)C(C)(C)c1ccccc1-2)c1ccc2c3ccccc3c3ccccc3c2c1 ; CC(C)(C)c1cc2c(c(C(C)(C)C)c1)C(C)(C)c1c-2cccc1N(c1ccc(-c2ccccc2)cc1)c1cccc(C2(c3ccccc3)c3ccccc3-c3ccccc32)c1]

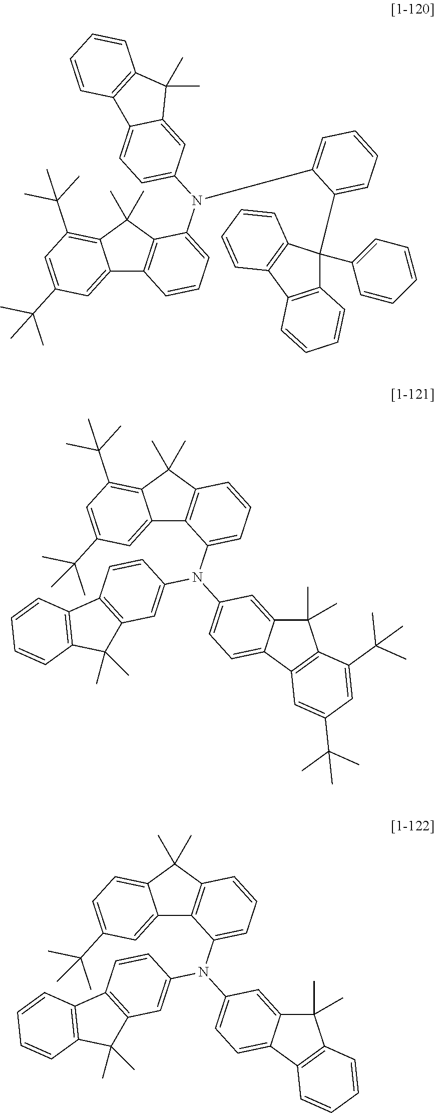 [SMILES: CC(C)(C)c1cc2c(c(C(C)(C)C)c1)C(C)(C)c1c-2cccc1N(c1ccc2c(c1)C(C)(C)c1ccccc1-2)c1ccccc1C1(c2ccccc2)c2ccccc2-c2ccccc21 ; CC(C)(C)c1cc2c(c(C(C)(C)C)c1)C(C)(C)c1cc(N(c3ccc4c(c3)C(C)(C)c3ccccc3-4)c3cccc4c3-c3cc(C(C)(C)C)cc(C(C)(C)C)c3C4(C)C)ccc1-2 ; CC(C)(C)c1ccc2c(c1)-c1c(N(c3ccc4c(c3)C(C)(C)c3ccccc3-4)c3ccc4c(c3)C(C)(C)c3ccccc3-4)cccc1C2(C)C]

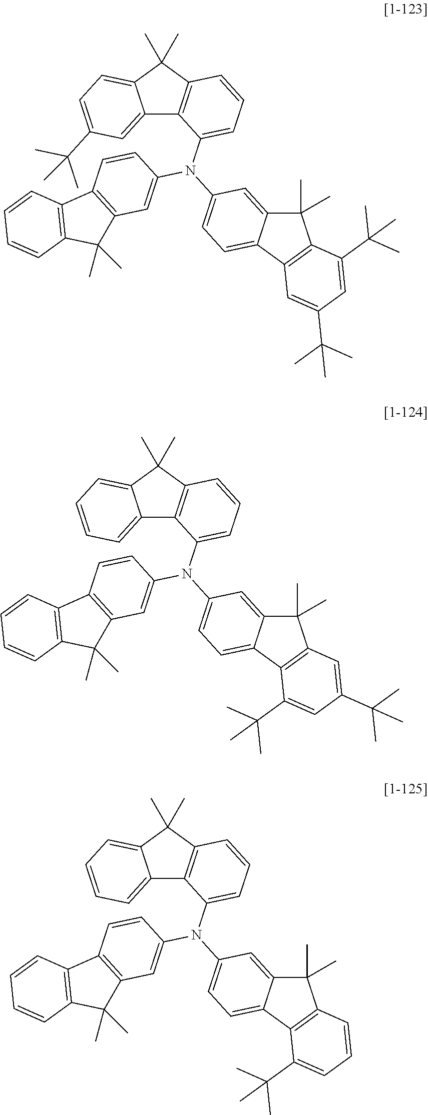 [SMILES: CC(C)(C)c1ccc2c(c1)-c1c(N(c3ccc4c(c3)C(C)(C)c3ccccc3-4)c3ccc4c(c3)C(C)(C)c3c-4cc(C(C)(C)C)cc3C(C)(C)C)cccc1C2(C)C ; CC(C)(C)c1cc(C(C)(C)C)c2c(c1)C(C)(C)c1cc(N(c3ccc4c(c3)C(C)(C)c3ccccc3-4)c3cccc4c3-c3ccccc3C4(C)C)ccc1-2 ; CC(C)(C)c1cccc2c1-c1ccc(N(c3ccc4c(c3)C(C)(C)c3ccccc3-4)c3cccc4c3-c3ccccc3C4(C)C)cc1C2(C)C]

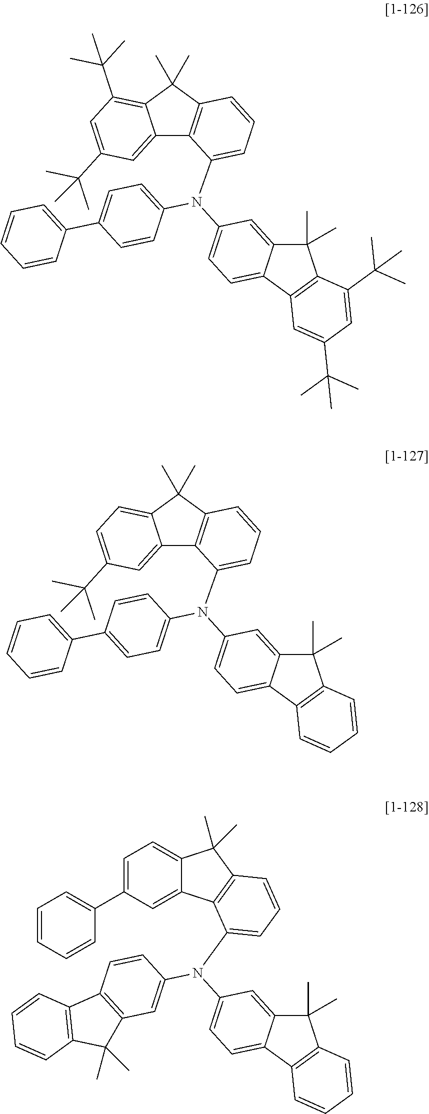 [SMILES: CC(C)(C)c1cc2c(c(C(C)(C)C)c1)C(C)(C)c1cc(N(c3ccc(-c4ccccc4)cc3)c3cccc4c3-c3cc(C(C)(C)C)cc(C(C)(C)C)c3C4(C)C)ccc1-2 ; CC(C)(C)c1ccc2c(c1)-c1c(N(c3ccc(-c4ccccc4)cc3)c3ccc4c(c3)C(C)(C)c3ccccc3-4)cccc1C2(C)C ; CC1(C)c2ccccc2-c2ccc(N(c3ccc4c(c3)C(C)(C)c3ccccc3-4)c3cccc4c3-c3cc(-c5ccccc5)ccc3C4(C)C)cc21]

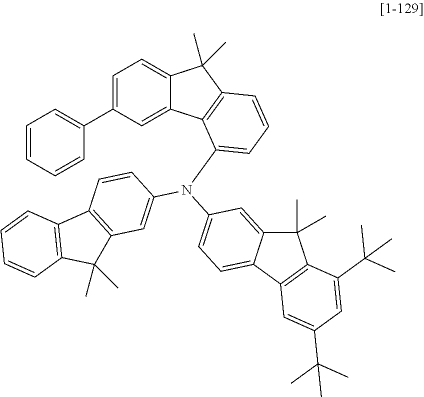 [SMILES: CC(C)(C)c1cc2c(c(C(C)(C)C)c1)C(C)(C)c1cc(N(c3ccc4c(c3)C(C)(C)c3ccccc3-4)c3cccc4c3-c3cc(-c5ccccc5)ccc3C4(C)C)ccc1-2]

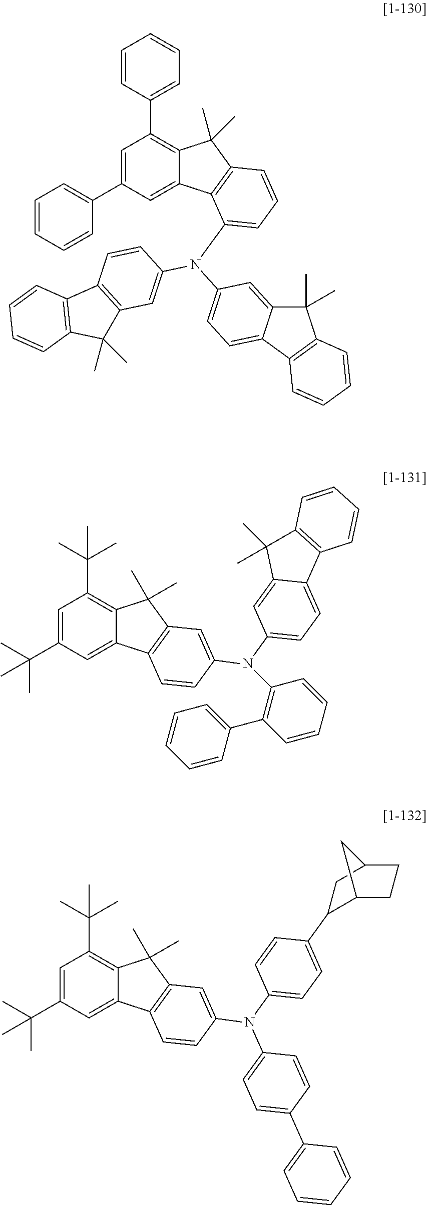 [SMILES: CC1(C)c2ccccc2-c2ccc(N(c3ccc4c(c3)C(C)(C)c3ccccc3-4)c3cccc4c3-c3cc(-c5ccccc5)cc(-c5ccccc5)c3C4(C)C)cc21 ; CC(C)(C)c1cc2c(c(C(C)(C)C)c1)C(C)(C)c1cc(N(c3ccc4c(c3)C(C)(C)c3ccccc3-4)c3ccccc3-c3ccccc3)ccc1-2 ; CC(C)(C)c1cc2c(c(C(C)(C)C)c1)C(C)(C)c1cc(N(c3ccc(-c4ccccc4)cc3)c3ccc(C4CC5CCC4C5)cc3)ccc1-2]

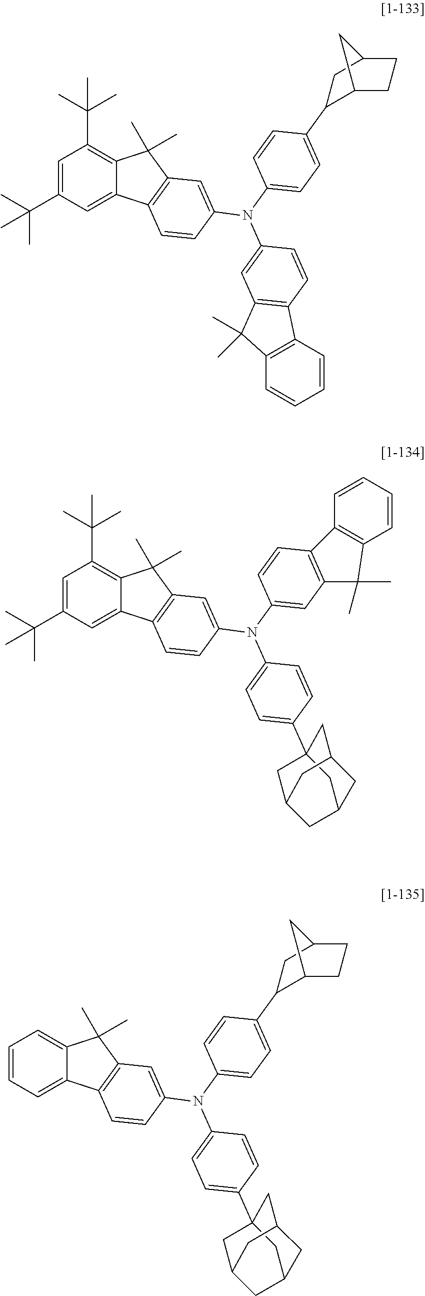 [SMILES: CC(C)(C)c1cc2c(c(C(C)(C)C)c1)C(C)(C)c1cc(N(c3ccc(C4CC5CCC4C5)cc3)c3ccc4c(c3)C(C)(C)c3ccccc3-4)ccc1-2 ; CC(C)(C)c1cc2c(c(C(C)(C)C)c1)C(C)(C)c1cc(N(c3ccc(C45CC6CC(CC(C6)C4)C5)cc3)c3ccc4c(c3)C(C)(C)c3ccccc3-4)ccc1-2 ; CC1(C)c2ccccc2-c2ccc(N(c3ccc(C4CC5CCC4C5)cc3)c3ccc(C45CC6CC(CC(C6)C4)C5)cc3)cc21]

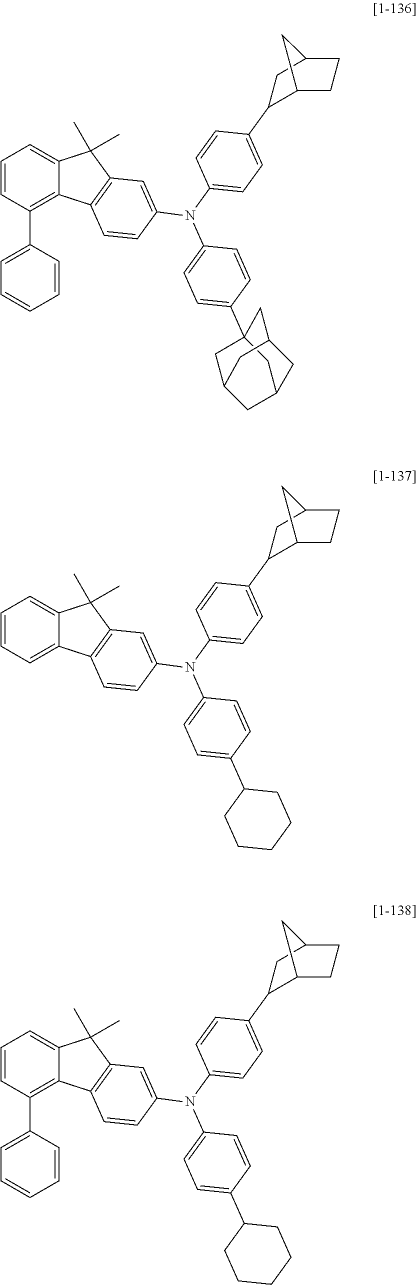 [SMILES: CC1(C)c2cc(N(c3ccc(C4CC5CCC4C5)cc3)c3ccc(C45CC6CC(CC(C6)C4)C5)cc3)ccc2-c2c(-c3ccccc3)cccc21 ; CC1(C)c2ccccc2-c2ccc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2c(-c3ccccc3)cccc21]

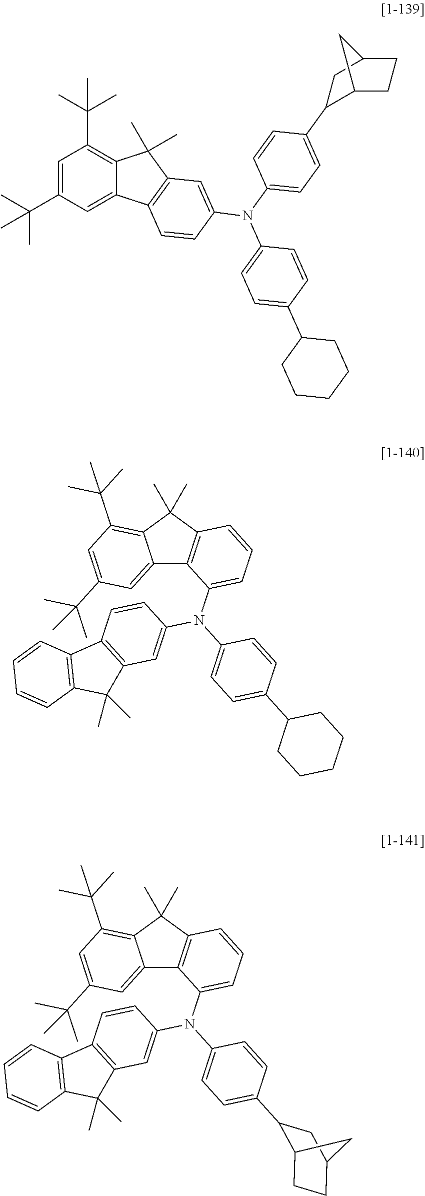 [SMILES: CC(C)(C)c1cc2c(c(C(C)(C)C)c1)C(C)(C)c1cc(N(c3ccc(C4CCCCC4)cc3)c3ccc(C4CC5CCC4C5)cc3)ccc1-2 ; CC(C)(C)c1cc2c(c(C(C)(C)C)c1)C(C)(C)c1cccc(N(c3ccc(C4CCCCC4)cc3)c3ccc4c(c3)C(C)(C)c3ccccc3-4)c1-2 ; CC(C)(C)c1cc2c(c(C(C)(C)C)c1)C(C)(C)c1cccc(N(c3ccc(C4CC5CCC4C5)cc3)c3ccc4c(c3)C(C)(C)c3ccccc3-4)c1-2]

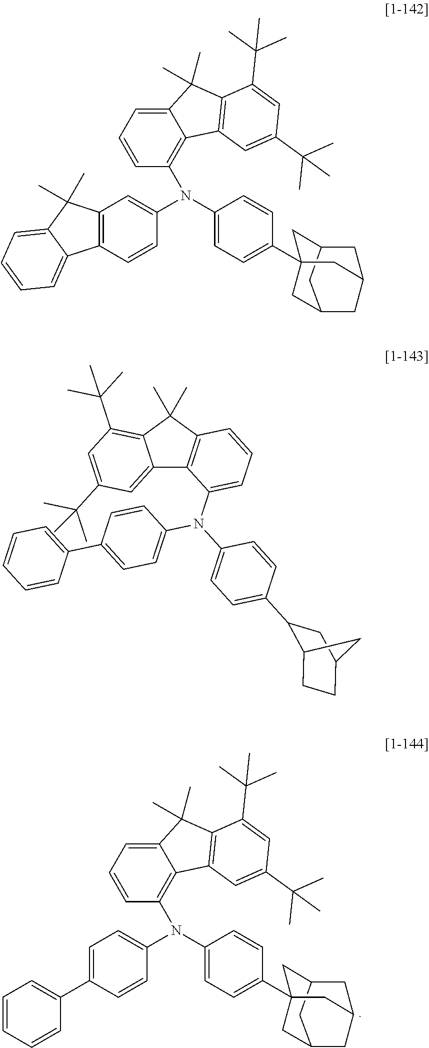 [SMILES: CC(C)(C)c1cc2c(c(C(C)(C)C)c1)C(C)(C)c1cccc(N(c3ccc(C45CC6CC(CC(C6)C4)C5)cc3)c3ccc4c(c3)C(C)(C)c3ccccc3-4)c1-2 ; CC(C)(C)c1cc2c(c(C(C)(C)C)c1)C(C)(C)c1cccc(N(c3ccc(-c4ccccc4)cc3)c3ccc(C4CC5CCC4C5)cc3)c1-2 ; CC(C)(C)c1cc2c(c(C(C)(C)C)c1)C(C)(C)c1cccc(N(c3ccc(-c4ccccc4)cc3)c3ccc(C45CC6CC(CC(C6)C4)C5)cc3)c1-2]

6. The organic optoelectronic device as claimed in claim 1, wherein:
Ar³ and Ar⁴ are each independently a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted anthracenyl group, a substituted or unsubstituted fluorenyl group, a substituted or unsubstituted spirofluorenyl group, a substituted or unsubstituted triphenylene group, a substituted or unsubstituted dibenzofuranyl group, a substituted or unsubstituted dibenzothiophenyl group, or a substituted or unsubstituted dibenzosilolyl group, and
at least one of Ar³ and Ar⁴ is a substituted or unsubstituted 9-fluorenyl group.

7. The organic optoelectronic device as claimed in claim 1, wherein:
moieties L⁵-Ar³ and L⁶-Ar⁴ are each independently a moiety of Group II,
at least one of Ar³ and Art is a substituted or unsubstituted 9-fluorenyl group, 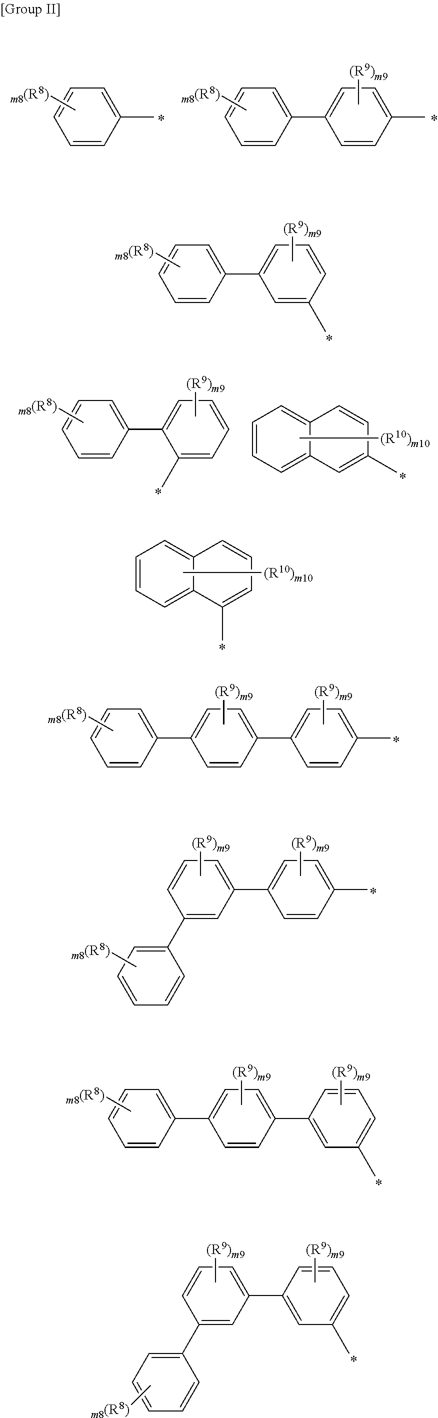 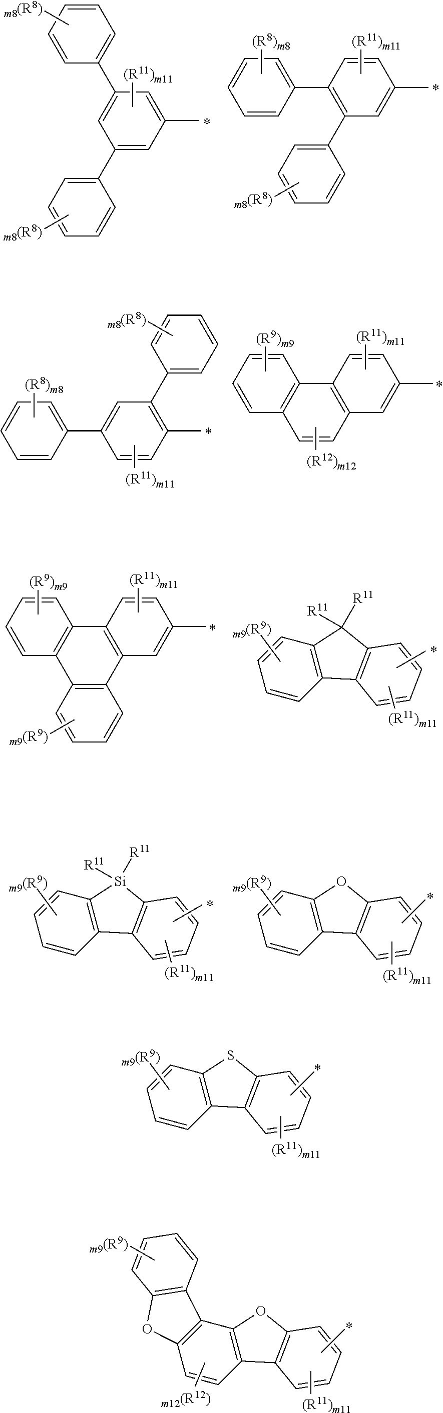 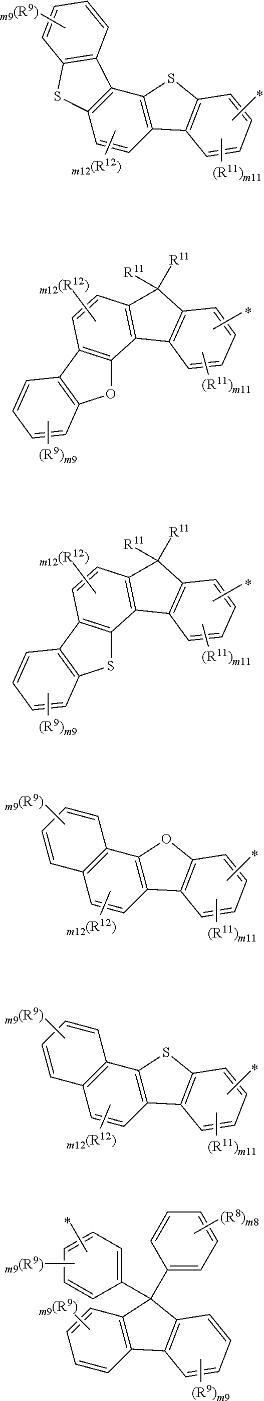 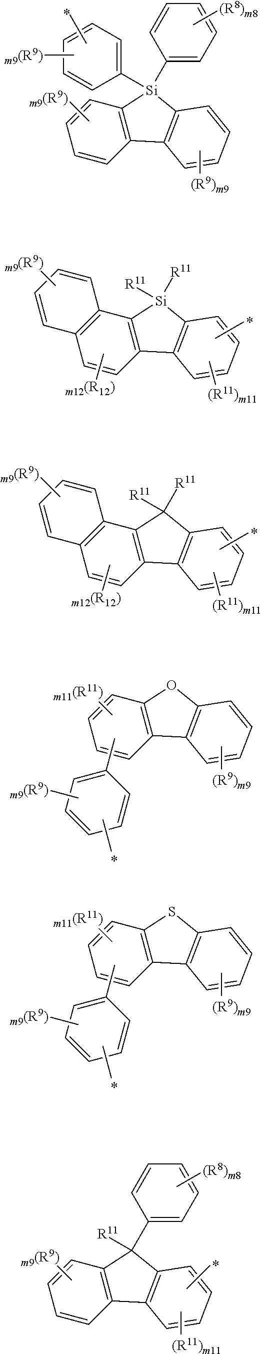 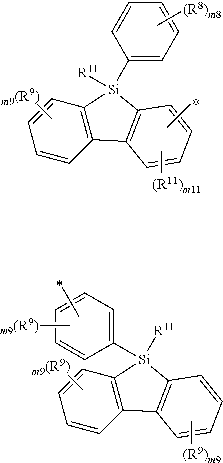
in Group II,
R⁸ to R¹² are each independently hydrogen, deuterium, a cyano group, a substituted or unsubstituted C1 to C10 alkyl group, or a substituted or unsubstituted C6 to C12 aryl group,
m8 is an integer of 1 to 5,
m9 is an integer of 1 to 4,
m10 is an integer of 1 to 7,
m11 is an integer of 1 to 3,
m12 is an integer of 1 or 2, and
* is a linking point.

8. The organic optoelectronic device as claimed in claim 7, wherein:
at least one of Ar³ and Ar⁴ is a group represented by Chemical Formula II-1, and 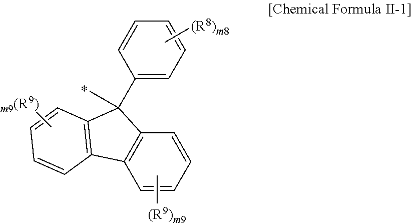
in Chemical Formula II-1, R⁸, R⁹, m8, and m9 are defined the same as those of Group II.

9. The organic optoelectronic device as claimed in claim 1, wherein the second compound is a compound of Group 2:

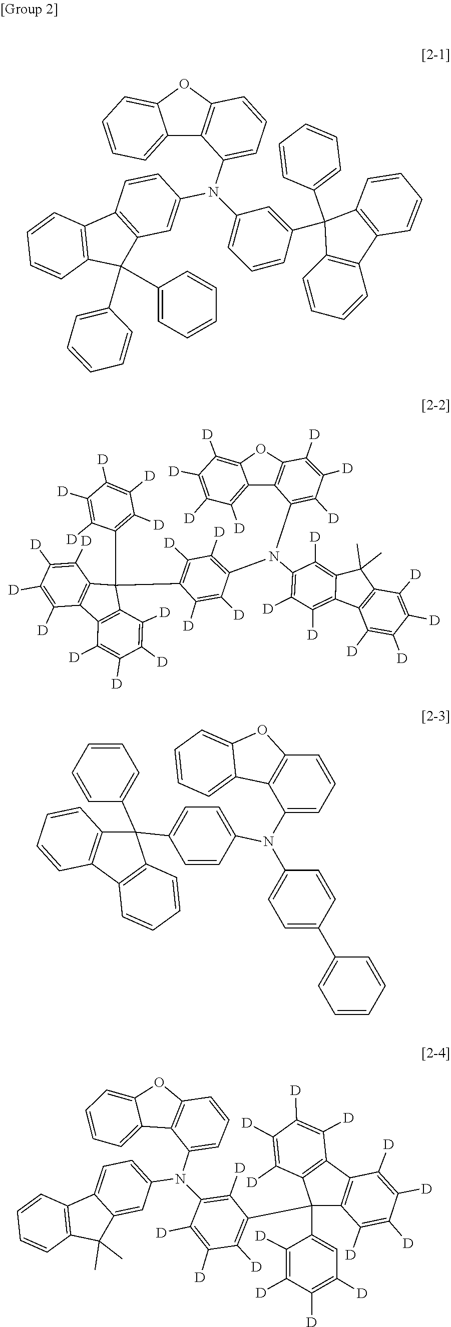

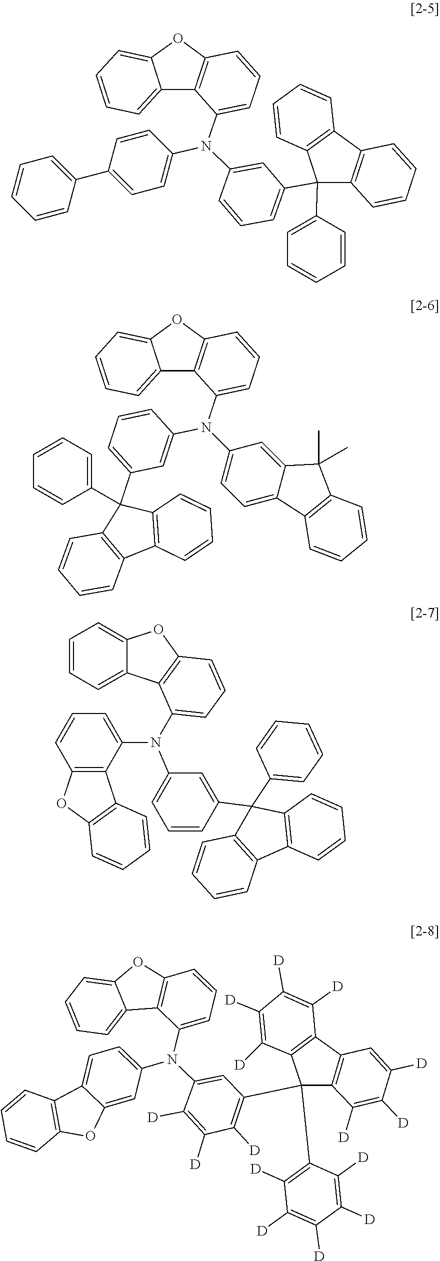

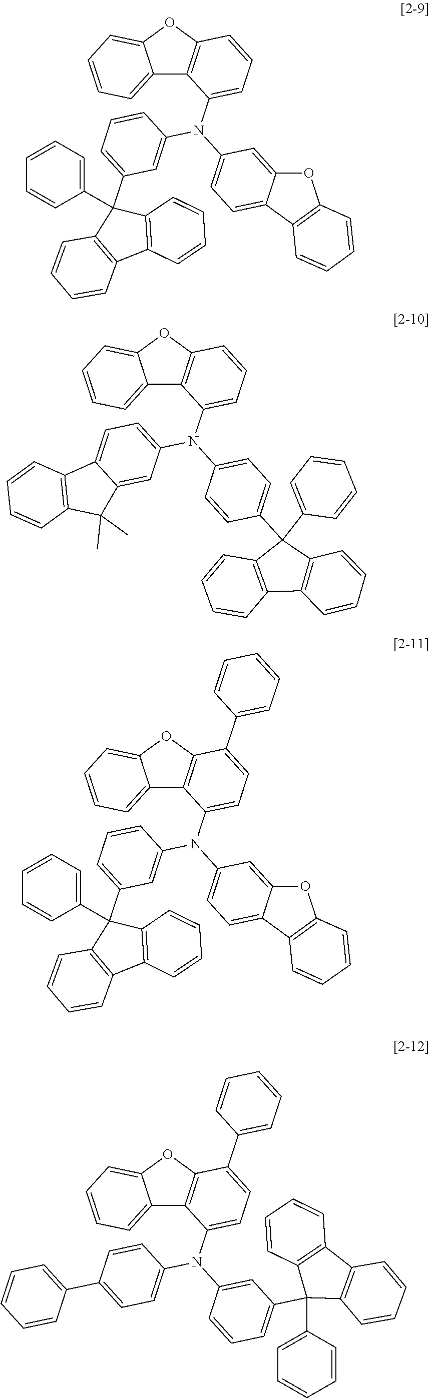

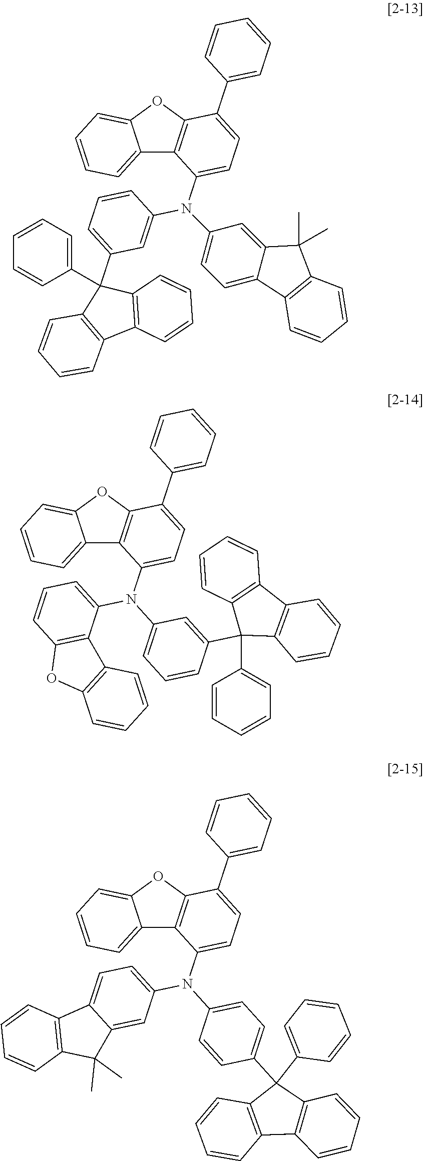 [SMILES: CC1(C)c2ccccc2-c2ccc(N(c3cccc(C4(c5ccccc5)c5ccccc5-c5ccccc54)c3)c3ccc(-c4ccccc4)c4oc5ccccc5c34)cc21 ; c1ccc(-c2ccc(N(c3cccc(C4(c5ccccc5)c5ccccc5-c5ccccc54)c3)c3cccc4oc5ccccc5c34)c3c2oc2ccccc23)cc1 ; CC1(C)c2ccccc2-c2ccc(N(c3ccc(C4(c5ccccc5)c5ccccc5-c5ccccc54)cc3)c3ccc(-c4ccccc4)c4oc5ccccc5c34)cc21]

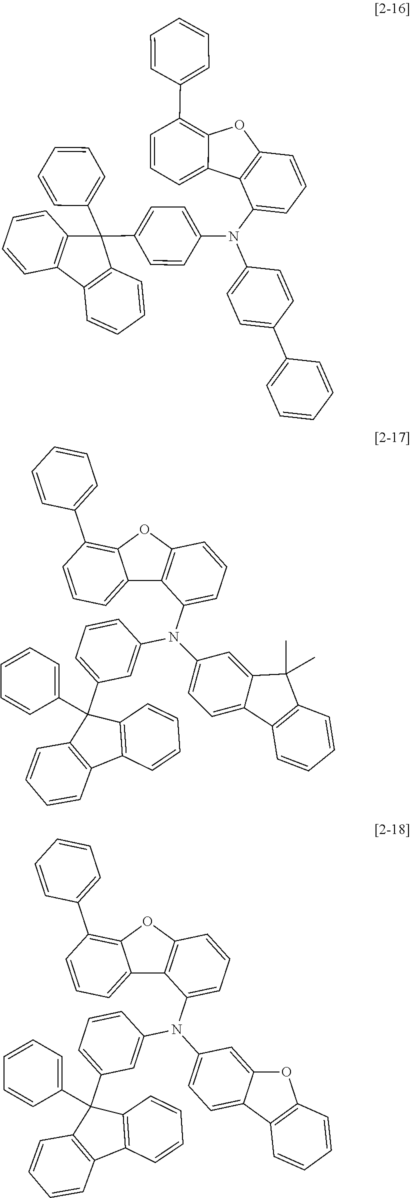

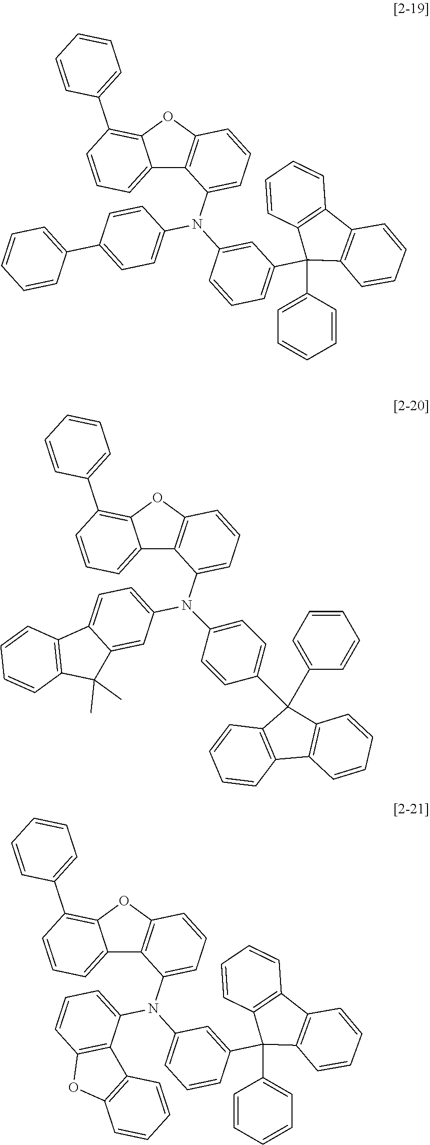 [SMILES: c1ccc(-c2ccc(N(c3cccc(C4(c5ccccc5)c5ccccc5-c5ccccc54)c3)c3cccc4oc5c(-c6ccccc6)cccc5c34)cc2)cc1 ; CC1(C)c2ccccc2-c2ccc(N(c3ccc(C4(c5ccccc5)c5ccccc5-c5ccccc54)cc3)c3cccc4oc5c(-c6ccccc6)cccc5c34)cc21 ; c1ccc(-c2cccc3c2oc2cccc(N(c4cccc(C5(c6ccccc6)c6ccccc6-c6ccccc65)c4)c4cccc5oc6ccccc6c45)c23)cc1]

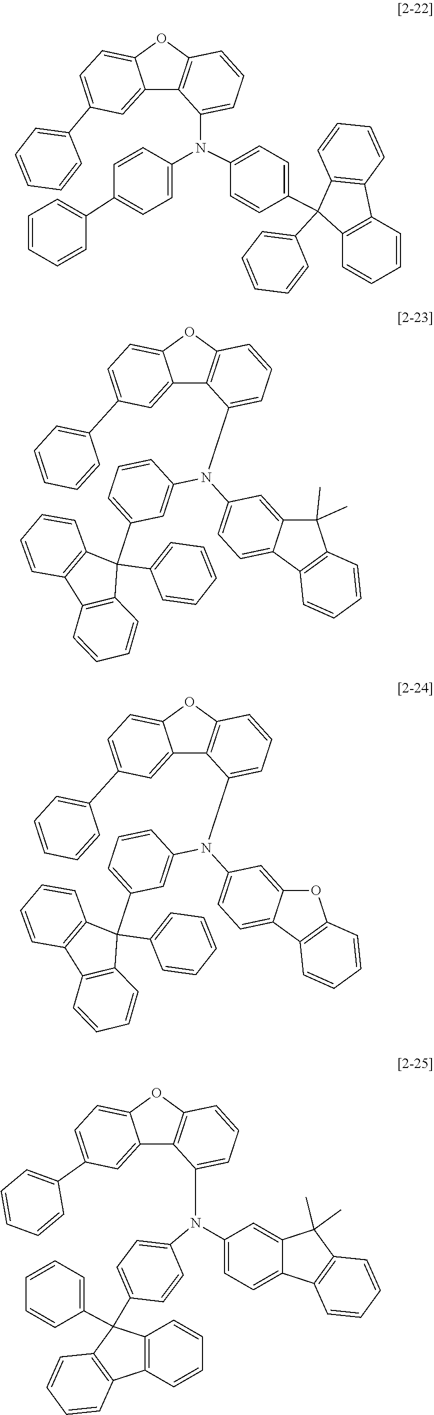 [SMILES: c1ccc(-c2ccc(N(c3ccc(C4(c5ccccc5)c5ccccc5-c5ccccc54)cc3)c3cccc4oc5ccc(-c6ccccc6)cc5c34)cc2)cc1 ; CC1(C)c2ccccc2-c2ccc(N(c3cccc(C4(c5ccccc5)c5ccccc5-c5ccccc54)c3)c3cccc4oc5ccc(-c6ccccc6)cc5c34)cc21 ; c1ccc(-c2ccc3oc4cccc(N(c5cccc(C6(c7ccccc7)c7ccccc7-c7ccccc76)c5)c5ccc6c(c5)oc5ccccc56)c4c3c2)cc1 ; CC1(C)c2ccccc2-c2ccc(N(c3ccc(C4(c5ccccc5)c5ccccc5-c5ccccc54)cc3)c3cccc4oc5ccc(-c6ccccc6)cc5c34)cc21]

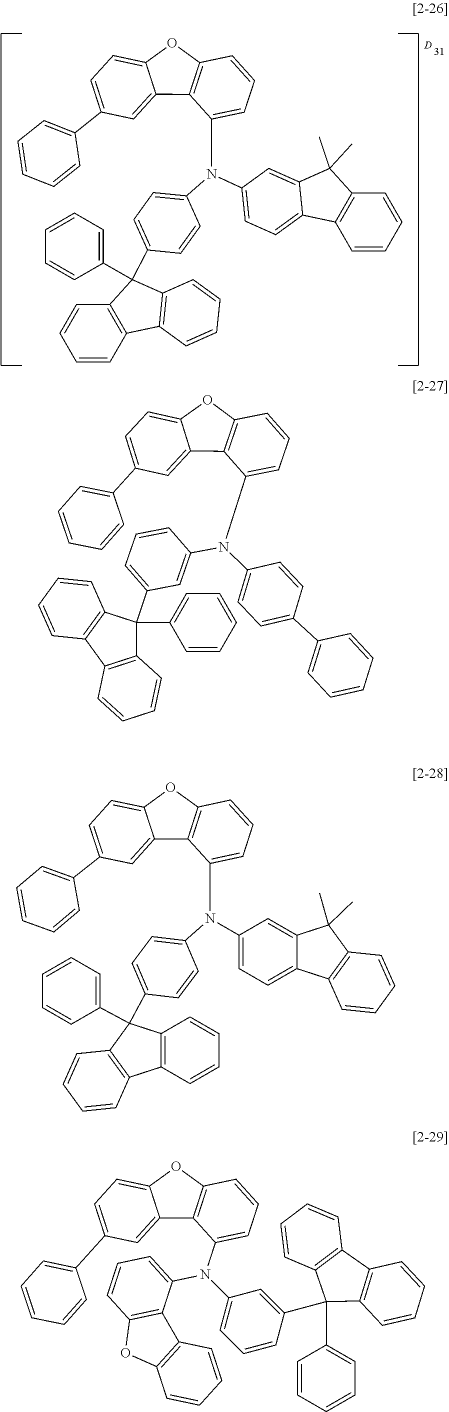

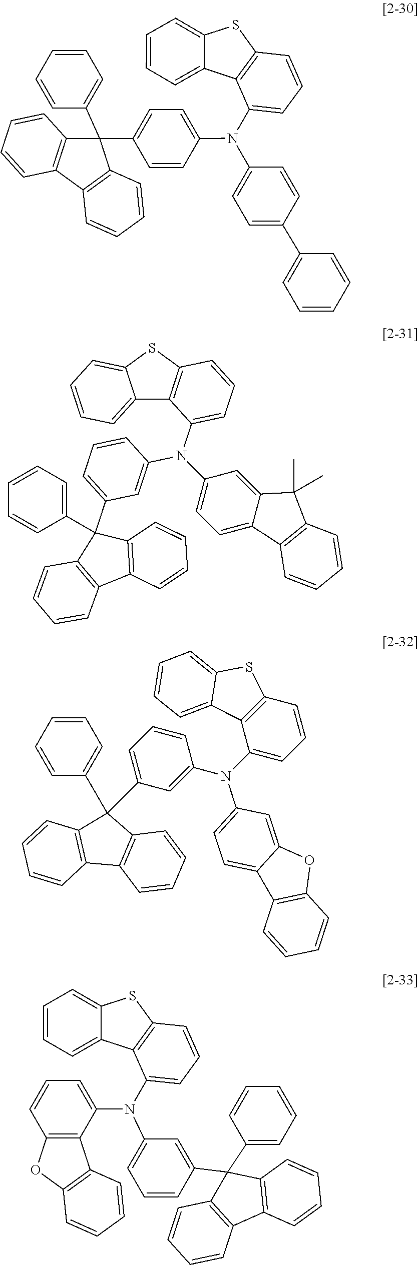 [SMILES: c1ccc(-c2ccc(N(c3ccc(C4(c5ccccc5)c5ccccc5-c5ccccc54)cc3)c3cccc4sc5ccccc5c34)cc2)cc1 ; CC1(C)c2ccccc2-c2ccc(N(c3cccc(C4(c5ccccc5)c5ccccc5-c5ccccc54)c3)c3cccc4sc5ccccc5c34)cc21 ; c1ccc(C2(c3cccc(N(c4ccc5c(c4)oc4ccccc45)c4cccc5sc6ccccc6c45)c3)c3ccccc3-c3ccccc32)cc1 ; c1ccc(C2(c3cccc(N(c4cccc5oc6ccccc6c45)c4cccc5sc6ccccc6c45)c3)c3ccccc3-c3ccccc32)cc1]

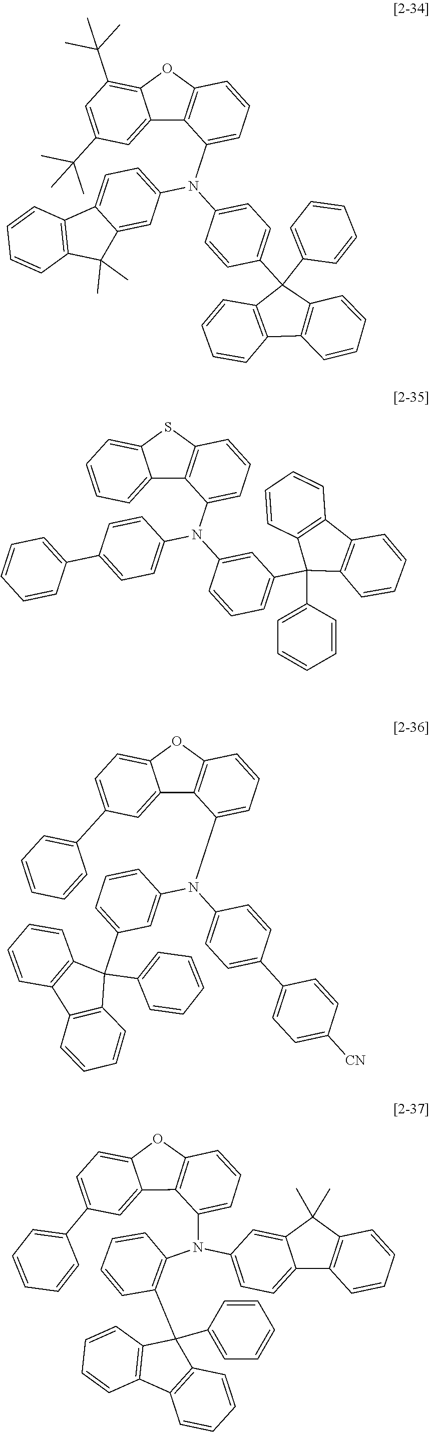 [SMILES: CC(C)(C)c1cc(C(C)(C)C)c2oc3cccc(N(c4ccc(C5(c6ccccc6)c6ccccc6-c6ccccc65)cc4)c4ccc5c(c4)C(C)(C)c4ccccc4-5)c3c2c1 ; c1ccc(-c2ccc(N(c3cccc(C4(c5ccccc5)c5ccccc5-c5ccccc54)c3)c3cccc4sc5ccccc5c34)cc2)cc1 ; N#Cc1ccc(-c2ccc(N(c3cccc(C4(c5ccccc5)c5ccccc5-c5ccccc54)c3)c3cccc4oc5ccc(-c6ccccc6)cc5c34)cc2)cc1 ; CC1(C)c2ccccc2-c2ccc(N(c3ccccc3C3(c4ccccc4)c4ccccc4-c4ccccc43)c3cccc4oc5ccc(-c6ccccc6)cc5c34)cc21]

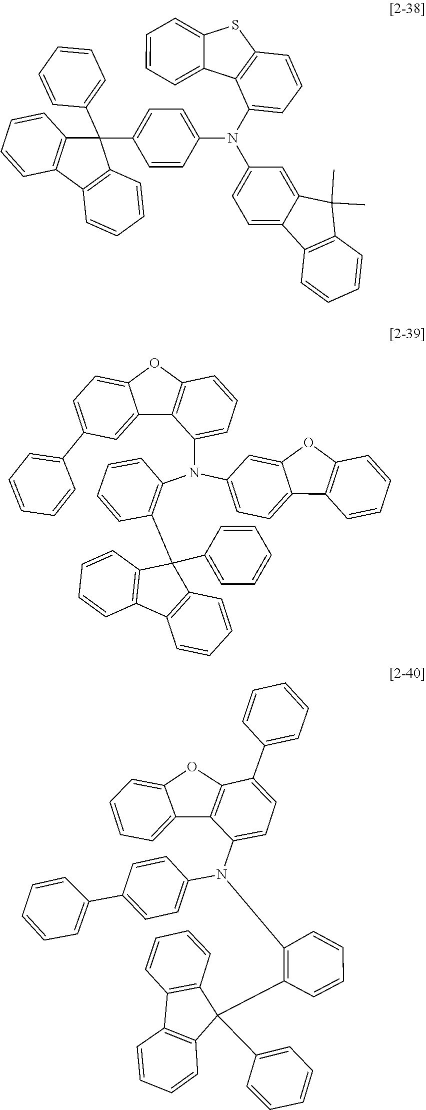 [SMILES: CC1(C)c2ccccc2-c2ccc(N(c3ccc(C4(c5ccccc5)c5ccccc5-c5ccccc54)cc3)c3cccc4sc5ccccc5c34)cc21 ; c1ccc(-c2ccc3oc4cccc(N(c5ccc6c(c5)oc5ccccc56)c5ccccc5C5(c6ccccc6)c6ccccc6-c6ccccc65)c4c3c2)cc1 ; c1ccc(-c2ccc(N(c3ccccc3C3(c4ccccc4)c4ccccc4-c4ccccc43)c3ccc(-c4ccccc4)c4oc5ccccc5c34)cc2)cc1]

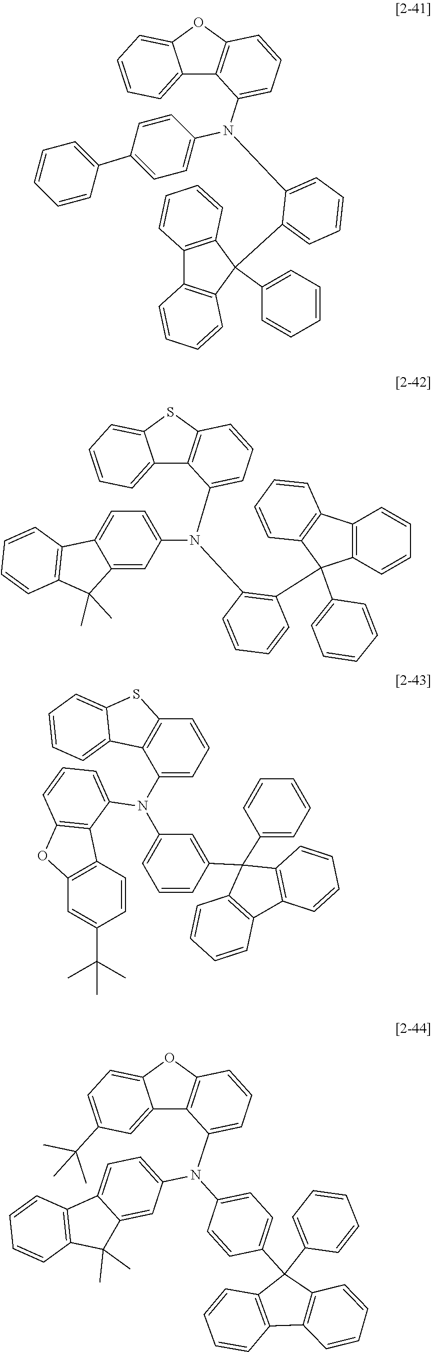 [SMILES: c1ccc(-c2ccc(N(c3ccccc3C3(c4ccccc4)c4ccccc4-c4ccccc43)c3cccc4oc5ccccc5c34)cc2)cc1 ; CC1(C)c2ccccc2-c2ccc(N(c3ccccc3C3(c4ccccc4)c4ccccc4-c4ccccc43)c3cccc4sc5ccccc5c34)cc21 ; CC(C)(C)c1ccc2c(c1)oc1cccc(N(c3cccc(C4(c5ccccc5)c5ccccc5-c5ccccc54)c3)c3cccc4sc5ccccc5c34)c12 ; CC(C)(C)c1ccc2oc3cccc(N(c4ccc(C5(c6ccccc6)c6ccccc6-c6ccccc65)cc4)c4ccc5c(c4)C(C)(C)c4ccccc4-5)c3c2c1]

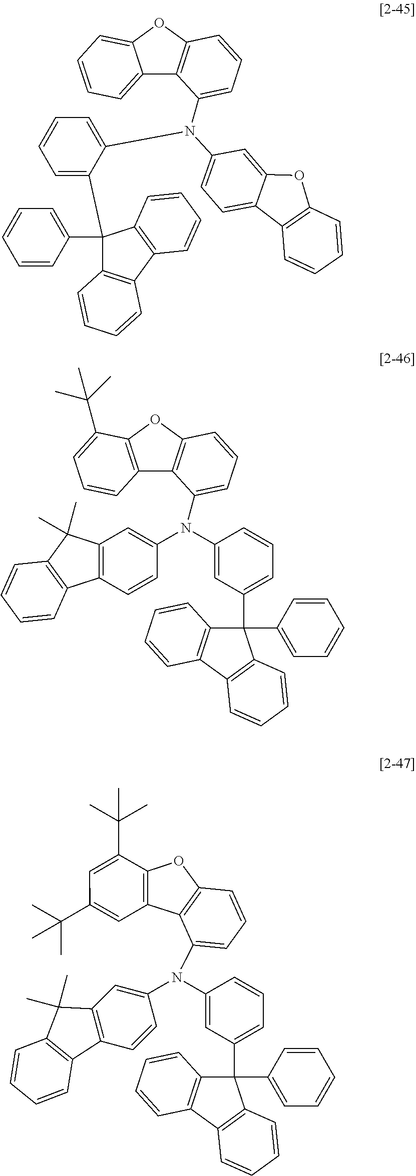

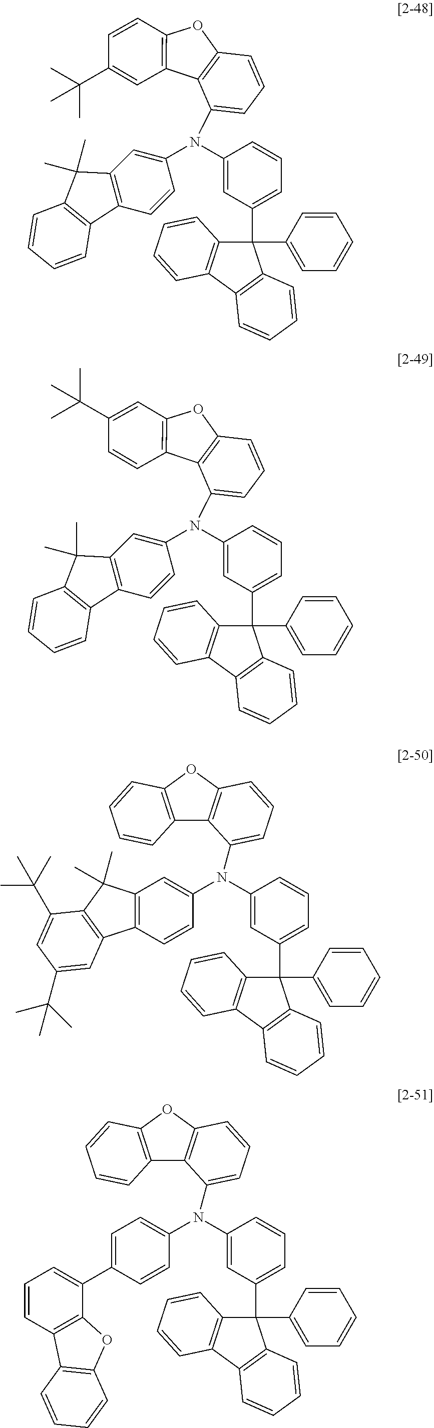 [SMILES: CC(C)(C)c1ccc2oc3cccc(N(c4cccc(C5(c6ccccc6)c6ccccc6-c6ccccc65)c4)c4ccc5c(c4)C(C)(C)c4ccccc4-5)c3c2c1 ; CC(C)(C)c1ccc2c(c1)oc1cccc(N(c3cccc(C4(c5ccccc5)c5ccccc5-c5ccccc54)c3)c3ccc4c(c3)C(C)(C)c3ccccc3-4)c12 ; CC(C)(C)c1cc2c(c(C(C)(C)C)c1)C(C)(C)c1cc(N(c3cccc(C4(c5ccccc5)c5ccccc5-c5ccccc54)c3)c3cccc4oc5ccccc5c34)ccc1-2 ; c1ccc(C2(c3cccc(N(c4ccc(-c5cccc6c5oc5ccccc56)cc4)c4cccc5oc6ccccc6c45)c3)c3ccccc3-c3ccccc32)cc1]

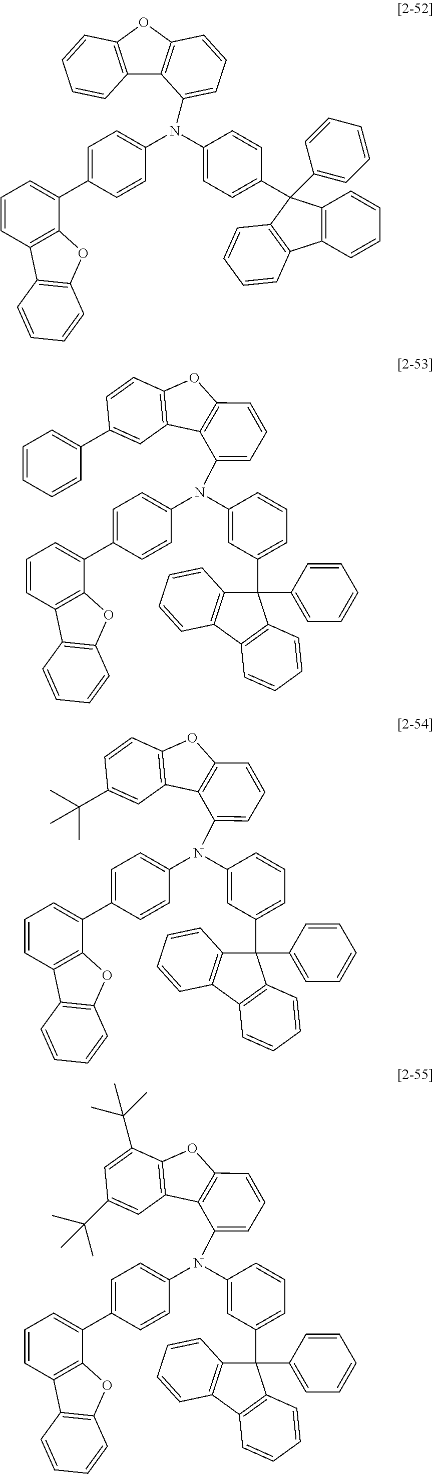

10. A display device comprising the organic optoelectronic device as claimed in claim 1.
